(19) United States
(12) Patent Application Publication
(10) Pub. No.: US 2012/0230951 A1
(43) Pub. Date: Sep. 13, 2012

(54) ANTIVIRAL COMPOUNDS

(76) Inventors: Muzaffar Alam, Clifton, NJ (US); Steven Joseph Berthel, Mendham Township, NJ (US); John A. Brinkman, West Caldwell, NJ (US); Ronald Charles Hawley, Glen Ridge, NJ (US); Hongju Li, Edison, NJ (US); Wylie Solang Palmer, Morristown, NJ (US); Sherrie Pietranico-Cole, Montclair, NJ (US); Ramakanth Sarabu, Towaco, NJ (US); Mark Smith, Jersey City, NJ (US); Sung-Sau So, Verona, NJ (US); Lin Yi, Basking Ridge, NJ (US); Yansheng Zhai, Nutley, NJ (US); Qiang Zhang, East Brunswick, NJ (US); Shu-Hai Zhao, Cupertino, CA (US)

(21) Appl. No.: 13/414,854

(22) Filed: Mar. 8, 2012

Related U.S. Application Data
(60) Provisional application No. 61/451,762, filed on Mar. 11, 2011.
(60) Provisional application No. 61/576,386, filed on Dec. 16, 2011.

(51) Int. Cl. A61K 31/55; A61P 37/02; A61P 31/14; A61K 38/21; C07D 487/06; A61K 31/551

(52) U.S. Cl. 424/85.4; 540/520; 514/212.04; 514/212.02; 540/500; 514/221

(57) ABSTRACT

The present invention provides compounds, compositions and methods for the treatment of hepatitis C virus (HCV) infection. Also disclosed are pharmaceutical compositions containing such compounds and methods for using these compounds in the treatment of HCV infection.

### PRIORITY TO RELATED APPLICATIONS

This application is entitled to the benefit of U.S. provisional patent application Ser. No. 61/451,762, filed on Mar. 11, 2011 and U.S. provisional patent application Ser. No. 61/576,386, filed on Dec. 16, 2011.

### FIELD OF THE INVENTION

The present invention provides non-nucleoside compounds of Formulae I, II, and III and certain derivatives thereof, which are useful as inhibitors of hepatitis C virus (HCV) NS5A protein, as inhibitors of HCV replication, and for the treatment of hepatitis C infection.

Hepatitis C virus (HCV) infection is a major health problem that leads to chronic liver disease, such as cirrhosis and hepatocellular carcinoma, in a substantial number of infected individuals. Current treatments for HCV infection include immunotherapy with recombinant interferon-α alone or in combination with the nucleoside-analog ribavirin.

Several virally-encoded enzymes are putative targets for therapeutic intervention, including a metalloprotease (NS2-3), a serine protease (NS3, amino acid residues 1-180), a helicase (NS3, full length), an NS3 protease cofactor (NS4A), a membrane protein (NS4B), a zinc metalloprotein (NS5A) and an RNA-dependent RNA polymerase (NS5B).

One identified target for therapeutic intervention is HCV NS5A non-structural protein. A non-structural protein, NS5A is an essential component for viral replication and assembly. Mutations in NS5A at or near known sites of phosphorylation can affect the ability for high-level replication in cell-culture systems, suggesting an important role for NS5A phosphorylation in viral replication efficiency. Inhibitors of the phosphorylation of NS5A can lead to reduced viral RNA replication.

There is a clear and long-felt need to develop effective therapeutics for treatment of HCV infection. Specifically, there is a need to develop compounds that are useful for treating HCV-infected patients and compounds that selectively inhibit HCV viral replication.

### SUMMARY OF THE INVENTION

The present application provides a compound of Formula I, II, or III

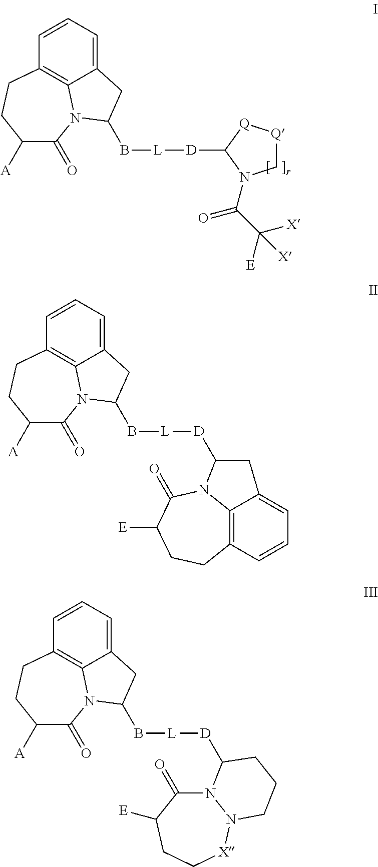

wherein:
each of A and E are independently selected from the group consisting of H, N(R)₂, NHC(═O)OR, NHCOR, and NHCONHR;

each R is independently H or lower alkyl;

B and D are independently selected from the group consisting of

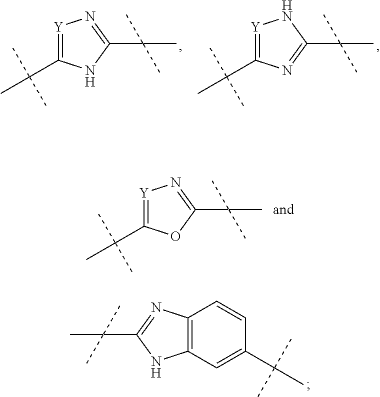

each Y is independently N or CY′;

Y′ is H or halo;

L is selected from the group consisting of

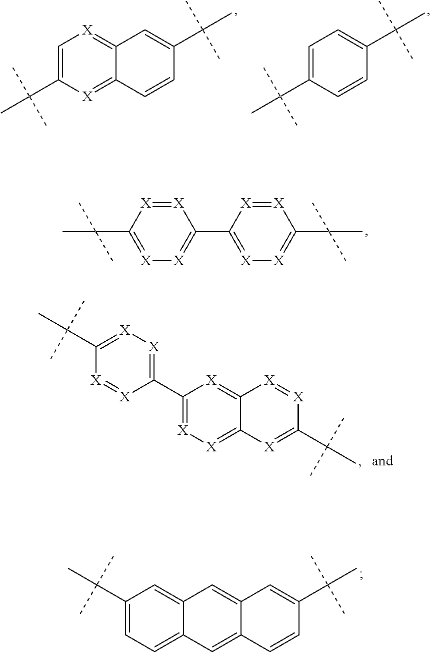

each X is independently, N, CH, C(F), C(OCF₃), or C(CN);

### Q is CH₂;

### Q′ is CH₂, CHOH, CHF, CH(CN), C(Q″)₂, O, or CF₂;

or Q and Q′ together form cycloalkyl;

both Q″ together form a spirocyclic heterocyclic ring;

r is 1 or 2;
each X′ is independently H, lower alkyl, lower alkoxy, hydroxy lower alkyl, heterocycloalkyl, lower alkyl sulfonyl lower alkyl, or (CH₂)ₙ-aryl;

or both X′ together form a spirocyclic heterocyclic ring;

n is 0, 1, or 2; and

### X″ is CH₂ or C(═O);

or a pharmaceutically acceptable salt thereof.

The application provides a method for treating a Hepatitis C Virus (HCV) infection comprising administering to a patient in need thereof a therapeutically effective amount of a compound of any one of Formulae I-III.

The application provides a composition comprising a compound of any one of Formulae I-III and a pharmaceutically acceptable excipient.

### DETAILED DESCRIPTION OF THE INVENTION

### Definitions

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

The phrase “as defined herein above” refers to the broadest definition for each group as provided in the Summary of the Invention or the broadest claim. In all other embodiments provided below, substituents which can be present in each embodiment and which are not explicitly defined retain the broadest definition provided in the Summary of the Invention.

As used in this specification, whether in a transitional phrase or in the body of the claim, the terms “comprise(s)” and “comprising” are to be interpreted as having an open-ended meaning That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

As used herein, unless specifically indicated otherwise, the word “or” is used in the “inclusive” sense of “and/or” and not the “exclusive” sense of “either/or”.

The term “independently” is used herein to indicate that a variable is applied in any one instance without regard to the presence or absence of a variable having that same or a different definition within the same compound. Thus, in a compound in which R″ appears twice and is defined as “independently carbon or nitrogen”, both R″s can be carbon, both R″s can be nitrogen, or one R″ can be carbon and the other nitrogen.

When any variable occurs more than one time in any moiety or formula depicting and describing compounds employed or claimed in the present invention, its definition on each occurrence is independent of its definition at every other occurrence. Also, combinations of substituents and/or variables are permissible only if such compounds result in stable compounds.

The symbols “*” at the end of a bond or “------” drawn through a bond each refer to the point of attachment of a functional group or other chemical moiety to the rest of the molecule of which it is a part. Thus, for example:
MeC(═O)OR⁴ wherein

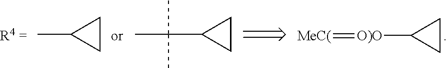

A bond drawn into ring system (as opposed to connected at a distinct vertex) indicates that the bond may be attached to any of the suitable ring atoms.

The term “optional” or “optionally” as used herein means that a subsequently described event or circumstance may, but need not, occur, and that the description includes instances where the event or circumstance occurs and instances in which it does not. For example, “optionally substituted” means that the optionally substituted moiety may incorporate a hydrogen atom or a substituent.

The phrase “optional bond” means that the bond may or may not be present, and that the description includes single, double, or triple bonds. If a substituent is designated to be a “bond” or “absent”, the atoms linked to the substituents are then directly connected.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

Certain compounds may exhibit tautomerism. Tautomeric compounds can exist as two or more interconvertable species. Prototropic tautomers result from the migration of a covalently bonded hydrogen atom between two atoms. Tautomers generally exist in equilibrium and attempts to isolate an individual tautomers usually produce a mixture whose chemical and physical properties are consistent with a mixture of compounds. The position of the equilibrium is dependent on chemical features within the molecule. For example, in many aliphatic aldehydes and ketones, such as acetaldehyde, the keto form predominates while; in phenols, the enol form predominates. Common prototropic tautomers include keto/enol (—C(═O)—CH—⇄—C(—OH)═CH—), amide/imidic acid (—C(═O)—NH—⇄—C(—OH)═N—) and amidine (—C(═NR)—NH—⇄—C(—NHR)═N—) tautomers. The latter two are particularly common in heteroaryl and heterocyclic rings and the present invention encompasses all tautomeric forms of the compounds.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of pharmacology include Goodman and Gilman's The Pharmacological Basis of Therapeutics, 10^{th} Ed., McGraw Hill Companies Inc., New York (2001). Any suitable materials and/or methods known to those of skill can be utilized in carrying out the present invention. However, preferred materials and methods are described. Materials, reagents and the like to which reference are made in the following description and examples are obtainable from commercial sources, unless otherwise noted.

The definitions described herein may be appended to form chemically-relevant combinations, such as “heteroalkylaryl,” “haloalkylheteroaryl,” “arylalkylheterocyclyl,” “alkylcarbonyl,” “alkoxyalkyl,” and the like. When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” refers to an alkyl group having one to two phenyl substituents, and thus includes benzyl, phenylethyl, and biphenyl. An “alkylaminoalkyl” is an alkyl group having one to two alkylamino substituents. “Hydroxyalkyl” includes 2-hydroxyethyl, 2-hydroxypropyl, 1-(hydroxymethyl)-2-methylpropyl, 2-hydroxybutyl, 2,3-dihydroxybutyl, 2-(hydroxymethyl), 3-hydroxypropyl, and so forth. Accordingly, as used herein, the term “hydroxyalkyl” is used to define a subset of heteroalkyl groups defined below. The term -(ar)alkyl refers to either an unsubstituted alkyl or an aralkyl group. The term (hetero)aryl or (het)aryl refers to either an aryl or a heteroaryl group.

The term “spirocycloalkyl”, as used herein, means a spirocyclic cycloalkyl group, such as, for example, spiro[3.3]heptane. The term spiroheterocycloalkyl, as used herein, means a spirocyclic heterocycloalkyl, such as, for example, 2,6-diaza spiro[3.3]heptane.

The term “acyl” as used herein denotes a group of formula —C(═O)R wherein R is hydrogen or lower alkyl as defined herein. The term or “alkylcarbonyl” as used herein denotes a group of formula C(═O)R wherein R is alkyl as defined herein. The term C₁₋₆ acyl refers to a group —C(═O)R contain 6 carbon atoms. The term “arylcarbonyl” as used herein means a group of formula C(═O)R wherein R is an aryl group; the term “benzoyl” as used herein an “arylcarbonyl” group wherein R is phenyl.

The term “ester” as used herein denotes a group of formula —C(═O)OR wherein R is lower alkyl as defined herein.

The term “alkyl” as used herein denotes an unbranched or branched chain, saturated, monovalent hydrocarbon residue containing 1 to 10 carbon atoms. The term “lower alkyl” denotes a straight or branched chain hydrocarbon residue containing 1 to 6 carbon atoms. “C₁₋₁₀ alkyl” as used herein refers to an alkyl composed of 1 to 10 carbons. Examples of alkyl groups include, but are not limited to, lower alkyl groups include methyl, ethyl, propyl, i-propyl, n-butyl, i-butyl, t-butyl or pentyl, isopentyl, neopentyl, hexyl, heptyl, and octyl.

When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” denotes the radical R′R″—, wherein R′ is a phenyl radical, and R″ is an alkylene radical as defined herein with the understanding that the attachment point of the phenylalkyl moiety will be on the alkylene radical. Examples of arylalkyl radicals include, but are not limited to, benzyl, phenylethyl, 3-phenylpropyl. The terms “arylalkyl” or “aralkyl” are interpreted similarly except R′ is an aryl radical. The terms “(het)arylalkyl” or “(het)aralkyl” are interpreted similarly except R′ is optionally an aryl or a heteroaryl radical.

The terms “haloalkyl” or “halo-lower alkyl” or “lower haloalkyl” refers to a straight or branched chain hydrocarbon residue containing 1 to 6 carbon atoms wherein one or more carbon atoms are substituted with one or more halogen atoms.

The term “alkylene” or “alkylenyl” as used herein denotes a divalent saturated linear hydrocarbon radical of 1 to 10 carbon atoms (e.g., (CH₂)ₙ) or a branched saturated divalent hydrocarbon radical of 2 to 10 carbon atoms (e.g., —CHMe- or —CH₂CH(i-Pr)CH₂—), unless otherwise indicated. Except in the case of methylene, the open valences of an alkylene group are not attached to the same atom. Examples of alkylene radicals include, but are not limited to, methylene, ethylene, propylene, 2-methyl-propylene, 1,1-dimethyl-ethylene, butylene, 2-ethylbutylene.

The term “alkoxy” as used herein means an —O-alkyl group, wherein alkyl is as defined above such as methoxy, ethoxy, n-propyloxy, i-propyloxy, n-butyloxy, i-butyloxy, t-butyloxy, pentyloxy, hexyloxy, including their isomers. “Lower alkoxy” as used herein denotes an alkoxy group with a “lower alkyl” group as previously defined. “C₁₋₁₀ alkoxy” as used herein refers to an-O-alkyl wherein alkyl is C₁₋₁₀.

The term “PCy₃” refers to a phosphine trisubstituted with three cyclic moieties.

The terms “haloalkoxy” or “halo-lower alkoxy” or “lower haloalkoxy” refers to a lower alkoxy group, wherein one or more carbon atoms are substituted with one or more halogen atoms.

The term “hydroxyalkyl” as used herein denotes an alkyl radical as herein defined wherein one to three hydrogen atoms on different carbon atoms is/are replaced by hydroxyl groups.

The terms “alkylsulfonyl” and “arylsulfonyl” as used herein refers to a group of formula —S(═O)₂R wherein R is alkyl or aryl respectively and alkyl and aryl are as defined herein. The term “heteroalkylsulfonyl” as used herein refers herein denotes a group of formula —S(═O)₂R wherein R is “heteroalkyl” as defined herein.

The terms “alkylsulfonylamino” and “arylsulfonylamino” as used herein refers to a group of formula —NR′S(═O)₂R wherein R is alkyl or aryl respectively, R′ is hydrogen or C₁₋₃ alkyl, and alkyl and aryl are as defined herein.

The term “cycloalkyl” as used herein refers to a saturated carbocyclic ring containing 3 to 8 carbon atoms, i.e. cyclopropyl, cyclobutyl, cyclopentyl, cyclohexyl, cycloheptyl or cyclooctyl. “C₃₋₇ cycloalkyl” as used herein refers to a cycloalkyl composed of 3 to 7 carbons in the carbocyclic ring.

The term carboxy-alkyl as used herein refers to an alkyl moiety wherein one, hydrogen atom has been replaced with a carboxyl with the understanding that the point of attachment of the heteroalkyl radical is through a carbon atom. The term “carboxy” or “carboxyl” refers to a —CO₂H moiety.

The term “heteroaryl” or “heteroaromatic” as used herein means a monocyclic or bicyclic radical of 5 to 12 ring atoms having at least one aromatic or partially unsaturated ring containing four to eight atoms per ring, incorporating one or more N, O, or S heteroatoms, the remaining ring atoms being carbon, with the understanding that the attachment point of the heteroaryl radical will be on an aromatic or partially unsaturated ring. As well known to those skilled in the art, heteroaryl rings have less aromatic character than their all-carbon counter parts. Thus, for the purposes of the invention, a heteroaryl group need only have some degree of aromatic character. Examples of heteroaryl moieties include monocyclic aromatic heterocycles having 5 to 6 ring atoms and 1 to 3 heteroatoms include, but is not limited to, pyridinyl, pyrimidinyl, pyrazinyl, oxazinyl, pyrrolyl, pyrazolyl, imidazolyl, oxazolyl, 4,5-Dihydro-oxazolyl, 5,6-Dihydro-4H-[1,3]oxazolyl, isoxazole, thiazole, isothiazole, triazoline, thiadiazole and oxadiaxoline which can optionally be substituted with one or more, preferably one or two substituents selected from hydroxy, cyano, alkyl, alkoxy, thio, lower haloalkoxy, alkylthio, halo, lower haloalkyl, alkylsulfinyl, alkylsulfonyl, halogen, amino, alkylamino, dialkylamino, aminoalkyl, alkylaminoalkyl, and dialkylaminoalkyl, nitro, alkoxycarbonyl and carbamoyl, alkylcarbamoyl, dialkylcarbamoyl, arylcarbamoyl, alkylcarbonylamino and arylcarbonylamino. Examples of bicyclic moieties include, but are not limited to, quinolinyl, isoquinolinyl, benzofuryl, benzothiophenyl, benzoxazole, benzisoxazole, benzothiazole, naphthyridinyl, 5,6,7,8-Tetrahydro-[1,6]naphthyridinyl, and benzisothiazole. Bicyclic moieties can be optionally substituted on either ring, however the point of attachment is on a ring containing a heteroatom.

The term “heterocyclyl”, “heterocycloalkyl” or “heterocycle” as used herein denotes a monovalent saturated cyclic radical, consisting of one or more rings, preferably one to two rings, including spirocyclic ring systems, of three to eight atoms per ring, incorporating one or more ring heteroatoms (chosen from N, O or S(O)₀₋₂), and which can optionally be independently substituted with one or more, preferably one or two substituents selected from hydroxy, oxo, cyano, lower alkyl, lower alkoxy, lower haloalkoxy, alkylthio, halo, lower haloalkyl, hydroxyalkyl, nitro, alkoxycarbonyl, amino, alkylamino, alkylsulfonyl, arylsulfonyl, alkylaminosulfonyl, arylaminosulfonyl, alkylsulfonylamino, arylsulfonylamino, alkylaminocarbonyl, arylaminocarbonyl, alkylcarbonylamino, arylcarbonylamino, and ionic forms thereof, unless otherwise indicated. Examples of heterocyclic radicals include, but are not limited to, morpholinyl, piperazinyl, piperidinyl, azetidinyl, pyrrolidinyl, hexahydroazepinyl, oxetanyl, tetrahydrofuranyl, tetrahydrothiophenyl, oxazolidinyl, thiazolidinyl, isoxazolidinyl, tetrahydropyranyl, thiomorpholinyl, quinuclidinyl and imidazolinyl, and ionic forms thereof. Examples may also be bicyclic, such as, for example, 3,8-diaza-bicyclo[3.2.1]octane, 2,5-diaza-bicyclo[2.2.2]octane, or octahydro-pyrazino[2,1-c][1,4]oxazine.

### Inhibitors of HCV NS5A

The application provides a compound of Formula I, II, or III

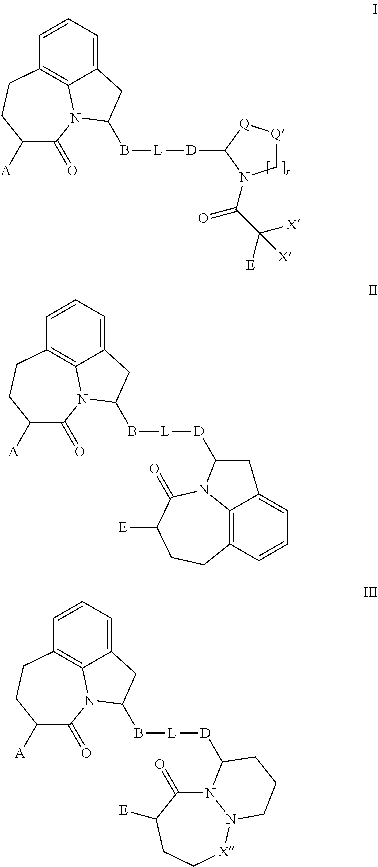

wherein:

each of A and E are independently selected from the group consisting of H, N(R)₂, NHC(═O)OR, NHCOR, and NHCONHR;
each R is independently H or lower alkyl;

B and D are independently selected from the group consisting of

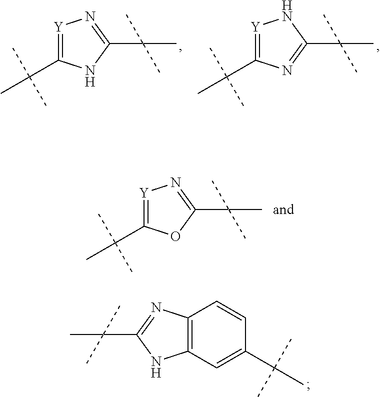

each Y is independently N or CY′;
   Y′ is H or halo;

L is selected from the group consisting of

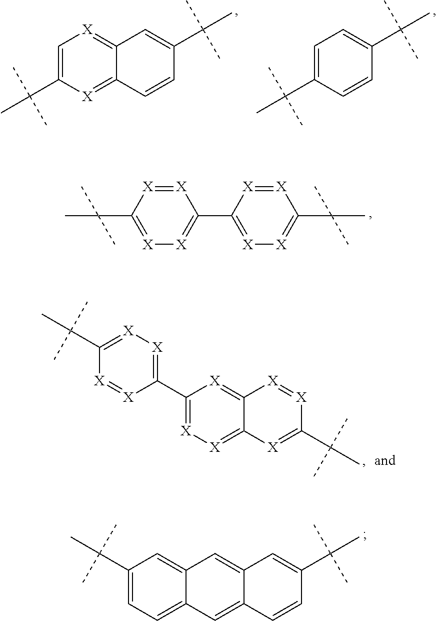

each X is independently, N, CH, C(F), C(OCF₃), or C(CN);

Q is CH₂;

Q′ is CH₂, CHOH, CHF, CH(CN), C(Q″)₂, O, or CF₂;
or Q and Q′ together form cycloalkyl;
both Q″ together form a spirocyclic heterocyclic ring;

r is 1 or 2;

each X′ is independently H, lower alkyl, lower alkoxy, hydroxy lower alkyl, heterocycloalkyl, lower alkyl sulfonyl lower alkyl, or (CH₂)ₙ-aryl;
or both X′ together form a spirocyclic heterocyclic ring;
n is 0, 1, or 2; and

X″ is CH₂ or C(═O);

or a pharmaceutically acceptable salt thereof.

The application provides the above compound, wherein B is

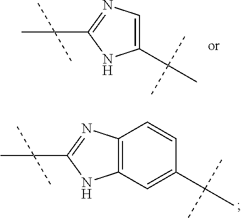

D is

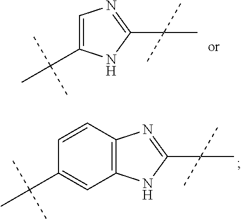

L is selected from a group consisting of

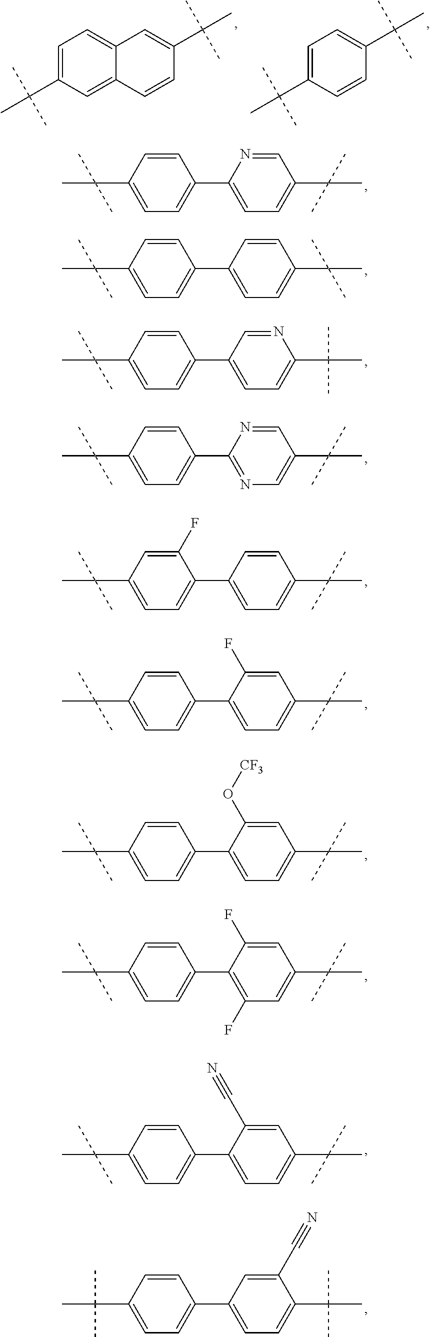

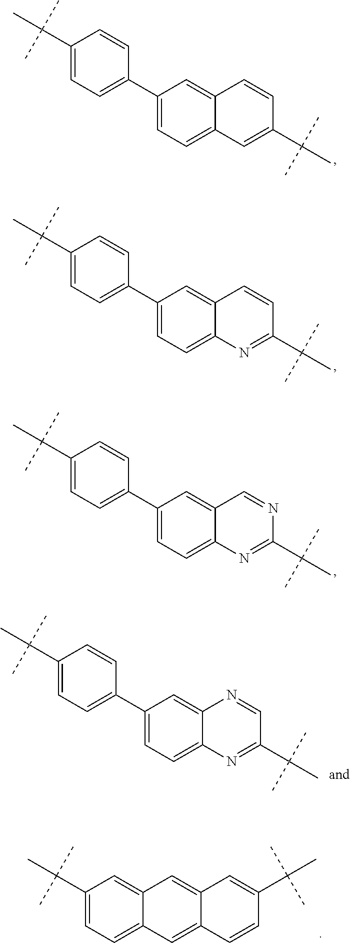

The application provides the compound of any one of Formulae I-III, wherein L is selected from a group consisting of

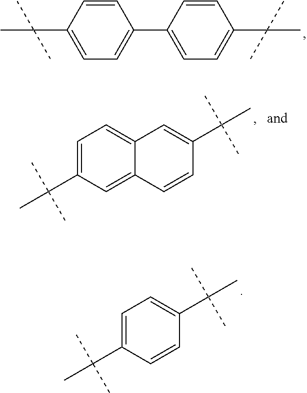

The application provides the compound of any one of Formulae I-III, wherein L is

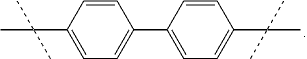

The application provides the compound of any one of Formulae I-III, wherein B is

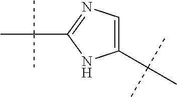

### and D is

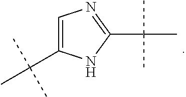

The application provides the compound of any one of Formulae I-III, wherein A is NHC(═O)OCH₃.

The application provides the compound of any one of Formulae I-III, wherein E is NHC(═O)OCH₃.

The application provides the compound of any one of Formulae I-III, wherein A is NHC(═O)OCH₃ and E is NHC(═O)OCH₃.

The application provides the compound of any one of Formulae I-III, wherein L is

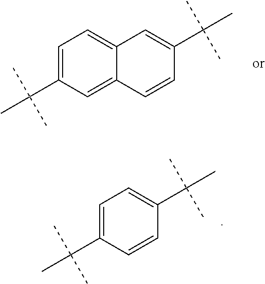

The application provides the compound of any one of Formulae I-III, wherein B is

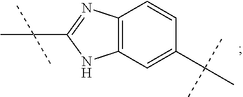

and

D is

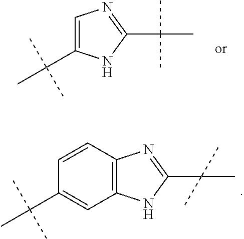

The application provides the compound of any one of Formulae I-III, wherein both A and E are NHC(═O)OCH₃, B is

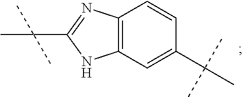

and

D is

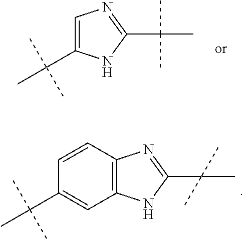

The application provides the compound of any one of Formulae I-III, wherein B is

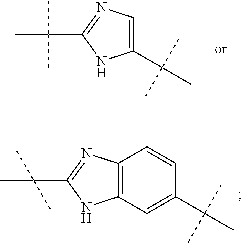

and

D is

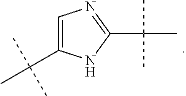

The application provides the compound of any one of Formulae I-III, wherein B is

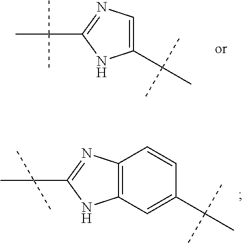

and

D is

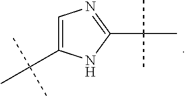

The application provides the compound of Formula I, wherein Q′ is CH₂, X′ is CH(CH₃)₂, and both A and E are NHC(═O)OCH₃.

The application provides the compound of Formula I, wherein Q′ is CH₂, X′ is CH(CH₃)₂, both A and E are NHC(═O)OCH₃, B is

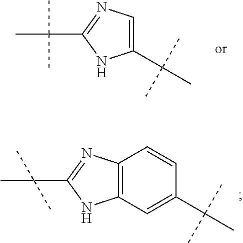

and

D is

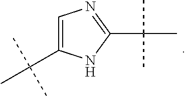

The application provides the compound of Formula I, wherein Q′ is CF₂, X′ is CH(CH₃)₂, and both A and E are NHC(═O)OCH₃.

The application provides the compound of Formula I, wherein Q′ is CF₂, X′ is CH(CH₃)₂, both A and E are NHC(═O)OCH₃, B is

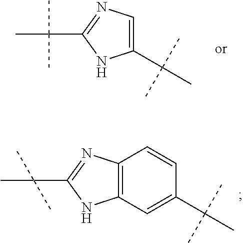

and

D is

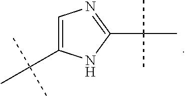

The application provides a compound selected from the group consisting of:
{(2S,5S)-2-[5-(4′-{2-[(2S,4S)-4-Fluoro-1-((S)-2-methoxycarbonylamino-3-methyl-butyryl)-pyrrolidin-2-yl]-3H-imidazol-4-yl}biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(4′-{2-[(2S,4R)-4-Fluoro-1-((S)-2-methoxycarbonylamino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(4-{2-[(S)-1-((S)-2-Methoxycarbonylamino-3-methyl-butyryl)-pyrrolidin-2-yl]-3H-benzoimidazol-5-yl}-phenyl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(4′-{2-[(S)-4,4-Difluoro-1-((S)-2-methoxycarbonylamino-3-methyl-butyryl)-pyrrolidin-2-yl]-3H-benzoimidazol-5-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[6-(4-{2-[(S)-1-((S)-2-Methoxycarbonylamino-3-methyl-butyryl)-pyrrolidin-2-yl]-3H-benzoimidazol-5-yl}-phenyl)-1H-benzoimidazol-2-yl]-4-oxo-1,2,4,5,6,7-hexahydro-azepino[3,2,1-hi]indol-5-yl}-carbamic acid methyl ester;
{(2S,5S)-2-[6-(2-{2-[(S)-1-((S)-2-Methoxycarbonylamino-3-methyl-butyryl)-pyrrolidin-2-yl]-3H-benzoimidazol-5-yl}-quinoxalin-6-yl)-1H-benzoimidazol-2-yl]-4-oxo-1,2,4,5,6,7-hexahydro-azepino[3,2,1-hi]indol-5-yl}-carbamic acid methyl ester;
((2S,5S)-2-{5-[4-(6-{2-[(S)-1-((S)-2-M ethoxycarbonylamino-3-methyl-butyryl)-pyrrolidin-2-yl]-3H-benzoimidazol-5-yl}-pyridin-3-yl)-phenyl]-1H-imidazol-2-yl}-4-oxo-1,2,4,5,6,7-hexahydro-azepino[3,2,1-hi]indol-5-yl)-carbamic acid methyl ester;
{(2S,5S)-2-[5-(4′-{2-[(S)-7-((S)-2-Methoxycarbonylamino-3-methyl-butyryl)-1,4-dioxa-7-aza-spiro[4.4]non-8-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(4′-{2-[2-((S)-2-Methoxycarbonylamino-3-methyl-butyryl)-8-oxa-2-aza-spiro[4.5]dec-3-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[6-(4-{2-[(S)-1-((S)-2-Methoxycarbonylamino-3-methyl-butyryl)-pyrrolidin-2-yl]-3H-imidazol-4-yl}-phenyl)-1H-benzoimidazol-2-yl]-4-oxo-1,2,4,5,6,7-hexahydro-azepino[3,2,1-hi]indol-5-yl}-carbamic acid methyl ester;
{(2S,5S)-2-[6-(4′-{2-[(S)-1-((S)-2-Methoxycarbonylamino-3-methyl-butyryl)-pyrrolidin-2-yl]-3H-imidazol-4-yl}-biphenyl-4-yl)-1H-benzoimidazol-2-yl]-4-oxo-1,2,4,5,6,7-hexahydro-azepino[3,2,1-hi]indol-5-yl}-carbamic methyl ester;
((2S,5S)-2-{6-[6-(4-{2-[(S)-1-((S)-2-M ethoxycarbonylamino-3-methyl-butyryl)-pyrrolidin-2-yl]-3H-imidazol-4-yl}-phenyl)-pyridin-3-yl]-1H-benzoimidazol-2-yl}-4-oxo-1,2,4,5,6,7-hexahydro-azepino[3,2,1-hi]indol-5-yl)-carbamic acid methyl ester;
((2S,5S)-2-{6-[6-(4-{2-[(S)-1-((S)-2-M ethoxycarbonylamino-3-methyl-butyryl)-4,4-difluoro-pyrrolidin-2-yl]-3H-imidazol-4-yl}-phenyl)-pyridin-3-yl]-1H-benzoimidazol-2-yl}-4-oxo-1,2,4,5,6,7-hexahydro-azepino[3,2,1-hi]indol-5-yl)-carbamic acid methyl ester;
{(2S,5S)-2-[6-(6-{2-[(S)-1-((S)-2-Methoxycarbonylamino-3-methyl-butyryl)-pyrrolidin-2-yl]-1H-imidazol-4-yl}-quinoxalin-2-yl)-1H-benzoimidazol-2-yl]-4-oxo-1,2,4,5,6,7-hexahydro-azepino[3,2,1-hi]indol-5-yl}-carbamic acid methyl ester;
((2S,5S)-2-{5-[2-(4-{2-[(S)-1-((S)-2-M ethoxycarbonylamino-3-methyl-butyryl)-pyrrolidin-2-yl]-3H-imidazol-4-yl}-phenyl)-pyrimidin-5-yl]-1H-imidazol-2-yl}-4-oxo-1,2,4,5,6,7-hexahydro-azepino[3,2,1-hi]indol-5-yl)-carbamic acid methyl ester;
((2S,5S)-2-{5-[2-(4-{2-[(S)-1-((S)-2-M ethoxycarbonylamino-3-methyl-butyryl)-4,4-difluoro-pyrrolidin-2-yl]-3H-imidazol-4-yl}-phenyl)-pyrimidin-5-yl]-1H-imidazol-2-yl}-4-oxo-1,2,4,5,6,7-hexahydro-azepino[3,2,1-hi]indol-5-yl)-carbamic acid methyl ester;
((2S,5S)-2-{5-[2-(6-{2-[(S)-1-((S)-2-M ethoxycarbonylamino-3-methyl-butyryl)-pyrrolidin-2-yl]-3H-imidazol-4-yl}-naphthalen-2-yl)-pyrimidin-5-yl]-1H-imidazol-2-yl}-4-oxo-1,2,4,5,6,7-hexahydro-azepino[3,2,1-hi]indol-5-yl)-carbamic acid methyl ester;
((2S,5S)-2-{5-[7-(4-{2-[(S)-1-((S)-2-M ethoxycarbonylamino-3-methyl-butyryl)-pyrrolidin-2-yl]-3H-imidazol-4-yl}-phenyl)-quinolin-3-yl]-1H-imidazol-2-yl}-4-oxo-1,2,4,5,6,7-hexahydro-azepino[3,2,1-hi]indol-5-yl)-carbamic acid methyl ester;
((2S,5S)-2-{5-[7-(4-{2-[(S)-1-((S)-2-M ethoxycarbonylamino-3-methyl-butyryl)-4,4-difluoro-pyrrolidin-2-yl]-3H-imidazol-4-yl}-phenyl)-quinolin-3-yl]-1H-imidazol-2-yl}-4-oxo-1,2,4,5,6,7-hexahydro-azepino[3,2,1-hi]indol-5-yl)-carbamic acid methyl ester;
((2S,5S)-2-{5-[7-(4-{5-Chloro-2-[(S)-1-((S)-2-methoxycarbonyl amino-3-methyl-butyryl)-pyrrolidin-2-yl]-3H-imidazol-4-yl}-phenyl)-quinolin-3-yl]-1H-imidazol-2-yl}-4-oxo-1,2,4,5,6,7-hexahydro-azepino[3,2,1-hi]indol-5-yl)-carbamic acid methyl ester;
((2S,5S)-2-{4-[4-(6-{2-[(S)-1-((S)-2-M ethoxycarbonylamino-3-methyl-butyryl)-pyrrolidin-2-yl]-1H-imidazol-4-yl}-quinolin-2-yl)-phenyl]-1H-imidazol-2-yl}-4-oxo-1,2,4,5,6,7-hexahydro-azepino [3,2,1-hi]indol-5-yl)-carbamic acid methyl ester;
((2S,5S)-2-{5-[2-(4-{2-[(S)-1-((S)-2-M ethoxycarbonylamino-3-methyl-butyryl)-pyrrolidin-2-yl]-1H-imidazol-4-yl}-phenyl)-quinolin-6-yl]-1H-imidazol-2-yl}-4-oxo-1,2,4,5,6,7-hexahydro-azepino[3,2,1-hi]indol-5-yl)-carbamic acid methyl ester;
((2S,5S)-2-{4-[6-(6-{2-[(S)-1-((S)-2-M ethoxycarbonylamino-3-methyl-butyryl)pyrrolidin-2-yl]-3H-imidazol-4-yl}-naphthalen-2-yl)-pyridin-3-yl]-1Himidazol-2-yl}-4-oxo-1,2,4,5,6,7-hexahydro-azepino[3,2,1-hi]indol-5-yl)-carbamic acid methyl ester;
((2S,5S)-2-{4-[6-(5-{2-[(S)-1-((S)-2-M ethoxycarbonylamino-3-methyl-butyryl)-pyrrolidin-2-yl]-3H-imidazol-4-yl}-pyridin-2-yl)-naphthalen-2-yl]-1H-imidazol-2-yl}-4-oxo-1,2,4,5,6,7-hexahydro-azepino[3,2,1-hi]indol-5-yl)-carbamic acid methyl ester;
{(2S,5S)-9-Bromo-2-[5-(4′-{2-[(R)-1-((R)-2-methoxycarbonylamino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-9-Cyano-2-[5-(4′-{2-[(R)-1-((R)-2-methoxycarbonylamino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-9-Carbamoyl-2-[5-(4′-{2-[(R)-1-((R)-2-methoxycarbonylamino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(4′-{2-[(S)-1-((S)-2-Methoxycarbonylamino-3-methyl-butyryl)-pyrrolidin-2-yl]-3H-imidazol-4-yl}-biphenyl-4-yl)-1H-imidazol-2-yl]-4,7-dioxo-1,2,4,5,6,7-hexahydro-azepino[3,2,1-hi]indol-5-yl}-carbamic acid methyl ester;
{(2S,5S)-7-Hydroxy-2-[5-(4′-{2-[(S)-1-((S)-2-methoxycarbonylamino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(4′-{5-Chloro-2-[(S)-1-((S)-2-methoxycarbonyl amino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(4-(6-{2-[(S)-1-((S)-2-Methoxycarbonylamino-3-methyl-butyryl)-pyrrolidin-2-yl]-3H-imidazol-4-yl}-naphthalen-2-yl)-phenyl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(4′-{5-Chloro-2-[(S)-4,4-difluoro-1-((S)-2-methoxycarbonyl amino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(4′-{2-[(2S,4S)-4-Hydroxy-1-((S)-2-methoxycarbonylamino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
((2S,5S)-2-{5-[4-(5-{2-[(S)-1-((S)-2-M ethoxycarbonylamino-3-methyl-butyryl)-pyrrolidin-2-yl]-3H-imidazol-4-yl}-pyridin-2-yl)-phenyl]-1H-imidazol-2-yl}-4-oxo-1,2,4,5,6,7-hexahydro-azepino[3,2,1-hi]indol-5-yl)-carbamic acid methyl ester;
{(2S,5S)-2-[5-(2-Fluoro-4′-{2-[(S)-1-((S)-2-methoxycarbonylamino-3-methyl-butyryl)-pyrrolidin-2-yl]-1H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(3-Fluoro-4′-{2-[(S)-1-((S)-2-methoxycarbonylamino-3-methyl-butyryl)-pyrrolidin-2-yl]-1H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(2′-Fluoro-4′-{2-[(S)-1-((S)-2-methoxycarbonylamino-3-methyl-butyryl)-pyrrolidin-2-yl]-1H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(2′-Cyano-4′-{2-[(S)-1-((S)-2-methoxycarbonylamino-3-methyl-butyryl)-pyrrolidin-2-yl]-1H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(4′-{2-[(2S,4R)-4-Cyano-1-((S)-2-methoxycarbonylamino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(4′-{2-[(2S,4S)-4-Cyano-1-((S)-2-methoxycarbonylamino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-{5-[4-(6-{2-[(2S,4S)-4-Cyano-1-((S)-2-methoxycarbonylamino-3-methyl-butyryl)-pyrrolidin-2-yl]-3H-imidazol-4-yl}-naphthalen-2-yl)-phenyl]-1H-imidazol-2-yl}-4-oxo-1,2,4,5,6,7-hexahydro-azepino[3,2,1-hi]indol-5-yl}-carbamic acid methyl ester;
{(2S,5S)-2-{5-[4-(6-{2-[(S)-4,4-Difluoro-1-((S)-2-methoxycarbonylamino-3-methyl-butyryl)-pyrrolidin-2-yl]-3H-imidazol-4-yl}-naphthalen-2-yl)-phenyl]-1H-imidazol-2-yl}-4-oxo-1,2,4,5,6,7-hexahydro-azepino[3,2,1-hi]indol-5-yl}-carbamic acid methyl ester;
{(2S,5S)-2-[5-(4-{2-[(S)-1-((S)-3-Methoxy-2-methoxycarbonylamino-propionyl)-pyrrolidin-2-yl]-3H-imidizol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
(2S,5S)-2-[5-(4-{2-[(S)-1-((S)-2-Cyclopropyl-2-methoxycarbonylamino-acetyl)-pyrrolidin-2-yl]-3H-imidizol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(4-{2-[(S)-1-((S)-2-Methoxycarbonylamino-butyryl)-pyrrolidin-2-yl]-3H-imidizol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(4-{2-[(S)-1-((S)-4-Methoxy-2-Methoxycarbonylamino-butyryl)-pyrrolidin-2-yl]-3H-imidizol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(4-{2-[(S)-1-(3-Methoxycarbonylamino-oxetane-3-carbonyl)-pyrrolidin-2-yl]-3H-imidizol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
((2S,5S)-2-{5-[4-(2-{(S)-1-[2-Methoxycarbonylamino-2-(tetrahydro-pyran-4-yl)-pyrrolidin-2-yl]-3H-imidizol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(4-{2-[(S)-1-((S)-2-Methoxycarbonylamino-propionyl)-pyrrolidin-2-yl]-3H-imidizol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(4-{2-[(S)-1-((S)-3-Hydroxy-2-methoxycarbonylamino-butyl)-pyrolidin-2-yl]-3H-imidizol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(4-{2-[(S)-1-((S)-4-Methanesulfonyl-2-methoxycarbonylamino-butyl)-pyrolidin-2-yl]-3H-imidizol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
((2S,5S)-2-{5-[4-(6-{2-[(S)-1-((S)-2-Methoxycarbonylamino-3-methyl-butyryl)-pyrrolidin-2-yl]-3H-imidazol-4-yl}-quinoxalin-2-yl)-phenyl]-1H-imidazol-2-yl}-4-oxo-1,2,4,5,6,7-hexahydro-azepino[3,2,1-hi]indol-5-yl)-carbamic acid methyl ester;
((2S,5S)-2-{5-[4-(6-{2-[(S)-1-((2S,3S)-2-Methoxycarbonylamino-3-methyl-pentanoyl)-pyrrolidin-2-yl]-3H-imidazol-4-yl}-quinoxalin-2-yl)-phenyl]-1H-imidazol-2-yl}-4-oxo-1,2,4,5,6,7-hexahydroazepino[3,2,1-hi]indol-5-yl)-carbamic acid methyl ester;
((2S,5S)-2-{5-[4-(6-{2-[(S)-1-((S)-2-Methoxycarbonylamino-3,3-dimethyl-butyryl)-pyrrolidin-2-yl]-3H-imidazol-4-yl}-quinoxalin-2-yl)-phenyl]-1H-imidazol-2-yl}-4-oxo-1,2,4,5,6,7-hexahydro-azepino[3,2,1-hi]indol-5-yl)-carbamic acid methyl ester;
((2S,5S)-2-{5-[4-(6-{2-[(S)-1-((R)-2-Methoxycarbonylamino-2-phenyl-acetyl)-pyrrolidin-2-yl]-3H-imidazol-4-yl}-quinoxalin-2-yl)-phenyl]-1H-imidazol-2-yl}-4-oxo-1,2,4,5,6,7-hexahydro-azepino[3,2,1-hi]indol-5-yl)-carbamic acid methyl ester;
((2S,5S)-2-{5-[2-(4-{2-[(S)-1-((2S,3S)-2-Methoxycarbonylamino-3 methyl-pentanoyl)-pyrrolidin-2-yl]-3H-imidazol-4-yl}-phenyl) quinoxalin-6-yl]-1H-imidazol-2-yl}-4-oxo-1,2,4,5,6,7-hexahydro-azepino[3,2,1-hi]indol-5-yl)-carbamic acid methyl ester;
((2S,5S)-2-{4-[4-(5-{2-[(S)-4,4-Difluoro-1-(2-methoxycarbonylamino-3-methyl-butyryl)-pyrrolidin-2-yl]-3H-imidazol-4-yl}-pyrimidin-2-yl)-phenyl]-1H-imidazol-2-yl}-4-oxo-1,2,4,5,6,7-hexahydro-azepino[3,2,1-hi]indol-5-yl)-carbamic acid methyl ester;
((1S,2S)-1-{(S)-4,4-Difluoro-2-[5-(2-{4-[2-((2S,5S)-5-methoxycarbonylamino-4-oxo-1,2,4,5,6,7-hexahydro-azepino[3,2,1-hi]indol-2-yl)-1H-imidazol-4-yl]-phenyl}-pyrimidin-5-yl)-1H-imidazol-2-yl]-pyrrolidine-1-carbonyl}-2-methyl-butyl)-carbamic acid methyl ester;
((2S,5S)-2-{5-[2-(4-{2-[(S)-1-((S)-2-Methoxycarbonylamino-3-methyl-butyryl)-pyrrolidin-2-yl]-3H-imidazol-4-yl}-phenyl)-quinoxalin-6-yl]-1H-imidazol-2-yl}-4-oxo-1,2,4,5,6,7-hexahydro-azepino[3,2,1-hi]indol-5-yl)-carbamic acid methyl ester;
{(2S,5S)-2-[5-(4′-{5-[(S)-1-((R)-2-Methoxycarbonylamino-2-phenyl-acetyl)-pyrrolidin-2-yl]-4H-[1,2,4]triazol-3-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(4′-{5-[(S)-1-((S)-2-Methoxycarbonylamino-3-methyl-butyryl)-pyrrolidin-2-yl]-4H-[1,2,4]triazol-3-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(4′-{5-[(S)-1-((S)-2-Methoxycarbonylamino-3-methyl-butyryl)-pyrrolidin-2-yl]-1H-imidazol-2-yl}-biphenyl-4-yl)-[1,3,4]oxadiazol-2-yl]-4-oxo-1,2,4,5,6,7-hexahydro-azepino[3,2,1-hi]indol-5-yl}-carbamic acid methyl ester;
{(2S,5S)-2-[5-(4′-{2-[(R)-4-((R)-2-Methoxycarbonylamino-2-phenyl-acetyl)-morpholin-3-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(4′-{2-[(R)-4-((S)-2-Methoxycarbonylamino-3-methyl-butyryl)-morpholin-3-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(4′-{2-[(S)-3-((R)-2-Methoxycarbonylamino-2-phenyl-acetyl)-3-aza-bicyclo [3.1.0]hex-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(4′-{2-[(S)-1-((S)-2-Methoxycarbonylamino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(4′-{2-[(1S,9S)-9-methoxycarbonylamino-6,10-dioxo-octahydro-pyridazino[1,2-a][1,2]diazepin-1-yl])-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
6-(6-{2-[(S)-1-((S)-2-Methoxycarbonylamino-3-methyl-butyryl)-pyrrolidin-2-yl]-1H-imidazol-4-yl}-naphthalen-2-yl)-2-((2S,5S)-5-methoxycarbonylamino-4-oxo-1,2,4,5,6,7-hexahydro-azepino[3,2,1-hi]indol-2-yl)-benzoimidazole-1-carboxylic acid methyl ester;
N,N′-[1,4-phenylenebis[1H-benzimidazole-6,2-diyl-(2S,5S)-1,2,4,5,6,7-hexahydro-4-oxoazepino[3,2,1-hi]indole-5,2-diyl]]biscarbamic acid C,C′ dimethyl ester; and
{(2S,5S)-2-[5-(4′-{2-[(S)-4,4-Difluoro-1-((S)-2-Methoxycarbonylamino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester.

The application provides a method for treating a Hepatitis C Virus (HCV) infection comprising administering to a patient in need thereof a therapeutically effective amount of a compound of any one of Formulae I-III.

The application provides the above method, further comprising administering an immune system modulator or an antiviral agent that inhibits replication of HCV, or a combination thereof.

The application provides the above method, wherein the immune system modulator is an interferon or chemically derivatized interferon.

The application provides the above methods, wherein the antiviral agent is selected from the group consisting of a HCV protease inhibitor, a HCV polymerase inhibitor, a HCV helicase inhibitor, a HCV primase inhibitor, a HCV fusion inhibitor, and a combination thereof.

The application provides a method for inhibiting replication of HCV in a cell comprising administering a compound of any one of Formulae I-III.

The application provides a composition comprising a compound of any one of Formulae I-III and a pharmaceutically acceptable excipient.

The application provides a use of the compound of any one of Formulae I-III in the manufacture of a medicament for the treatment of HCV.

The application provides a compound, composition, or method as described herein.

### Compounds

Examples of representative compounds encompassed by the present invention and within the scope of the invention are provided in the following Table. These examples and preparations which follow are provided to enable those skilled in the art to more clearly understand and to practice the present invention. They should not be considered as limiting the scope of the invention, but merely as being illustrative and representative thereof.

In general, the nomenclature used in this Application is based on AUTONOMTM v.4.0, a Beilstein Institute computerized system for the generation of IUPAC systematic nomenclature. If there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

TABLE I depicts examples of compounds according to generic Formulae I, II, and III.

TABLE I

| # | Structure | Nomenclature |
|---|---|---|
| I-1 | 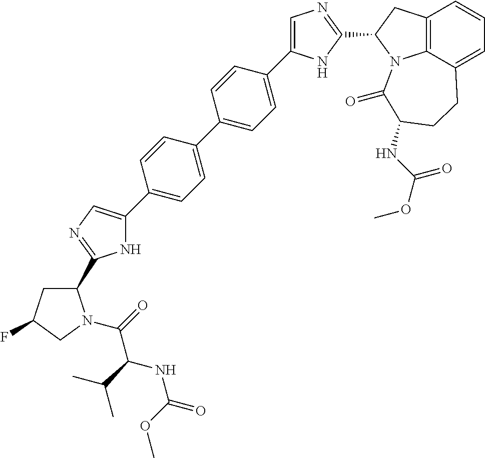 | {(2S,5S)-2-[5-(4′-{2- [(2S,4S)-4-Fluoro-1- ((S)-2- methoxycarbonylamino- 3-methyl-butyryl)- pyrrolidin-2-yl]-3H- imidazol-4-yl}biphenyl- 4-yl)-1H-imidazol-2-yl]- 4-oxo-1,2,4,5,6,7- hexahydro- azepino[3,2,1-hi]indol-5- yl}-carbamic acid methyl ester |
| I-2 | 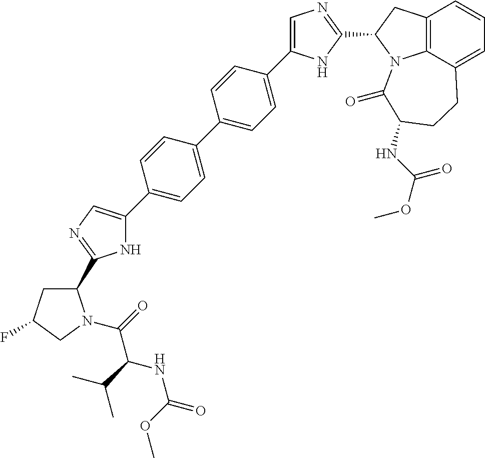 | {(2S,5S)-2-[5-(4′-{2- [(2S,4R)-4-Fluoro-1- ((S)-2- methoxycarbonylamino- 3-methyl-butyryl)- pyrrolidin-2-yl]-3H- imidazol-4-yl}-biphenyl- 4-yl)-1H-imidazol-2-yl]- 4-oxo-1,2,4,5,6,7- hexahydro- azepino[3,2,1-hi]indol-5- yl}-carbamic acid methyl ester |
| I-3 | 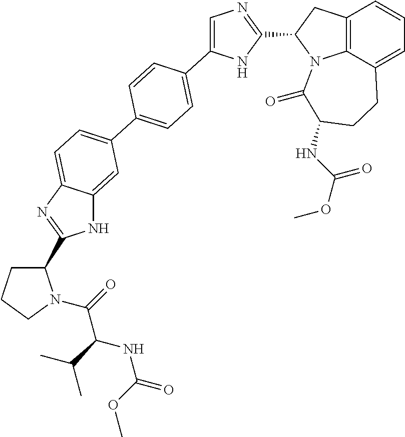 | {(2S,5S)-2-[5-(4-{2- [(S)-1-((S)-2- Methoxycarbonylamino- 3-methyl-butyryl)- pyrrolidin-2-yl]-3H- benzoimidazol-5-yl}- phenyl)-1H-imidazol-2- yl]-4-oxo-1,2,4,5,6,7- hexahydro- azepino[3,2,1-hi]indol-5- yl}-carbamic acid methyl ester |
| I-4 | 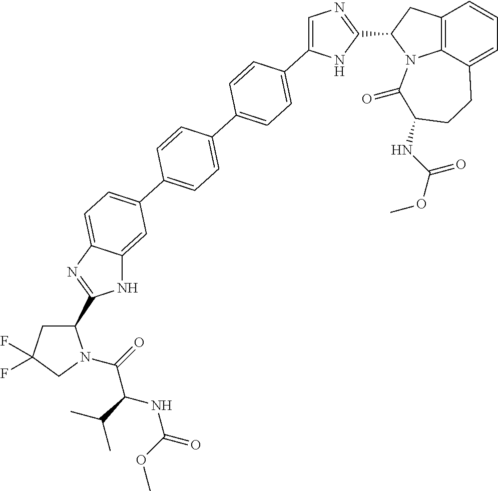 | {(2S,5S)-2-[5-(4′-{2- [(S)-4,4-Difluoro-1-((S)- 2- methoxycarbonylamino- 3-methyl-butyryl)- pyrrolidin-2-yl]-3H- benzoimidazol-5-yl}- biphenyl-4-yl)-1H- imidazol-2-yl]-4-oxo- 1,2,4,5,6,7-hexahydro- azepino[3,2,1-hi]indol-5- yl}-carbamic acid methyl ester |
| I-5 | 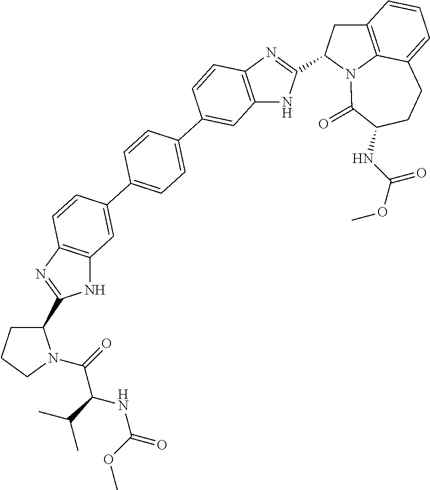 | {(2S,5S)-2-[6-(4-{2- [(S)-1-((S)-2- Methoxycarbonylamino- 3-methyl-butyryl)- pyrrolidin-2-yl]-3H- benzoimidazol-5-yl}- phenyl)-1H- benzoimidazol-2-yl]-4- oxo-1,2,4,5,6,7- hexahydro- azepino[3,2,1-hi]indol-5- yl}-carbamic acid methyl ester |
| I-6 | 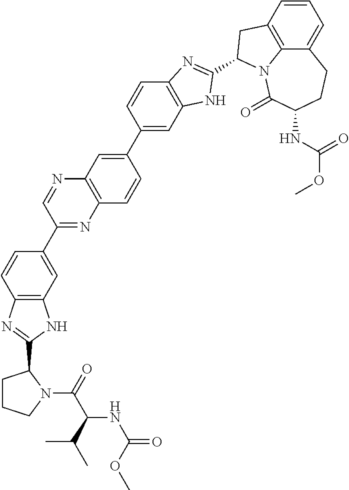 | {(2S,5S)-2-[6-(4-{2- [(S)-1-((S)-2- Methoxycarbonylamino- 3-methyl-butyryl)- pyrrolidin-2-yl]-3H- benzoimidazol-5-yl}- quinoxalin-6-yl)-1H- benzoimidazol-2-yl]-4- oxo-1,2,4,5,6,7- hexahydro- azepino[3,2,1-hi]indol-5- yl}-carbamic acid methyl ester |
| I-7 | 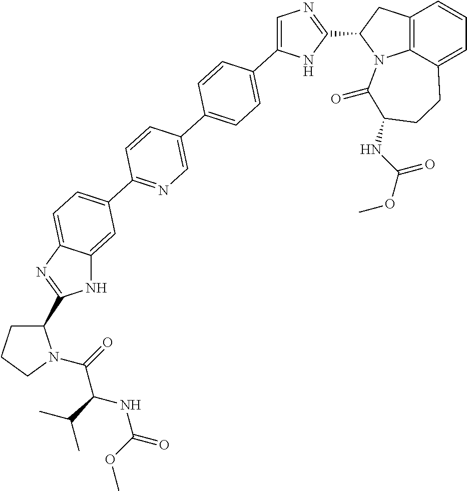 | ((2S,5S)-2-{5-[4-(6-{2- [(S)-1-((S)-2- Methoxycarbonylamino- 3-methyl-butyryl)- pyrrolidin-2-yl]-3H- benzoimidazol-5-yl}- pyridin-3-yl)-phenyl]- 1H-imidazol-2-yl}-4- oxo-1,2,4,5,6,7- hexahydro- azepino[3,2,1-hi]indol-5- yl)-carbamic acid methyl ester |
| I-8 | 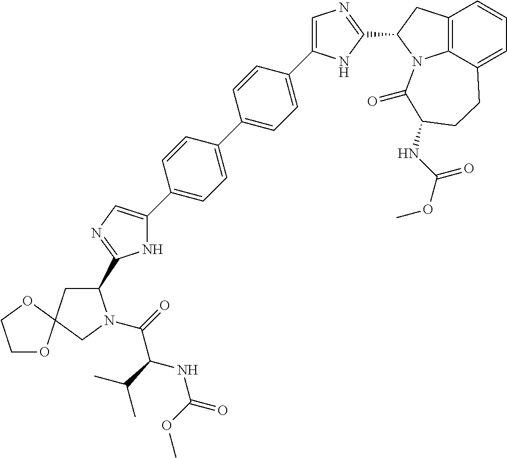 | {(2S,5S)-2-[5-(4′-{2- [(S)-7-((S)-2- Methoxycarbonylamino- 3-methyl-butyryl)-1,4- dioxa-7-aza- spiro[4.4]non-8-yl]-3H- imidazol-4-yl}-biphenyl- 4-yl)-1H-imidazol-2-yl]- 4-oxo-1,2,4,5,6,7- hexahydro- azepino[3,2,1-hi]indol-5- yl}-carbamic acid methyl Ester |
| I-9 | 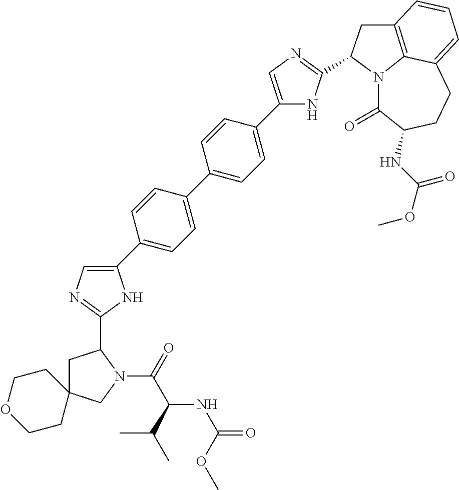 | {(2S,5S)-2-[5-(4′-{2-[2- ((S)-2- Methoxycarbonylamino- 3-methyl-butyryl)-8-oxa- 2-aza-spiro[4.5]dec-3- yl]-3H-imidazol-4-yl}- biphenyl-4-yl)-1H- imidazol-2-yl]-4-oxo- 1,2,4,5,6,7-hexahydro- azepino[3,2,1-hi]indol-5- yl}-carbamic acid methyl ester |
| I-10 | 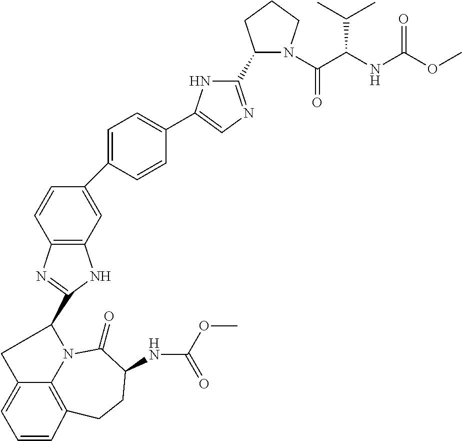 | {(2S,5S)-2-[6-(4-{2- [(S)-1-((S)-2- Methoxycarbonylamino- 3-methyl-butyryl)- pyrrolidin-2-yl]-3H- imidazol-4-yl}-phenyl)- 1H-benzoimidazol-2-yl]- 4-oxo-1,2,4,5,6,7- hexahydro- azepino[3,2,1-hi]indol-5- yl}-carbamic acid methyl ester |
| I-11 | 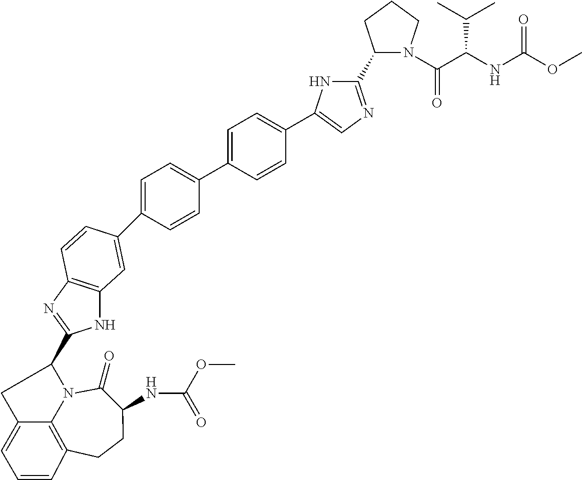 | {(2S,5S)-2-[6-(4′-{2- [(S)-1-((S)-2- Methoxycarbonylamino- 3-methyl-butyryl)- pyrrolidin-2-yl]-3H- imidazol-4-yl}-biphenyl- 4-yl)-1H-benzoimidazol- 2-yl]-4-oxo-1,2,4,5,6,7- hexahydro- azepino[3,2,1-hi]indol-5- yl}-carbamic methyl ester |
| I-12 | 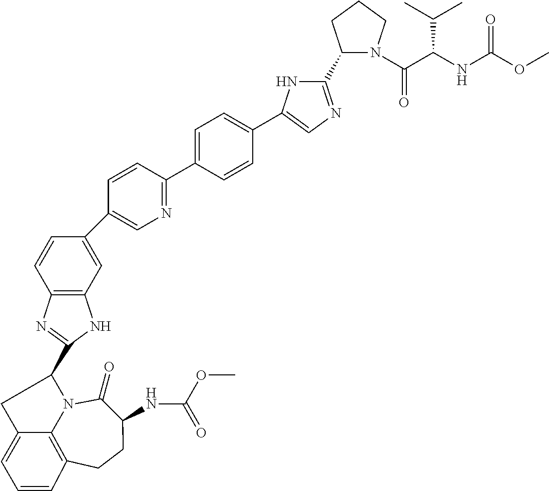 | ((2S,5S)-2-{6-[6-(4-{2- [(S)-1-((S)-2- Methoxycarbonylamino- 3-methyl-butyryl)- pyrrolidin-2-yl]-3H- imidazol-4-yl}-phenyl)- pyridin-3-yl]-1H- benzoimidazol-2-yl}-4- oxo-1,2,4,5,6,7- hexahydro- azepino[3,2,1-hi]indol-5- yl)-carbamic acid methyl ester |
| I-13 | 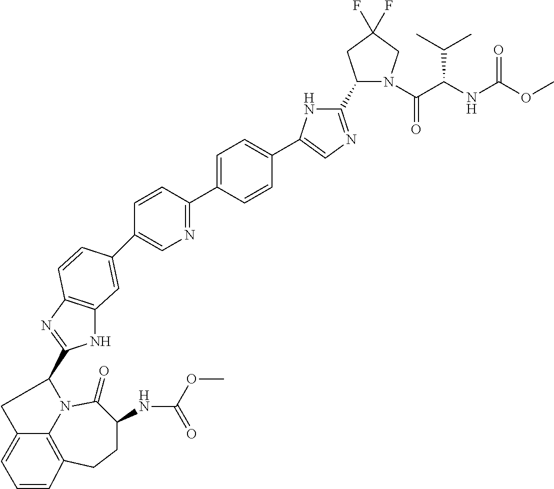 | ((2S,5S)-2-{6-[6-(4-{2- [(S)-1-((S)-2- Methoxycarbonylamino- 3-methyl-butyryl)-4,4- difluoro-pyrrolidin-2- yl]-3H-imidazol-4-yl}- phenyl)-pyridin-3-yl]- 1H-benzoimidazol-2- yl}-4-oxo-1,2,4,5,6,7- hexahydro- azepino[3,2,1-hi]indol-5- yl)-carbamic acid methyl ester |
| I-14 | 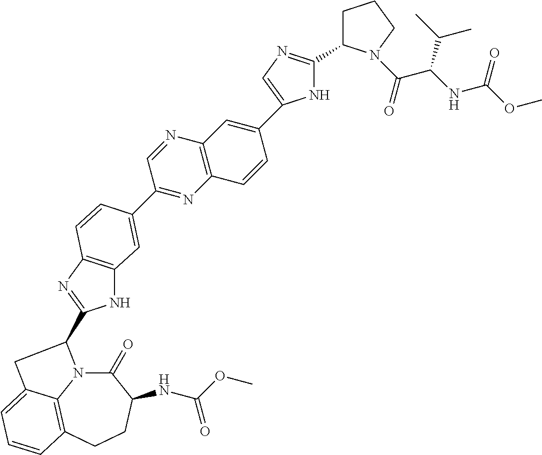 | {(2S,5S)-2-[6-(6-{2- [(S)-1-((S)-2- Methoxycarbonylamino- 3-methyl-butyryl)- pyrrolidin-2-yl]-1H- imidazol-4-yl}- quinoxalin-2-yl)-1H- benzoimidazol-2-yl]-4- oxo-1,2,4,5,6,7- hexahydro- azepino[3,2,1-hi]indol-5- yl}-carbamic acid methyl ester |
| I-15 | 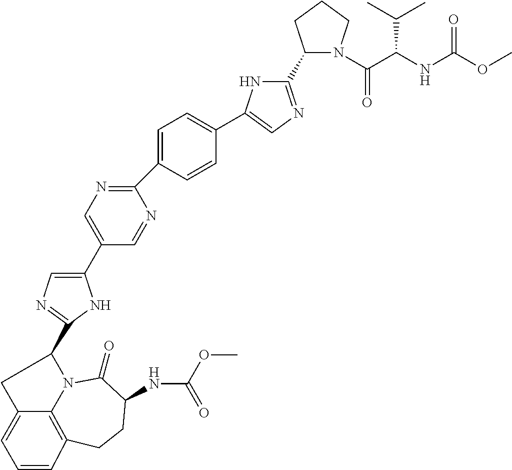 | ((2S,5S)-2-{5-[2-(4-{2- [(S)-1-((S)-2- Methoxycarbonylamino- 3-methyl-butyryl)- pyrrolidin-2-yl]-3H- imidazol-4-yl}-phenyl)- pyrimidin-5-yl]-1H- imidazol-2-yl}-4-oxo- 1,2,4,5,6,7-hexahydro- azepino[3,2,1-hi]indol-5- yl)-carbamic acid methyl ester |
| I-16 | 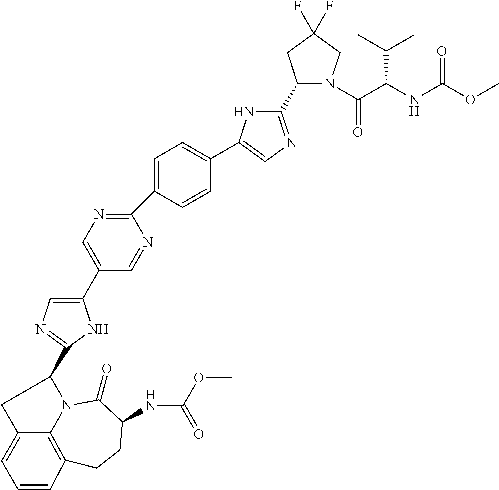 | ((2S,5S)-2-{5-[2-(4-{2- [(S)-1-((S)-2- Methoxycarbonylamino- 3-methyl-butyryl)-4,4- difluoro-pyrrolidin-2- yl]-3H-imidazol-4-yl}- phenyl)-pyrimidin-5-yl]- 1H-imidazol-2-yl}-4- oxo-1,2,4,5,6,7- hexahydro- azepino[3,2,1-hi]indol-5- yl)-carbamic acid methyl ester |
| I-17 | 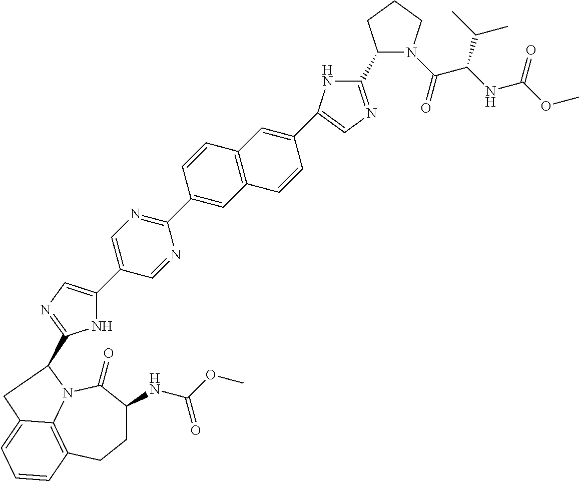 | ((2S,5S)-2-{5-[2-(6-{2- [(S)-1-((S)-2- Methoxycarbonylamino- 3-methyl-butyryl)- pyrrolidin-2-yl]-3H- imidazol-4-yl}- naphthalen-2-yl)- pyrimidin-5-yl]-1H- imidazol-2-yl}-4-oxo- 1,2,4,5,6,7-hexahydro- azepino[3,2,1-hi]indol-5- yl)-carbamic acid methyl ester |
| I-18 | 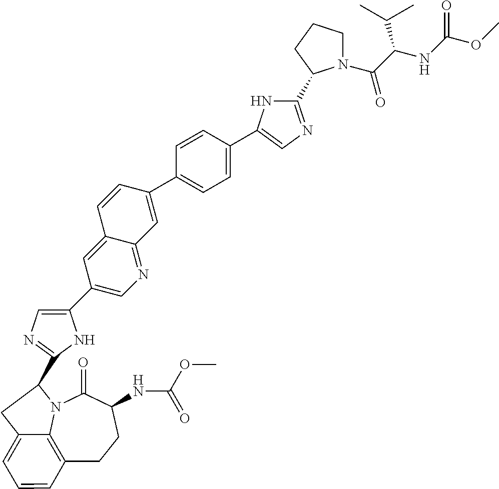 | ((2S,5S)-2-{5-[7-(4-{2- [(S)-1-((S)-2- Methoxycarbonylamino- 3-methyl-butyryl)- pyrrolidin-2-yl]-3H- imidazol-4-yl}-phenyl)- quinolin-3-yl]-1H- imidazol-2-yl}-4-oxo- 1,2,4,5,6,7-hexahydro- azepino[3,2,1-hi]indol-5- yl)-carbamic acid methyl ester trifluoacetic acid salt |
| I-19 | 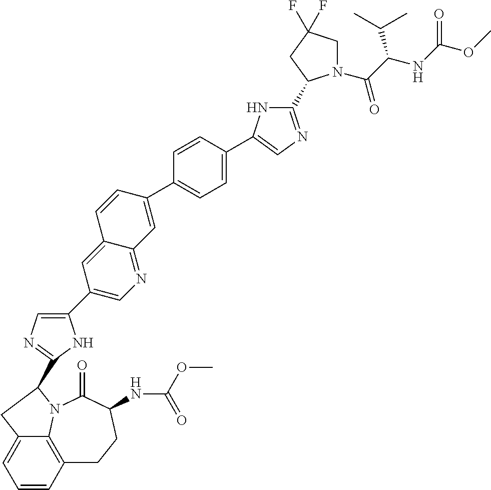 | ((2S,5S)-2-{5-[7-(4-{2- [(S)-1-((S)-2- Methoxycarbonylamino- 3-methyl-butyryl)-4,4- difluoro-pyrrolidin-2- yl]-3H-imidazol-4-yl}- phenyl)-quinolin-3-yl]- 1H-imidazol-2-yl}-4- oxo-1,2,4,5,6,7- hexahydro- azepino[3,2,1-hi]indol-5- yl)-carbamic acid methyl ester trifluroacetic acid |
| I-20 | 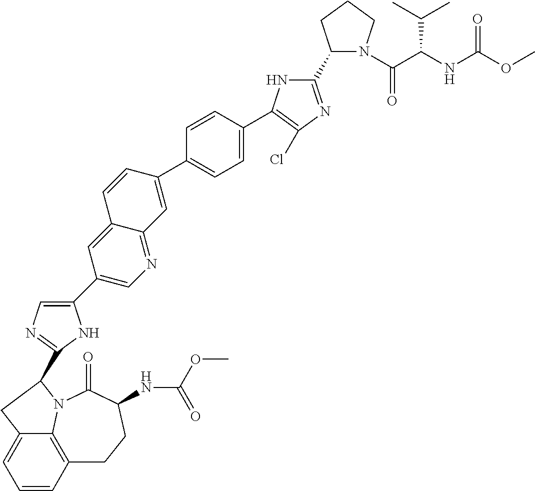 | ((2S,5S)-2-{5-[7-(4-{5- Chloro-2-[(S)-1-((S)-2- methoxycarbonylamino- 3-methyl-butyryl)- pyrrolidin-2-yl]-3H- imidazol-4-yl}-phenyl)- quinolin-3-yl]-1H- imidazol-2-yl}-4-oxo- 1,2,4,5,6,7-hexahydro- azepino[3,2,1-hi]indol-5- yl)-carbamic acid methyl ester trifluroacetic acid |
| I-21 | 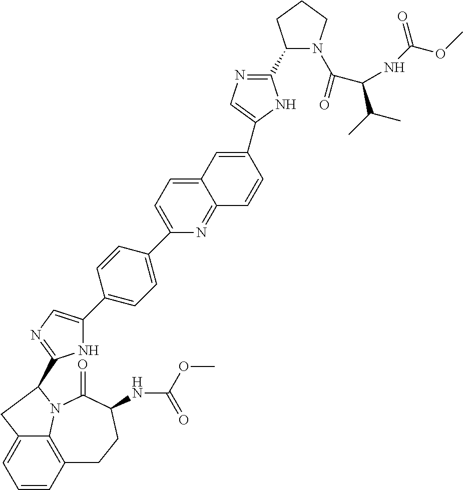 | ((2S,5S)-2-{4-[4-(6-{2- [(S)-1-((S)-2- Methoxycarbonylamino- 3-methyl-butyryl)- pyrrolidin-2-yl]-1H- imidazol-4-yl}-quinolin- 2-yl)-phenyl]-1H- imidazol-2-yl}-4-oxo- 1,2,4,5,6,7-hexahydro- azepino[3,2,1-hi]indol-5- yl)-carbamic acid methyl ester trifluroacetic acid |
| I-22 | 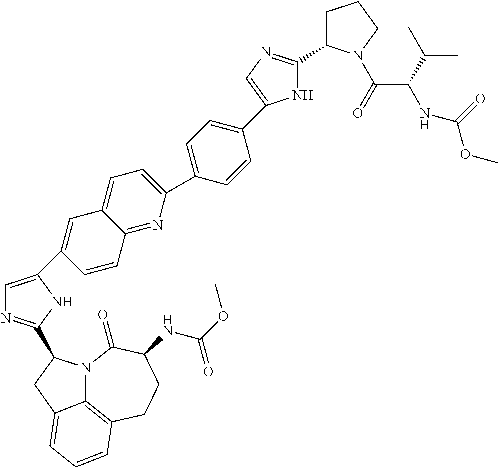 | ((2S,5S)-2-{5-[2-(4-{2- [(S)-1-((S)-2- Methoxycarbonylamino- 3-methyl-butyryl)- pyrrolidin-2-yl]-1H- imidazol-4-yl}-phenyl)- quinolin-6-yl]-1H- imidazol-2-yl}-4-oxo- 1,2,4,5,6,7-hexahydro- azepino[3,2,1-hi]indol-5- yl)-carbamic acid methyl ester trifluroacetic acid |
| I-23 | 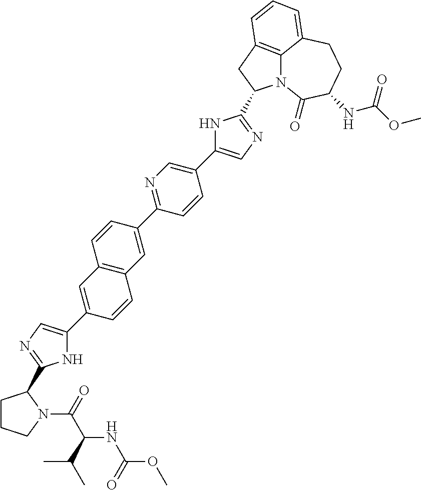 | ((2S,5S)-2-{4-[6-(6-{2- [(S)-1-((S)-2- Methoxycarbonylamino- 3-methyl- butyryl)pyrrolidin-2-yl]- 3H-imidazol-4-yl}- naphthalen-2-yl)- pyridin-3-yl]- 1Himidazol-2-yl}-4- oxo-1,2,4,5,6,7- hexahydro- azepino[3,2,1-hi]indol-5- yl)-carbamic acid methyl ester di-trifluoroacetic acid salt |
| I-24 | 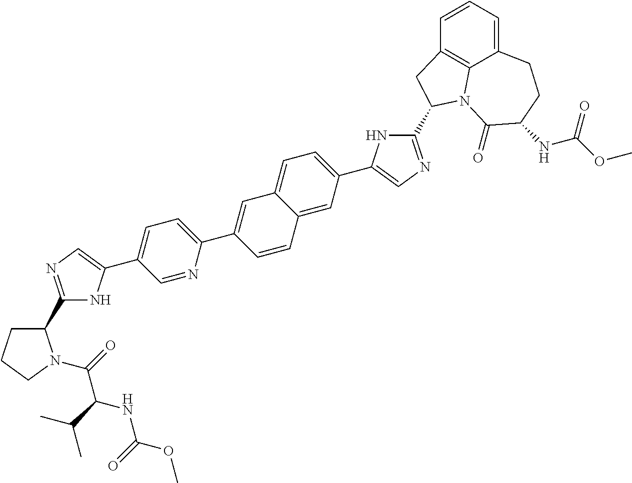 | ((2S,5S)-2-{4-[6-(5-{2- [(S)-1-((S)-2- Methoxycarbonylamino- 3-methyl-butyryl)- pyrrolidin-2-yl]-3H- imidazol-4-yl}-pyridin- 2-yl)-naphthalen-2-yl]- 1H-imidazol-2-yl}-4- oxo-1,2,4,5,6,7- hexahydro- azepino[3,2,1-hi]indol-5- yl)-carbamic acid methyl ester di-trifluoroacetic acid salt |
| I-25 | 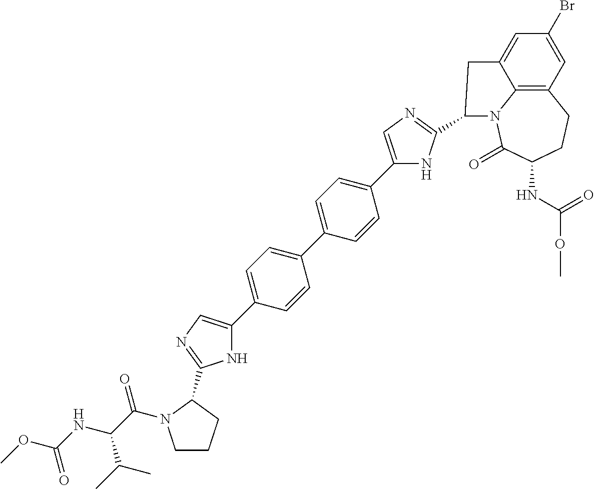 | {(2S,5S)-9-Bromo-2-[5- (4′-{2-[(R)-1-((R)-2- methoxycarbonylamino- 3-methyl-butyryl)- pyrrolidin-2-yl]-3H- imidazol-4-yl}-biphenyl- 4-yl)-1H-imidazol-2-yl]- 4-oxo-1,2,4,5,6,7- hexahydro- azepino[3,2,1-hi]indol-5- yl}-carbamic acid methyl ester |
| I-26 | 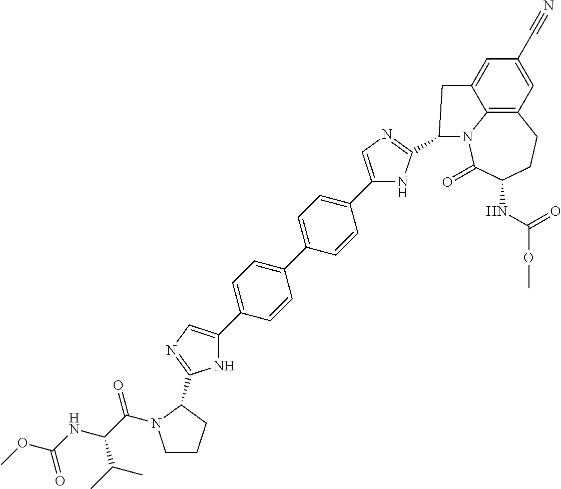 | {(2S,5S)-9-Cyano-2-[5- (4′-{2-[(R)-1-((R)-2- methoxycarbonylamino- 3-methyl-butyryl)- pyrrolidin-2-yl]-3H- imidazol-4-yl}-biphenyl- 4-yl)-1H-imidazol-2-yl]- 4-oxo-1,2,4,5,6,7- hexahydro- azepino[3,2,1-hi]indol-5- yl}-carbamic acid methyl ester |
| I-27 | 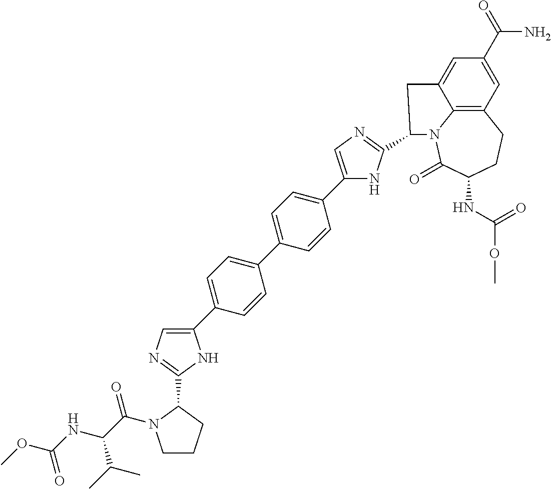 | {(2S,5S)-9-Carbamoyl- 2-[5-(4′-{2-[(R)-1-((R)- 2- methoxycarbonylamino- 3-methyl-butyryl)- pyrrolidin-2-yl]-3H- imidazol-4-yl}-biphenyl- 4-yl)-1H-imidazol-2-yl]- 4-oxo-1,2,4,5,6,7- hexahydro- azepino[3,2,1-hi]indol-5- yl}-carbamic acid methyl ester |
| I-28 | 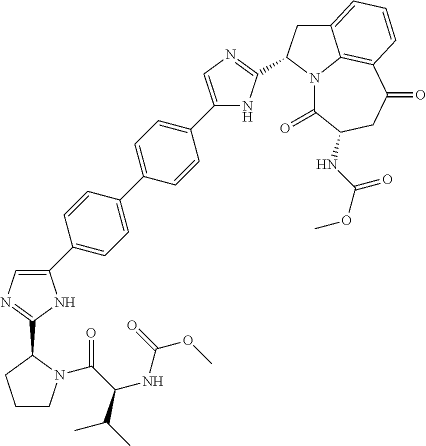 | {(2S,5S)-2-[5-(4′-{2- [(S)-1-((S)-2- Methoxycarbonylamino- 3-methyl-butyryl)- pyrrolidin-2-yl]-3H- imidazol-4-yl}-biphenyl- 4-yl)-1H-imidazol-2-yl]- 4,7-dioxo-1,2,4,5,6,7- hexahydro- azepino[3,2,1-hi]indol-5- yl}-carbamic acid methyl ester |
| I-29 | 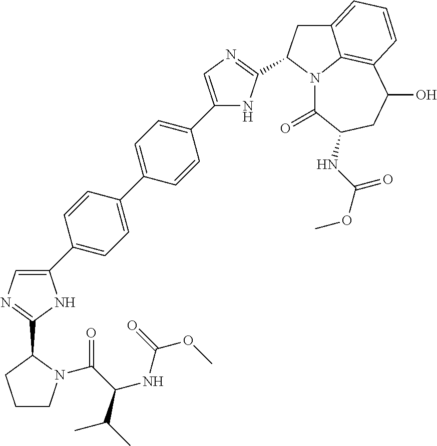 | {(2S,5S)-7-Hydroxy-2- [5-(4′-{2-[(S)-1-((S)-2- methoxycarbonylamino- 3-methyl-butyryl)- pyrrolidin-2-yl]-3H- imidazol-4-yl}-biphenyl- 4-yl)-1H-imidazol-2-yl]- 4-oxo-1,2,4,5,6,7- hexahydro- azepino[3,2,1-hi]indol-5- yl}-carbamic acid methyl ester |
| I-30 | 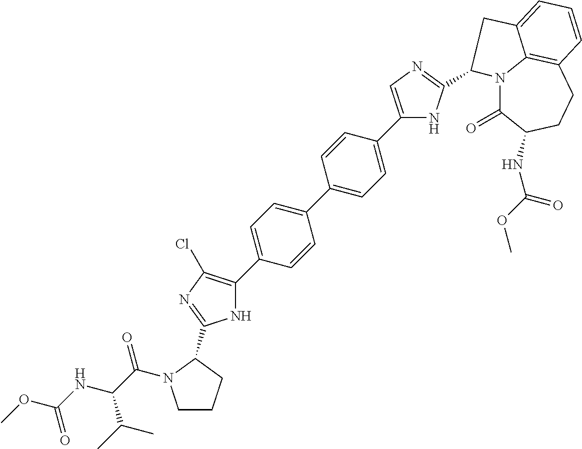 | {(2S,5S)-2-[5-(4′-{5- Chloro-2-[(S)-1-((S)-2- methoxycarbonyl amino- 3-methyl-butyryl)- pyrrolidin-2-yl]-3H- imidazol-4yl}-biphenyl- 4-yl)-1H-imidazol-2-yl]- 4-oxo-1,2,4,5,6,7- hexahydro- azepino[3,2,1-hi]indol-5- yl}-carbamic acid methyl ester |
| I-31 | 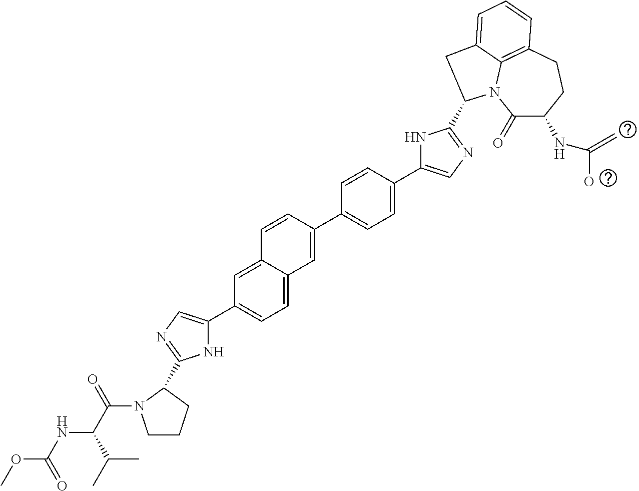 | {(2S,5S)-2-[5-(4-(6-{2- [(S)-1-((S)-2- Methoxycarbonylamino- 3-methyl-butyryl)- pyrrolidin-2-yl]-3H- imidazol-4-yl}- naphthalen-2-yl)- phenyl)-1H-imidazol-2- yl]-4-oxo-1,2,4,5,6,7- hexahydro- azepino[3,2,1-hi]indol-5- yl}-carbamic acid methyl ester |
| I-32 | 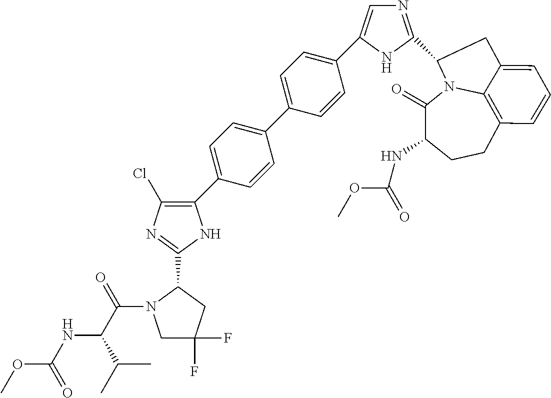 | {(2S,5S)-2-[5-(4′-{5- Chloro-2-[(S)-4,4- difluoro-1-((S)-2- methoxycarbonyl amino- 3-methyl-butyryl)- pyrrolidin-2-yl]-3H- imidazol-4yl}-biphenyl- 4-yl)-1H-imidazol-2-yl]- 4-oxo-1,2,4,5,6,7- hexahydro- azepino[3,2,1-hi]indol-5- yl}-carbamic acid methyl ester |
| I-33 | 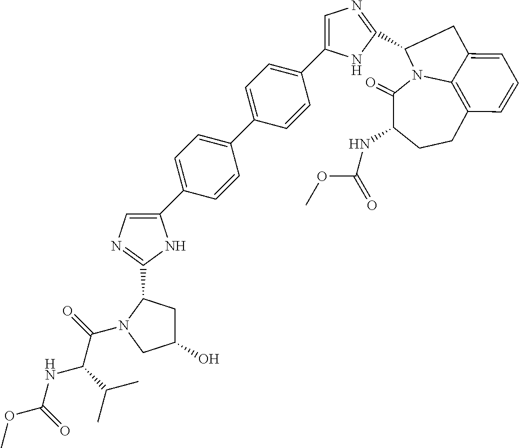 | {(2S,5S)-2-[5-(4′-{2- [(2S,4S)-4-Hydroxy-1- ((S)-2- methoxycarbonylamino- 3-methyl-butyryl)- pyrrolidin-2-yl]-3H- imidazol-4-yl}-biphenyl- 4-yl)-1H-imidazol-2-yl]- 4-oxo-1,2,4,5,6,7- hexahydro- azepino[3,2,1-hi]indol-5- yl}-carbamic acid methyl ester |
| I-34 | 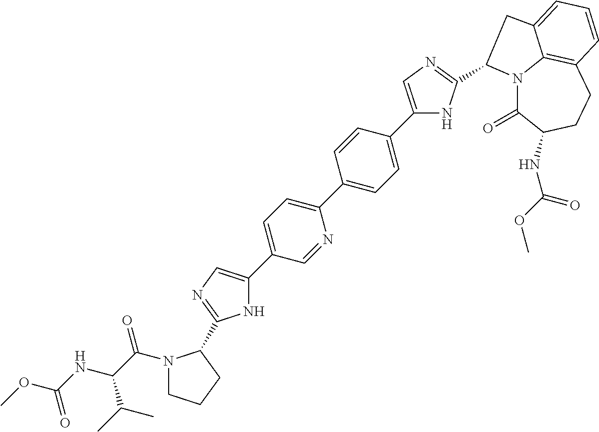 | ((2S,5S)-2-{5-[4-(5-{2- [(S)-1-((S)-2- Methoxycarbonylamino- 3-methyl-butyryl)- pyrrolidin-2-yl]-3H- imidazol-4-yl}-pyridin- 2-yl)-phenyl]-1H- imidazol-2-yl}-4-oxo- 1,2,4,5,6,7-hexahydro- azepino[3,2,1-hi]indol-5- yl)-carbamic acid methyl ester |
| I-35 | 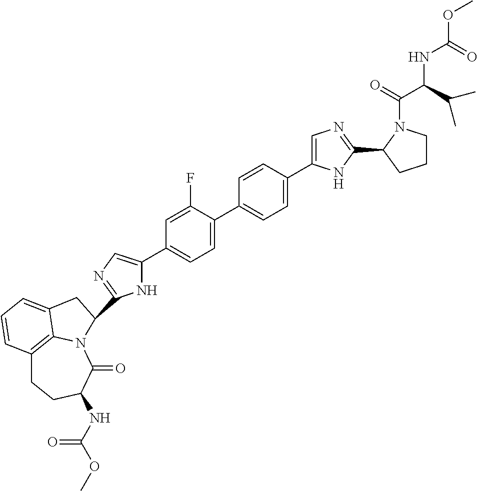 | {(2S,5S)-2-[5-(2-Fluoro- 4′-{2-[(S)-1-((S)-2- methoxycarbonylamino- 3-methyl-butyryl)- pyrrolidin-2-yl]-1H- imidazol-4-yl}-biphenyl- 4-yl)-1H-imidazol-2-yl]- 4-oxo-1,2,4,5,6,7- hexahydro- azepino[3,2,1-hi]indol-5- yl}-carbamic acid methyl ester |
| I-36 | 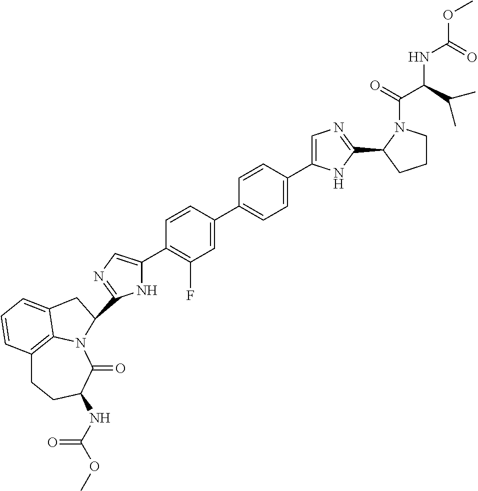 | {(2S,5S)-2-[5-(3-Fluoro- 4′-{2-[(S)-1-((S)-2- methoxycarbonylamino- 3-methyl-butyryl)- pyrrolidin-2-yl]-1H- imidazol-4-yl}-biphenyl- 4-yl)-1H-imidazol-2-yl]- 4-oxo-1,2,4,5,6,7- hexahydro- azepino[3,2,1-hi]indol-5- yl}-carbamic acid methyl ester |
| I-37 | 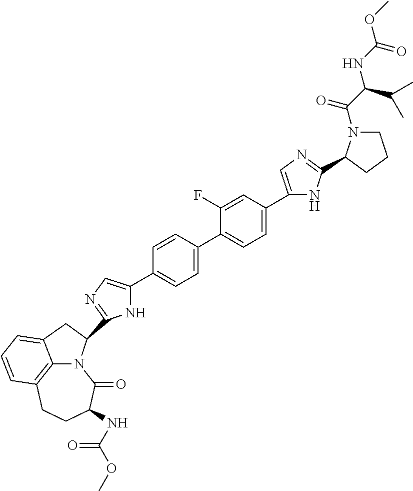 | {(2S,5S)-2-[5-(2′- Fluoro-4′-{2-[(S)-1-((S)- 2- methoxycarbonylamino- 3-methyl-butyryl)- pyrrolidin-2-yl]-1H- imidazol-4-yl}-biphenyl- 4-yl)-1H-imidazol-2-yl]- 4-oxo-1,2,4,5,6,7- hexahydro- azepino[3,2,1-hi]indol-5- yl}-carbamic acid methyl ester |
| I-38 | 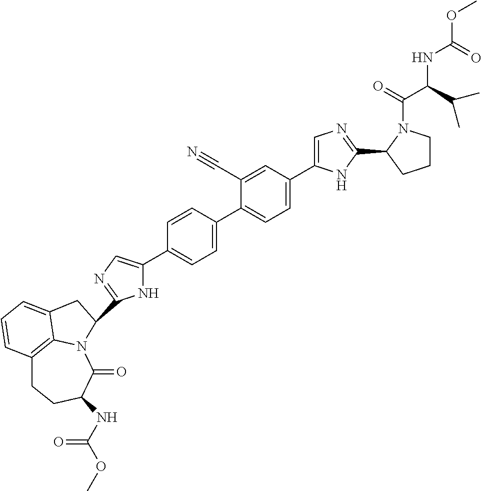 | {(2S,5S)-2-[5-(2′- Cyano-4′-{2-[(S)-1-((S)- 2- methoxycarbonylamino- 3-methyl-butyryl)- pyrrolidin-2-yl]-1H- imidazol-4-yl}-biphenyl- 4-yl)-1H-imidazol-2-yl]- 4-oxo-1,2,4,5,6,7- hexahydro- azepino[3,2,1-hi]indol-5- yl}-carbamic acid methyl ester |
| I-39 | 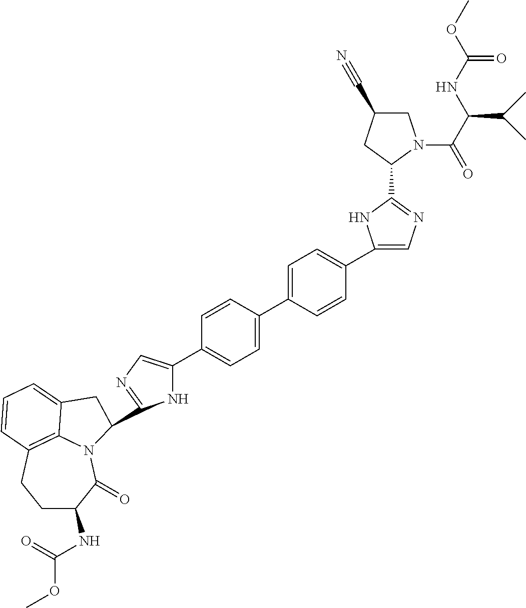 | {(2S,5S)-2-[5-(4′-{2- [(2S,4R)-4-Cyano-1- ((S)-2- methoxycarbonylamino- 3-methyl-butyryl)- pyrrolidin-2-yl]-3H- imidazol-4-yl}-biphenyl- 4-yl)-1H-imidazol-2-yl]- 4-oxo-1,2,4,5,6,7- hexahydro- azepino[3,2,1-hi]indol-5- yl}-carbamic acid methyl ester |
| I-40 | 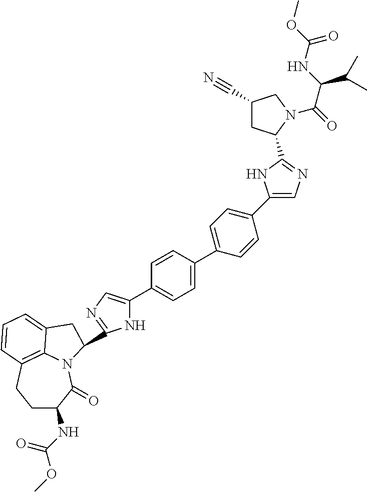 | {(2S,5S)-2-[5-(4′-{2- [(2S,4S)-4-Cyano-1- ((S)-2- methoxycarbonylamino- 3-methyl-butyryl)- pyrrolidin-2-yl]-3H- imidazol-4-yl}-biphenyl- 4-yl)-1H-imidazol-2-yl]- 4-oxo-1,2,4,5,6,7- hexahydro- azepino[3,2,1-hi]indol-5- yl}-carbamic acid methyl ester |
| I-41 | 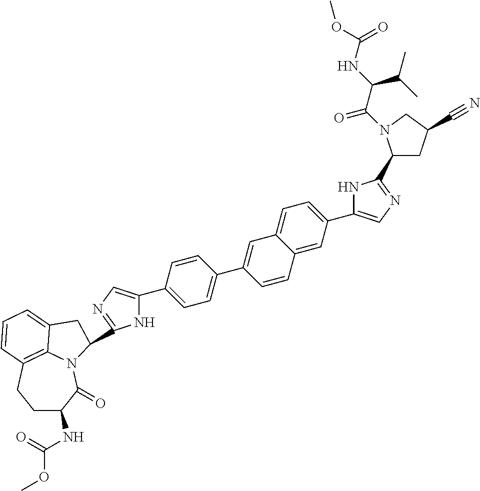 | {(2S,5S)-2-{5-[4-(6-{2- [(2S,4S)-4-Cyano-1- ((S)-2- methoxycarbonylamino- 3-methyl-butyryl)- pyrrolidin-2-yl]-3H- imidazol-4-yl}- naphthalen-2-yl)- phenyl]-1H-imidazol-2- yl]-4-oxo-1,2,4,5,6,7- hexahydro- azepino[3,2,1-hi]indol-5- yl}-carbamic acid methyl ester |
| I-42 | 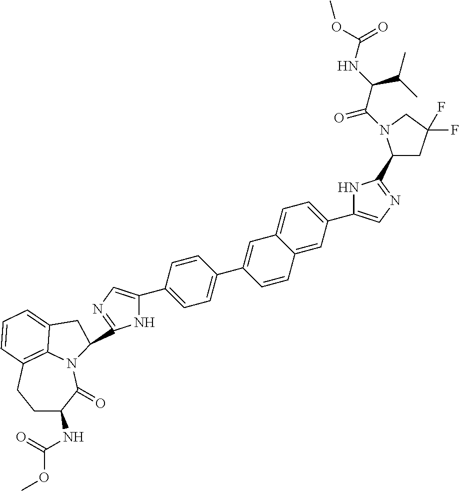 | {(2S,5S)-2-{5-[4-(6-{2- [(S)-4,4-Difluoro-1-((S)- 2- methoxycarbonylamino- 3-methyl-butyryl)- pyrrolidin-2-yl]-3H- imidazol-4-yl}- naphthalen-2-yl)- phenyl]-1H-imidazol-2- yl}-4-oxo-1,2,4,5,6,7- hexahydo-azepino[3,2,1- hi]indol-5-yl}-carbamic acid methyl ester |
| I-43 | 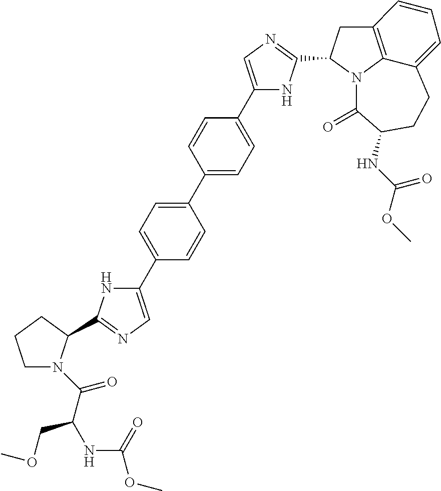 | {(2S,5S)-2-[5-(4-{2- [(S)-1-((S)-3-Methoxy- 2- methoxycarbonylamino- propionyl)-pyrrolidin-2- yl]-3H-imidazol-4-yl}- biphenyl-4-yl)-1H- imidazol-2-yl]-4-oxo- 1,2,4,5,6,7-hexahydro- azepino[3,2,1-hi]indol-5- yl}-carbamic acid methyl ester |
| I-44 | 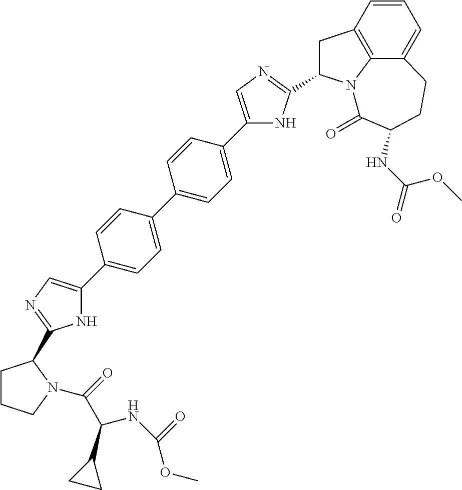 | (2S,5S)-2-[5-(4-{2- [(S)-1-((S)-2- Cyclopropyl-2- methoxycarbonylamino- acetyl)-pyrrolidin-2-yl]- 3H-imidazol-4-yl}- biphenyl-4-yl)-1H- imidazol-2-yl]-4-oxo- 1,2,4,5,6,7-hexahydro- azepino[3,2,1-hi]indol-5- yl}-carbamic acid methyl ester |
| I-45 | 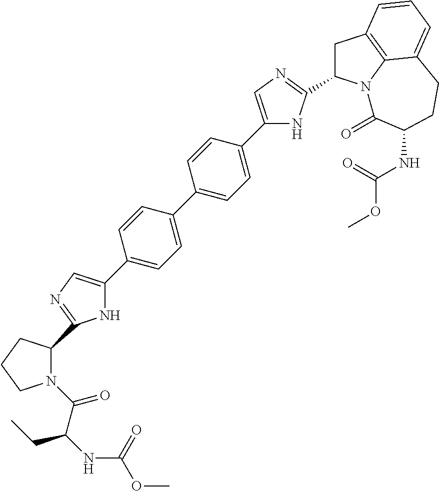 | {(2S,5S)-2-[5-(4-{2- [(S)-1-((S)-2- Methoxycarbonylamino- butyryl)-pyrrolidin-2- yl]-3H-imidazol-4-yl}- biphenyl-4-yl)-1H- imidazol-2-yl]-4-oxo- 1,2,4,5,6,7-hexahydro- azepino[3,2,1-hi]indol-5- yl}-carbamic acid methyl ester |
| I-46 | 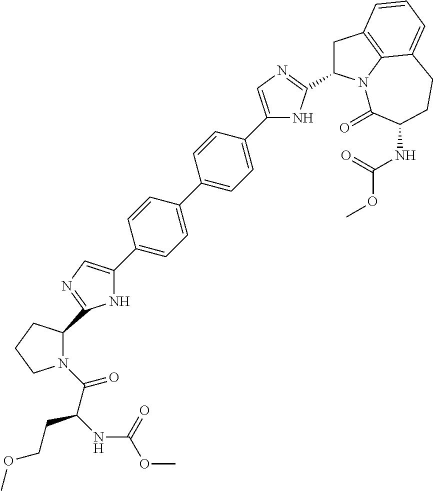 | {(2S,5S)-2-[5-(4-{2- [(S)-1-((S)-4-Methoxy- 2- Methoxycarbonylamino- butyryl)-pyrrolidin-2- yl]-3H-imidazol-4-yl}- biphenyl-4-yl)-1H- imidazol-2-yl]-4-oxo- 1,2,4,5,6,7-hexahydro- azepino[3,2,1-hi]indol-5- yl}-carbamic acid methyl ester |
| I-47 | 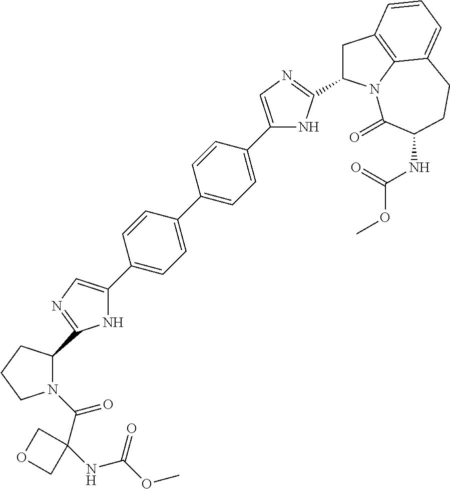 | {(2S,5S)-2-[5-(4-{2- [(S)-1-(3- Methoxycarbonylamino- oxetane-3-carbonyl)- pyrrolidin-2-yl]-3H- imidazol-4-yl}-biphenyl- 4-yl)-1H-imidazol-2-yl]- 4-oxo-1,2,4,5,6,7- hexahydro- azepino[3,2,1-hi]indol-5- yl}-carbamic acid methyl ester |
| I-48 | 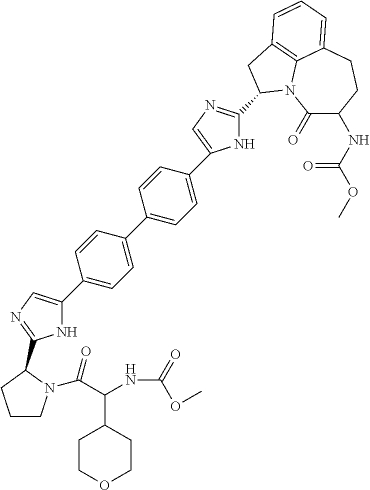 | ((2S,5S)-2-{5-[4-(2- {(S)-1-[2- Methoxycarbonylamino- 2-(tetrahydro-pyran-4- yl)-pyrrolidin-2-yl]-3H- imidazol-4-yl}-biphenyl- 4-yl)-1H-imidazol-2-yl]- 4-oxo-1,2,4,5,6,7- hexahydro- azepino[3,2,1-hi]indol-5- yl}-carbamic acid methyl ester |
| I-49 | 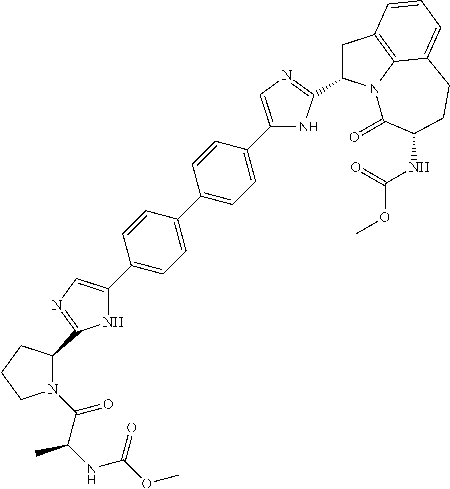 | {(2S,5S)-2-[5-(4-{2- [(S)-1-((S)-2- Methoxycarbonylamino- propionyl)-pyrolidin-2- yl]-3H-imidazol-4-yl}- biphenyl-4-yl)-1H- imidazol-2-yl]-4-oxo- 1,2,4,5,6,7-hexahydro- azepino[3,2,1-hi]indol-5- yl}-carbamic acid methyl ester |
| I-50 | 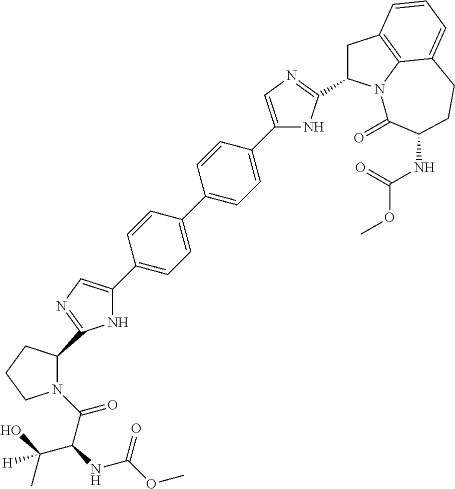 | {(2S,5S)-2-[5-(4-{2- [(S)-1-((S)-3-Hydroxy- 2- methoxycarbonylamino- butyl)-pyrolidin-2-yl]- 3H-imidazol-4-yl}- biphenyl-4-yl)-1H- imidazol-2-yl]-4-oxo- 1,2,4,5,6,7-hexahydro- azepino[3,2,1-hi]indol-5- yl}-carbamic acid methyl ester |
| I-51 | 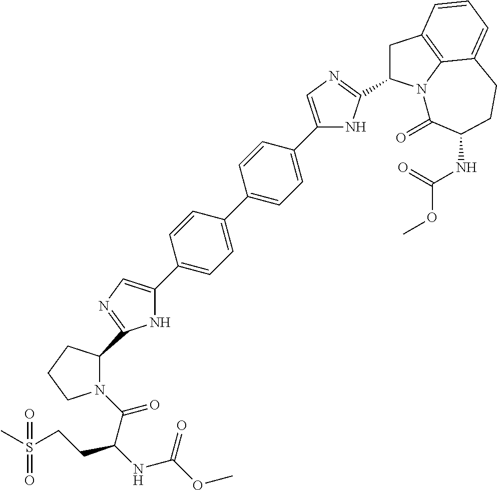 | {(2S,5S)-2-[5-(4-{2- [(S)-1-((S)-4- Methanesulfonyl-2- methoxycarbonylamino- butyl)-pyrolidin-2-yl]- 3H-imidazol-4-yl}- biphenyl-4-yl)-1H- imidazol-2-yl]-4-oxo- 1,2,4,5,6,7-hexahydro- azepino[3,2,1-hi]indol-5- yl}-carbamic acid methyl ester |
| I-52 | 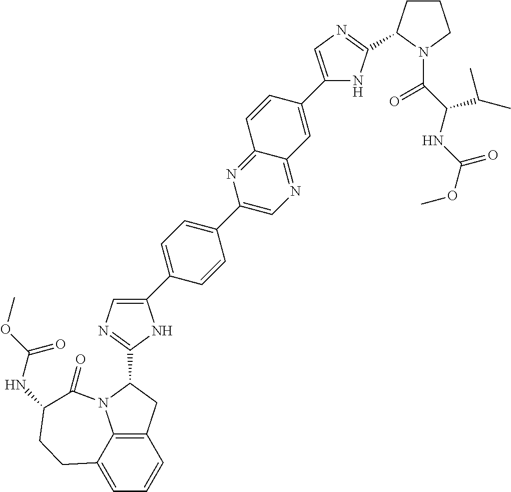 | ((2S,5S)-2-{5-[4-(6-{2- [(S)-1-((S)-2- Methoxycarbonylamino- 3-methyl-butyryl)- pyrrolidin-2-yl]-3H- imidazol-4-yl}- quinoxalin-2-yl)- phenyl]-1H-imidazol-2- yl}-4-oxo-1,2,4,5,6,7- hexahydro- azepino[3,2,1-hi]indol-5- yl)-carbamicacid methyl ester |
| I-53 | 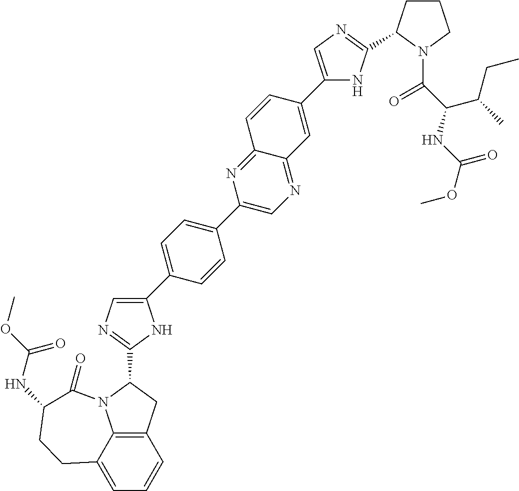 | ((2S,5S)-2-{5-[4-(6-{2- [(S)-1-((2S,3S)-2- Methoxycarbonylamino- 3-methyl-pentanoyl)- pyrrolidin-2-yl]-3H- imidazol-4-yl}- quinoxalin-2-yl)- phenyl]-1H-imidazol-2- yl}-4-oxo-1,2,4,5,6,7- hexahydroazepino[3,2,1- hi]indol-5yl)-carbamic acid methyl ester |
| I-54 | 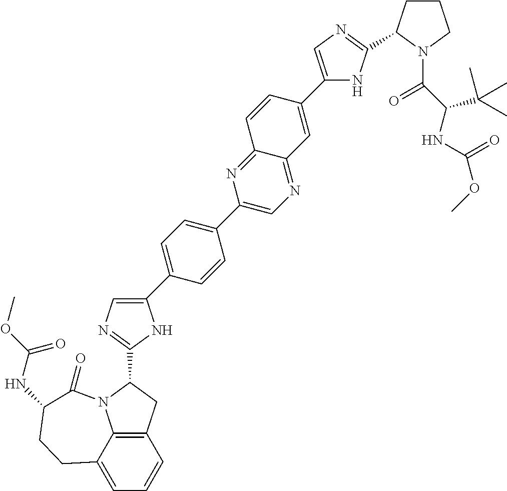 | ((2S,5S)-2-{5-[4-(6-{2- [(S)-1-((S)-2- Methoxycarbonylamino- 3,3-dimethyl-butyryl)- pyrrolidin-2-yl]-3H- imidazol-4-yl}- quinoxalin-2-yl)- phenyl]-1H-imidazol-2- yl}-4-oxo-1,2,4,5,6,7- hexahydro- azepino[3,2,1-hi]indol-5- yl)-carbamic acid methyl ester |
| I-55 | 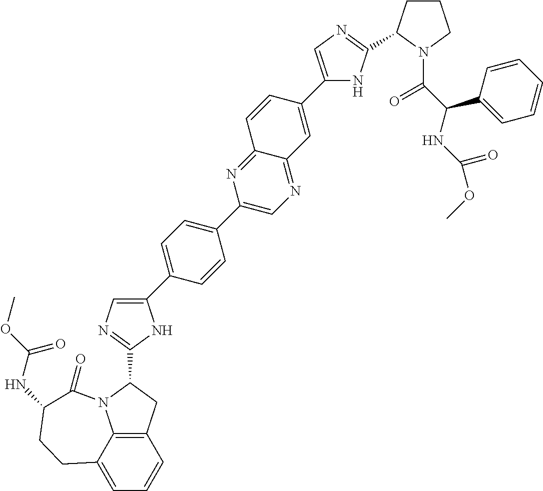 | ((2S,5S)-2-{5-[4-(6-{2- [(S)-1-((R)-2- Methoxycarbonylamino- 2-phenyl-acetyl)- pyrrolidin-2-yl]-3H- imidazol-4-yl}- quinoxalin-2-yl)- phenyl]-1H-imidazol-2- yl}-4-oxo-1,2,4,5,6,7- hexahydro- azepino[3,2,1-hi]indol-5- yl)-carbamic acid methyl ester |
| I-56 | 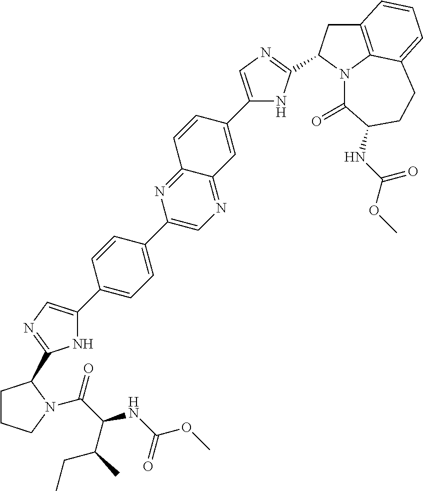 | ((2S,5S)-2-{5-[2-(4-{2- [(S)-1-((2S,3S)-2- Methoxycarbonylamino- 3 methyl-pentanoyl)- pyrrolidin-2-yl]-3H- imidazol-4-yl}-phenyl) quinoxalin-6-yl]-1H- imidazol-2-yl}-4-oxo- 1,2,4,5,6,7-hexahydro- azepino[3,2,1-hi]indol-5- yl)-carbamic acid methyl ester |
| I-57 | 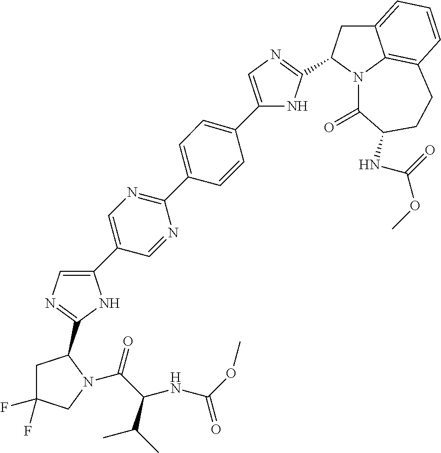 | ((2S,5S)-2-{4-[4-(5-{2- [(S)-4,4-Difluoro-1-(2- methoxycarbonylamino- 3-methyl-butyryl)- pyrrolidin-2-yl]-3H- imidazol-4-yl}- pyrimidin-2-yl)-phenyl]- 1H-imidazol-2-yl}-4- oxo-1,2,4,5,6,7- hexahydro- azepino[3,2,1-hi]indol-5- yl)-carbamic acid methyl ester |
| I-58 | 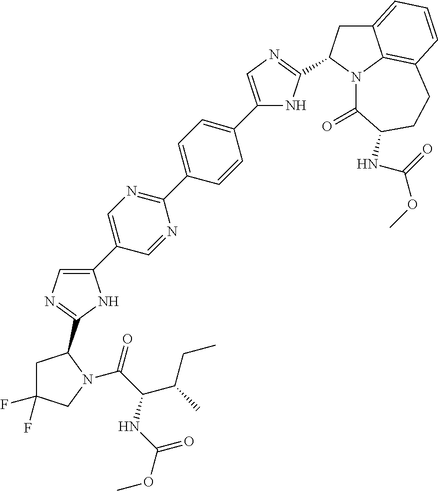 | ((1S,2S)-1-{(S)-4,4- Difluoro-2-[5-(2-{4-[2- ((2S,5S)-5- methoxycarbonylamino- 4-oxo-1,2,4,5,6,7- hexahydro- azepino[3,2,1-hi]indol-2- yl)-1H-imidazol-4-yl]- phenyl}-pyrimidin-5-yl)- 1H-imidazol-2-yl]- pyrrolidine-1-carbonyl}- 2-methyl-butyl)- carbamic acid methyl ester |
| I-59 | 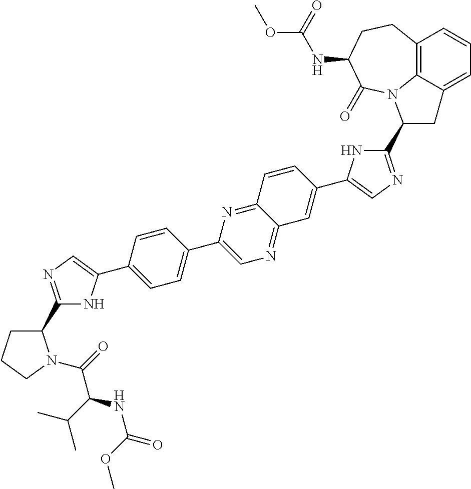 | ((2S,5S)-2-{5-[2-(4-{2- [(S)-1-((S)-2- Methoxycarbonylamino- 3-methyl-butyryl)- pyrrolidin-2-yl]-3H- imidazol-4-yl}-phenyl)- quinoxalin-6-yl]-1H- imidazol-2-yl}-4-oxo- 1,2,4,5,6,7-hexahydro- azepino[3,2,1-hi]indol-5- yl)-carbamic acid methyl ester |
| I-60 | 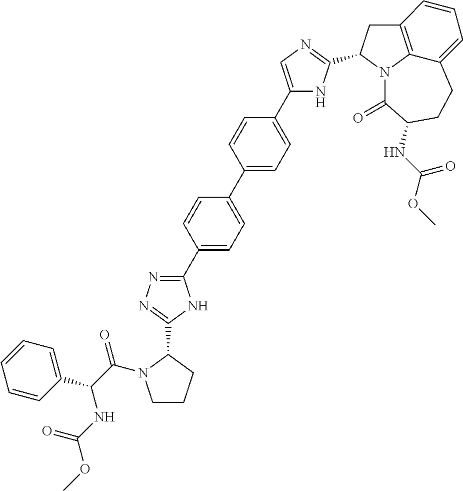 | {(2S,5S)-2-[5-(4′-{5- [(S)-1-((R)-2- Methoxycarbonylamino- 2-phenyl-acetyl)- pyrrolidin-2-yl]-4H- [1,2,4]triazol-3-yl}- biphenyl-4-yl)-1H- imidazol-2-yl]-4-oxo- 1,2,4,5,6,7-hexahydro- azepino[3,2,1-hi]indol-5- yl}-carbamic acid methyl ester |
| I-61 | 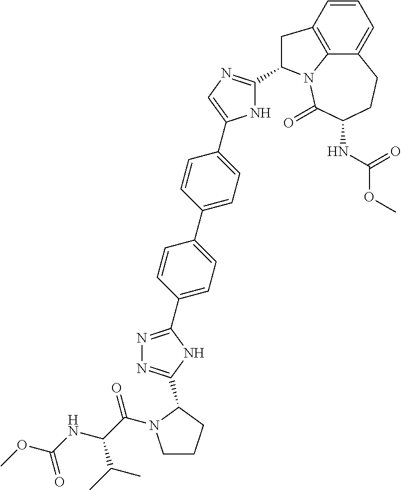 | {(2S,5S)-2-[5-(4′-{5- [(S)-1-((S)-2- Methoxycarbonylamino- 3-methyl-butyryl)- pyrrolidin-2-yl]-4H- [1,2,4]triazol-3-yl}- biphenyl-4-yl)-1H- imidazol-2-yl]-4-oxo- 1,2,4,5,6,7-hexahydro- azepino[3,2,1-hi]indol-5- yl}-carbamic acid methyl ester |
| I-62 | 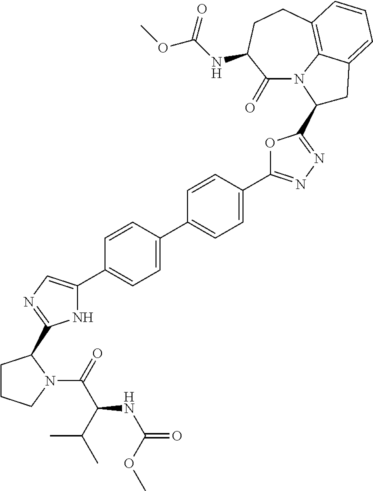 | {(2S,5S)-2-[5-(4′-{5- [(S)-1-((S)-2- Methoxycarbonylamino- 3-methyl-butyryl)- pyrrolidin-2-yl]-1H- imidazol-2-yl}-biphenyl- 4-yl)-[1,3,4]oxadiazol-2- yl]-4-oxo-1,2,4,5,6,7- hexahydro- azepino[3,2,1-hi]indol-5- yl}-carbamic acid methyl ester |
| I-63 | 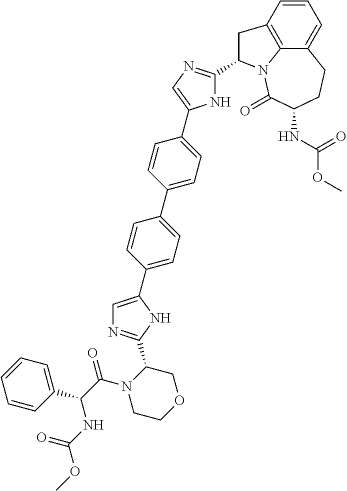 | {(2S,5S)-2-[5-(4′-{2- [(R)-4-((S)-2- Methoxycarbonylamino- 2-phenyl-acetyl)- morpholin-3-yl]-3H- imidazol-4-yl}-biphenyl- 4-yl)-1H-imidazol-2-yl]- 4-oxo-1,2,4,5,6,7- hexahydro- azepino[3,2,1-hi]indol-5- yl}-carbamic acid methyl ester |
| I-64 | 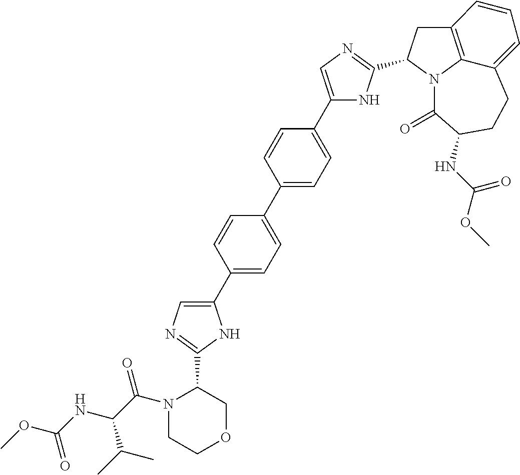 | {(2S,5S)-2-[5-(4′-{2- [(R)-4-((S)-2- Methoxycarbonylamino- 3-methyl-butyryl)- morpholin-3-yl]-3H- imidazol-4-yl}-biphenyl- 4-yl)-1H-imidazol-2-yl]- 4-oxo-1,2,4,5,6,7- hexahydro- azepino[3,2,1-hi]indol-5- yl}-carbamic acid methyl ester |
| I-65 | 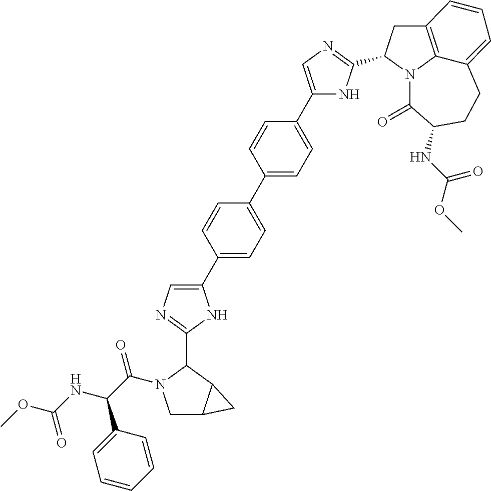 | {(2S,5S)-2-[5-(4′-{2- [(S)-3-((R)-2- Methoxycarbonylamino- 2-phenyl-acetyl)-3-aza- bicyclo[3.1.0]hex-2-yl]- 3H-imidazol-4-yl}- biphenyl-4-yl)-1H- imidazol-2-yl]-4-oxo- 1,2,4,5,6,7-hexahydro- azepino[3,2,1-hi]indol-5- yl}-carbamic acid methyl ester |
| I-66 | 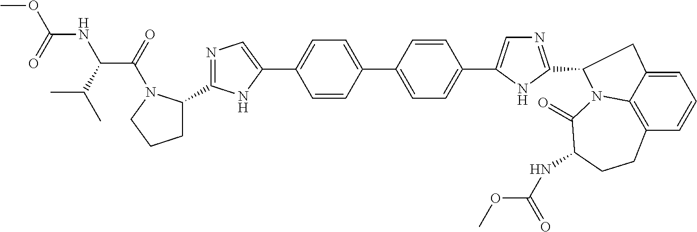 | {(2S,5S)-2-[5-(4′-{2- [(S)-1-((S)-2- Methoxycarbonylamino- 3-methyl-butyryl)- pyrrolidin-2-yl]-3H- imidazol-4-yl}-biphenyl- 4-yl)-1H-imidazol-2-yl]- 4-oxo-1,2,4,5,6,7- hexahydro- azepino[3,2,1-hi]indol-5- yl}-carbamic acid methyl ester |
| I-67 | 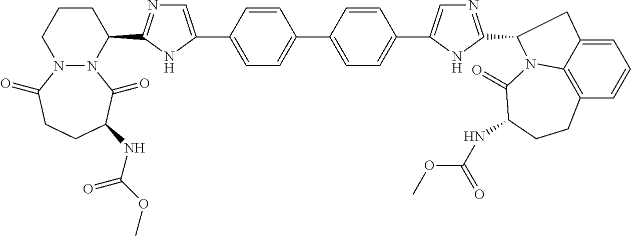 | {(2S,5S)-2-[5-(4′-{2- [(1S,9S)-9- methoxycarbonylamino- 6,10-dioxo-octahydro- pyridazino[1,2- a][1,2]diazepin-1-yl])- 3H-imidazol-4-yl}- biphenyl-4-yl)-1H- imidazol-2-yl]-4-oxo- 1,2,4,5,6,7-hexahydro- azepino[3,2,1-hi]indol-5- yl}-carbamic acid methyl ester |
| I-68 | 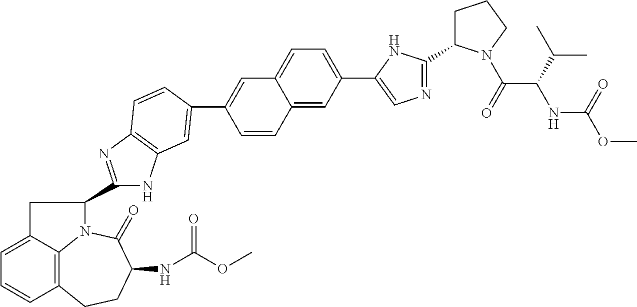 | 6-(6-{2-[(S)-1-((S)-2- Methoxycarbonylamino- 3-methyl-butyryl)- pyrrolidin-2-yl]-1H- imidazol-4-yl}- naphthalen-2-yl)-2- ((2S,5S)-5- methoxycarbonylamino- 4-oxo-1,2,4,5,6,7- hexahydro- azepino[3,2,1-hi]indol-2- yl)-benzoimidazole-1- carboxylic acid methyl ester |
| I-69 | 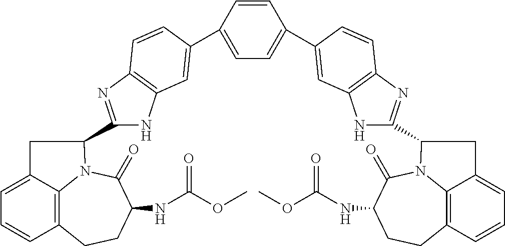 | N,N′-[1,4- phenylenebis[1H- benzimidazole-6,2-diyl- (2S,5S)-1,2,4,5,6,7- hexahydro-4- oxoazepino[3,2,1- hi]indole-5,2- diyl]]biscarbamic acid C,C′ dimethyl ester |
| I-70 | 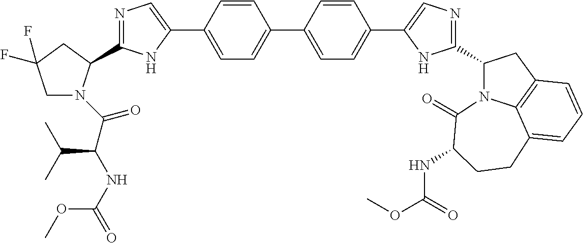 | {(2S,5S)-2-[5-(4′-{2- [(S)-4,4-Difuoro-1-((S)- 2- Methoxycarbonylamino- 3-methyl-butyryl)- pyrrolidin-2-yl]-3H- imidazol-4-yl}-biphenyl- 4-yl)-1H-imidazol-2-yl]- 4-oxo-1,2,4,5,6,7- hexahydro- azepino[3,2,1-hi]indol-5- yl}-carbamic acid methyl ester |

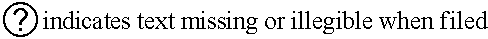

### Synthesis

### General Schemes

The following schemes depict general methods for obtaining compounds of Formulae I-III.

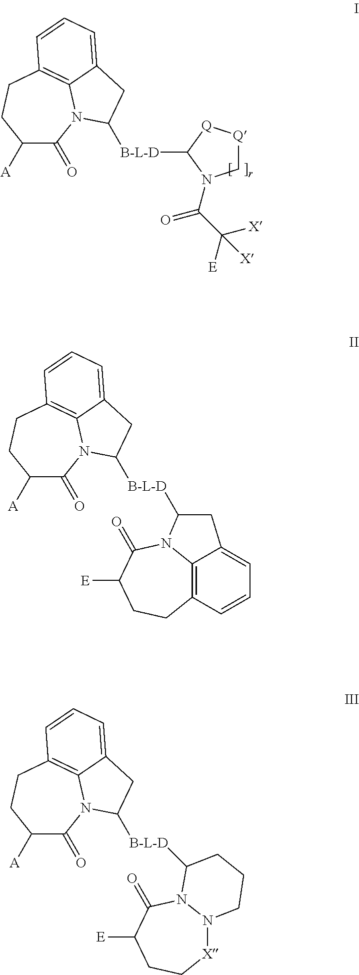

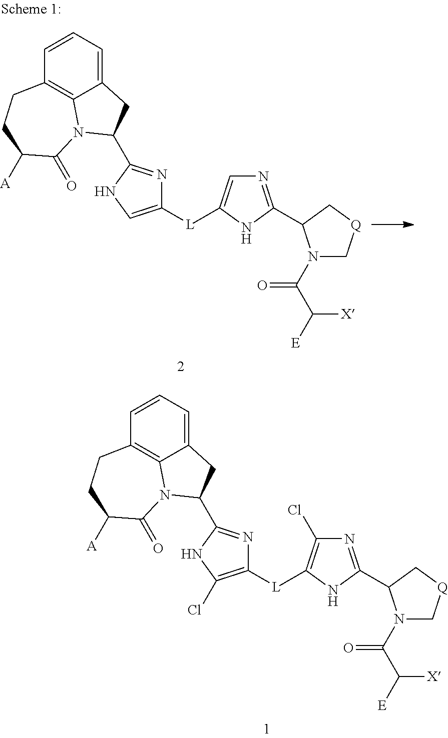

Compounds of formula I, wherein B and D can be chloroimidazoles, and L can be independently aryl or quinolinyl, naphthyl, quinazolinyl, or quinaxolinyl, 1 can be prepared from the corresponding imidazoles 2 using standard reaction conditions of chlorination of imidazole derivatives described for example, in Journal of Medicinal Chemistry (1986), 29(6), 1065-80; Journal of the Chemical Society, Perkin Transactions 1: Organic and Bio-Organic Chemistry (1972-1999) (1983) (4), 809-11; Eur. Pat. Appl. (1990) EP 365030 A1 19900425; Journal of Heterocyclic Chemistry (1994), 31(5), 1121-3; PCT Int. Appl. (2007), WO 2007070433.

### (Scheme 1)

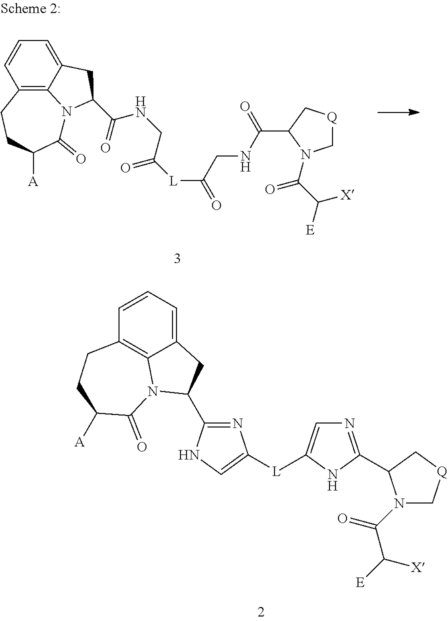

The imidazoles corresponding to 2 can be formed by the reaction of ammonium acetate on the keto-amide intermediates 3, as shown Scheme 2, using standard reaction conditions described, for example in, Journal of Organic Chemistry (1937), 2, 319-27; Synlett (2001), (2), 218-221.

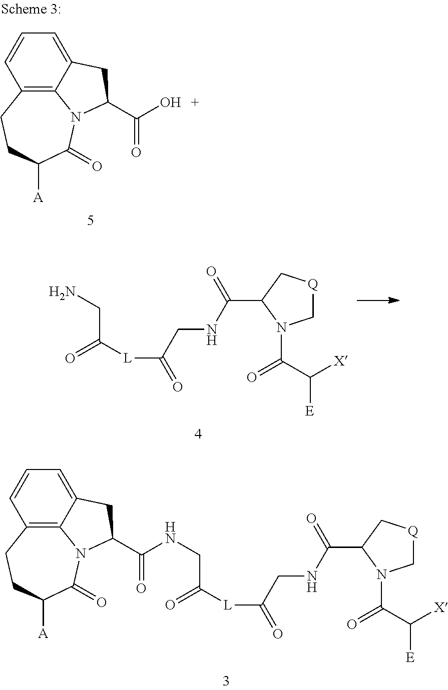

The keto-amide compounds of general formula 3, can be prepared from the corresponding carboxylic acid 5, and the amines of the formula 4, using standard methods of amide coupling. (Scheme 3)

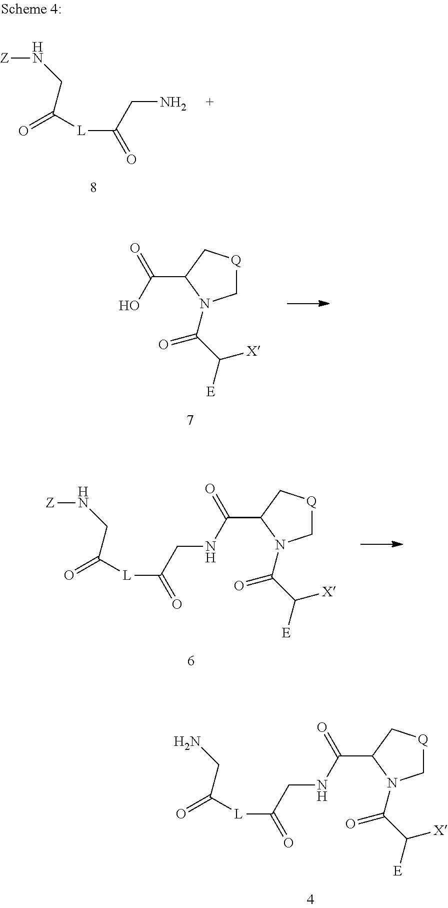

The amino-keto amides of formula 4 can be prepared from the corresponding bezyloxycarbonyl (Z) protected amine derivatives 6, via hydrogenation. The compounds of formula 6 can be assembled together via the coupling of carboxylic acid derivatives 7 and the differentially protected bis-amine derivatives 8 using standard methods of amide synthesis. (Scheme 4)

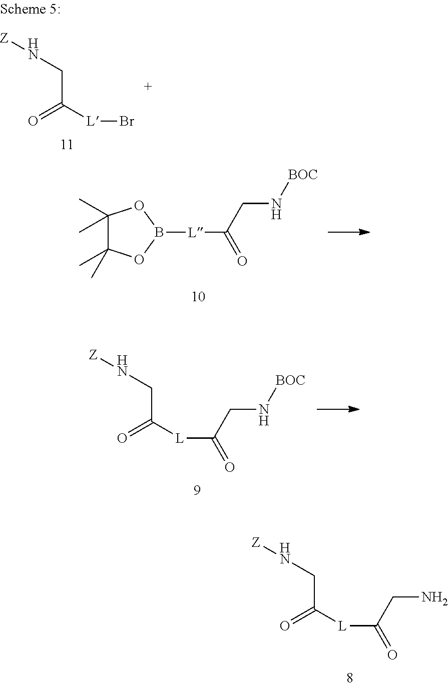

Compounds of formula 8 may be prepared from a differentially protected bis-amine derivatives 9 with a selective removal of BOC protecting group using standard methods of deprotection of a BOC group. Compounds of formula 9 may in turn be prepared in various methods including, for example the one shown in Scheme 5. In this method, an aryl bromide derivative 11 may be coupled with a boronate ester derivative 10 under Pd⁰-coupling conditions to provide compounds of formula 9. The boronate esters 10 and the bromo aryl derivatives 9 can be prepared starting from corresponding bromo-aryl acetophenones via reported methods.

The compounds of formula 5 are can be prepared starting from commercially available FMOC-Haic-COOH via its conversion to the amino acid 12, which can be transformed into compounds of formula 5 via standard alkylation, acylation and urea forming reactions. (Scheme 6)

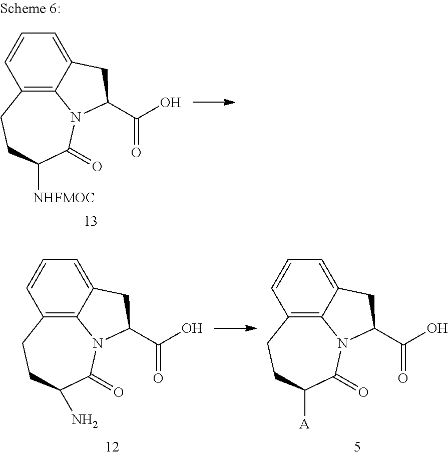

Compounds of formula 7 can be prepared starting from commercially available proline derivatives such as H-Pro-OMe or 3,3-difluoro-Pro-OH and their coupling with various amino acid derivatives using standard methods of amide coupling.

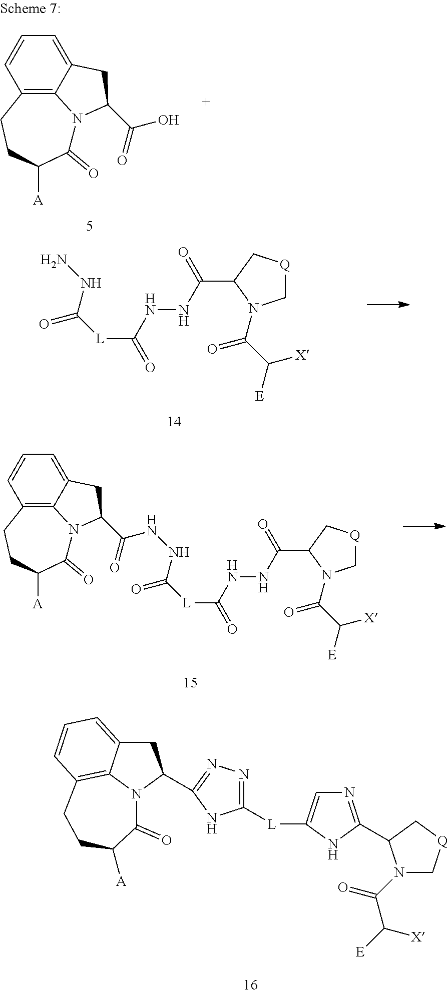

Compounds of formula I, wherein B and D are trizoles can be prepared from the reaction sequence shown in Scheme 7. The key triazole forming reaction can be accomplished using the reaction conditions described in the literature, for example, in Journal of Medicinal Chemistry (2007), 50, 3086-3100, using the intermediate 15. The intermediate 15 can be prepared from 5 and 14. The intermediate 14, can be prepared by adopting similar differential protecting strategy employed in the synthesis of the intermediate 8. Similar strategies described earlier for imidazole moieties at B and D, can be adopted for the synthesis of compounds of formula II and III wherein the B and D moieties can independently be a triazole.

### Pharmaceutical Compositions and Administration

Pharmaceutical compositions of the subject Compounds for administration via several routes were prepared as described in this Example.

Composition for Oral Administration (A)

| Ingredient | % wt./wt. |
|---|---|
| Active ingredient | 20.0% |
| Lactose | 79.5% |
| Magnesium stearate | 0.5% |

The ingredients are mixed and dispensed into capsules containing about 100 mg each; one capsule would approximate a total daily dosage.

Composition for Oral Administration (B)

| Ingredient | % wt./wt. |
|---|---|
| Active ingredient | 20.0% |
| Magnesium stearate | 0.5% |
| Crosscarmellose | 2.0% |
| sodium | |
| Lactose | 76.5% |
| PVP | 1.0% |
| (polyvinylpyrrolidine) | |

The ingredients are combined and granulated using a solvent such as methanol. The formulation is then dried and formed into tablets (containing about 20 mg of active compound) with an appropriate tablet machine.

Composition for Oral Administration (C)

| Ingredient | % wt./wt. | |
|---|---|---|
| Active compound | 1.0 | g |
| Fumaric acid | 0.5 | g |
| Sodium chloride | 2.0 | g |
| Methyl paraben | 0.15 | g |
| Propyl paraben | 0.05 | g |
| Granulated sugar | 25.5 | g |
| Sorbitol (70% solution) | 12.85 | g |
| Veegum K (Vanderbilt Co.) | 1.0 | g |
| Flavoring | 0.035 | ml |
| Colorings | 0.5 | mg |

| | |
|---|---|
| Distilled water | q.s. to 100 ml |

The ingredients are mixed to form a suspension for oral administration.

Parenteral Formulation (D)

| Ingredient | % wt./wt. |
|---|---|
| Active ingredient | 0.25 g |
| Sodium Chloride | qs to make isotonic |
| Water for injection to | 100 ml |

The active ingredient is dissolved in a portion of the water for injection. A sufficient quantity of sodium chloride is then added with stirring to make the solution isotonic. The solution is made up to weight with the remainder of the water for injection, filtered through a 0.2 micron membrane filter and packaged under sterile conditions.

### Dosage and Administration:

The compounds of the present invention may be formulated in a wide variety of oral administration dosage forms and carriers. Oral administration can be in the form of tablets, coated tablets, dragées, hard and soft gelatin capsules, solutions, emulsions, syrups, or suspensions. Compounds of the present invention are efficacious when administered by other routes of administration including continuous (intravenous drip) topical parenteral, intramuscular, intravenous, subcutaneous, transdermal (which may include a penetration enhancement agent), buccal, nasal, inhalation and suppository administration, among other routes of administration. The preferred manner of administration is generally oral using a convenient daily dosing regimen which can be adjusted according to the degree of affliction and the patient's response to the active ingredient.

A compound or compounds of the present invention, as well as their pharmaceutically useable salts, together with one or more conventional excipients, carriers, or diluents, may be placed into the form of pharmaceutical compositions and unit dosages. The pharmaceutical compositions and unit dosage forms may be comprised of conventional ingredients in conventional proportions, with or without additional active compounds or principles, and the unit dosage forms may contain any suitable effective amount of the active ingredient commensurate with the intended daily dosage range to be employed. The pharmaceutical compositions may be employed as solids, such as tablets or filled capsules, semisolids, powders, sustained release formulations, or liquids such as solutions, suspensions, emulsions, elixirs, or filled capsules for oral use; or in the form of suppositories for rectal or vaginal administration; or in the form of sterile injectable solutions for parenteral use. A typical preparation will contain from about 5% to about 95% active compound or compounds (w/w). The term “preparation” or “dosage form” is intended to include both solid and liquid formulations of the active compound and one skilled in the art will appreciate that an active ingredient can exist in different preparations depending on the target organ or tissue and on the desired dose and pharmacokinetic parameters.

The term “excipient” as used herein refers to a compound that is useful in preparing a pharmaceutical composition, generally safe, non-toxic and neither biologically nor otherwise undesirable, and includes excipients that are acceptable for veterinary use as well as human pharmaceutical use. The compounds of this invention can be administered alone but will generally be administered in admixture with one or more suitable pharmaceutical excipients, diluents or carriers selected with regard to the intended route of administration and standard pharmaceutical practice.

“Pharmaceutically acceptable” means that which is useful in preparing a pharmaceutical composition that is generally safe, non-toxic, and neither biologically nor otherwise undesirable and includes that which is acceptable for veterinary as well as human pharmaceutical use.

A “pharmaceutically acceptable salt” form of an active ingredient may also initially confer a desirable pharmacokinetic property on the active ingredient which were absent in the non-salt form, and may even positively affect the pharmacodynamics of the active ingredient with respect to its therapeutic activity in the body. The phrase “pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

Solid form preparations include powders, tablets, pills, capsules, cachets, suppositories, and dispersible granules. A solid carrier may be one or more substances which may also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. Suitable carriers include but are not limited to magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Solid form preparations may contain, in addition to the active component, colorants, flavors, stabilizers, buffers, artificial and natural sweeteners, dispersants, thickeners, solubilizing agents, and the like.

Liquid formulations also are suitable for oral administration include liquid formulation including emulsions, syrups, elixirs, aqueous solutions, aqueous suspensions. These include solid form preparations which are intended to be converted to liquid form preparations shortly before use. Emulsions may be prepared in solutions, for example, in aqueous propylene glycol solutions or may contain emulsifying agents such as lecithin, sorbitan monooleate, or acacia. Aqueous solutions can be prepared by dissolving the active component in water and adding suitable colorants, flavors, stabilizing, and thickening agents. Aqueous suspensions can be prepared by dispersing the finely divided active component in water with viscous material, such as natural or synthetic gums, resins, methylcellulose, sodium carboxymethylcellulose, and other well known suspending agents.

The compounds of the present invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilization from solution for constitution before use with a suitable vehicle, e.g., sterile, pyrogen-free water.

The compounds of the present invention may be formulated for topical administration to the epidermis as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or coloring agents. Formulations suitable for topical administration in the mouth include lozenges comprising active agents in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

The compounds of the present invention may be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The compounds of the present invention may be formulated for vaginal administration. Pessaries, tampons, creams, gels, pastes, foams or sprays containing in addition to the active ingredient such carriers as are known in the art to be appropriate.

The compounds of the present invention may be formulated for nasal administration. The solutions or suspensions are applied directly to the nasal cavity by conventional means, for example, with a dropper, pipette or spray. The formulations may be provided in a single or multidose form. In the latter case of a dropper or pipette, this may be achieved by the patient administering an appropriate, predetermined volume of the solution or suspension. In the case of a spray, this may be achieved for example by means of a metering atomizing spray pump.

The compounds of the present invention may be formulated for aerosol administration, particularly to the respiratory tract and including intranasal administration. The compound will generally have a small particle size for example of the order of five (5) microns or less. Such a particle size may be obtained by means known in the art, for example by micronization. The active ingredient is provided in a pressurized pack with a suitable propellant such as a chlorofluorocarbon (CFC), for example, dichlorodifluoromethane, trichlorofluoromethane, or dichlorotetrafluoroethane, or carbon dioxide or other suitable gas. The aerosol may conveniently also contain a surfactant such as lecithin. The dose of drug may be controlled by a metered valve. Alternatively the active ingredients may be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition may be presented in unit dose form for example in capsules or cartridges of e.g., gelatin or blister packs from which the powder may be administered by means of an inhaler.

When desired, formulations can be prepared with enteric coatings adapted for sustained or controlled release administration of the active ingredient. For example, the compounds of the present invention can be formulated in transdermal or subcutaneous drug delivery devices. These delivery systems are advantageous when sustained release of the compound is necessary and when patient compliance with a treatment regimen is crucial. Compounds in transdermal delivery systems are frequently attached to a skin-adhesive solid support. The compound of interest can also be combined with a penetration enhancer, e.g., Azone (1-dodecylaza-cycloheptan-2-one). Sustained release delivery systems are inserted subcutaneously into to the subdermal layer by surgery or injection. The subdermal implants encapsulate the compound in a lipid soluble membrane, e.g., silicone rubber, or a biodegradable polymer, e.g., polylactic acid.

Suitable formulations along with pharmaceutical carriers, diluents and excipients are described in Remington: The Science and Practice of Pharmacy 1995, edited by E. W. Martin, Mack Publishing Company, 19th edition, Easton, Pa. A skilled formulation scientist may modify the formulations within the teachings of the specification to provide numerous formulations for a particular route of administration without rendering the compositions of the present invention unstable or compromising their therapeutic activity.

The modification of the present compounds to render them more soluble in water or other vehicle, for example, may be easily accomplished by minor modifications (salt formulation, esterification, etc.), which are well within the ordinary skill in the art. It is also well within the ordinary skill of the art to modify the route of administration and dosage regimen of a particular compound in order to manage the pharmacokinetics of the present compounds for maximum beneficial effect in patients.

The term “therapeutically effective amount” as used herein means an amount required to reduce symptoms of the disease in an individual. The dose will be adjusted to the individual requirements in each particular case. That dosage can vary within wide limits depending upon numerous factors such as the severity of the disease to be treated, the age and general health condition of the patient, other medicaments with which the patient is being treated, the route and form of administration and the preferences and experience of the medical practitioner involved. For oral administration, a daily dosage of between about 0.01 and about 1000 mg/kg body weight per day should be appropriate in monotherapy and/or in combination therapy. A preferred daily dosage is between about 0.1 and about 500 mg/kg body weight, more preferred 0.1 and about 100 mg/kg body weight and most preferred 1.0 and about 10 mg/kg body weight per day. Thus, for administration to a 70 kg person, the dosage range would be about 7 mg to 0.7 g per day. The daily dosage can be administered as a single dosage or in divided dosages, typically between 1 and 5 dosages per day. Generally, treatment is initiated with smaller dosages which are less than the optimum dose of the compound. Thereafter, the dosage is increased by small increments until the optimum effect for the individual patient is reached. One of ordinary skill in treating diseases described herein will be able, without undue experimentation and in reliance on personal knowledge, experience and the disclosures of this application, to ascertain a therapeutically effective amount of the compounds of the present invention for a given disease and patient.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

### Indications and Method of Treatment

### Indications

The compounds of the invention and their isomeric forms and pharmaceutically acceptable salts thereof are useful in treating and preventing HCV infection.

The application provides a method for treating a Hepatitis C Virus (HCV) infection comprising administering to a patient in need thereof a therapeutically effective amount of a compound of any one of Formulae I-III.

The application provides a method for inhibiting replication of HCV in a cell comprising administering a compound of any one of Formulae I-III.

### Combination Therapy

The compounds of the invention and their isomeric forms and pharmaceutically acceptable salts thereof are useful in treating and preventing HCV infection alone or when used in combination with other compounds targeting viral or cellular elements or functions involved in the HCV lifecycle. Classes of compounds useful in the invention include, without limitation, all classes of HCV antivirals.

For combination therapies, mechanistic classes of agents that can be useful when combined with the compounds of the invention include, for example, nucleoside and non-nucleoside inhibitors of the HCV polymerase, protease inhibitors, helicase inhibitors, NS4B inhibitors and medicinal agents that functionally inhibit the internal ribosomal entry site (IRES) and other medicaments that inhibit HCV cell attachment or virus entry, HCV RNA translation, HCV RNA transcription, replication or HCV maturation, assembly or virus release. Specific compounds in these classes and useful in the invention include, but are not limited to, macrocyclic, heterocyclic and linear HCV protease inhibitors such as telaprevir (VX-950), boceprevir (SCH-503034), narlaprevir (SCH-9005 18), ITMN-191 (R-7227), TMC-435350 (a.k.a. TMC-435), MK-7009, BI-201335, BI-2061 (ciluprevir), BMS-650032, ACH-1625, ACH-1095 (HCV NS4A protease co-factor inhibitor), VX-500, VX-8 13, PHX-1766, PHX2054, IDX-136, IDX-3 16, ABT-450 EP-0 13420 (and congeners) and VBY-376; the Nucleosidic HCV polymerase (replicase) inhibitors useful in the invention include, but are not limited to, R7128, PSI-785 1, IDX-184, IDX-102, R1479, UNX-08 189, PSI-6130, PSI-938 and PSI-879 and various other nucleoside and nucleotide analogs and HCV inhibitors including (but not limited to) those derived as 2′-C-methyl modified nucleos(t)ides, 4′-aza modified nucleos(t)ides, and 7′-deaza modified nucleos(t)ides. Non-nucleosidic HCV polymerase (replicase) inhibitors useful in the invention, include, but are not limited to, HCV-796, HCV-371, VCH-759, VCH-916, VCH-222, ANA-598, MK-3281, ABT-333, ABT-072, PF-00868554, BI-207127, GS-9190, A-837093, JKT-109, GL-59728 and GL-60667.

In addition, compounds of the invention can be used in combination with cyclophyllin and immunophyllin antagonists (e.g., without limitation, DEBIO compounds, NM-811 as well as cyclosporine and its derivatives), kinase inhibitors, inhibitors of heat shock proteins (e.g., HSP90 and HSP70), other immunomodulatory agents that can include, without limitation, interferons (-alpha, -beta, -omega, -gamma, -lambda or synthetic) such as Intron A, Roferon-A, Canferon-A300, Advaferon, Infergen, Humoferon, Sumiferon MP, Alfaferone, IFN-β, Feron and the like; polyethylene glycol derivatized (pegylated) interferon compounds, such as PEG interferon-α-2a (Pegasys), PEG interferon-α-2b (PEGIntron), pegylated IFN-α-con1 and the like; long acting formulations and derivatizations of interferon compounds such as the albumin-fused interferon, Albuferon, Locteron, and the like; interferons with various types of controlled delivery systems (e.g., ITCA-638, omega-interferon delivered by the DUROS subcutaneous delivery system); compounds that stimulate the synthesis of interferon in cells, such as resiquimod and the like; interleukins; compounds that enhance the development of type 1 helper T cell response, such as SCV-07 and the like; TOLL-like receptor agonists such as CpG-10101 (actilon), isotorabine, ANA773 and the like; thymosin α-1; ANA-245 and ANA-246; histamine dihydrochloride; propagermanium; tetrachlorodecaoxide; ampligen; IMP-321; KRN-7000; antibodies, such as civacir, XTL-6865 and the like and prophylactic and therapeutic vaccines such as InnoVac C, HCV E1E2/MF59 and the like. In addition, any of the above-described methods involving administering an NS5A inhibitor, a Type I interferon receptor agonist (e.g., an IFN-α) and a Type II interferon receptor agonist (e.g., an IFN-γ) can be augmented by administration of an effective amount of a TNF-α antagonist. Exemplary, non-limiting TNF-α antagonists that are suitable for use in such combination therapies include ENBREL, REMICADE, and HUMIRA.

In addition, compounds of the invention can be used in combination with antiprotozoans and other antivirals thought to be effective in the treatment of HCV infection such as, without limitation, the prodrug nitazoxanide. Nitazoxanide can be used as an agent in combination with the compounds disclosed in this invention as well as in combination with other agents useful in treating HCV infection such as peginterferon α-2a and ribavirin.

Compounds of the invention can also be used with alternative forms of interferons and pegylated interferons, ribavirin or its analogs (e.g., tarabavarin, levoviron), microRNA, small interfering RNA compounds (e.g., SIRPLEX-140-N and the like), nucleotide or nucleoside analogs, immunoglobulins, hepatoprotectants, anti-inflammatory agents and other inhibitors of NS5A. Inhibitors of other targets in the HCV lifecycle include NS3 helicase inhibitors; NS4A co-factor inhibitors; antisense oligonucleotide inhibitors, such as ISIS-14803, AVI-4065 and the like; vector-encoded short hairpin RNA (shRNA); HCV specific ribozymes such as heptazyme, RPI, 13919 and the like; entry inhibitors such as HepeX-C, HuMax-HepC and the like; alpha glucosidase inhibitors such as celgosivir, UT-231B and the like; KPE-02003002 and BIVN 401 and IMPDH inhibitors. Other illustrative HCV inhibitor compounds include those disclosed in the following publications: U.S. Pat. Nos. 5,807,876; 6,498,178; 6,344,465; and 6,054,472; PCT Patent Application Publication Nos. WO97/40028; WO98/4038 1; WO00/56331, WO02/04425; WO03/007945; WO03/010141; WO03/000254; WO01/32153; WO00/06529; WO00/18231; WO00/10573; WO00/13708; WO01/85172; WO03/037893; WO03/037894; WO03/037895; WO02/100851; WO02/100846; WO99/01582; WO00/09543; WO02/18369; WO98/17679, WO00/056331; WO98/22496; WO99/07734; WO05/073216, WO05/073195 and WO08/021,927.

Additionally, combinations of, for example, ribavirin and interferon, may be administered as multiple combination therapy with at least one of the compounds of the invention. The present invention is not limited to the aforementioned classes or compounds and contemplates known and new compounds and combinations of biologically active agents. It is intended that combination therapies of the present invention include any chemically compatible combination of a compound of this inventive group with other compounds of the inventive group or other compounds outside of the inventive group, as long as the combination does not eliminate the anti-viral activity of the compound of this inventive group or the anti-viral activity of the pharmaceutical composition itself.

Combination therapy can be sequential, that is treatment with one agent first and then a second agent (for example, where each treatment comprises a different compound of the invention or where one treatment comprises a compound of the invention and the other comprises one or more biologically active agents) or it can be treatment with both agents at the same time (concurrently). Sequential therapy can include a reasonable time after the completion of the first therapy before beginning the second therapy. Treatment with both agents at the same time can be in the same daily dose or in separate doses. Combination therapy need not be limited to two agents and may include three or more agents. The dosages for both concurrent and sequential combination therapy will depend on absorption, distribution, metabolism and excretion rates of the components of the combination therapy as well as other factors known to one of skill in the art. Dosage values will also vary with the severity of the condition to be alleviated. It is to be further understood that for any particular subject, specific dosage regimens and schedules may be adjusted over time according to the individual's need and the judgment of the one skilled in the art administering or supervising the administration of the combination therapy.

The application provides a method for treating a Hepatitis C Virus (HCV) infection comprising administering to a patient in need thereof a therapeutically effective amount of a compound of any one of Formulae I-III.

The application provides the above method, further comprising administering an immune system modulator or an antiviral agent that inhibits replication of HCV, or a combination thereof. The application provides the above method, wherein the immune system modulator is an interferon or chemically derivatized interferon.

The application provides the above methods, wherein the antiviral agent is selected from the group consisting of a HCV protease inhibitor, a HCV polymerase inhibitor, a HCV helicase inhibitor, a HCV primase inhibitor, a HCV fusion inhibitor, and a combination thereof.

### EXAMPLES

### Abbreviations

Commonly used abbreviations include: acetyl (Ac), azo-bis-isobutyrylnitrile (AIBN), atmospheres (Atm), 9-borabicyclo[3.3.1]nonane (9-BBN or BBN), 2,2′-bis(diphenylphosphino)-1,1′-binaphthyl (BINAP), tert-butoxycarbonyl (Boc), di-tert-butyl pyrocarbonate or boc anhydride (BOC₂O), benzyl (Bn), butyl (Bu), Chemical Abstracts Registration Number (CASRN), benzyloxycarbonyl (CBZ or Z), carbonyl diimidazole (CDI), 1,4-diazabicyclo[2.2.2]octane (DABCO), diethylaminosulfur trifluoride (DAST), dibenzylideneacetone (dba), 1,5-diazabicyclo[4.3.0]non-5-ene (DBN), 1,8-diazabicyclo[5.4.0]undec-7-ene (DBU), N,N′-dicyclohexylcarbodiimide (DCC), 1,2-dichloroethane (DCE), dichloromethane (DCM), 2,3-Dichloro-5,6-dicyano-1,4-benzoquinone (DDQ), diethyl azodicarboxylate (DEAD), di-iso-propylazodicarboxylate (DIAD), di-iso-butylaluminumhydride (DIBAL or DIBAL-H), di-iso-propylethylamine (DIPEA), N,N-dimethyl acetamide (DMA), 4-N,N-dimethylaminopyridine (DMAP), N,N-dimethylformamide (DMF), dimethyl sulfoxide (DMSO), 1,1′-bis-(diphenylphosphino)ethane (dppe), 1,1′-bis-(diphenylphosphino)ferrocene (dppf), 1-(3-dimethylaminopropyl)-3-ethylcarbodiimide hydrochloride (EDCI), 2-ethoxy-1-ethoxycarbonyl-1,2-dihydroquinoline (EEDQ), ethyl (Et), ethyl acetate (EtOAc), ethanol (EtOH), 2-ethoxy-2H-quinoline-1-carboxylic acid ethyl ester (EEDQ), diethyl ether (Et₂O), ethyl isopropyl ether (EtOiPr), 0-(7-azabenzotriazole-1-yl)-N, N,N′N′-tetramethyluronium hexafluorophosphate acetic acid (HATU), acetic acid (HOAc), 1-N-hydroxybenzotriazole (HOBt), high pressure liquid chromatography (HPLC), iso-propanol (IPA), isopropylmagnesium chloride (iPrMgC1), hexamethyl disilazane (HMDS), liquid chromatography mass spectrometry (LCMS), lithium hexamethyl disilazane (LiHMDS), meta-chloroperoxybenzoic acid (m-CPBA), methanol (MeOH), melting point (mp), MeSO₂— (mesyl or Ms), methyl (Me), acetonitrile (MeCN), m-chloroperbenzoic acid (MCPBA), mass spectrum (ms), methyl t-butyl ether (MTBE), methyl tetrahydrofuran (MeTHF), N-bromosuccinimide (NBS), n-Butyllithium (nBuLi), N-carboxyanhydride (NCA), N-chlorosuccinimide (NCS), N-methylmorpholine (NMM), N-methylpyrrolidone (NMP), pyridinium chlorochromate (PCC), Dichloro-((bis-diphenylphosphino)ferrocenyl) palladium(II) (Pd(dppf)Cl₂), palladium(II) acetate (Pd(OAc)₂), tris(dibenzylideneacetone)dipalladium(0) (Pd₂(dba)₃), pyridinium dichromate (PDC), phenyl (Ph), propyl (Pr), iso-propyl (i-Pr), pounds per square inch (psi), pyridine (pyr), 1,2,3,4,5-Pentaphenyl-1′-(di-tert-butylphosphino)ferrocene (Q-Phos), room temperature (ambient temperature, rt or RT), sec-Butyllithium (sBuLi), tert-butyldimethylsilyl or t-BuMe₂Si (TBDMS), tetra-n-butylammonium fluoride (TBAF), triethylamine (TEA or Et₃N), 2,2,6,6-tetramethylpiperidine 1-oxyl (TEMPO), triflate or CF₃SO₂— (Tf), trifluoroacetic acid (TFA), 1,1′-bis-2,2,6,6-tetramethylheptane-2,6-dione (TMHD), O-benzotriazol-1-yl-N,N,N′,N′-tetramethyluronium tetrafluoroborate (TBTU), thin layer chromatography (TLC), tetrahydrofuran (THF), trimethylsilyl or Me₃Si (TMS), p-toluenesulfonic acid monohydrate (TsOH or pTsOH), 4-Me-C₆H₄SO₂— or tosyl (Ts), and N-urethane-N-carboxyanhydride (UNCA). Conventional nomenclature including the prefixes normal (n), iso (i-), secondary (sec-), tertiary (tert-) and neo have their customary meaning when used with an alkyl moiety. (J. Rigaudy and D. P. Klesney, Nomenclature in Organic Chemistry, IUPAC 1979 Pergamon Press, Oxford.).

### General Conditions

Compounds of the invention can be made by a variety of methods depicted in the illustrative synthetic reactions described below in the Examples section.

The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, 1991, Volumes 1-15; Rodd's Chemistry of Carbon Compounds, Elsevier Science Publishers, 1989, Volumes 1-5 and Supplementals; and Organic Reactions, Wiley & Sons: New York, 1991, Volumes 1-40. It should be appreciated that the synthetic reaction schemes shown in the Examples section are merely illustrative of some methods by which the compounds of the invention can be synthesized, and various modifications to these synthetic reaction schemes can be made and will be suggested to one skilled in the art having referred to the disclosure contained in this application.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specified to the contrary, the reactions described herein are typically conducted under an inert atmosphere at atmospheric pressure at a reaction temperature range of from about −78° C. to about 150° C., often from about 0° C. to about 125° C., and more often and conveniently at about room (or ambient) temperature, e.g., about 20° C.

Various substituents on the compounds of the invention can be present in the starting compounds, added to any one of the intermediates or added after formation of the final products by known methods of substitution or conversion reactions. If the substituents themselves are reactive, then the substituents can themselves be protected according to the techniques known in the art. A variety of protecting groups are known in the art, and can be employed. Examples of many of the possible groups can be found in “Protective Groups in Organic Synthesis” by Green et al., John Wiley and Sons, 1999. For example, nitro groups can be added by nitration and the nitro group can be converted to other groups, such as amino by reduction, and halogen by diazotization of the amino group and replacement of the diazo group with halogen. Acyl groups can be added by Friedel-Crafts acylation. The acyl groups can then be transformed to the corresponding alkyl groups by various methods, including the Wolff-Kishner reduction and Clemmenson reduction. Amino groups can be alkylated to form mono- and di-alkylamino groups; and mercapto and hydroxy groups can be alkylated to form corresponding ethers. Primary alcohols can be oxidized by oxidizing agents known in the art to form carboxylic acids or aldehydes, and secondary alcohols can be oxidized to form ketones. Thus, substitution or alteration reactions can be employed to provide a variety of substituents throughout the molecule of the starting material, intermediates, or the final product, including isolated products.

### PREPARATIVE EXAMPLES

### Intermediate 1

### Methyl(S)-1-((S)-4,4-difluoro-2-(5-(4-(4,4,5,5-tetramethyl-1,3,2-dioxaborolan-2-yl)phenyl)-1H-imidazol-2-yl)pyrrolidin-1-yl)-3-methyl-1-oxobutan-2-yl-carbamate

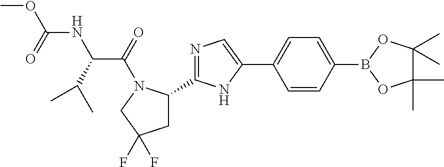

In a 50 mL pear-shaped flask, (S)-1-(tert-butoxycarbonyl)-4,4-difluoropyrrolidine-2-carboxylic acid (1 g, 3.98 mmol, Eq: 1.00) and HATU (1.51 g, 3.98 mmol, Eq: 1.00) were combined with DMF (15 ml) to give a colorless solution and stirred at rt for 10 min. 2-Amino-1-(4-bromophenyl)ethanone HCl (995 mg, 3.98 mmol, Eq: 1.00) was added followed by dropwise addition of N,N′-diisopropylethylamine (1.54 g, 2.09 ml, 11.9 mmol, Eq: 3). The suspension became a orange solution once the addition of the amine was completed. It was stirred at room temperature for 1 hr and diluted with brine (100 ml) and H₂O (50 ml). The precipitate was filtered, washed with H₂O and dried to afford (S)-tert-butyl2-(2-(4-bromophenyl)-2-oxoethylcarbamoyl)-4,4-difluoropyrrolidine-1-carboxylate as a light yellow solid. (1.8 g, >96%): ESI-LRMS m/e calcd for C₁₈H₂₁BrF₂N₂O₄ [M⁺] 447, found 448 [M+H⁺].

In a 50 mL seal tube, (S)-tert-butyl 2-(2-(4-bromophenyl)-2-oxoethylcarbamoyl)-4,4-difluoropyrrolidine-1-carboxylate (1.8 g, 4.02 mmol, Eq: 1.00) and acetic acid, ammonia acetate salt (1.55 g, 20.1 mmol, Eq: 5.00) were combined with xylene (16 ml). The reaction mixture was heated to 140° C. and stirred for 4 hr. The reaction mixture was cooled and diluted with EtOAc (50 ml). It was washed with water and brine, dried with MgSO₄, concentrated and purified on a silica gel column (CH₂Cl₂, 30%, 50%, 80% EtOAc/CH₂Cl₂) to afford (S)-tert-butyl 24544-bromophenyl)-1H-imidazol-2-yl)-4,4-difluoropyrrolidine-1-carboxylate as a yellow solid (1.77 g, 74%): ESI-LRMS m/e calcd for C₁₈H₂₂BrF₂N₃O₂ [M⁺] 428, found 429 [M+H⁺].

In a 10 mL pear-shaped flask, (S)-tert-butyl 2-(5-(4-bromophenyl)-1H-imidazol-2-yl)-4,4-difluoropyrrolidine-1-carboxylate (1.0 g, 2.33 mmol, Eq: 1.00) was combined with CH₂Cl₂ (6 ml) to give a light yellow solution. TFA (2.96 g, 2 mL, 26.0 mmol, Eq: 11.1) was added and stirred for 2 hr. It was concentrated in vacuo to afford (S)-5-(4-bromophenyl)-2-(4,4-difluoropyrrolidin-2-yl)-1H-imidazole as a viscous oil and used for the next step without further purification.

In a 20 mL pear-shaped flask, (S)-2-(methoxycarbonylamino)-3-methylbutanoic acid (416 mg, 2.38 mmol, Eq: 1.3) and HATU (695 mg, 1.83 mmol, Eq: 1.00) were combined with DMF (10 ml) to give a colorless solution. (S)-5-(4-bromophenyl)-2-(4,4-difluoropyrrolidin-2-yl)-1H-imidazole (600 mg, 1.83 mmol, Eq: 1.00) in 2 ml of DMF was added and followed by drop wise addition of N,N′-diisopropylethylamine (1.18 g, 1.6 ml, 9.14 mmol, Eq: 5). It was stirred at room temperature for 1 hr then poured into ice/water. It was extracted with EtOAc (2×30 ml), washed with brine and water. The organic layer was dried over MgSO₄, concentrated and purified on a silica gel column (CH₂Cl₂, 1%, 2%, 5% MeOH/CH₂Cl₂) to afford methyl (S)-1-((S)-2-(5-(4-bromophenyl)-1H-imidazol-2-yl)-4,4-difluoropyrrolidin-1-yl)-3-methyl-1-oxobutan-2-yl-carbamate as an orange foaming solid. (650 mg, 73%): ESI-LRMS m/e calcd for C₂₀H₂₅BrF₂N₄O₃ [M⁺] 485, found 486 [M+H⁺].

In a 20 mL seal tube, methyl (S)-1-((S)-2-(5-(4-bromophenyl)-1H-imidazol-2-yl)-4,4-difluoropyrrolidin-1-yl)-3-methyl-1-oxobutan-2-yl-carbamate (300 mg, 618 μmol, Eq: 1.00), 4,4,4′,4′,5,5,5′,5′-octamethyl-2,2′-bi(1,3,2-dioxaborolane) (471 mg, 1.85 mmol, Eq: 3.0) and potassium acetate (303 mg, 3.09 mmol, Eq: 5.0) were combined with 1,4-dioxane (6 ml) to give a light yellow suspension. It was degassed for 20 min. 1,1′-Bis(diphenylphosphino)ferrocene-palladium(II)dichloride dichloromethane complex (50.5 mg, 61.8 μmol, Eq: 0.10) was added, flushed with N₂, sealed heating at 80° C. for 16 hr. It was cooled and diluted with EtOAc (40 ml). The mixture was washed with brine and water, dried with MgSO₄, concentrated and purified on a silica gel column (CH₂Cl₂, 1%, 2%, 3% to 5% MeOH/CH₂Cl₂) to afford methyl (S)-1-((S)-4,4-difluoro-2-(5-(4-(4,4,5,5-tetramethyl-1,3,2-dioxaborolan-2-yl)phenyl)-1H-imidazol-2-yl)pyrrolidin-1-yl)-3-methyl-1-oxobutan-2-yl-carbamate as an orange solid. (300 mg, 87%): ESI-LRMS m/e calcd for C₂₆H₃₇BF₂N₄O₅ [M⁺] 532, found 533 [M+H⁺].

### Intermediate 2

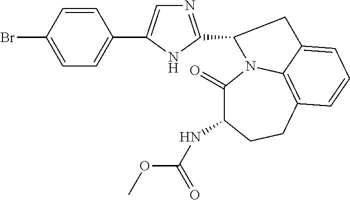

N,N′ diisopropylethylamine (414 mg, 3.20 mmol) was added dropwise at room temperature to a heterogeneous mixture of (2S,5S)-FMOC-5-amino-1,2,4,5,6,7-hexahydroazepino[3,2,1-Hi]indol-4-one-2-carboxylic acid (500 mg, 1.07 mmol), 2-amino-1-(4-bromo-phenyl)-ethanone hydrochloride (267 mg, 1.07 mmol), HATU (406 mg, 1.07 mmol) and DMF (17 ml). After addition was complete the reaction was stirred at room temperature for 16 h. The reaction mixture was diluted with ethyl acetate and washed with water, 1N hydrochloric acid, a saturated sodium bicarbonate solution, a saturated sodium chloride solution and dried over magnesium sulfate, filtered and concentrated to afford, {(2S,5S)-2-[2-(4-bromo-phenyl)-2-oxo-ethylcarbamoyl]-4-oxo-1,2,4,5,6,7-hexahydro-azepino[3,2,1-hi]indol-5-yl]carbamic acid (9H-fluoren-9-yl)methyl ester as a light yellow solid, (602 mg, 85%): ESI-LRMS m/e calcd for C₃₆H₃₀BrN₃O₅ [M⁺] 664, found 665 [M+H⁺].

To a stirred mixture of {(2S,5S)-2-[2-(4-bromo-phenyl)-2-oxo-ethylcarbamoyl]-4-oxo-1,2,4,5,6,7-hexahydro-azepino[3,2,1-hi]indol-5-yl]carbamic acid (9H-fluoren-9-yl)methyl ester (600 mg, 0.90 mmol) dissolved in DMF (10 ml) was added piperidine (2 ml). The mixture was stirred at room temperature for 1 h and then concentrated in vacuo to afford, (2S,5S)-5-amino-4-oxo-1,2,4,5,6,7-hexahydro-azepino[3,2,1-hi]indole-2-carboxylic acid [2-(4-bromo-phenyl)-2-oxo-ethyl]-amide as a yellow powder, (400 mg, 100%): ESI-LRMS m/e calcd for C₂₁H₂₀BrN₃O₃ [M⁺] 442, found 443 [M+H⁺].

To an iced cooled solution of (2S,5S)-5-amino-4-oxo-1,2,4,5,6,7-hexahydro-azepino[3,2,1-hi]indole-2-carboxylic acid [2-(4-bromo-phenyl)-2-oxo-ethyl]-amide (400 mg, 0.90 mmol) dissolved in DMF (12 ml) was added sodium carbonate (115 mg, 1.09 mmol) and methyl chloroformate (94 mg, 1.00 mmol). After the addition was complete the ice bath was removed and the reaction stirred at room temperature for 1 h. The reaction mixture was diluted with ethyl acetate and washed with water, a saturated sodium chloride solution and dried over magnesium sulfate, filtered and concentrated. The crude product obtained was purified by ISCO flash chromatography (Teledyne Isco RediSep Flash Column 40 g; (0% to 100% ethyl acetate/hexane) to afford, {(2S,5S)-2-[2-(4-bromo-phenyl)-2-oxo-ethylcarbamoyl]-4-oxo-1,2,4,5,6,7-hexahydro-azepino[3,2,1-hi]indol-5-yl}-carbamic acid methyl ester as a white solid, (175 mg, 39%): ESI-LRMS m/e calcd for C₂₃H₂₃BrN₃O₅ [M⁺] 500, found 501 [M+H⁺].

A mixture of {(2S,5S)-2-[2-(4-bromo-phenyl)-2-oxo-ethylcarbamoyl]-4-oxo-1,2,4,5,6,7-hexahydro-azepino[3,2,1-hi]indol-5-yl}-carbamic acid methyl ester (174 mg, 0.35 mmol) and ammonium acetate (134 mg, 1.74 mmol) in xylenes (10 ml) was heated in a sealed tube at 140° C. for 4 h. The reaction was then cooled to room temperature and diluted with ethyl acetate. The organic fraction was washed with a saturated sodium bicarbonate solution, a saturated sodium chloride solution and dried over magnesium sulfate, filtered and concentrated. The crude product obtained was purified by ISCO flash chromatography (Teledyne Isco RediSep Flash Column 40 g; (30% to 100% ethyl acetate/hexane) to afford, {(2S,5S)-2-[5-(4-bromo-phenyl)-1H-imidazol-2-yl]-4-oxo-1,2,4,5,6,7-hexahydro-azepino[3,2,1-hi]indol-5-yl}-carbamic acid methyl ester as a white solid, (175 mg, 39%): ESI-LRMS m/e calcd for C₂₃H₂₁BrN₄O₃ [M⁺]481, found 482 [M+H⁺].

### Intermediate 3

### Methyl (S)-1-((2S,4S)-4-fluoro-2-(5-(4-(4,4,5,5-tetramethyl-1,3,2-dioxaborolan-2-yl)phenyl)-1H-imidazol-2-yl)pyrrolidin-1-yl)-3-methyl-1-oxobutan-2-ylcarbamate

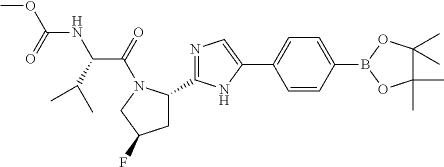

Intermediate 3 was prepared according to the procedure outlined in the preparation of Intermediate 1.

In a 20 mL seal tube, methyl (S)-1-((2S,4S)-2-(5-(4-bromophenyl)-1H-imidazol-2-yl)-4-fluoropyrrolidin-1-yl)-3-methyl-1-oxobutan-2-ylcarbamate (300 mg, 642 μmol, Eq: 1.00), 4,4,4′,4′,5,5,5′,5′-octamethyl-2,2′-bi(1,3,2-dioxaborolane) (489 mg, 1.93 mmol, Eq: 3.0) and potassium acetate (315 mg, 3.21 mmol, Eq: 5.0) were combined with 1,4-dioxane (6.00 ml) to give a light yellow suspension and degassed for 20 min. 1,1′-Bis(diphenylphosphino)ferrocene-palladium(II)dichloride dichloromethane complex (52.4 mg, 64.2 μmol, Eq: 0.10) was added and flushed with N₂. It was sealed and stirred at 80° C. for 16 hr. It was cooled and diluted with EtOAc (40 ml). The reaction mixture was filtered through celite, concentrated and purified on a silica gel column (CH₂Cl₂, 1%, 2%, 3% to 5% MeOH/CH₂Cl₂) to afford methyl (S)-1-((2S,4S)-4-fluoro-2-(5-(4-(4,4,5,5-tetramethyl-1,3,2-dioxaborolan-2-yl)phenyl)-1H-imidazol-2-yl)pyrrolidin-1-yl)-3-methyl-1-oxobutan-2-ylcarbamate as a light brown solid (300 mg, 90.9%). ESI-LRMS m/e calcd for C₂₆H₃₈BF₁N₄O₅ [M⁺]514, found 515 [M+H⁺].

### Intermediate 4

### Methyl (S)-1-((2S,4R)-4-fluoro-2-(5-(4-(4,4,5,5-tetramethyl-1,3,2-dioxaborolan-2-yl)phenyl)-1H-imidazol-2-yl)pyrrolidin-1-yl)-3-methyl-1-oxobutan-2-ylcarbamate

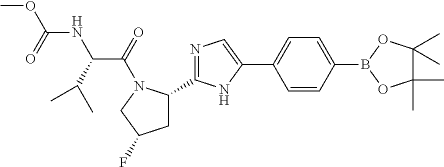

Intermediate 4 was prepared according to the procedure outlined in the preparation of Intermediate 1.

In a 20 mL seal tube, methyl (S)-1-((2S,4R)-2-(5-(4-bromophenyl)-1H-imidazol-2-yl)-4-fluoropyrrolidin-1-yl)-3-methyl-1-oxobutan-2-ylcarbamate (300 mg, 642 μmol, Eq: 1.00), 4,4,4′,4′,5,5,5′,5′-octamethyl-2,2′-bi(1,3,2-dioxaborolane) (489 mg, 1.93 mmol, Eq: 3.0) and potassium acetate (315 mg, 3.21 mmol, Eq: 5.0) were combined with 1,4-dioxane (6.00 ml) to give a light yellow suspension and degassed for 20 min. 1,1′-Bis(diphenylphosphino)ferrocene-palladium(II)dichloride dichloromethane complex (52.4 mg, 64.2 μmol, Eq: 0.10) was added and flushed with N₂. It was sealed and stirred at 80° C. for 16 hr. It was cooled and diluted with EtOAc (20 ml). The reaction mixture was filtered, concentrated and purified on a silica gel column (CH₂Cl₂, 1%, 2%, 3% to 5% MeOH/CH₂Cl₂) to afford methyl (S)-1-((2S,4R)-4-fluoro-2-(5-(4-(4,4,5,5-tetramethyl-1,3,2-dioxaborolan-2-yl)phenyl)-1H-imidazol-2-yl)pyrrolidin-1-yl)-3-methyl-1-oxobutan-2-ylcarbamate as an orange solid (265 mg, 80.3%). ESI-LRMS m/e calcd for C₂₆H₃₈BF₁N₄O₅ [M⁺] 514, found 515 [M+H⁺].

### Intermediate 5

### Methyl (S)-3-methyl-1-oxo-1-((S)-2-(5-(4-(4,4,5,5-tetramethyl-1,3,2-dioxaborolan-2-yl)phenyl)-1H-imidazol-2-yl)pyrrolidin-1-yl)butan-2-ylcarbamate

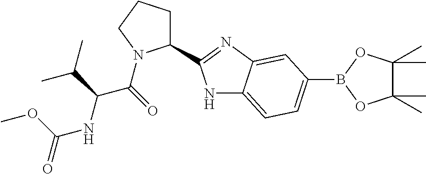

In a 10 mL pear-shaped flask, 4-bromobenzene-1,2-diamine (400 mg, 2.14 mmol, Eq: 1.00), (S)-1-(tert-butoxycarbonyl)pyrrolidine-2-carboxylic acid (414 mg, 1.92 mmol, Eq: 0.9) and N,N′-diisopropylethylamine (829 mg, 1.12 ml, 6.42 mmol, Eq: 3.00) were combined with DMF (3 ml) to give a red solution. HATU (813 mg, 2.14 mmol, Eq: 1.00) was added and stirred at room temperature for 2 hr. It was diluted with brine and H₂O and extracted with EtOAc (2×50 ml). The combined organic layers was washed with brine and water, dried over MgSO₄ concentrated in vacuo. The crude mixture was purified on a silica gel column (CH₂Cl₂, 20%, 50% EtOAc/CH₂Cl₂) to afford (S)-tert-butyl-2-(amino-4-bromophenylcarbamoyl)pyrrolidine-1-carboxylate as a red solid (730 mg, 88.8%). ESI-LRMS m/e calcd for C₂₆H₃₈BF₁N₄O₅ [M⁺] 514, found 515 [M+H⁺].

In a 10 mL pear-shaped flask, (S)-tert-butyl 2-(2-amino-4-bromophenyl carbamoyl)pyrrolidine-1-carboxylate (100 mg, 260 μmol, Eq: 1.00), ammonium acetate (200 mg, 2600 mmol, Eq: 10.0), were combined with acetic acid (1.05 g, 1.0 ml, 17.5 mmol, Eq: 67.1) to give a light red solution. The reaction mixture was heated at 90° C. and stirred for 60 min. The reaction mixture was cooled and diluted with EtOAc (60 ml), washed with saturated NaHCO₃ solution (50 ml) and water. It was dried and concentrated in vacuo to afford (S)-tert-butyl 2-(5-bromo-1H-benzo[d]imidazol-2-yl)pyrrolidine-1-carboxylate and used for the next reaction without further purification (3.0 g, 95%).

In a 10 mL round-bottomed flask, (S)-tert-butyl 2-(5-bromo-1H-benzo[d]imidazol-2-yl)pyrrolidine-1-carboxylate (3.0 g, 8.19 mmol, Eq: 1.00) was combined with TFA (9.34 g, 6.31 ml, 81.9 mmol, Eq: 10) in CH₂Cl₂ (15 ml) to give a black solution. It was stirred at room temperature for 2 hr. The mixture was concentrated in vacuo to afford (S)-2-(methoxycarbonylamino)-3-methylbutanoic acid as an oil and used without further purification. (1.97 g, 85%)

In a 100 mL round-bottomed flask, (S)-2-(methoxycarbonylamino)-3-methylbutanoic acid (1.97 g, 11.3 mmol, Eq: 1.5) and HATU (2.86 g, 7.51 mmol, Eq: 1.00) were combined with DMF (15 ml) to give a colorless solution. (S)-5-bromo-2-(pyrrolidin-2-yl)-1H-benzo[d]imidazole (2.0 g, 7.51 mmol, Eq: 1.00) in 5 ml DMF added and cooled to 0° C. followed by addition of N,N′-diisopropylethylamine (4.86 g, 6.56 ml, 37.6 mmol, Eq: 5). It was stirred at room temperature for 1 hr then diluted with brine and extracted with EtOAc (2×50 ml). The combined organic layers were washed with brine and H₂O, dried over MgSO₄, concentrated in vacuo and purified on a silica gel column (CH₂Cl₂, 2%, 4%, 5% MeOH/CH₂Cl₂) to afford methyl (S)-1-((S)-2-(5-bromo-1H-benzo[d]imidazol-2-yl)pyrrolidin-1-yl)-3-methyl-1-oxobutan-2-ylcarbamate as a solid (2 g, 62.9%). ESI-LRMS m/e calcd for C₁₈H₂₃BrN₄O₃ [M⁺] 423, found 424 [M+H⁺].

In a 20 mL seal tube, methyl (S)-1-((S)-2-(5-bromo-1H-benzo[d]imidazol-2-yl)-pyrrolidin-1-yl)-3-methyl-1-oxobutan-2-ylcarbamate (300 mg, 709 μmol, Eq: 1.00), 4,4,4′,4′,5,5,5′,5′-octamethyl-2,2′-bi(1,3,2-dioxaborolane) (540 mg, 2.13 mmol, Eq: 3) and potassium acetate (348 mg, 3.54 mmol, Eq: 5) were combined with 1,4-dioxane (6 ml) to give a light yellow suspension. It was degassed for 20 min and 1,1′ bis(diphenyl-phosphino)-ferrocene-palladium(II)dichloride dichloromethane complex (57.9 mg, 70.9 μmol, Eq: 0.1) was added. The reaction mixture was flushed with N₂, sealed and stirred at 80° C. for 16 hr. It was cooled, diluted with EtOAc (40 ml) and washed with H₂O. The organic layers were dried with MgSO₄, filtered and concentrated in vacuo. The crude reaction mixture was purified on a silica gel column (CH₂Cl₂, 1%, 2%, 5% to 8% MeOH/CH₂Cl₂) to afford methyl (S)-3-methyl-1-oxo-1-((S)-2-(5-(4,4,5,5-tetramethyl-1,3,2-dioxaborolan-2-yl)-1H-benzo[d]imidazol-2-yl)pyrrolidin-1-yl)butan-2-ylcarbamate as a brown solid (290 mg, 87%). ESI-LRMS. m/e calcd for C₂₄H₃₅BN₄O₅ [M⁺] 470, found 471 [M+H⁺].

### Intermediate 6

### Methyl(S)-1-((S)-4,4-difluoro-2-(5-(4-(4,4,5,5-tetramethyl-1,3,2-dioxaborolan-2-yl)phenyl)-1H-benzo[d]imidazol-2-yl)pyrrolidin-1-yl)-3-methyl-1-oxobutan-2-ylcarbamate

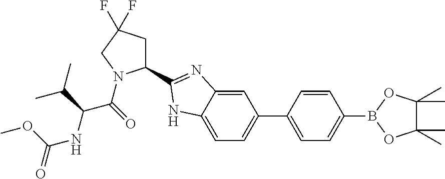

In a 10 mL pear-shaped flask, (S)-1-(tert-butoxycarbonyl)-4,4-difluoropyrrolidine-2-carboxylic acid (1.0 g, 3.98 mmol, Eq: 1.00) and HATU (1.51 g, 3.98 mmol, Eq: 1.00) were combined with DMF (3.0 ml) to give a red solution. 4-Bromobenzene-1,2-diamine (819 mg, 4.38 mmol, Eq: 1.1) and N,N′-diisopropylethylamine (1.54 g, 2.09 ml, 11.9 mmol, Eq: 3.00) were added and stirred for 2 hr. It was diluted with brine and H₂O and extracted with EtOAc (2×50 ml). The organic layer was washed with brine and water, dried over MgSO₄ and concentrated in vacuo. The crude was purified on a silica gel column (CH₂Cl₂, 20%, 50% EtOAc/CH₂Cl₂) to afford (S)-tert-butyl 2-(2-amino-4-bromophenylcarbamoyl)-4,4-difluoropyrrolidine-1-carboxylate as a light brown solid (1.5 g, 89%). ESI-LRMS m/e calcd for C₁₈H₂₀BrF₂N₃O₃ [M⁺] 420, found 421 [M+H⁺].

In a 10 mL pear-shaped flask, (S)-tert-butyl 2-(2-amino-4-bromophenylcarbamoyl)-4,4-difluoropyrrolidine-1-carboxylate (1.5 g, 3.57 mmol, Eq: 1.00) and ammonium acetate (1.4 g, 17.8 mmol, Eq: 5.00) were combined with acetic acid (5.24 g, 5 ml, 87.3 mmol, Eq: 24.5) to give a light red color solution. The reaction mixture was heated to 90° C. and stirred for 1 h. The crude reaction mixture was concentrated in vacuo, diluted with H₂O and extracted with EtOAc (2×50 ml). The combined organic layers were washed with sat. NaHCO₃ solution and H₂O, dried and concentrated to afford (S)-tert-butyl 2-(5-bromo-1H-benzo[d]imidazol-2-yl)-4,4-difluoropyrrolidine-1-carboxylate as a waxy solid (1.26 g, 87.8%). ESI-LRMS m/e calcd for C₁₆H₁₈BrF₂N₃O₂ [M⁺] 402, found 403 [M+H⁺].

In a 10 mL round-bottomed flask, (S)-tert-butyl 2-(5-bromo-1H-benzo[d]imidazol-2-yl)-4,4-difluoropyrrolidine-1-carboxylate (1.26 g, 3.13 mmol, Eq: 1.00) was combined with TFA (3.57 g, 2.41 ml, 31.3 mmol, Eq: 10) in CH₂Cl₂ (6 ml) to give a black solution and stirred at room temperature for 2 hr. It was concentrated in vacuo to afford (S)-5-bromo-2-(4,4-difluoropyrrolidin-2-yl)-1H-benzo[d]imidazole and used for next reaction without further purification (920 mg, 97.2%).

In a 100 mL round-bottomed flask, (S)-2-(methoxycarbonylamino)-3-methylbutanoic acid (640 mg, 3.65 mmol, Eq: 1.2) and HATU (1.27 g, 3.35 mmol, Eq: 1.1) were combined with DMF (15.0 ml) to give a colorless solution. (S)-5-Bromo-2-(4,4-difluoropyrrolidin-2-yl)-1H-benzo[d]imidazole (920 mg, 3.05 mmol, Eq: 1) in 5 ml DMF was added and cooled to 0° C. It was followed by addition of methylenechloride (1.97 g, 2.66 ml, 15.2 mmol, Eq: 5). The reaction was stirred for 1 hr and diluted with brine and water, extracted with EtOAc (2×50 ml). The organic layer was washed with brine and dried with MgSO₄, concentrated in vacuo and purified on a silica gel column (CH₂Cl₂, 20%, 40%, EtOAc/CH₂Cl₂) to afford methyl (S)-1-((S)-2-(5-bromo-1H-benzo[d]imidazol-2-yl)-4,4-difluoropyrrolidin-1-yl)-3-methyl-1-oxobutan-2-ylcarbamate as a solid (948 mg, 67.8%). ESI-LRMS m/e calcd for C₁₈H₂₁BrF₂N₄O₃ [M⁺] 459, found 460 [M+H⁺].

In a 20 mL seal tube, methyl (S)-1-((S)-2-(5-bromo-1H-benzo[d]imidazol-2-yl)-4,4-difluoropyrrolidin-1-yl)-3-methyl-1-oxobutan-2-ylcarbamate (300 mg, 653 μmol, Eq: 1), 4-chlorophenylboronic acid (102 mg, 653 μmol, Eq: 1) and sodium carbonate (saturated solution, 1.0 ml, excess) were combined with 1,4 dioxane (6.00 ml) to give a light yellow solution and degassed for 10 min. Tetrakis(triphenylphosphine)palladium (0) (45.0 mg, 38.9 μmol, Eq: 0.0596) was added and heated at 80° C. for 16 hr. It was cooled and diluted with EtOAc (50 ml). The organic layer was washed with brine and H₂O, dried with MgSO₄, concentrated and purified on a silica gel column (CH₂Cl₂, 2%, 5% and 8% MeOH/CH₂Cl₂) to afford (S)-1-((S)-2-(5-(4-chlorophenyl)-1H-benzo[d]imidazol-2-yl)-4,4-difluoropyrrolidin-1-yl)-3-methyl-1-oxobutan-2-ylcarbamate as an orange solid (170 mg, 53%). ESI-LRMS m/e calcd for C₂₄H₂₅ClF₂N₄O₃ [M⁺] 490, found 491 [M+H⁺].

In a 10 mL seal tube, methyl (S)-1-((S)-2-(5-(4-chlorophenyl)-1H-benzo[d]imidazol-2-yl)-4,4-difluoropyrrolidin-1-yl)-3-methyl-1-oxobutan-2-ylcarbamate (170 mg, 346 μmol, Eq: 1.00), 4,4,4′,4′,5,5,5′,5′-octamethyl-2,2′-bi(1,3,2-dioxaborolane) (264 mg, 1.04 mmol, Eq: 3) and potassium acetate (170 mg, 1.73 mmol, Eq: 5) were combined with 1,4-dioxane (6 ml) to give a yellow suspension. It was degassed for 10 min and tricyclohexylphosphine (120 mg, 429 μmol, Eq: 1.24) and Pd₂(dba)₃ (22.2 mg, 24.2 μmol, Eq: 0.07) were added. The tube was sealed after flushing with N₂ and heated at 90° C. for 50 hr. It was cooled and diluted with EtOAc, filtered through celite and concentrated in vacuo. The residue was purified on a silica gel column (CH₂Cl₂, 2%, 3%, 5% MeOH/CH₂Cl₂) to afford methyl (S)-1-((S)-4,4-difluoro-2-(5-(4-(4,4,5,5-tetramethyl-1,3,2-dioxaborolan-2-yl)phenyl)-1H-benzo[d]imidazol-2-yl)pyrrolidin-1-yl)-3-methyl-1-oxobutan-2-ylcarbamate as a solid (118 mg, 58.5%). ESI-LRMS m/e calcd for C₃₀H₃₇BF₂N₄O₅ [M⁺] 582, found 583 [M+H⁺].

### Intermediate 7

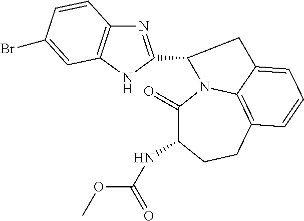

(2S,5S)-Fmoc-5-amino-1,2,4,5,6,7-hexahydro-azepino[3,2,1-HI]indole-4-one-2-carboxylic acid (from a commercial source, 0.8 g, 1.71 mmol), HATU (714 mg, 1.88 mmol) and 4-bromo-1,2-benzenediamine (319 mg, 1.71 mmol) were combined with DMF (20 ml) in a round bottomed flask. N,N-diisopropylethylamine (441 mg, 585 μA, 3.42 mmol, Eq: 2) was added, the resulting mixture was stirred at room temperature for 2 h. The reaction mixture was poured into water and extracted with EtOAc, The organic layers were washed with brine and dried over sodium sulfate and concentrated in vacuo. The crude material was purified by flash chromatography (silica gel, 50% to 80% EtOAc in Hexane) to give a brown solid as compound 2 (a mixture of regioisomers by LCMS) which is used in the next step.

A mixture of (9H-fluoren-9-yl)methyl (2S,5S)-2-(2-amino-4-bromophenylcarbamoyl)-4-oxo-1,2,4,5,6,7-hexahydroazepino[3,2,1-hi]indol-5-ylcarbamate and its regioisomer (1.09 g, 1.71 mmol) was combined with acetic acid (15 ml) to give a brown suspension. The reaction mixture was heated at 90° C. for one hour, then poured into ice water. The pH was adjusted to 6-7 with 3 N NaOH aqueous solution (about 90 ml), collected the solid by filtration, washed with water, the solid was dissolved in dichloromethane, the organic solution were dried over Na₂SO₄ and concentrated in vacuo. Brown solid obtained. The crude material was purified by flash chromatography (silica gel, 20% to 50% EtOAc in hexanes) to give compound 3 as yellow foam (0.877 g, 82% in 2 steps). M+1=619/621.

In a round-bottomed flask, a compound of (9H-fluoren-9-yl)methyl (2S,5S)-2-(6-bromo-1H-benzo[d]imidazol-2-yl)-4-oxo-1,2,4,5,6,7-hexahydroazepino[3,2,1-hi]indol-5-ylcarbamate (0.875 g, 1.41 mmol, Eq: 1.00), were combined with 20% piperidine in DMF (10 ml) to give a yellow solution. The reaction mixture was stirred at RT for 3 h. The reaction mixture was poured into water, and extracted with EtOAc. The organic layers were washed with brine and dried over Na₂SO₄ and concentrated in vacuo. The crude material was purified by silica gel plug (silica gel, 7% MeOH in DCM/conc. NH₄OH) to give compound 4 as yellow foam (0.57 g, 100%). [M⁺+]-397/399.

In a round-bottomed flask, a compound of (2S,5S)-5-amino-2-(6-bromo-1H-benzo[d]imidazol-2-yl)-1,2,6,7-tetrahydroazepino[3,2,1-hi]indol-4(5H)-one (0.57 g, 1.43 mmol, Eq: 1.00), was combined with dichloromethane (20 ml) to give a yellow solution. Pyridine (227 mg, 232 μl, 2.87 mmol, Eq: 2) and methyl chloroformate (155 mg, 1.65 mmol, Eq: 1.15) were added at 0° C., the mixture was stirred in ice bath about 2 hours, TLC showed there was still some SM left, extra 0.1 ml of methyl chloroformate added, stirred in ice bath about one hour, LC-Mass showed the formation of compound 5, the reaction mixture was poured into water, and extracted with dichloromethane. The organic layers were washed with brine and dried over Na₂SO₄ and concentrated in vacuo, the residue was used for the next step.

The crude material was dissolved in the mixture of THF (10 ml) and conc. NH₄OH aq solution (2 ml), and stirred at RT for 2 hours. The reaction mixture was poured into water and extracted with EtOAc, the organic layers were washed with brine and dried over Na₂SO₄ and concentrated in vacuo. The crude material was purified by silica gel plug (5% MeOH in DCM/Conc. NH₄OH) to give compound 6 as pale yellow foam (0.65 g, 99% in 2 steps). [M+H]⁺-455/457.

### Intermediate 8

### Methyl (S)-3-methyl-1-oxo-1-((S)-2-(6-(4-(4,4,5,5-tetramethyl-1,3,2-dioxaborolan-2-yl)phenyl)-1H-benzo[d]imidazol-2-yl)pyrrolidin-1-yl)butan-2-ylcarbamate

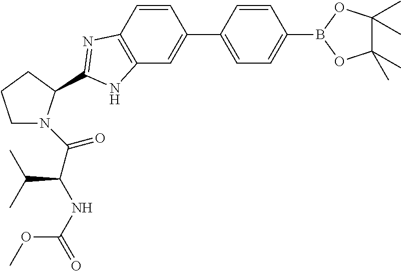

In a 20 mL seal tube, methyl (S)-1-((S)-2-(5-bromo-1H-benzo[d]imidazol-2-yl)pyrrolidin-1-yl)-3-methyl-1-oxobutan-2-ylcarbamate (200 mg, 472 μmol, Eq: 1.00), 4-chlorophenylboronic acid (85.0 mg, 543 μmol, Eq: 1.15) and sodium carbonate (2 ml, excess) were combined with 1,4 dioxane (6 ml) to give a light yellow solution and degassed for 10 min. Tetrakis(triphenylphosphine)palladium (0) (45 mg, 38.9 μmol, Eq: 0.0824) was added and heated at 80° C. for 16 hr. It was cooled, diluted with EtOAc (50 ml), washed with brine and H₂O. The organic layer was dried over MgSO₄, concentrated in vacuo and purified on a silica gel column (CH₂Cl₂, 2%, 5% and 8% MeOH/CH₂Cl₂) to afford methyl (S)-1-((S)-2-(5-(4-chlorophenyl)-1H-benzo[d]imidazol-2-yl)pyrrolidin-1-yl)-3-methyl-1-oxobutan-2-ylcarbamate as an orange solid (135 mg, 62.8%). ESI-LRMS m/e calcd for C₂₄H₂₇ClN₄O₃ [M⁺] 454, found 455 [M+H⁺].

In a 10 mL seal tube, methyl (S)-1-((S)-2-(5-(4-chlorophenyl)-1H-benzo[d]imidazol-2-yl)pyrrolidin-1-yl)-3-methyl-1-oxobutan-2-ylcarbamate (60 mg, 132 μmol, Eq: 1.00), 4,4,4′,4′,5,5,5′,5′-octamethyl-2,2′-bi(1,3,2-dioxaborolane) (100 mg, 396 μmol, Eq: 3) and potassium acetate (64.7 mg, 659 μmol, Eq: 5) and tricyclohexylphosphine (46 mg, 164 μmol, Eq: 1.24) were combined with 1,4-dioxane (6 ml) to give a yellow suspension. It was degassed for 10 min and Pd₂(dba)₃ (8 mg, 8.74 μmol, Eq: 0.0662) was added and sealed after flushing with N₂. It was stirred at room temperature for 30 min and then heated at 80° C. for 30 hr. It was diluted with CH₂Cl₂, filtered through Ceilite, filtered, concentrated in vacuo and purified on a silica gel column (CH₂Cl₂, 1%, 3%, 5%, 8% MeOH/CH₂Cl₂) to afford methyl (S)-3-methyl-1-oxo-1-((S)-2-(6-(4-(4,4,5,5-tetramethyl-1,3,2-dioxaborolan-2-yl)phenyl)-1H-benzo[d]imidazol-2-yl)pyrrolidin-1-yl)butan-2-ylcarbamate as a light yellow solid (90 mg, 93.7%). ESI-LRMS m/e calcd for C₃₀H₃₉BN₄O₅ [M⁺] 546, found 547 [M+H⁺].

### Intermediate 9

### Methyl (S)-3-methyl-1-oxo-1-((S)-2-(6-(6-(4,4,5,5-tetramethyl-1,3,2-dioxaborolan-2-yl)quinoxalin-2-yl)-1H-benzo[d]imidazol-2-yl)pyrrolidin-1-yl)butan-2-ylcarbamate

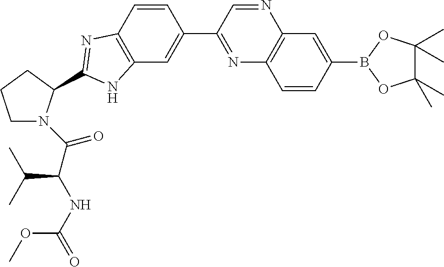

In a 10 mL seal tube, methyl (S)-3-methyl-1-oxo-1-((S)-2-(5-(4,4,5,5-tetramethyl-1,3,2-dioxaborolan-2-yl)-1H-benzo[d]imidazol-2-yl)pyrrolidin-1-yl)butan-2-ylcarbamate (100 mg, 213 mmol, Eq: 1.00), 6-bromo-2-chloroquinoxaline (51.8 mg, 213 μmol, Eq: 1.00) and cesium carbonate (139 mg, 425 μmol, Eq: 2.0) were combined with 1,4 dioxane (2.00 ml) and water (0.4 ml) to give a light brown solution. It was degassed for 10 min and tetrakis(triphenylphosphine)palladium (0) (24.6 mg, 21.3 μmol, Eq: 0.1) was added. The reaction mixture was heated to 80° C. and stirred for 16 h. It was diluted with EtOAc (6 ml), filtered through celite, concentrated in vacuo and purified on a silica gel column (CH₂Cl₂, 2%, 3%, 5%, 8%, 10% MeOH/CH₂Cl₂) to afford methyl (S)-1-((S)-2-(5-(6-bromoquinoxalin-2-yl)-1H-benzo[d]imidazol-2-yl)pyrrolidin-1-yl)-3-methyl-1-oxobutan-2-ylcarbamate as a solid (100 mg, 85.3%). ESI-LRMS m/e calcd for C₂₆H₂₇BrN₆O₃ [M⁺] 551, found 552 [M+H⁺].

In a 20 mL seal tube, methyl (S)-1-((S)-2-(5-(6-bromoquinoxalin-2-yl)-1H-benzo[d]imidazol-2-yl)pyrrolidin-1-yl)-3-methyl-1-oxobutan-2-ylcarbamate (100 mg, 181 μmol, Eq: 1.00), 4,4,4′,4′,5,5,5′,5′-octamethyl-2,2′-bi(1,3,2-dioxaborolane) (138 mg, 544 mmol, Eq: 3) and potassium acetate (89.0 mg, 907 μmol, Eq: 5) were combined with 1,4-dioxane (6 ml) to give a light yellow suspension. It was degassed for 20 min. 1,1′-Bis(diphenylphosphino)ferrocene-palladium(II)dichloride dichloromethane complex (14.8 mg, 18.1 μmol, Eq: 0.1) was added and flushed with N₂. It was sealed and stirred at 80° C. for 16 hr, then cooled and diluted with EtOAc (40 ml). The reaction mixture was filtered, concentrated in vacuo and purified on a silica gel column (CH₂Cl₂, 1%, 2%, 5% to 8% MeOH/CH₂Cl₂) to afford methyl (S)-3-methyl-1-oxo-1-((S)-2-(6-(6-(4,4,5,5-tetramethyl-1,3,2-dioxaborolan-2-yl)quinoxalin-2-yl)-1H-benzo[d]imidazol-2-yl)pyrrolidin-1-yl)butan-2-ylcarbamate as a brown solid [(65 mg, 60% yield), (70% boronic ester and 30% boronic acid (m=516) by LC-MS)]. ESI-LRMS m/e calcd for C₃₂H₃₉BN₆O₅ [M⁺] 598, found 599 [M+H⁺].

### Intermediate 10

### Methyl (S)-3-methyl-1-oxo-1-((S)-2-(5-(5-(4,4,5,5-tetramethyl-1,3,2-dioxaborolan-2-yl)pyridin-2-yl)-1H-benzo[d]imidazol-2-yl)pyrrolidin-1-yl)butan-2-ylcarbamate

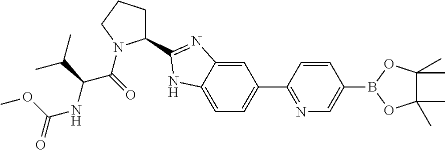

In a 10 mL seal tube, methyl (S)-3-methyl-1-oxo-1-((S)-2-(5-(4,4,5,5-tetramethyl-1,3,2-dioxaborolan-2-yl)-1H-benzo[d]imidazol-2-yl)pyrrolidin-1-yl)butan-2-ylcarbamate (100 mg, 213 mmol, Eq: 1.00), 2-bromo-5-chloropyridine (40.9 mg, 213 μmol, Eq: 1.00) and cesium carbonate (139 mg, 425 μmol, Eq: 2.0) were combined with 1,4 dioxane (2.00 ml) and water (400 μA) to give a light brown solution. It was degassed for 10 min and tetrakis(triphenylphosphine)palladium (0) (24.6 mg, 21.3 μmol, Eq: 0.1) was added. The reaction mixture was heated to 80° C. and stirred for 16 hr. It was diluted with EtOAc (6 ml), concentrated in vacuo and purified on a silica gel column (CH₂Cl₂, 2%, 3%, 5%, 8%, 10% MeOH/CH₂Cl₂) to afford methyl (S)-1-((S)-2-(5-(5-chloropyridin-2-yl)-1H-benzo[d]imidazol-2-yl)pyrrolidin-1-yl)-3-methyl-1-oxobutan-2-ylcarbamate as a solid (86 mg, 88%). ESI-LRMS m/e calcd for C₂₃H₂₆ClN₅O₃ [M⁺] 455, found 456 [M+H⁺].

In a 10 mL seal tube, methyl (S)-1-((S)-2-(5-(5-chloropyridin-2-yl)-1H-benzo[d]imidazol-2-yl)pyrrolidin-1-yl)-3-methyl-1-oxobutan-2-ylcarbamate (86 mg, 189 μmol, Eq: 1.00), 4,4,4′,4′,5,5,5′,5′-octamethyl-2,2′-bi(1,3,2-dioxaborolane) (144 mg, 566 μmol, Eq: 3) and potassium acetate (92.6 mg, 943 μmol, Eq: 5) were combined with 1,4-dioxane (2 ml) to give a yellow suspension. It was degassed for 10 min and tricyclohexylphosphine (65.6 mg, 234 mmol, Eq: 1.24) and Pd₂(dba)₃ (12.1 mg, 13.2 μmol, Eq: 0.07) were added and stirred at room temperature for 10 min. It was sealed after flushing with N₂ and heated at 90° C. for 50 hr. It was cooled and diluted with EtOAc, filtered through celite and concentrated in vacuo to afford methyl (S)-3-methyl-1-oxo-1-((S)-2-(5-(5-(4,4,5,5-tetramethyl-1,3,2-dioxaborolan-2-yl)-1H-benzo[d]imidazol-2-yl)pyrrolidin-1-yl)butan-2-ylcarbamate as a solid which was used for the next reaction without further purification (74 mg, 71.7%). ESI-LRMS m/e calcd for C₂₉H₃₈BN₅O₅ [M⁺] 547, found 548 [M+H⁺].

### Intermediate 11

### Methyl(S)-3-methyl-1-oxo-1-((S)-8-(5-(4-(4,4,5,5-tetramethyl-1,3,2-dioxaborolan-2-yl)phenyl)-1H-imidazol-2-yl)-1,4-dioxa-7-azaspiro[4.4]nonan-7-yl)butan-2-yl-carbamate

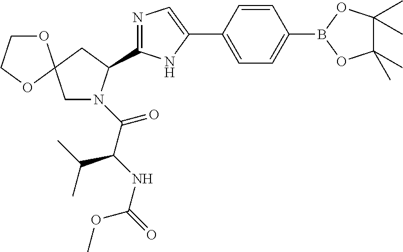

In a 500 ml, round-bottomed flask, (S)-2-(methoxycarbonylamino)-3-methylbutanoic acid (2.90 g, 16.6 mmol, Eq: 1.00) and HATU (6.29 g, 16.6 mmol, Eq: 1.00) were combined with CH₂Cl₂ (300 ml) to give a colorless suspension. TEA (5.03 g, 6.92 ml, 49.7 mmol, Eq: 3.00) was added and stirred at rt for 30 min. (2S,4R)-methyl 4-hydroxypyrrolidine-2-carboxylate HCl (3.01 g, 16.6 mmol, Eq: 1.00) was added and stirred at room temperature for 5 hr. It was diluted with saturated NaHCO₃ (100 ml) and organic phase was separated. The aqueous layer was extracted with CH₂Cl₂ (2×100 ml) and the organic layer was dried with MgSO₄, filtered and concentrated in vacuo. The crude material was purified by a silica gel column (CH₂Cl₂, 20%, 40%, 60%, 80% EtOAc/CH₂Cl₂) to afford (2S,4R)-methyl 4-hydroxy-1-((S)-2-(methoxycarbonylamino)-3-methylbutanoyl)pyrrolidine-2-carboxylate as an oil (4.0 g, 79.9%). ¹H NMR (400 MHz, DMSO-d₆) δ ppm 0.88 (dd, J=17.32, 6.78 Hz, 17H) 1.75-1.96 (m, 5H) 2.10 (br. S., 3 H) 3.46-3.62 (m, 16H) 3.63-3.77 (m, 5H) 4.33 (d, J=8.03 Hz, 5H) 5.21 (d, J=3.76 Hz, 3H) 7.30 (d, J=8.78 Hz, 2H).

In a 100 mL round-bottomed flask, (2S,4R)-methyl 4-hydroxy-1-((S)-2-(methoxycarbonylamino)-3-methylbutanoyl)pyrrolidine-2-carboxylate (0.8 g, 2.65 mmol, Eq: 1.00) was combined with CH₂Cl₂ (20 ml) to give a colorless solution. Dess-Martin periodinane (2.24 g, 5.29 mmol, Eq: 2) was added and stirred at room temperature for 2 hr. It was quenched with 5% sodium thiosulfate (80 ml), NaHCO₃ (sat. solution 100 ml) and stirred for 20 min. The reaction mixture was extracted with CH₂Cl₂ (2×100 ml) and the combined organic layers were dried with MgSO₄, filtered and concentrated in vacuo. The crude product was purified on a silica gel column (CH₂Cl₂, 20%, 40%, 60% EtOAc/CH₂Cl₂) to afford (methoxycarbonylamino)-3-methylbutanoyl)-4-oxopyrrolidine-2-carboxylate as a colorless oil (230 mg, 28.9%). ¹H NMR (400 MHz, CHLOROFORM-d) δ ppm 0.91-1.11 (m, 24H) 1.97-2.11 (m, 10H) 2.56-2.76 (m, 4H) 2.87-3.09 (m, 4H) 3.67 (s, 12H) 3.77 (s, 12H) 4.33-4.58 (m, 3H) 5.03-5.16 (m, 3H) 5.21-5.51 (m, 3H).

In a 50 mL round-bottomed flask, (S)-methyl 1-((S)-2-(methoxycarbonylamino)-3-methylbutanoyl)-4-oxopyrrolidine-2-carboxylate (230 mg, 766 μmol, Eq: 1.00) and ethane-1,2-diol (238 mg, 214 μl, 3.83 mmol, Eq: 5) were combined with toluene (15 ml) to give a colorless solution. P-toluenesulfonic acid monohydrate (29.1 mg, 153 μmol, Eq: 0.2) was added and the reaction mixture was heated to 110° C. with a Dean-Stark apparatus for 20 hr. After cooling, it was diluted with EtOAc (50 ml), washed with NaHCO₃ (sat solution, 200 ml), dried and concentrated in vacuo. The crude mixture was purified on a silica gel column (CH₂Cl₂, 20%, 30%, 40% and 60% EtOAc/CH₂Cl₂) to afford (S)-methyl 7-((S)-2-(methoxycarbonylamino)-3-methylbutanoyl)-1,4-dioxa-7-azaspiro[4.4]nonane-8-carboxylate as an oil (110 mg, 41.7%). ¹H NMR (400 MHz, CHLOROFORM-d) δ ppm 0.95 (d, J=6.78 Hz, 8H) 1.05 (d, J=6.78 Hz, 7H) 1.57 (s, 2H) 2.22 (dd, J=13.18, 6.90 Hz, 3H) 2.37 (dd, J=13.05, 8.78 Hz, 2H) 3.62-3.70 (m, 11H) 3.71-3.87 (m, 10H) 3.93-4.03 (m, 11H) 4.25 (d, J=2.76 Hz, 4 H) 4.59-4.78 (m, 2H) 5.23-5.51 (m, 2H) 5.26-5.51 (m, 2H).

(S)-Methyl 7-((S)-2-(methoxycarbonylamino)-3-methylbutanoyl)-1,4-dioxa-7-azaspiro[4.4]nonane-8-carboxylate (150 mg, 436 μmol) were combined with and tert-butanol (250 μA) to give a colorless solution. LiOH (1M, 871 μA, Eq: 2) was added and stirred at room temperature for 2 hr. It was acidified with 1N HCl to pH 3 and diluted with 100 ml of EtOAc. It was washed with brine (5 ml), dried with MgSO₄, filtered and concentrated in vacuo to afford (S)-7-((S)-2-(methoxycarbonylamino)-3-methylbutanoyl)-1,4-dioxa-7-azaspiro[4.4]nonane-8-carboxylic acid as a white solid which was used for the next reaction without further purification. (75 mg, 52.1%).

In a 50 mL flask, (S)-7-((S)-2-(methoxycarbonylamino)-3-methylbutanoyl)-1,4-dioxa-7-azaspiro[4.4]nonane-8-carboxylic acid (110 mg, 333 μmol, Eq: 1.00) was combined with DMF (5.00 ml) to give a colorless solution. HATU (127 mg, 333 μmol, Eq: 1.00) was added and stirred at room temperature for 10 min followed by addition of 2-amino-1-(4-bromophenyl)ethanone HCl (83.3 mg, 333 μmol, Eq: 1.00) diisopropylethylamine (129 mg, 174 μl, 999 μmol, Eq: 3) was then added dropwise and the suspension became a yellow solution once the methylenechloride was added. It was stirred at room temperature for 1 hr then poured into brine solution (80 ml). The precipitate was filtered and washed with H₂O then dissolved in CH₂Cl₂ (50 ml), It was dried with MgSO₄, filtered and concentrated in vacuo to afford methyl(S)-1-((S)-8-(2-(4-bromophenyl)-2-oxoethylcarbamoyl)-1,4-dioxa-7-azaspiro[4.4]nonan-7-yl)-3-methyl-1-oxobutan-2-ylcarbamate as a yellow solid (75 mg, 42.8%). ESI-LRMS m/e calcd for C₂₂H₂₈BrN₃O₇ [M⁺] 526, found 527 [M+H⁺].

In a 5 mL seal tube, methyl (S)-1-((S)-8-(2-(4-bromophenyl)-2-oxoethylcarbamoyl)-1,4-dioxa-7-azaspiro[4.4]nonan-7-yl)-3-methyl-1-oxobutan-2-ylcarbamate (75 mg, 142 μmol, Eq: 1.00) was combined with 1,4-dioxane (2 ml) to give a light yellow solution. Ammonium acetate (110 mg, 1.42 mmol, Eq: 10) was added and it was stirred at 110° C. overnight. It was cooled and diluted with EtOAc (10 ml), filtered and concentrated in vacuo to afford methyl (S)-1-((S)-8-(5-(4-bromophenyl)-1H-imidazol-2-yl)-1,4-dioxa-7-azaspiro[4.4]nonan-7-yl)-3-methyl-1-oxobutan-2-ylcarbamate as a light brownish solid (70 mg, 96.8%). ESI-LRMS m/e calcd for C₂₂H₂₇BrN₄O₅ [M⁺] 507, found 508 [M+H⁺].

In a 20 mL seal tube, methyl (S)-1-((S)-8-(5-(4-bromophenyl)-1H-imidazol-2-yl)-1,4-dioxa-7-azaspiro[4.4]nonan-7-yl)-3-methyl-1-oxobutan-2-ylcarbamate (70 mg, 138 μmol, Eq: 1.00), 4,4,4′,4′,5,5,5′,5′-octamethyl-2,2′-bi(1,3,2-dioxaborolane) (105 mg, 414 μmol, Eq: 3.0) and potassium acetate (67.7 mg, 690 μmol, Eq: 5.0) were combined with 1,4-dioxane (2 ml) to give a light yellow suspension. It was degassed for 20 min and 1,1′-bis(diphenylphosphino)ferrocene-palladium(II)dichloride dichloromethane complex (11.3 mg, 13.8 μmol, Eq: 0.10) was added. It was flushed with N₂, sealed and stirred at 80° C. for 16 hr. The reaction mixture was cooled and diluted with EtOAc (10 ml). It was filtered through celite, concentrated in vacuo and purified on a silica gel column (CH₂Cl₂, 1%, 2%, 3% to 5% MeOH/CH₂Cl₂) to afford methyl (S)-3-methyl-1-oxo-1-((S)-8-(5-(4-(4,4,5,5-tetramethyl-1,3,2-dioxaborolan-2-yl)phenyl)-1H-imidazol-2-yl)-1,4-dioxa-7-azaspiro[4.4]nonan-7-yl)butan-2-ylcarbamate as a light brownish solid. (31 mg, 41.5%). ESI-LRMS m/e calcd for C₂₈H₃₉BN₄O₇ [M⁺] 554, found 555 [M+H⁺].

### Intermediate 12

### Methyl(2S)-3-methyl-1-oxo-1-(3-(5-(4-(4,4,5,5-tetramethyl-1,3,2-dioxaborolan-2-yl)phenyl)-1H-imidazol-2-yl)-8-oxa-2-azaspiro[4.5]decan-2-yl)butan-2-ylcarbamate

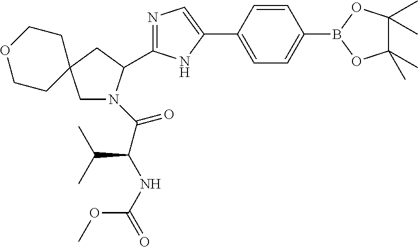

In a 50 ml round-bottomed flask, (S)-2-(methoxycarbonylamino)-3-methylbutanoic acid (813 mg, 4.64 mmol, Eq: 1.1) was combined with DMF (10 ml) to give a colorless solution. HATU (1.76 g, 4.64 mmol, Eq:1.1) was added and stirred at room temperature for 10 min. Ethyl 8-oxa-2-azaspiro[4.5]decane-3-carboxylate (prepared according to the patent procedure described in WO 98/08850) (900 mg, 4.22 mmol, Eq: 1.00) and methylenechloride (1.91 g, 2.58 ml, 14.8 mmol, Eq: 3.5) were added and stirred overnight. It was diluted with H₂O and extracted with EtOAc (2×100 ml). The organic layer was washed with brine and H₂O, dried with MgSO₄, filtered and concentrated in vacuo to afford (S)-ethyl 2-((S)-2-(methoxycarbonylamino)-3-methylbutanoyl)-8-oxa-2-azaspiro[4.5]decane-3-carboxylate as a viscous oil (1.3 g, 83.2%). ESI-LRMS m/e calcd for C₁₈H₃₀N₂O₆ [M⁺] 370, found 371 [M+H⁺].

In a 10 mL round-bottomed flask, (S)-ethyl 2-((S)-2-(methoxycarbonylamino)-3-methylbutanoyl)-8-oxa-2-azaspiro[4.5]decane-3-carboxylate (300 mg, 810 μmol) were combined with THF 6 (ml) and tert-butanol (1 ml) to give a colorless solution. LiOH (1.0M, 1.62 ml, 1.62 mmol, Eq: 2) was added and stirred at room temperature for 2 hr. Then more LiOH (1.0M, 1 ml, 1 mmol) was added and stirred for additional 2 hr. THF was removed and the aqueous was acidified with 1N HCl to pH=3 and diluted with EtOAc (100 ml). It was washed with brine (10 ml), dried with MgSO₄, filtered and concentrated to afford 2-((S)-2-(methoxycarbonylamino)-3-methylbutanoyl)-8-oxa-2-azaspiro[4.5]decane-3-carboxylic acid as a white solid (270 mg, 97.4%). ESI-LRMS m/e calcd for C₁₆H₂₆N₂O₆ [M⁺] 342, found 341 [M−H⁺].

In a 50 mL flask, 2-((S)-2-(methoxycarbonylamino)-3-methylbutanoyl)-8-oxa-2-azaspiro[4.5]decane-3-carboxylic acid (200 mg, 584 μmol, Eq: 1.00) was dissolved in DMF (5 ml) to give a colorless solution. HATU (222 mg, 584 μmol, Eq: 1.00) was added and stirred at room temperature for 10 min followed by addition of 2-amino-1-(4-bromophenyl)ethanone HCl (146 mg, 584 μmol, Eq: 1.00). N,N′-diisopropylethylamine (264 mg, 357 μl, 2.04 mmol, Eq: 3.5) was then added dropwise and the suspension became a yellow solution once the methylenechloride was added. It was stirred at room temperature for 1 hr and poured into brine solution (80 ml) and extracted with EtOAc (2×60 ml). The organic layer was washed with brine and H₂O, dried with MgSO₄, filtered and concentrated in vacuo to give a yellow solid. It was purified on a silica gel column (CH₂Cl₂, 2%, 4%, 6% MeOH/CH₂Cl₂) to afford methyl (2S)-1-(3-(2-(4-bromophenyl)-2-oxoethylcarbamoyl)-8-oxa-2-azaspiro[4.5]decan-2-yl)-3-methyl-1-oxobutan-2-ylcarbamate as a solid (200 mg, 63.6%). ESI-LRMS m/e calcd for C₂₄H₃₂BrN₃O₆ [M⁺] 538, found 539 [M+H⁺].

In a 10 ml seal tube, methyl (2S)-1-(3-(2-(4-bromophenyl)-2-oxoethylcarbamoyl)-8-oxa-2-azaspiro[4.5]decan-2-yl)-3-methyl-1-oxobutan-2-ylcarbamate (200 mg, 371 μmol) was combined with dioxane (6 ml) to give a colorless solution. Ammonium acetate (286 mg, 3.71 mmol, Eq: 10) was added and it was stirred at 110° C. overnight. It was cooled and diluted with EtOAc (10 ml). The mixture was filtered and concentrated in vacuo to afford methyl (2S)-1-(3-(5-(4-bromophenyl)-1H-imidazol-2-yl)-8-oxa-2-azaspiro[4.5]decan-2-yl)-3-methyl-1-oxobutan-2-ylcarbamate as a yellow solid (170 mg, 88.1%). ESI-LRMS m/e calcd for C₂₄H₃₁BrN₄O₄ [M⁺] 519, found 520 [M+H⁺].

In a 20 mL seal tube, methyl (2S)-1-(3-(5-(4-bromophenyl)-1H-imidazol-2-yl)-8-oxa-2-azaspiro[4.5]decan-2-yl)-3-methyl-1-oxobutan-2-ylcarbamate (170 mg, 327 μmol, Eq: 1.00), 4,4,4′,4′,5,5,5′,5′-octamethyl-2,2′-bi(1,3,2-dioxaborolane) (249 mg, 982 μmol, Eq: 3.0) and potassium acetate (161 mg, 1.64 mmol, Eq: 5.0) were combined with 1,4-dioxane (2.00 ml) to give a light yellow suspension. It was degassed for 20 min. 1,1′-Bis(diphenylphosphino)ferrocene-palladium(II)dichloride dichloromethane complex (26.7 mg, 32.7 μmol, Eq: 0.10) was added, flushed with N₂. It was sealed and stirred at 80° C. for 16 hr, then cooled and diluted with EtOAc (40 ml). The reaction mixture was filtered through celite, concentrated in vacuo and purified on a silica gel column (CH₂Cl₂, 1%, 2%, 3% to 5% MeOH/CH₂Cl₂) to afford methyl (2S)-3-methyl-1-oxo-1-(3-(5-(4-(4,4,5,5-tetramethyl-1,3,2-dioxaborolan-2-yl)phenyl)-1H-imidazol-2-yl)-8-oxa-2-azaspiro[4.5]decan-2-yl)butan-2-ylcarbamate as a light brown solid (110 mg, 59.3%). ESI-LRMS m/e calcd for C₃₀H₄₃BN₄O₆ [M⁺] 566, found 567 [M+H⁺].

### Intermediate 13

### 6-Bromo-2-chloro-quinoxaline

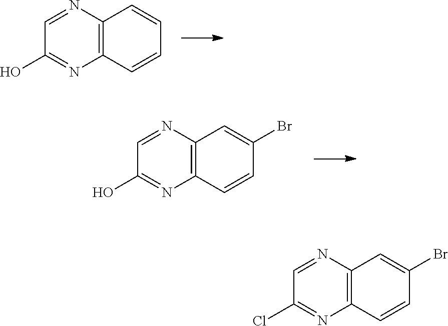

A 3-neck 3 L round bottom flask equipped with paddle stirring was charged with quinoxalin-2-ol (100 g, 684 mmol). To this was added concentrated sulfuric acid (560 mL) having been cooled to 10° C. The temperature rose to 35° C. and a solution formed within a few minutes. Powered silver sulfate (107 g, 343 mmol) was added and the mixture became a solution. Bromine (107 g, 34.5 ml, 669 mmol) was added dropwise at a rate that kept the temperature between 30-35° C. (30 minutes). The temperature was raised to 45° C. for 2.5 hr with strong stirring. To this was added chloroform (1 L) and after a few minutes of stirring, the mixture was filtered through a course sintered glass funnel. The filter cake (AgBr) was washed with sulfuric acid (2×200 mL) and chloroform (3×300 mL). The dark sulfuric acid residue was separated from the chloroform and treated with ice (1 kg) during which a heavy precipitate formed. This was filtered, washed with cold 1N sulfuric acid (200 mL), chloroform (2×200 mL) and ethyl ether (2×200 ml). This afforded 108 g of 6-bromo-quinoxalin-2-ol as a gray solid. ¹H NMR (400 MHz, DMSO-d₆) δ ppm ¹H NMR (400 MHz, DMSO-d₆) δ ppm 7.24 (d, J=8.5 Hz, 1H) 7.65-7.74 (m, 1H) 7.95 (d, J=2.2 Hz, 1H) 8.19 (s, 1H).

To a well stirred suspension of 6-bromo-quinoxalin-2-ol (120 g, 528 mmol) in phosphorus oxychloride (450 ml, 4.8 mol) was added DMF (3 mL). The temperature was raised slowly to 80° C. and HCl evolved vigorously. The temperature was raised slowly to 120° C. for 1.5 hr. The solution was cooled and reduced in volume to ˜150 mL. This was neutralized by pouring the dark residue over ice containing NaHCO₃ and keeping the temperature below 15° C. The solid was filtered, washed with water and the solids dissolved in dichloromethane. This was dried (MgSO₄), treated with Norite, and filtered through celite. The solvent was removed in vacuo to give 80 g of 6-bromo-2-chloro-quinoxaline (yield −58%). ¹H NMR (400 MHz, DMSO-d₆) ppm 8.97 (s, 1H) 8.35 (d, J=2.2 Hz, 1H) 7.99-8.02 (m, 1H) 7.94 (d, J=9 Hz, 1H).

### Intermediate 14

### 2-Bromo-1-[4′-(2-bromo-acetyl)-biphenyl-4-yl]ethanone

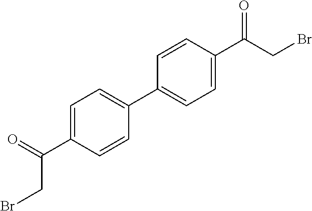

A solution of bromine (13.4 g, 4.32 mL, 83.9 mmol) in glacial acetic acid (30 mL) was added drop wise to a mixture of 1,1-(biphenyl-4-4-diyl)diethanone (10.0 g, 42.0 mmol) in glacial acetic acid (100 mL) at 50 C. After addition was complete, the mixture was stirred at room temperature for 24 hours. The solid was filtered and washed with chloroform to afford 1,1-(biphenyl-4,4-diyl)-bis(2-bromoethanone as white solid, (15.9 g, 96%). ¹H NMR (400 MHz, DMSO-d₆) δ ppm 4.98 (s, 4H) 7.85-8.25 (m, 8H).

### Intermediate 15

### (S)-2-(2-(4-(2-Bromoacetyl)biphenyl-4-yl)-2-oxoethyl) 1-tert-butyl pyrrolidine-1,2-dicarboxylate

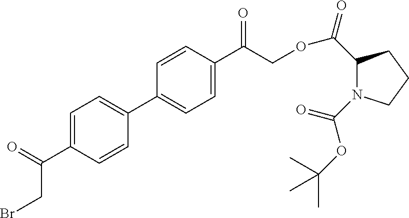

To a stirred mixture of 1,1-(biphenyl-4,4-dyl)bis(2-bromoethanone) (5.52 g, 13.9 mmol) and L-boc proline (1.0 g, 4.65 mmol) in DMF (40 mL) was added diisopropylethylamine (1.62 mL, 9.29 mmol). After addition was complete, the mixture was stirred at room temperature overnight. The reaction was diluted with ethyl acetate, washed with water and dried over magnesium sulfate. The crude product obtained was purified by ISCO flash chromatography (Teledyne isco RediSep Flash Column 120 g; (0% to 50%, ethyl acetate/hexane) to afford (S)-2-(2-(4-(2-Bromoacetyl)biphenyl-4-yl)-2-oxoethyl) 1-tert-butyl pyrrolidine-1,2-dicarboxylate as a white solid, (1.2 g, 49%). ESI-LRMS m/e calcd for C₂₈H₂₂BrN₃O₄ [M⁺] 530, found 531 [M⁺H].

### Intermediate 16

### (S)-Tert-butyl-2-(2(4-(2-((2S,5S)-5-(methoxy-carbonylamino)-4-oxo-1,2,4,5,6,7-hexahydroazeazepino[3,2,1-hi]indole-2-carbonyloxy)acetyl)biphenyl-4-yl)-2-oxoethyl)pyrrolidine-1,2 dicarboxylate

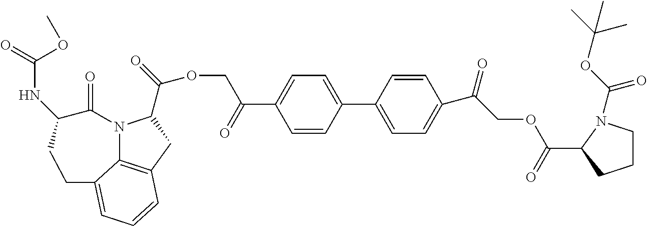

To a stirred mixture of (S)-2-(4-(2-bromoacetyl)biphenyl-4-yl)-2-oxoethyl) 1-tert-butyl pyrrolidine-1,2-dicarboxylate (0.53 g, 0.999 mmol) and (2S,5S)-5 (methoxycarbonylamino)-4-oxo-1,2,4,5,6,7-hexahydroazepino[3,2,1-hi]indole-2-carboxylic acid (0.304 g, 0.999 mmol) in acetonitrile (10 mL) was added diisopropylethylamine (0.262 mL, 1.50 mmol). After the addition was complete, the mixture was stirred at room temperature overnight. The reaction was diluted in ethyl acetate, washed with water, 1N HCl and dried over magnesium sulfate. The crude product obtained was purified by ISCO flash chromatography (Teledyne isco RediSep Flash Column 120 g; (0% to 50%, ethyl acetate/hexane) to afford (S)-1-tert-butyl 2-(2-(4-(2-((2S,5S)-5-(methoxycarbonylamino)-4-oxo-1,2,4,5,6,7-hexahydroazepino[3,2,1-hi]indole-2-carbonyloxy)acetyl)biphenyl-4-yl)-2-oxoethyl)pyrrolidine-1,2dicarboxylate as a white solid (700 mg, 62%): ESI-LRMS m/e calcd for C₄₁H₄₃N₃O₁₁ [M⁺] 753, found 754 [M⁺+H].

### Intermediate 17

### (S)-Tert-butyl 2-(4-(4-(2-((2S,5S)-5-(methoxycar-bonylamino)-4-oxo-1,2,4,5,6,7-hexahydroazepino[3,2,1-hi]indol-2-yl)-1H-imidazol-5-yl)biphenyl-4-yl)-1H-imidazol-2-yl)pyrrolidine-1-carboxylate

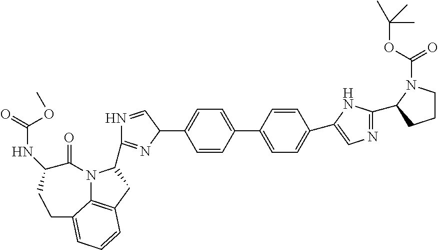

To a of mixture of (S)-1-tert-butyl 2-(2-((2S,5S)-5-methoxycarbonylamino)-4-oxo-1,2,4,5,6,7-hexahydroazepino[3,2,1-hi]indole-2-carbonyloxy)acetyl)biphenyl-4-yl)-2-oxoethyl)pyrrolidine-1,2 carboxylate (1.2 g, 1.59 mmol) and ammonium acetate (2.45 g, 31.8 mmol) was added dioxane (12 mL) in a sealed tube. The mixture was heated at 110 C for 3 hours. The reaction was diluted in ethyl acetate washed with saturated sodium bicarbonate, brine and concentrated in vacuo to afford(S)-tert-butyl 2-(4-(4-(2-((2S,5S)-5-(methoxycar-bonylamino)-4-oxo-1,2,4,5,6,7-hexahydroazepino[3,2,1-hi]indol-2-yl)-1H-imidazol-5-yl)biphenyl-4-yl)-1H-imidazol-2-yl)pyrrolidine-1-carboxylate as a light orange solid, (700 mg, 62%): ESI-LRMS m/e calcd for C₄₁H₄₃N₇O₅ [M⁺] 713, found 714 [M⁺+H].

### Intermediate 18

### (S)-2-(5-(4-(2-((2S,5S)-5-(Methoxycarbonylamino)-4-oxo-1,2,4,5,7-hexahydroazepino[3,2,1-hi]indol-2-yl)-1H-imidazol-5-yl)biphenyl-4-yl)-1H-imidazol-2-yl)pyrrolidinium chloride

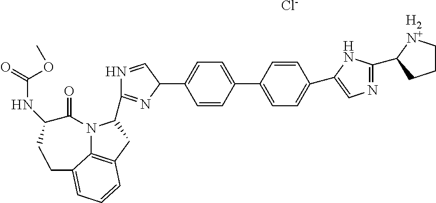

To a stirred mixture of (S)-tert-butyl 2-(4-(4-(2-((2S,5S)-5-(methoxycar-bonylamino)-4-oxo-1,2,4,5,6,7-hexahydroazepino[3,2,1-hi]indol-2-yl)-1H-imidazol-5-yl)biphenyl-4-yl)-1H-imidazol-2-yl)pyrrolidine-1-carboxylate (0.65 g, 0.911 mmol) dissolved in 1:1 mixture of methanol/dioxane (10 mL) was added 4.0 M HCl/dioxane (0.7 mL, 2.8 mmol). After addition was complete, the reaction stirred at room temperature for 3 hours. Concentrate to dryness to afford (S)-2-(5-(4-(2-((2S,5S)-5-(methoxycarbonylamino)-4-oxo-1,2,4,5,7-hexahydroazepino[3,2,1-hi]indol-2-yl)-1H-imidazol-5-yl)biphenyl-4-yl)-1H-imidazol-2-yl)pyrrolidinium chloride, (630 mg): ESI-LRMS m/e calcd for C₃₆H₃₆N₇O₃C1 [M⁺] 614, found 615 [M⁺+H].

### Intermediate 19

### tert-Butyl 2-(4-bromophenyl)-2-oxoethylcarbamate

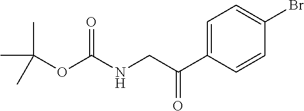

To a stirred solution of 2-amino-1-(4-bromophenyl)ethanone hydrochloride (19.94 g, 79.6 mmol, Eq: 1.00) in THF (200 ml) was added N,N-diisopropylethylamine (12.3 g, 16.7 ml, 95.5 mmol, Eq: 1.2). R×n. Mixture stirred at rt. For 30 min. then di-tert-butyl dicarbonate (17.4 g, 18.5 ml, 79.6 mmol, Eq: 1.00) was added. Continue to stir at rt. For an additional 6 hrs. Concentrated, EtOAc/water workup gave tert-butyl 2-(4-bromophenyl)-2-oxoethylcarbamate (24.88 g, 79.2 mmol, 99.5% yield) a light yellow powder. LC/MS (M⁺+H)=315.

### Intermediate 20

### Preparation of tert-butyl 2-oxo-2-(4-(4,4,5,5-tetramethyl-1,3,2-dioxaborolan-2yl)phenyl)ethylcarbamate

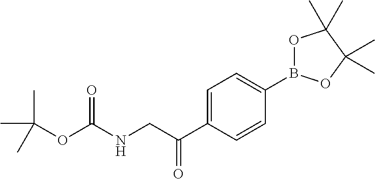

A suspension of tert-butyl 2-(4-bromophenyl)-2-oxoethylcarbamate (13.44 g, 42.8 mmol, Eq: 1.00), bis(pinacolato)diboron (11.4 g, 44.9 mmol, Eq: 1.05), [1,1′-BIS(DIPHENYLPHOSPHINO)FERROCENE]DICHLOROPALLADIUM(II) (3.13 g, 4.28 mmol, Eq: 0.1) and Potassium acetate (12.6 g, 128 mmol, Eq: 3) in Dioxane (50 ml) was purged with nitrogen for 10 min. and r×n. Mixture heated at 90° C. for 16 hrs. Solvent removed in vacuo, the black residue diluted with methylenechloride, filtered through a pad of Celite, washed with methylenechloride, concentrated, chromatographed through a small silica gel column, eluting with 20% EtOAc-Hexane mixture to obtain tert-butyl 2-oxo-2-(4-(4,4,5,5-tetramethyl-1,3,2-dioxaborolan-2-yl)phenyl)ethylcarbamate (15.2 g, 42.1 mmol, 98.4% yield) as an oil. LC/MS (M 4H)=362

### Intermediate 21

### Preparation of tert-butyl 2-(4-(6-bromoquinoxalin-2-yl)phenyl)-2-oxoethylcarbamate

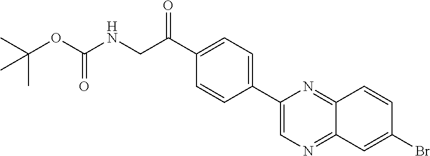

A suspension of tert-butyl 2-oxo-2-(4-(4,4,5,5-tetramethyl-1,3,2-dioxaborolan-2-yl)phenyl)ethylcarbamate (1.5 g, 4.15 mmol, Eq: 1.00), 6-bromo-2-chloroquinoxaline (1.01 g, 4.15 mmol, Eq: 1.00), cesium carbonate (2.71 g, 8.3 mmol, Eq: 2) and tetrakis(triphenylphosphine)palladium (0) (480 mg, 415 μmol, Eq: 0.1) in Dioxane (20 ml) and Water (2 ml) was purged with nitrogen for 10 min. then r×n. Mixture was heated at 80° C. for 16 hrs. Solvent removed in vacuo, the black residue filtered through a pad of Celite, washed with EtOAc, concentrated, triturated with ether, light yellow solid filtered, dried to obtain tert-butyl 2-(4-(6-bromoquinoxalin-2-yl)phenyl)-2-oxoethylcarbamate (1.7 g, 3.84 mmol, 92.6% yield) as a light yellow powder. LC/MS (M⁺+H)=443

### Intermediate 22

### 2-Amino-1-(4-(6-bromoquinoxalin-2-yl)phenyl)ethanone 2,2,2-trifluoroacetate

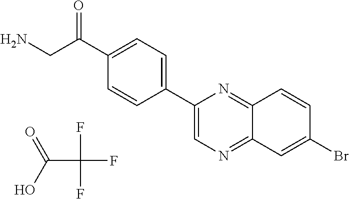

To a solution of tert-butyl 2-(4-(6-bromoquinoxalin-2-yl)phenyl)-2-oxoethylcarbamate (1.81 g, 4.09 mmol, Eq: 1.00) in methylenechloride (25 ml) was added TFA (11.7 g, 7.88 ml, 102 mmol, Eq: 25) and r×n. Mixture was stirred at rt. For 1 hr. Solvent removed in vacuo to obtain light brown solid, triturated with ether, filtered, dried to obtain 2-amino-1-(4-(6-bromoquinoxalin-2-yl)phenyl)ethanone 2,2,2-trifluoroacetate (1.66 g, 3.64 mmol, 88.9% yield) as a yellow powder. LC/MS (M⁺+H) 343

### Intermediate 23

### Methyl (2S,5S)-2-(2-(4-(6-bromoquinoxalin-2-yl)phenyl)-2-oxoethylcarbamoyl)-4-oxo-1,2,4,5,6,7-hexahydroazepino[3,2,1-hi]indol-5-ylcarbamate

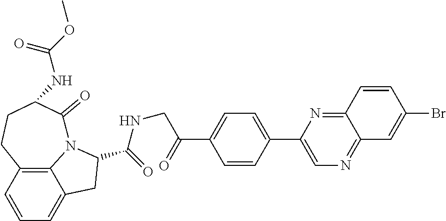

To a suspension of (2S,5S)-5-(methoxycarbonylamino)-4-oxo-1,2,4,5,6,7-hexahydroazepino[3,2,1-hi]indole-2-carboxylic acid (630 mg, 2.07 mmol, Eq: 1.00), 2-amino-1-(4-(6-bromoquinoxalin-2-yl)phenyl)ethanone 2,2,2-trifluoroacetate (1.61 g, 3.52 mmol, Eq: 1.7) and HATU (787 mg, 2.07 mmol, Eq: 1.00) in DMF (25 ml) at rt. Was added dropwise N,N-methylenechloride (803 mg, 1.08 ml, 6.21 mmol, Eq: 3.00). R×n. Mixture stirred at rt. For o/n. Solvent removed in vacuo, the residue triturated with MeOH, the light yellow solid formed was filtered, washed with ether, dried to obtain methyl (2S,5S)-2-(2-(4-(6-bromoquinoxalin-2-yl)phenyl)-2-oxoethylcarbamoyl)-4-oxo-1,2,4,5,6,7-hexahydroazepino[3,2,1-hi]indol-5-ylcarbamate (470 mg, 748 μmol, 36.1% yield) as a light yellow powder. LC/MS (M⁺+H)=629

### Intermediate 24

### Methyl (2S,5S)-2-(5-(4-(6-bromoquinoxalin-2-yl)phenyl)-1H-imidazol-2-yl)-4-oxo-1,2,4,5,6,7-hexahydroazepino[3,2,1-hi]indol-5-ylcarbamate

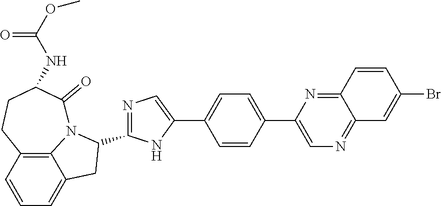

A mixture of methyl (2S,5S)-2-(2-(4-(6-bromoquinoxalin-2-yl)phenyl)-2-oxoethylcarbamoyl)-4-oxo-1,2,4,5,6,7-hexahydroazepino[3,2,1-hi]indol-5-ylcarbamate (3.9 g, 6.21 mmol, Eq: 1.00) and ammonium acetate (9.57 g, 124 mmol, Eq: 20) in Xylene (150 ml) was heated at 140° C. for 1 hr. R×n. Mixture cooled to rt, white solid ppted. At the bottom of the flask, filtered (discarded), xylene removed in vacuo, the residue triturated with ether, light brown solid formed was washed with ether, dried to obtain methyl (2S,5S)-2-(5-(4-(6-bromoquinoxalin-2-yl)phenyl)-1H-imidazol-2-yl)-4-oxo-1,2,4,5,6,7-hexahydroazepino[3,2,1-hi]indol-5-ylcarbamate (2.9 g, 4.76 mmol, 76.7% yield) as a light brown powder. LC/MS (M⁺+H)=610

### Intermediate 25

### Methyl (2S,5S)-4-oxo-2-(5-(4-(6-(4,4,5,5-tetramethyl-1,3,2-dioxaborolan-2-yl)quinoxalin-2-yl)phenyl)-1H-imidazol-2-yl)-1,2,4,5,6,7-hexahydroazepino[3,2,1-hi]indol-5-ylcarbamate

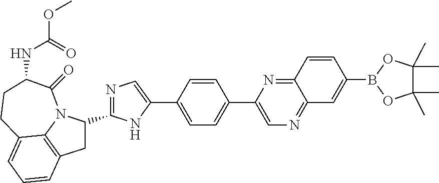

A suspension of methyl (2S,5S)-2-(5-(4-(6-bromoquinoxalin-2-yl)phenyl)-1H-imidazol-2-yl)-4-oxo-1,2,4,5,6,7-hexahydroazepino[3,2,1-hi]indol-5-ylcarbamate (273 mg, 448 μmol, Eq: 1.00), bis(pinacolato)diboron (136 mg, 538 μmol, Eq: 1.2), [1,1′-BIS(DIPHENYLPHOSPHINO)FERROCENE]DICHLOROPALLADIUM(II) (32.8 mg, 44.8 μmol, Eq: 0.1) and potassium acetate (132 mg, 1.34 mmol, Eq: 3.0) in Dioxane (10 ml) was purged with nitrogen for 5 min. then r×n. Mixture heated at 90° C. for 2 hrs. Cooled, diluted with water, extracted with methylenechloride, dried (MgSO₄). Concentrated, triturated with ether, filter, filtrate was concentrated to obtain crude methyl (2S,5S)-4-oxo-2-(5-(4-(6-(4,4,5,5-tetramethyl-1,3,2-dioxaborolan-2-yl)quinoxalin-2-yl)phenyl)-1H-imidazol-2-yl)-1,2,4,5,6,7-hexahydroazepino[3,2,1-hi]indol-5-ylcarbamate (294 mg, 448 μmol, 100% yield) as a light yellow foam, LC/MS (M⁺+H)=657, used as such in the next step.

### Intermediate 26

### (S)-tert-Butyl 2-(5-(2-(4-(2-((2S,5S)-5-(methoxycarbonylamino)-4-oxo-1,2,4,5,6,7-hexahydroazepino[3,2,1-hi]indol-2-yl)-1H-imidazol-5-yl)phenyl)quinoxalin-6-yl)-1-((2-(trimethylsilyl)ethoxy)methyl)-1H-imidazol-2-yl)pyrrolidine-1-carboxylate

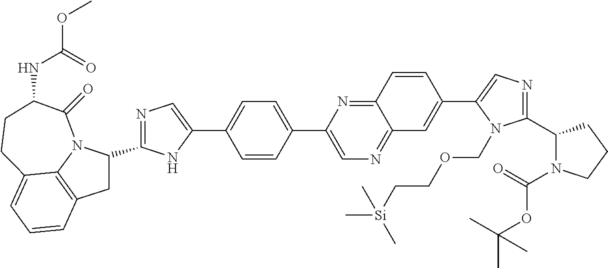

A suspension of methyl (2S,5S)-4-oxo-2-(5-(4-(6-(4,4,5,5-tetramethyl-1,3,2-dioxaborolan-2-yl)quinoxalin-2-yl)phenyl)-1H-imidazol-2-yl)-1,2,4,5,6,7-hexahydroazepino[3,2,1-hi]indol-5-ylcarbamate (294 mg, 448 μmol, Eq: 1.00), (S)-tert-butyl 2-(5-iodo-1-((2-(trimethylsilyl)ethoxy)methyl)-1H-imidazol-2-yl)pyrrolidine-1-carboxylate (265 mg, 537 μmol, Eq: 1.2), cesium carbonate (292 mg, 896 μmol, Eq: 2) and tetrakis(triphenylphosphine)palladium (0) (51.7 mg, 44.8 μmol, Eq: 0.1) in Dioxane (4 ml) and Water (0.5 ml) was purged with nitrogen for 10 min., then heated at 90° C. for 5 hrs. Cooled, diluted with water, extracted with methylenechloride, dried (MgSO₄). Concentrated, chromatographed (silica gel, gradient 0-10% MeOH-methylenechloride) to obtain (S)-tert-butyl 2-(5-(2-(4-(2-((2S,5S)-5-(methoxycarbonylamino)-4-oxo-1,2,4,5,6,7-hexahydroazepino[3,2,1-hi]indol-2-yl)-1H-imidazol-5-yl)phenyl)quinoxalin-6-yl)-1-((2-(trimethylsilyl)ethoxy)methyl)-1H-imidazol-2-yl)pyrrolidine-1-carboxylate (97 mg, 108 μmol, 24.2% yield) as a light yellow viscous oil. LC/MS (M⁺+H)=897.

### Intermediate 27

### Methyl (2S,5S)-4-oxo-2-(5-(4-(6-(2-((S)-pyrrolidin-2-yl)-1H-imidazol-5-yl)quinoxalin-2-yl)phenyl)-1H-imidazol-2-yl)-1,2,4,5,6,7-hexahydroazepino[3,2,1-hi]indol-5-ylcarbamate hydrochloride

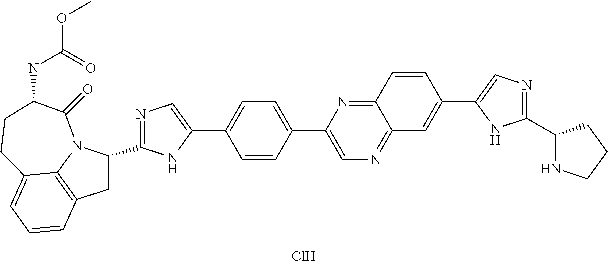

To a solution of (S)-tert-butyl 2-(5-(2-(4-(2-((2S,5S)-5-(methoxycarbonylamino)-4-oxo-1,2,4,5,6,7-hexahydroazepino[3,2,1-hi]indol-2-yl)-1H-imidazol-5-yl)phenyl)quinoxalin-6-yl)-1-((2-(trimethylsilyl)ethoxy)methyl)-1H-imidazol-2-yl)pyrrolidine-1-carboxylate (135.4 mg, 151 mmol, Eq: 1.00) in EtOH (4 ml) was added HYDROCHLORIC ACID, 4.0 M solution in 1,4-dioxane (7.55 ml, 30.2 mmol, Eq: 200). The r×n. mixture was stirred at 65° C. for 16 hrs. Solvent removed in vacuo, the residue triturated with ether, filtered, dried to obtain methyl (25,55)-4-oxo-2-(5-(4-(6-(2-((S)-pyrrolidin-2-yl)-1H-imidazol-5-yl)quinoxalin-2-yl)phenyl)-1H-imidazol-2-yl)-1,2,4,5,6,7-hexahydroazepino[3,2,1-hi]indol-5-ylcarbamate hydrochloride (100 mg, 89% yield) as a dark red powder. LC/MS (M⁺+H)=666.

### Intermediate 28

### Methyl (2S,5S)-4-oxo-2-(4-(4-(4,4,5,5-tetramethyl-1,3,2-dioxaborolan-2-yl)phenyl)-1H-imidazol-2-yl)-1,2,4,5,6,7-hexahydroazepino[3,2,1-hi]indol-5-ylcarbamate

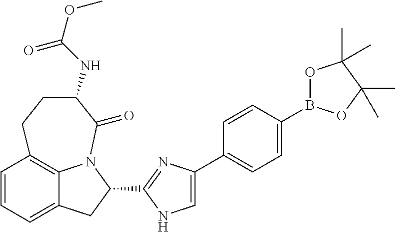

A suspension of methyl (2S,5S)-2-(4-(4-bromophenyl)-1H-imidazol-2-yl)-4-oxo-1,2,4,5,6,7-hexahydroazepino[3,2,1-hi]indol-5-ylcarbamate (40 mg, 83.1 μmol, Eq: 1.00), BIS(PINACOLATO)DIBORON (63.3 mg, 249 μmol, Eq: 3), potassium acetate (40.8 mg, 416 mmol, Eq: 5) and [1,1′-BIS(DIPHENYLPHOSPHINO)-FERROCENE]DICHLOROPALLADIUM(II) (6.08 mg, 8.31 μmol, Eq: 0.1) in Dioxane (3 ml) was purged with nitrogen and heated at 80° C. for 18 hrs. Reaction mixture filtered through Celite, concentrated, subjected to methylenechloride/water workup, organic layer washed with brine, dried (MgSO₄). Concentrated to obtain crude methyl (2S,5S)-4-oxo-2-(4-(4-(4,4,5,5-tetramethyl-1,3,2-dioxaborolan-2-yl)phenyl)-1H-imidazol-2-yl)-1,2,4,5,6,7-hexahydroazepino[3,2,1-hi]indol-5-ylcarbamate (35 mg, 66.2 μmol, 79.7% yield). LC/MS (M⁺+H)-529.

### Intermediate 29

### Methyl (2S,5S)-2-(4-(4-(5-(2-((8)-4,4-difluoropyrrolidin-2-yl)-1H-imidazol-5-yl)pyrimidin-2-yl)phenyl)-1H-imidazol-2-yl)-4-oxo-1,2,4,5,6,7-hexahydroazepino[3,2,1-hi]indol-5-ylcarbamate hydrochloride

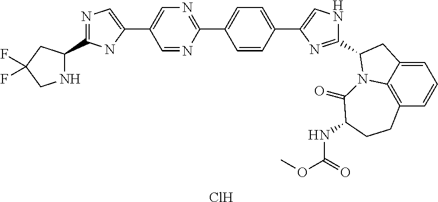

A suspension of methyl (2S,5S)-4-oxo-2-(4-(4-(4,4,5,5-tetramethyl-1,3,2-dioxaborolan-2-yl)phenyl)-1H-imidazol-2-yl)-1,2,4,5,6,7-hexahydroazepino[3,2,1-hi]indol-5-ylcarbamate (57.5 mg, 109 μmol, Eq: 1.2), (S)-tert-butyl 2-(5-(2-chloropyrimidin-5-yl)-1H-imidazol-2-yl)-4,4-difluoropyrrolidine-1-carboxylate (35.0 mg, 90.7 μmol, Eq: 1.00), cesium carbonate (59.1 mg, 181 μmol, Eq: 2.00) and tetrakis(triphenylphosphine)palladium (0) (10.5 mg, 9.07 μmol, Eq: 0.1) in Dioxane (2 ml) and Water (0.50 ml) was purged with nitrogen, then heated at 100° C. for 4 hrs. R×n. Mixture filtered through a small silica gel column, washed with methylenechloride, concentrated, triturated with ether, filtered, dried to obtain (S)-tert-butyl 4,4-difluoro-2-(5-(2-(4-(2-((2S,5S)-5-(methoxycarbonylamino)-4-oxo-1,2,4,5,6,7-hexahydroazepino[3,2,1-hi]indol-2-yl)-1H-imidazol-4-yl)phenyl)pyrimidin-5-yl)-1H-imidazol-2-yl)pyrrolidine-1-carboxylate (37.5 mg, 49.9 μmol, 55.0% yield) as a light brown powder. LC/MS (M⁺+H)-752.

To a solution of (S)-tert-butyl 4,4-difluoro-2-(5-(2-(4-(2-((2S,5S)-5-(methoxycarbonylamino)-4-oxo-1,2,4,5,6,7-hexahydroazepino[3,2,1-hi]indol-2-yl)-1H-imidazol-4-yl)phenyl)pyrimidin-5-yl)-1H-imidazol-2-yl)pyrrolidine-1-carboxylate (37.5 mg, 49.9 μmol, Eq: 1.00) in EtOH (4 ml) was added hydrochloric acid, 4M solution in 1,4-Dioxane (1.25 ml, 4.99 mmol, Eq: 100) and heated at 50° C. for 2 hrs. Solvent removed in vacuo, the residue triturated with ether, filter, dried to obtain methyl (25,55)-2-(4-(4-(5-(2-((S)-4,4-difluoropyrrolidin-2-yl)-1H-imidazol-5-yl)pyrimidin-2-yl)phenyl)-1H-imidazol-2-yl)-4-oxo-1,2,4,5,6,7-hexahydroazepino[3,2,1-hi]indol-5-ylcarbamate hydrochloride (30.2 mg, 43.9 μmol, 88.0% yield) as an off-white powder. LC/MS (M⁺+H)-652.

### Intermediate 30

### Methyl (S)-1-08)-2-(5-(4-(6-bromoquinoxalin-2-yl)phenyl)-1H-imidazol-2-yl)pyrrolidin-1-yl)-3-methyl-1-oxobutan-2-ylcarbamate

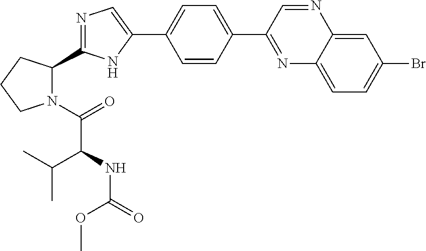

### (S)-2-Methoxycarbonylamino-3-methyl-butyric acid

To a mixture of (S)-2-amino-3-methylbutanoic acid (50 g, 427 mmol), sodium hydroxide (37.6 g, 939 mmol), water (250 mL), and toluene (250 mL) cooled in ice-water bath was added dropwise methyl chloroformate (36.1 mL, 469 mmol). The mixture was then warmed to room temperature and stirred for 12 hours. The organic phase was separated. The aqueous phase was acidified with 6 N HCl at 0° C., and extracted with EtOAc. The EtOAc layer was washed with water and brine, dried over sodium sulfate, filtered and evaporated to give a white solid product. The product was recrystallized from ethyl acetate to give pure white crystalline solid, (S)-2-Methoxycarbonylamino-3-methyl-butyric acid (61 g, 81.6%): ¹H NMR (400 MHz, CDCl₃) δ ppm 0.95, 1.02 (2d, 6H) 2.23 (m, 1H) 3.69, 3.74 (2s, 3H) 4.13, 4.33 (2m, 1H) 5.35, 6.39 (d, 1H) 11.71 (s, 1H).

### (S)-2-(2-(4-bromophenyl)-2-oxoethyl) 1-tert-butyl pyrrolidine-1,2-dicarboxylate

To a solution of Boc-L-proline (50 g, 232 mmol) in dichloromethane (400 mL) was added 2,4′-dibromoacetophenone (67.8 g, 244 mmol) at 0° C., followed by dropwise addition of N,N′-diisopropylethylamine (44.6 mL, 256 mmol). The resulting solution was allowed to warm to room temperature and stirred for a further 3 hours, followed by washing with water, saturated sodium bicarbonate, water and brine. The organic phase was dried over sodium sulfate, filtered and evaporated to give an oily product, (S)-2-(2-(4-bromophenyl)-2-oxoethyl) 1-tert-butyl pyrrolidine-1,2-dicarboxylate, which was used directly for next.

### (S)-2-[5-(4-Bromo-phenyl)-1H-imidazol-2-yl]-pyrrolidine-1-carboxylic acid tert-butyl ester

To a solution of (S)-2-(2-(4-bromophenyl)-2-oxoethyl) 1-tert-butyl pyrrolidine-1,2-dicarboxylate (95.6 g, 232 mmol) in xylene (400 mL) was added ammonium acetate (89.4 g, 1.16 mol). The resulting mixture was heated at 150° C. for 5 hours, followed by cooling to room temperature, diluting with EtOAc (400 mL), washing with water (×3) and brine (×2), drying over sodium sulfate, filtration, and evaporation on rotary evaporator under reduced pressure. The residue was stirred with TBME (120 mL). The yellow solid was collected by filtration and washing with diethyl ether to give (S)-2-[5-(4-Bromo-phenyl)-1H-imidazol-2-yl]-pyrrolidine-1-carboxylic acid tert-butyl ester (77 g, 84.6%): ESI-LRMS m/e calcd for C₁₈H₂₂BrN₃O₂ [M⁺] 393.0, found 393.9 [M+H⁺].

### (S)-2-(4-bromophenyl)-5-(pyrrolidin-2-yl)-1H-imidazole

A solution of (S)-2-[5-(4-Bromo-phenyl)-1H-imidazol-2-yl]-pyrrolidine-1-carboxylic acid tert-butyl ester (20 g, 51.0 mmol) in methylene chloride (50 mL) was added slowly to a stirring solution of TFA (5 mL) at room temperature. The resulting solution was stirred at room temperature for 1 hour, evaporated via rotary evaporator under reduced pressure with toluene, treated with water (50 mL), adjusted the pH-8 with sodium bicarbonate, and extracted with EtOAc (×2). The combined EtOAc solution was washed with brine, dried over sodium sulfate, filtered and evaporated to give title compound as a yellow solid, (S)-2-(4-bromophenyl)-5-(pyrrolidin-2-yl)-1H-imidazole (quantitive): ESI-LRMS m/e calcd for C₁₃H₁₄BrN₃ [M⁺]292.0, found 292.9 [M+H⁺].

### Methyl (S)-1-((S)-2-(2-(4-bromophenyl)-1H-imidazol-5-yl)pyrrolidin-1-yl)-3-methyl-1-oxobutan-2-ylcarbamate

To a solution of HATU (23.4 g, 61.6 mmol) and (S)-2-(methoxycarbonylamino)-3-methylbutanoic acid (5.94 g, 33.9 mmol) and HATU (23.4 g, 61.6 mmol) in DMF (30 mL) was added a solution of (S)-2-(4-bromophenyl)-5-(pyrrolidin-2-yl)-1H-imidazole (9 g, 30.8 mmol) in DMF (20 mL) at room temperature. The resulting solution was stayed at room temperature overnight. The solution was then diluted with EtOAc (400 mL), washed with water (×2) and brine (×2), dried over sodium sulfate, filtered and evaporated. The residue was purified by column chromatography (0-100% EtOAc in hexane) to give methyl (S)-1-((S)-2-(2-(4-bromophenyl)-1H-imidazol-5-yl)pyrrolidin-1-yl)-3-methyl-1-oxobutan-2-ylcarbamate (5.2 g, 37.6%): ESI-LRMS m/e calcd for C₂₀H₂₅BrN₄O₃ [M⁺]450, found 451.1 [M+H⁺].

### Methyl (S)-3-methyl-1-oxo-1-((S)-2-(5-(4-(4,4,5,5-tetramethyl-1,3,2-dioxaborolan-2-yl)phenyl)-1H-imidazol-2-yl)pyrrolidin-1-yl)butan-2-ylcarbamate

A mixture of methyl (S)-1-((S)-2-(2-(4-bromophenyl)-1H-imidazol-5-yl)pyrrolidin-1-yl)-3-methyl-1-oxobutan-2-ylcarbamate (1.5 g, 3.34 mmol), 4,4,4′,4′,5,5,5′,5′-octamethyl-2,2′-bi(1,3,2-dioxaborolane) (1.7 g, 6.68 mmol), KOAc (0.82 g, 8.35 mmol), and Pd(dppf)Cl₂—CH₂Cl₂ adduct (73.3 mg, 0.1 mmol) in dioxane (7.5 mL) was degassed and flashed with nitrogen three times, followed by heating via microwave at 120° C. for 2 hours. The solvent was removed by rotary evaporation and the residue was purified by column (20% EtOAc in hexane) to give methyl (S)-3-methyl-1-oxo-1-((S)-2-(5-(4-(4,4,5,5-tetramethyl-1,3,2-dioxaborolan-2-yl)phenyl)-1H-imidazol-2-yl)pyrrolidin-1-yl)butan-2-ylcarbamate (1.5 g, 45.3%): ESI-LRMS m/e calcd for C₂₆H₃₇BN₄O₅ [M⁺] 496, found 497.3 [M+H⁺].

### Methyl (S)-1-((S)-2-(5-(4-(6-bromoquinoxalin-2-yl)phenyl)-1H-imidazol-2-yl)pyrrolidin-1-yl)-3-methyl-1-oxobutan-2-ylcarbamate

A mixture of methyl (S)-3-methyl-1-oxo-1-((S)-2-(5-(4-(4,4,5,5-tetramethyl-1,3,2-dioxaborolan-2-yl)phenyl)-1H-imidazol-2-yl)pyrrolidin-1-yl)butan-2-ylcarbamate (5 g, 10.1 mmol), 6-bromo-2-chloroquinoxaline (2.94 g, 12.1 mmol), cesium carbonate (6.51 g, 20.1 mmol), dioxane (100 mL) and water (10 mL) was degassed and flashed with nitrogen three times. Tetrakis(triphenylphosphine)palladium (0) (1.16 g, 1.01 mmol) was then added to the mixture. The mixture was then heated at 80° C. overnight while stirring. Solvent was then removed by evaporation in vacuo. The solid residue was treated with DCM and water to make a biphasic solution. The dichloromethane layer was separated, dried over sodium sulfate, filtered and evaporated. The black crude product was purified by column (0-5% MeOH in DCM) to give a deep colored solid product, methyl (S)-1-((S)-2-(5-(4-(6-bromoquinoxalin-2-yl)phenyl)-1H-imidazol-2-yl)pyrrolidin-1-yl)-3-methyl-1-oxobutan-2-ylcarbamate (5.47 g, 94%): ESI-LRMS m/e calcd for C₂₈H₂₉BrN₆O₃ [M⁺] 578, found 579 [M+H⁺].

### Intermediate 31

### (S)-tert-butyl 2-(5-(4′-(2-((2S,5S)-5-(methoxycarbonylamino)-4-oxo-1,2,4,5,6,7-hexahydroazepino[3,2,1-hi]indol-2-yl)-1H-imidazol-5-yl)biphenyl-4-yl)-4H-1,2,4-triazol-3-yl)pyrrolidine-1-carboxylate

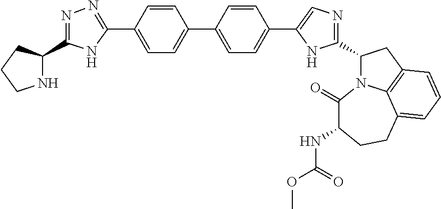

### (S)-tert-butyl 2-(2-(4-bromobenzoyl) hydrazinecarbonyl)pyrrolidine-1-carboxylate

A mixture of (S)-1-(tert-butoxycarbonyl)pyrrolidine-2-carboxylic acid (10 g, 46.5 mmol), TBTU (17.9 g, 55.8 mmol), N-methylmorpholine (7.66 mL, 69.7 mmol) and DMF (100 mL) was stayed at room temperature for 30 minutes, followed by addition of 4-bromobenzohydrazide (9.99 g, 46.5 mmol). The resulting solution was stayed at room temperature for 12 hours, followed by dilution with EtOAc (350 mL) and water (400 mL). The organic layer was separated, washed with water and brine, dried over sodium sulfate, filtered and evaporated. The residue was washed with minimal methanol to give pure (S)-tert-butyl 2-(2-(4-bromobenzoyl) hydrazinecarbonyl)pyrrolidine-1-carboxylate (14.2 g, 74.1%): ESI-LRMS m/e calcd for C₁₇H₂₂BrN₃O₄ [M⁺]413, found 414 [M+H⁺].

### (S)-tert-butyl2-(5-(4-bromophenyl)-1,3,4-oxadiazol-2-yl)pyrrolidine-1-carboxylate

To a suspension of (S)-tert-butyl 2-(2-(4-bromobenzoyl)hydrazinecarbonyl)pyrrolidine-1-carboxylate (13.8 g, 33.5 mmol), PPh₃ (13.2 g, 50.2 mmol) and N,N′-diisopropylethylamine (17.5 mL, 100 mmol) in acetonitrile (250 mL) at room temperature was added hexachloroethane (11.1 g, 46.9 mmol). The mixture was stirred at room temperature overnight. Solvent was then removed by evaporation. The residue was partitioned between EtOAc and water. The organic layer was separated, washed with water and brine, dried over sodium sulfate, filtered and evaporated. The residue was purified by column chromatography (0-80% EtOAc in hexane) to give (S)-tert-butyl 2-(5-(4-bromophenyl)-1,3,4-oxadiazol-2-yl)pyrrolidine-1-carboxylate, (10 g, 75.8%): ESI-LRMS m/e calcd for C₁₇H₂₀BrN₃O₃ [M⁺]395, found 396 [M+H⁺].

### (S)-tert-butyl 2-(5-(4-bromophenyl)-4H-1,2,4-triazol-3-yl)pyrrolidine-1-carboxylate

A mixture of (S)-tert-butyl 2-(5-(4-bromophenyl)-1,3,4-oxadiazol-2-yl)pyrrolidine-1-carboxylate (4.5 g, 11.4 mmol), ammonium acetate (4.4 g, 57.1 mmol) and toluene was heated to reflux for 3 days. The mixture was then cooled to room temperature, diluted with ethyl acetate, washed with water and brine, dried over sodium sulfate, filtered and evaporated. The residue was purified by reverse phase column chromatography (20-100% acetonitrile in water) to give (S)-tert-butyl 2-(5-(4-bromophenyl)-4H-1,2,4-triazol-3-yl)pyrrolidine-1-carboxylate (0.8 g, 17.8%): ESI-LRMS m/e calcd for C₁₇H₂₁BrN₄O₂ [M⁺]392, found 393 [M+H⁺].

### (S)-tert-butyl 2-(5-(4-(4,4,5,5-tetramethyl-1,3,2-dioxaborolan-2-yl)phenyl)-4H-1,2,4-triazol-3-yl)pyrrolidine-1-carboxylate

To a 20-mL microwave reaction vial was added (S)-tert-butyl 2-(5-(4-bromophenyl)-4H-1,2,4-triazol-3-yl)pyrrolidine-1-carboxylate (800 mg, 2.03 mmol), 4,4,4′,4′,5,5,5′,5′-octamethyl-2,2′-bi(1,3,2-dioxaborolane) (1.55 g, 6.1 mmol), PdCl₂(dppf)-CH₂Cl₂ adduct (149 mg, 0.203 mmol), potassium acetate (599 mg, 6.1 mmol) and dioxane (12 mL). The vial was sealed, degassed and flashed with nitrogen three times and heated to 150° C. for 2 hours. The mixture was treated with EtOAc (300 mL) and water. The biphasic mixture was stirred with charcoal for 1 hour, followed by filtration through celite. The organic phase was separated, washed with brine, dried over sodium sulfate, filtered, and evaporated. The residue was purified by column (0-10% MeOH in methylene chloride) to give (S)-tert-butyl 2-(5-(4-(4,4,5,5-tetramethyl-1,3,2-dioxaborolan-2-yl)phenyl)-4H-1,2,4-triazol-3-yl)pyrrolidine-1-carboxylate as a yellow solid (750 mg, 83.7%): ESI-LRMS m/e calcd for C₂₃H₃₃BN₄O₄ [M⁺] 440, found 441 [M+H⁺].

### (S)-tert-butyl 2-(5-(4′-(2-((2S,5S)-5-(methoxycarbonylamino)-4-oxo-1,2,4,5,6,7-hexahydroazepino[3,2,1-hi]indol-2-yl)-1H-imidazol-5-yl)biphenyl-4-yl)-4H-1,2,4-triazol-3-yl)pyrrolidine-1-carboxylate

In a microwave reactor vessel was added methyl (2S,5S)-2-(5-(4-bromophenyl)-1H-imidazol-2-yl)-4-oxo-1,2,4,5,6,7-hexahydroazepino[3,2,1-hi]indol-5-ylcarbamate (85 mg, 0.18 mmol), (S)-tert-butyl 2-(5-(4-(4,4,5,5-tetramethyl-1,3,2-dioxaborolan-2-yl)phenyl)-4H-1,2,4-triazol-3-yl)pyrrolidine-1-carboxylate (93 mg, 0.21 mmol), sodium carbonate (37.4 mg, 0.35 mmol), PdCl₂(dppf)-CH₂Cl₂ adduct (12.9 mg, 0.018 mmol), DMF (5 mL) and water (0.5 mL). The mixture was then degassed, flashed with nitrogen, and heated at 135° C. for 15 min. The mixture was then treated with EtOAc (150 mL) and water (20 mL), followed by stirring with charcoal for 1 hour and filtration through celite. The organic layer was separated, washed with brine, dried over sodium sulfate, filtered, and evaporated to give crude product, (S)-tert-butyl 2-(5-(4′-(2-((2S,5S)-5-(methoxycarbonylamino)-4-oxo-1,2,4,5,6,7-hexahydroazepino[3,2,1-hi]indol-2-yl)-1H-imidazol-5-yl)biphenyl-4-yl)-4H-1,2,4-triazol-3-yl)pyrrolidine-1-carboxylate, which was used directly for next step: ESI-LRMS m/e calcd for C₄₀H₄₂N₈O₅ [M⁺] 714, found 715 [M+H⁺].

### (S)-tert-butyl2-(5-(4′-(2-((2S,5S)-5-(methoxycarbonylamino)-4-oxo-1,2,4,5,6,7-hexahydroazepino[3,2,1-hi]indol-2-yl)-1H-imidazol-5-yl)biphenyl-4-yl)-4H-1,2,4-triazol-3-yl)pyrrolidine-1-carboxylate

A solution of (S)-tert-butyl 2-(5-(4′-(2-((2S,5S)-5-(methoxycarbonylamino)-4-oxo-1,2,4,5,6,7-hexahydroazepino[3,2,1-hi]indol-2-yl)-1H-imidazol-5-yl)biphenyl-4-yl)-4H-1,2,4-triazol-3-yl)pyrrolidine-1-carboxylate, trifluoroacetic acid (5 mL) and dichloromethane (5 mL) was stirred at room temperature for 2 h. The solution was evaporated and the residue was purified by preparative HPLC (5-100% ACN with 0.1% TFA in water with 0.1% TFA) to give (S)-tert-butyl 2-(5-(4′-(2-((2S,5S)-5-(methoxycarbonylamino)-4-oxo-1,2,4,5,6,7-hexahydroazepino[3,2,1-hi]indol-2-yl)-1H-imidazol-5-yl)biphenyl-4-yl)-4H-1,2,4-triazol-3-yl)pyrrolidine-1-carboxylate (7 mg, 6.4%): ESI-LRMS m/e calcd for C₃₅H₃₄N₈O₃ [M⁺] 614, found 615 [M+H⁺].

### Example 1

### {(2S,5S)-2-[5-(4′-{2-[(2S,4S)-4-Fluoro-1-((S)-2-methoxycarbonylamino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester

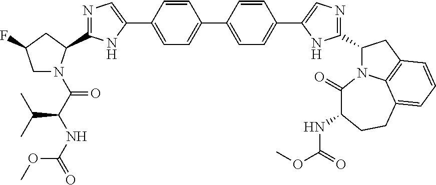

In a 10 mL seal tube, methyl (S)-1-((2S,4S)-4-fluoro-2-(5-(4-(4,4,5,5-tetramethyl-1,3,2-dioxaborolan-2-yl)phenyl)-1H-imidazol-2-yl)pyrrolidin-1-yl)-3-methyl-1-oxobutan-2-ylcarbamate (32.1 mg, 62.3 μmol, Eq: 1.00) (intermediate 3), methyl (2S,5S)-2-(5-(4-bromophenyl)-1H-imidazol-2-yl)-4-oxo-1,2,4,5,6,7-hexahydroazepino[3,2,1-hi]indol-5-ylcarbamate (30 mg, 62.3 μmol, Eq: 1.00) (intermediate 2) and sodium bicarbonate (sat. solution, 400 μl, excess) were combined with tert-butanol (2.00 ml) to give a light brown solution and degassed for 5 min. PdCl₂(DPPF) (4.56 mg, 6.23 μmol, Eq: 0.1) was added, flushed with nitrogen. It was sealed heating at 90° C. for 5 hr then diluted with EtOAc, concentrated and purified on a silica gel column (CH₂Cl₂, 2%, 3%, 5% MeOH/CH₂Cl₂)to afford {(2S,5S)-2-[5-(4′-{2-[(2S,4S)-4-Fluoro-1-((S)-2-methoxycarbonylamino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester as a light yellow solid (20 mg, 38.6%). ESI-LRMS m/e calcd for C₄₃H₄₅F₁N₈O₆, [M⁺] 788, found 789. ¹H NMR (400 MHz, DMSO-d₆) δ ppm 0.73-1.16 (m, 12H) 1.87-2.34 (m, 7H) 3.09 (br. s., 4H) 3.42-3.73 (m, 13H) 3.90-4.43 (m, 7H) 5.13-5.61 (m, 4H) 6.77-7.26 (m, 7H) 7.34-7.96 (m, 20H) 11.43-11.66 (m, 1H) 11.73-12.00 (m, 2H).

### Example 2

### {(2S,5S)-2-[5-(4′-{2-[(2S,4R)-4-Fluoro-1-((S)-2-methoxycarbonylamino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester

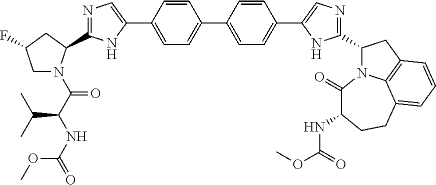

In a 10 mL seal tube, methyl (S)-1-((2S,4R)-4-fluoro-2-(5-(4-(4,4,5,5-tetramethyl-1,3,2-dioxaborolan-2-yl)phenyl)-1H-imidazol-2-yl)pyrrolidin-1-yl)-3-methyl-1-oxobutan-2-ylcarbamate (32.1 mg, 62.3 μmol, Eq: 1.00) (Intermediate 4), methyl (2S,5S)-2-(5-(4-bromophenyl)-1H-imidazol-2-yl)-4-oxo-1,2,4,5,6,7-hexahydroazepino[3,2,1-hi]indol-5-ylcarbamate (30 mg, 62.3 μmol, Eq: 1.00) (Intermediate 2) and sodium bicarbonate (400 μl, excess) were combined with tert-butanol (2.00 ml) to give a light brown solution and degassed for 5 min. PdCl₂ (DPPF) (4.56 mg, 6.23 μmol, Eq: 0.1) was added and flushed with N₂. It was sealed heating at 90° C. for 5 hr then diluted with EtOAc, filtered and concentrated. The crude mixture was purified on a silica gel column (CH₂Cl₂, 2%, 3%, 5% MeOH/CH₂Cl₂) to afford {(2S,5S)-2-[5-(4′-{2-[(2S,4R)-4-Fluoro-1-((S)-2-methoxycarbonylamino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester as a light yellow solid (17 mg, 32.8%). ESI-LRMS m/e calcd for C₄₃H₄₅F₁N₈O₆, [M⁺]788, found 789. ¹H NMR (400 MHz, DMSO-d₆) δ ppm 0.81 (d, J=5.77 Hz, 11H) 1.69-2.39 (m, 7H) 3.00-3.19 (m, 4H) 3.55 (d, J=6.02 Hz, 12H) 3.80-4.39 (m, 8H) 4.96-5.20 (m, 2H) 5.36-5.61 (m, 2H) 5.67-5.89 (m, 2 H) 6.82-7.39 (m, 9H) 7.43-7.94 (m, 18H) 11.70-12.10 (m, 3H).

### Example 3

### {(2S,5S)-2-[5-(4-{2-[(S)-1-((S)-2-Methoxycarbonylamino-3-methyl-butyryl)-pyrrolidin-2-yl]-3H-benzoimidazol-5-yl}-phenyl)-1H-imidazol-2-yl]-4-oxo-1,2,4,5,6,7-hexahydro-azepino[3,2,1-hi]indol-5-yl}-carbamic acid methyl ester

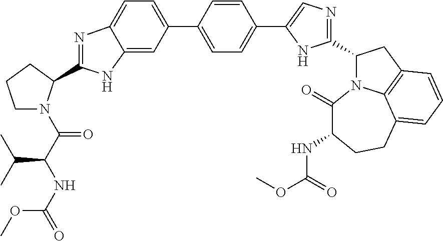

In a 10 mL seal tube, methyl (S)-3-methyl-1-oxo-1-((S)-2-(5-(4,4,5,5-tetramethyl-1,3,2-dioxaborolan-2-yl)-1H-benzo[d]imidazol-2-yl)pyrrolidin-1-yl)butan-2-ylcarbamate (63.5 mg, 135 μmol, Eq: 1.00) (Intermediate 5), methyl (2S,5S)-2-(5-(4-bromophenyl)-1H-imidazol-2-yl)-4-oxo-1,2,4,5,6,7-hexahydroazepino[3,2,1-hi]indol-5-ylcarbamate (65 mg, 135 μmol, Eq: 1.00) (Intermediate 2) and sodium bicarbonate (saturated solution, 400 μA, excess) were combined with tert-butanol (2.00 ml) to give a light brown solution and degassed for 5 min. PdCl₂ (DPPF) (9.88 mg, 13.5 μmol, Eq: 0.1) was added, flushed with nitrogen. It was sealed heating at 90° C. for 5 hr then diluted with EtOAc, filtered and concentrated. The crude material was purified on column (CH₂Cl₂, 3%, 5%, 8%, 10% MeOH/CH₂Cl₂) to afford {(2S,5S)-2-[5-(4-{2-[(S)-1-((S)-2-Methoxycarbonylamino-3-methyl-butyryl)-pyrrolidin-2-yl]-3H-benzoimidazol-5-yl}-phenyl)-1H-imidazol-2-yl]-4-oxo-1,2,4,5,6,7-hexahydro-azepino[3,2,1-hi]indol-5-yl]-carbamic acid methyl ester as a yellow solid (35 mg, 33.1%). ESI-LRMS m/e calcd for C₄ᵢH₄₄N₈O₆, [M⁺] 744, found 745 [M+H⁺]. ¹H NMR (400 MHz, DMSO-d₆) δ ppm 0.84 (dd, J=12.80, 6.53 Hz, 15H) 1.78-2.09 (m, 3H) 2.23 (br. s., 3H) 2.95-3.19 (m, 5H) 3.36-3.71 (m, 20H) 3.85 (br. s., 4H) 4.02-4.32 (m, 4H) 5.08-5.28 (m, 2H) 5.76 (s, 3H) 6.94-7.21 (m, 8H) 7.25-7.89 (m, 23H) 11.82 (br. s., 2H) 12.04-12.33 (m, 2H).

### Example 4

### {(2S,5S)-2-[5-(4′-{2-[(S)-4,4-Difluoro-1-((S)-2-methoxycarbonylamino-3-methyl-butyryl)-pyrrolidin-2-yl]-3H-benzoimidazol-5-yl}-biphenyl-4-yl)-1H-imidazol-2-yl]-4-oxo-1,2,4,5,6,7-hexahydro-azepino[3,2,1-hi]indol-5-yl}-carbamic acid methyl ester

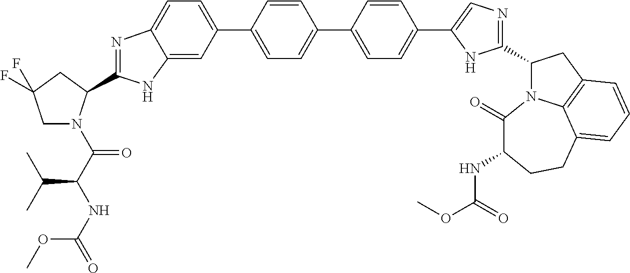

In a 10 mL seal tube, methyl (S)-1-((S)-4,4-difluoro-2-(5-(4-(4,4,5,5-tetramethyl-1,3,2-dioxaborolan-2-yl)phenyl)-1H-benzo[d]imidazol-2-yl)pyrrolidin-1-yl)-3-methyl-1-oxobutan-2-ylcarbamate (78.7 mg, 135 μmol, Eq: 1.00) (Intermediate 6), methyl (2S,5S)-2-(5-(4-bromophenyl)-1H-imidazol-2-yl)-4-oxo-1,2,4,5,6,7-hexahydroazepino[3,2,1-hi]indol-5-ylcarbamate (65 mg, 135 μmol, Eq: 1.00) (Intermediate 2) and sodium bicarbonate (sat. solution, 400 μl, excess) were combined with tert-butanol (2.00 ml) to give a light brownish solution and degassed for 5 min. PdCl₂ (DPPF) (9.88 mg, 13.5 μmol, Eq: 0.1) was added and flushed with nitrogen. It was sealed heating at 90° C. for 5 hr then diluted with EtOAc, filtered, washed with EtOAc. The filtrate was concentrated and purified on a silica gel column (CH₂Cl₂, 3%, 5%, 8%, 10% MeOH/CH₂Cl₂) to afford {(2S,5S)-2-[5-(4′-{2-[(S)-4,4-Difluoro-1-((S)-2-methoxycarbonylamino-3-methyl-butyryl)-pyrrolidin-2-yl]-3H-benzoimidazol-5-yl}-biphenyl-4-yl)-1H-imidazol-2-yl]-4-oxo-1,2,4,5,6,7-hexahydro-azepino[3,2,1-hi]indol-5-yl]-carbamic acid methyl ester (90% pure). It was further purified on prep-HPLC to afford a white solid (28 mg, 23%). ESI-LRMS m/e calcd for C₄₇H₄₆F₂N₈O₆, [M⁺] 856, found 857 [M+H⁺]. ¹H NMR (400 MHz, DMSO-d₆) ppm 0.83 (br. s., 14H) 3.09 (br. s., 3H) 3.21-3.45 (m, 17H) 3.97 (br. s., 1H) 5.29-6.01 (m, 3H) 6.89-7.30 (m, 4H) 7.37-8.06 (m, 35H) 11.86 (br. s., 2H) 12.40 (d, J=11.04 Hz, 2H).

### Example 5

### {(2S,5S)-2-[6-(4-{2-[(S)-1-((S)-2-Methoxycarbonylamino-3-methyl-butyryl)-pyrrolidin-2-yl]-3H-benzoimidazol-5-yl}-phenyl)-1H-benzoimidazol-2-yl]-4-oxo-1,2,4,5,6,7-hexahydro-azepino[3,2,1-hi]indol-5-yl}-carbamic acid methyl ester

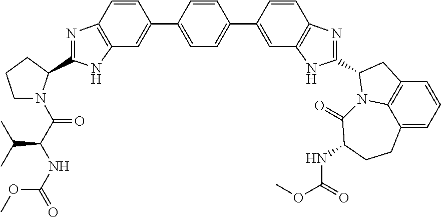

In a 10 mL seal tube, methyl (S)-3-methyl-1-oxo-1-((S)-2-(6-(4-(4,4,5,5-tetramethyl-1,3,2-dioxaborolan-2-yl)phenyl)-1H-benzo[d]imidazol-2-yl)pyrrolidin-1-yl)butan-2-ylcarbamate (90 mg, 124 μmol, Eq: 1.00) (Intermediate 6), methyl (2S,5S)-2-(5-bromo-1H-benzo[d]imidazol-2-yl)-4-oxo-1,2,4,5,6,7-hexahydroazepino[3,2,1-hi]indol-5-ylcarbamate (56.2 mg, 124 μmol, Eq: 1.00) (Intermediate 7) and sodium bicarbonate solution (sat. solution, 0.4 ml, excess) were combined with tert-butanol (2.00 ml) to give a light brown solution and degassed for 10 min. PdCl₂ (DPPF) (9.04 mg, 12.4 μmol, Eq: 0.1) was added and flushed with N₂. It was sealed heating at 90° C. for 5 hr. It was diluted with CH₂Cl₂, concentrated and purified on a silica gel column (CH₂Cl₂, 2%, 3%, 5%, 8% MeOH/CH₂Cl₂) to afford {(2S,5S)-2-[6-(4-{2-[(S)-1-((S)-2-Methoxycarbonylamino-3-methyl-butyryl)-pyrrolidin-2-yl]-3H-benzoimidazol-5-yl}-phenyl)-1H-benzoimidazol-2-yl]-4-oxo-1,2,4,5,6,7-hexahydro-azepino[3,2,1-hi]indol-5-yl]-carbamic acid methyl ester as a light yellow solid (18 mg, 18.3%). ESI-LRMS m/e calcd for C₄₅H₄₆N₈O₆, [M⁺] 794, found 795 [M+H⁺]. ¹H NMR (400 MHz, METHANOL-d₄) δ ppm 0.78-1.11 (m, 7 H) 1.99-2.55 (m, 9H) 3.69 (d, J=7.03 Hz, 6H) 3.88-4.11 (m, 2H) 4.23-4.46 (m, 2H) 5.21-5.37 (m, 1H) 5.51 (s, 2H) 6.00-6.22 (m, 1H) 6.93-7.29 (m, 3H) 7.74 (br. s., 11H).

### Example 6

### {(2S,5S)-2-[6-(2-{2-[(S)-1-((S)-2-Methoxycarbonylamino-3-methyl-butyryl)-pyrrolidin-2-yl]-3H-benzoimidazol-5-yl}-quinoxalin-6-yl)-1H-benzoimidazol-2-yl]-4-oxo-1,2,4,5,6,7-hexahydro-azepino[3,2,1-hi]indol-5-yl}-carbamic acid methyl ester

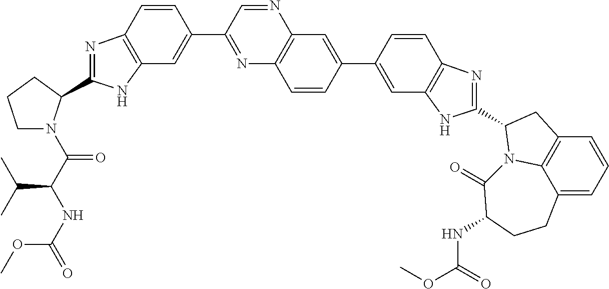

In a 10 mL seal tube, methyl (S)-3-methyl-1-oxo-1-((S)-2-(6-(6-(4,4,5,5-tetramethyl-1,3,2-dioxaborolan-2-yl)quinoxalin-2-yl)-1H-benzo[d]imidazol-2-yl)pyrrolidin-1-yl)butan-2-ylcarbamate (65 mg, 109 μmol, Eq: 1.00) (Intermediate 9), methyl (2S,5S)-2-(5-bromo-1H-benzo[d]imidazol-2-yl)-4-oxo-1,2,4,5,6,7-hexahydroazepino[3,2,1-hi]indol-5-ylcarbamate (49.4 mg, 109 μmol, Eq: 1.00) (Intermediate 7) and sodium bicarbonate solution (sat. solution, 0.4 ml, excess) were combined with tert-butanol (2.00 ml) to give a light brown solution and degassed for 10 min. Pd(DPPF)Cl₂ (7.95 mg, 10.9 μmol, Eq: 0.1) was added and flushed with nitrogen. It was sealed heating at 90° C. for 5 hr. The reaction mixture was diluted with CH₂Cl₂, concentrated in vacuo and purified on a silica gel column (CH₂Cl₂, 2%, 3%, 5%, 8%, and 10% MeOH/CH₂Cl₂) to afford {(2S,5S)-2-[6-(2-{2-[(S)-1-((S)-2-Methoxycarbonylamino-3-methyl-butyryl)-pyrrolidin-2-yl]-3H-benzoimidazol-5-yl}-quinoxalin-6-yl)-1H-benzoimidazol-2-yl]-4-oxo-1,2,4,5,6,7-hexahydro-azepino[3,2,1-hi]indol-5-yl}-carbamic acid methyl ester as a yellow solid (38 mg, 39.2%). ESI-LRMS m/e calcd for C₄₇H₄₆N₁₀O₆, [M⁺] 846, found 847 [M+H⁺]. ¹H NMR (400 MHz, DMSO-d₆) δ ppm 0.80-1.12 (m, 10H) 1.34 (br. s., 1H) 2.09-2.47 (m, 4 H) 3.22 (br. s., 2H) 3.56-4.43 (m, 13H) 5.33 (br. s., 1H) 6.06 (d, J=10.04 Hz, 1H) 7.00-7.91 (m, 9H) 7.97-8.85 (m, 7H) 9.75 (dd, J=11.17, 6.65 Hz, 1H) 12.39-12.70 (m, 2H).

### Example 7

### ((2S,5S)-2-{5-[4-(6-{2-[(S)-1-((S)-2-Methoxycarbonylamino-3-methyl-butyryl)-pyrrolidin-2-yl]-3H-benzoimidazol-5-yl}-pyridin-3-yl)-phenyl]-1H-imidazol-2-yl}-4-oxo-1,2,4,5,6,7-hexahydro-azepino[3,2,1-hi]indol-5-yl)-carbamic acid methyl ester

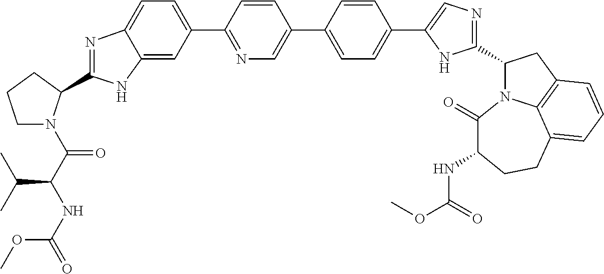

In a 10 mL seal tube, methyl (S)-3-methyl-1-oxo-1-((S)-2-(5-(5-(4,4,5,5-tetramethyl-1,3,2-dioxaborolan-2-yl)pyridin-2-yl)-1H-benzo[d]imidazol-2-yl)pyrrolidin-1-yl)butan-2-ylcarbamate (73.9 mg, 135 μmol, Eq: 1.00) (Intermediate 10), methyl (2S,5S)-2-(5-(4-bromophenyl)-1H-imidazol-2-yl)-4-oxo-1,2,4,5,6,7-hexahydroazepino[3,2,1-hi]indol-5-ylcarbamate (65 mg, 135 μmol, Eq: 1.00) (Intermediate 2) and sodium bicarbonate (sat. solution, 400 μl, excess) were combined with tert-butanol (2.00 ml) to give a light brown solution and degassed for 5 min. PdCl₂ (DPPF) (12 mg, 16.4 μmol, Eq: 0.121) was added and flushed with nitrogen. It was sealed heating at 90° C. for 5 hr. The reaction mixture was diluted with EtOAc, filtered, concentrated and purified on a silica gel column (CH₂Cl₂, 3%, 5%, 8%, 10% MeOH/CH₂Cl₂) to afford ((2S,5S)-2-{5-[4-(6-{2-[(S)-1-((S)-2-methoxycarbonyl amino-3-methyl-butyryl)-pyrrolidin-2-yl]-3H-benzoimidazol-5-yl}-pyridin-3-yl)phenyl]-1H-imidazol-2-yl}-4-oxo-1,2,4,5,6,7-hexahydro-azepino[3,2,1-hi]indol-5-yl)-carbamic acid methyl ester as a light brown solid (13 mg, 11%). ESI-LRMS m/e calcd for C₄₆H₄₇N₉O₆, [M⁺] 821, found 822 [M+H⁺]. ¹H NMR (400 MHz, DMSO-d₆) δ ppm 0.65-1.00 (m, 11H) 1.26 (d, J=13.05 Hz, 5H) 1.55-2.41 (m, 17H) 2.97-3.20 (m, 3H) 3.47-3.71 (m, 12H) 4.03-4.32 (m, 3H) 5.04-5.34 (m, 1H) 6.90-7.39 (m, 6H) 7.43-7.94 (m, 11H) 7.99-8.41 (m, 5H) 8.98 (br. s., 2H) 11.68-12.51 (m, 2H).

### Example 8

### {(2S,5S)-2-[5-(4′-{2-[(S)-7-((S)-2-Methoxycarbonylamino-3-methyl-butyryl)-1,4-dioxa-7-aza-spiro[4.4]non-8-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl Ester

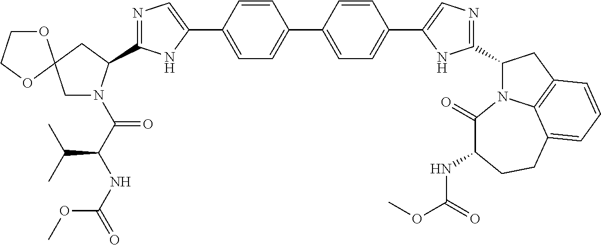

In a 10 mL seal tube, methyl (S)-3-methyl-1-oxo-1-((S)-8-(5-(4-(4,4,5,5-tetramethyl-1,3,2-dioxaborolan-2-yl)phenyl)-1H-imidazol-2-yl)-1,4-dioxa-7-azaspiro[4.4]nonan-7-yl)butan-2-ylcarbamate (30 mg, 54.1 μmol, Eq: 1.00) (Intermediate 11), methyl (2S,5S)-2-(5-(4-bromophenyl)-1H-imidazol-2-yl)-4-oxo-1,2,4,5,6,7-hexahydroazepino[3,2,1-hi]indol-5-ylcarbamate (30 mg, 62.3 μmol, Eq: 1.15) (Intermediate 2) and sodium bicarbonate (sat solution, 400 μl, excess)) were combined with tert-butanol (2.00 ml) to give a light brown solution and degassed for 5 min. PdCl₂ (DPPF) (4.56 mg, 6.23 μmol, Eq: 0.115) was added and flushed with nitrogen. It was sealed heating at 90° C. for 5 hr. The mixture was diluted with EtOAc, filtered, concentrated and purified on a silica gel column (CH₂Cl₂, 2%, 3%, 4%, 5% MeOH/CH₂Cl₂) to afford {(2S,5S)-2-[5-(4′-{2-[(S)-7-((S)-2-Methoxycarbonylamino-3-methyl-butyryl)-1,4-dioxa-7-aza-spiro[4.4]non-8-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl Ester as a light yellow solid (18 mg, 38.1%). ESI-LRMS m/e calcd for C₄₅H₄₈N₈O₈, [M⁺] 828, found 829 [M+H]. ¹H NMR (400 MHz, DMSO-d₆) δ ppm 0.82 (d, J=12.55 Hz, 11H) 1.75-2.36 (m, 7H) 3.08 (br. s., 3H) 3.55 (br. s., 11H) 3.76-4.28 (m, 13H) 5.06 (br. s., 2H) 5.64-5.93 (m, 3H) 6.88-7.35 (m, 8H) 7.39-7.96 (m, 17H) 11.84 (br. s., 2H).

### Example 9

### {(2S,5S)-2-[5-(4′-{2-[2-((S)-2-Methoxycarbonylamino-3-methyl-butyryl)-8-oxa-2-aza-spiro[4.5]dec-3-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester

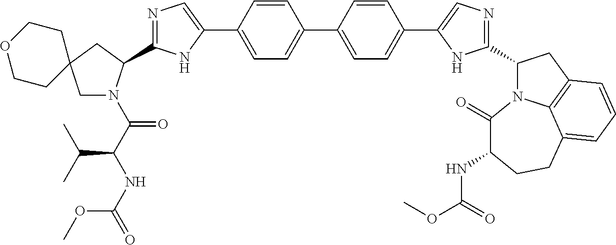

In a 10 mL seal tube, methyl (2S)-3-methyl-1-oxo-1-(3-(5-(4-(4,4,5,5-tetramethyl-1,3,2-dioxaborolan-2-yl)phenyl)-1H-imidazol-2-yl)-8-oxa-2-azaspiro[4.5]decan-2-yl)butan-2-ylcarbamate (50 mg, 88.3 μmol, Eq: 1.00) (Intermediate 12), methyl (2S,5S)-2-(5-(4-bromophenyl)-1H-imidazol-2-yl)-4-oxo-1,2,4,5,6,7-hexahydroazepino[3,2,1-hi]indol-5-ylcarbamate (42.5 mg, 88.3 μmol, Eq: 1) (Intermediate 2) and sodium bicarbonate (sat. solution, 400 μl, excess)) were combined with tert-butanol (2.00 ml) to give a light brown solution and degassed for 5 min. PdCl₂(DPPF) (6.45 mg, 8.83 μmol, Eq: 0.1) was added and flushed with nitrogen. It was sealed heating at 90° C. for 5 hr, then was diluted with EtOAc, filtered, concentrated and purified on a silica gel column (CH₂Cl₂, 2%, 3%, 4%, 5% MeOH/CH₂Cl₂) to afford {(2S,5S)-2-[5-(4′-{2-[2-((S)-2-Methoxycarbonylamino-3-methyl-butyryl)-8-oxa-2-aza-spiro[4.5]dec-3-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester as a light yellow solid (25 mg, 33.7%). ESI-LRMS m/e calcd for C₄₇H₅₂N₈O₇, [M⁺] 840, found 841 [M+H]. ¹H NMR (400 MHz, DMSO-d₆) δ ppm 0.04-0.22 (m, 1H) 0.55-0.71 (m, 1H) 0.87 (d, J=13.30 Hz, 5H) 1.34-1.74 (m, 5H) 2.07-2.41 (m, 3H) 2.96-3.16 (m, 2H) 3.46-3.74 (m, 14H) 4.09-4.28 (m, 2 H) 5.65-5.93 (m, 1H) 6.81-7.23 (m, 4H) 7.24-7.90 (m, 14H) 11.72-11.95 (m, 1H).

### Example 10

### {(2S,5S)-2-[6-(4-{2-[(S)-1-((S)-2-Methoxycarbonylamino-3-methyl-butyryl)-pyrrolidin-2-yl]-3H-imidazol-4-yl}-phenyl)-1H-benzoimidazol-2-yl]-4-oxo-1,2,4,5,6,7-hexahydro-azepino[3,2,1-hi]indol-5-yl}-carbamic acid methyl ester

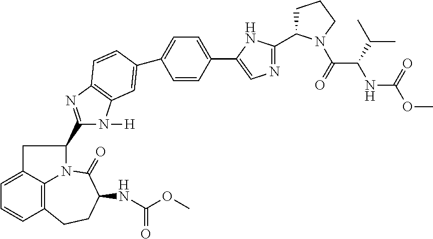

In a vial, methyl (2S,5S)-2-(6-bromo-1H-benzo[d]imidazol-2-yl)-4-oxo-1,2,4,5,6,7-hexahydroazepino[3,2,1-hi]indol-5-ylcarbamate (27.5 mg, 60.4 μmol) (Intermediate 7), methyl (S)-3-methyl-1-oxo-1-((S)-2-(5-(4-(4,4,5,5-tetramethyl-1,3,2-dioxaborolan-2-yl)phenyl)-1H-imidazol-2-yl)pyrrolidin-1-yl)butan-2-ylcarbamate (30 mg, 60.4 μmol) and sodium bicarbonate (15.2 mg, 181 μmol) were combined with Toluene (1.5 ml), Ethanol (1.00 ml) and water (500 μA) to give a light yellow suspension, 1,1′-bis(diphenylphosphino)-ferrocene-palladium(ii) dichloride dichloromethane complex (4.94 mg, 6.04 μmol) was added while the reaction mixture was degassed with nitrogen. The reaction mixture was heated to 90° C. and stirred for over night. The reaction mixture was poured into water and extracted with ethyl acetate, the organic layers were dried over sodium sulfate and concentrated in vacuo. The crude material was purified by flash chromatography (silica gel, 5% methanol in 1:1 dichloromethane/ethyl acetate). Re-purified by preparative TLC (silica gel, 6% methanol/dichloromethane, eluted twice) to afford {(2S,5S)-2-[6-(4-{2-[(S)-1-((S)-2-Methoxycarbonylamino-3-methyl-butyryl)-pyrrolidin-2-yl]-3H-imidazol-4-yl}-phenyl)-1H-benzoimidazol-2-yl]-4-oxo-1,2,4,5,6,7-hexahydro-azepino[3,2,1-hi]indol-5-yl}-carbamic acid methyl ester as yellow solid (27 mgs, 60%). Calculated for C₄₁H₄₄N₈O₆ [M⁺] 744.3, found [M+H⁺]745.

¹H NMR (400 MHz, CHLOROFORM-d) δ ppm 0.74-0.86 (m, 6H) 0.94-1.04 (m, 1H) 1.86-1.96 (m, 2H) 1.99-2.19 (m, 2H) 2.23-2.37 (m, 2H) 2.87-3.02 (m, 2H) 3.10-3.28 (m, 1H) 3.44-3.54 (m, 1H) 3.63 (s, 3H) 3.67 (s, 3H) 3.72-3.82 (m, 1H) 4.16-4.38 (m, 3H) 5.16-5.22 (m, 1H) 5.34-5.47 (m, 1H) 5.91-6.11 (m, 2H) 6.86-7.06 (m, 2H) 7.15 (s, 1H) 7.16-7.19 (m, 1H) 7.25-8.02 (m, 8H) 10.23 (br. s., 1H).

### Example 11

### {(2S,5S)-2-[6-(4′-{2-[(S)-1-((8)-2-Methoxycarbonylamino-3-methyl-butyryl)-pyrrolidin-2-yl]-3H-imidazol-4-yl}-biphenyl-4-yl)-1H-benzoimidazol-2-yl]-4-oxo-1,2,4,5,6,7-hexahydro-azepino[3,2,1-hi]indol-5-yl}-carbamic methyl ester

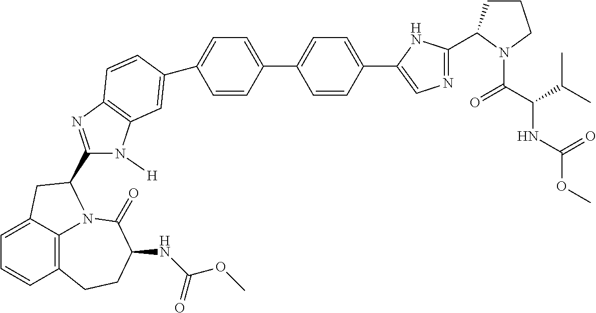

In a vial, 1,4-phenyldiboronic acid, bis(pinacol) ester (73.4 mg, 223 μmol), methyl (S)-1-((S)-2-(5-(4-bromophenyl)-1H-imidazol-2-yl)pyrrolidin-1-yl)-3-methyl-1-oxobutan-2-ylcarbamate (100 mg, 223 μmol) and carbonic acid, sodium salt (56.8 mg, 668 μmol) were combined with toluene (3 ml), ethanol (2.00 ml) and water (1.00 ml) to give a light yellow suspension. Tetrakis(triphenylphosphine)palladium (0) (18.0 mg, 15.6 μmol) was added while the reaction mixture was degassed with nitrogen. The reaction mixture was heated to 90° C. and stirred for over night. The reaction mixture was poured into water and extracted with ethyl acetate, The organic layers were dried over sodium sulfate and concentrated in vacuo. The crude material was purified by flash chromatography (silica gel, 5% methanol/dichloromethane). The impure material was re-purified by flash chromatography (silica gel, 100% ethyl acetate) to afford methyl (S)-3-methyl-1-oxo-1-((S)-2-(5-(4′-(4,4,5,5-tetramethyl-1,3,2-dioxaborolan-2-yl)biphenyl-4-yl)-1H-imidazol-2-yl)pyrrolidin-1-yl)butan-2-ylcarbamate as pale yellow solid (65 mgs, 51%). Calculated for C₃₂H₄₁BN₄O₅ [M⁺] 572.3, found [M+H⁺] 573.

In a vial, methyl (2S,5S)-2-(6-bromo-1H-benzo[d]imidazol-2-yl)-4-oxo-1,2,4,5,6,7-hexahydroazepino[3,2,1-hi]indol-5-ylcarbamate (51.7 mg, 114 μmol) (Intermediate 7), methyl (S)-3-methyl-1-oxo-1-((S)-2-(5-(4′-(4,4,5,5-tetramethyl-1,3,2-dioxaborolan-2-yl)biphenyl-4-yl)-1H-imidazol-2-yl)pyrrolidin-1-yl)butan-2-ylcarbamate (65 mg, 114 μmol) and sodium bicarbonate (28.6 mg, 341 μmol) were combined with Toluene (1.5 ml), Ethanol (1.00 ml) and Water (500 μA) to give a light yellow suspension, 1,1′-Bis(diphenylphosphino)ferrocene-palladium(ii) dichloride dichloromethane complex (9.27 mg, 11.4 μmol) was added while the reaction mixture was degassed with nitrogen, The reaction mixture was heated to 90° C. and stirred for over night. The reaction mixture was poured into water and extracted with ethyl acetate, the organic layers were dried over sodium sulfate and concentrated in vacuo. The crude material was purified by flash chromatography (silica gel, 5% methanol in 1:1 dichloromethane/ethyl acetate); re-purified by preparative TLC (silica gel, 5% methanol/dichloromethane, eluted for 4 times), the impure material was dissolved in ethyl acetate, washed with 3N sodium hydroxide aqueous solution twice, the organic solution was dried over sodium sulfate, and concentrated in vacuo. The material was re-purified by flash chromatography (silica gel, 5% methanol/dichloromethane) to afford {(2S,5S)-2-[6-(4′-{2-[(S)-1-((S)-2-Methoxycarbonylamino-3-methyl-butyryl)-pyrrolidin-2-yl]-3H-imidazol-4-yl}-biphenyl-4-yl)-1H-benzoimidazol-2-yl]-4-oxo-1,2,4,5,6,7-hexahydro-azepino[3,2,1-hi]indol-5-yl}-carbamic methyl ester as light yellow solid (13 mgs, 13%). Calculated for C₄₇H₄₈N₈O₆ [M⁺] 820.3, found [M+H⁺] 821. ¹H NMR (300 MHz, DMSO-d₆) δ ppm 0.88 (m, J=15.50, 6.40 Hz, 6 H) 1.80-2.36 (m, 7H) 3.11 (t, J=6.42 Hz, 2H) 3.39-3.45 (m, 2H) 3.55 (s, 6H) 3.62-3.89 (m, 3H) 4.01-4.11 (m, 1H) 4.12-4.23 (m, 1H) 5.04-5.14 (m, 1H) 5.93 (d, J=9.82 Hz, 1H) 6.95-7.38 (m, 4H) 7.43-7.93 (m, 12H) 11.79 (br. s., 1H) 12.27 (s, 1H).

### Example 12

### ((2S,5S)-2-{6-[6-(4-{2-[(S)-1-((S)-2-Methoxycarbonylamino-3-methyl-butyryl)-pyrrolidin-2-yl]-3H-imidazol-4-yl}-phenyl)-pyridin-3-yl]-1H-benzoimidazol-2-yl}-4-oxo-1,2,4,5,6,7-hexahydro-azepino[3,2,1-hi]indol-5-yl)-carbamic acid methyl ester

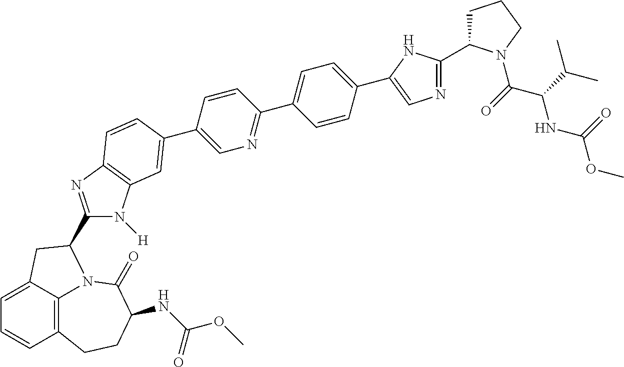

In a vial, methyl (2S,5S)-2-(6-bromo-1H-benzo[d]imidazol-2-yl)-4-oxo-1,2,4,5,6,7-hexahydroazepino[3,2,1-hi]indol-5-ylcarbamate (60 mg, 132 μmol) (Intermediate 7), bis(pinacolato)diboron (43.5 mg, 171 μmol) and potassium acetate (38.8 mg, 395 μmol) were combined with dioxane (5 ml) to give a yellow solution. 1,1′-bis(diphenylphosphino)ferrocene-palladium(ii) dichloride dichloromethane complex (10.8 mg, 13.2 μmol) was added while degassed with nitrogen. The reaction mixture was heated to 90° C. and stirred for over night. The reaction mixture was poured into water and extracted with ethyl acetate, the organic layers were dried over sodium sulfate and concentrated in vacuo. The crude material was purified by flash chromatography (silica gel, 30% to 50% ethyl acetate/hexanes) to afford methyl (2S,5S)-4-oxo-2-(6-(4,4,5,5-tetramethyl-1,3,2-dioxaborolan-2-yl)-1H-benzo[d]imidazol-2-yl)-1,2,4,5,6,7-hexahydroazepino[3,2,1-hi]indol-5-ylcarbamate as yellow solid obtained (54 mgs, 81%).

Calculated for C₂₇H₃₁BN₄O₅ [M⁺] 502.2, found [M+H⁺] 503.

In a vial, methyl (2S,5S)-4-oxo-2-(6-(4,4,5,5-tetramethyl-1,3,2-dioxaborolan-2-yl)-1H-benzo[d]imidazol-2-yl)-1,2,4,5,6,7-hexahydroazepino-[3,2,1-hi]indol-5-ylcarbamate (44 mg, 87.6 μmol), 2,5-dibromopyridine (30 mg, 127 μmol) and carbonic acid, sodium salt (22.3 mg, 263 μmol) were combined with toluene (3 ml), ethanol (2.00 ml) and water (1.00 ml) to give a light yellow suspension. Tetrakis(triphenylphosphine)palladium (0) (10.1 mg, 8.76 μmol) was added while the reaction mixture was degassed with nitrogen. The reaction mixture was heated to 90° C. and stirred for over night. The reaction mixture was poured into saturated sodium chloride aqueous solution and extracted with ethyl acetate, the organic layers were dried over sodium sulfate and concentrated in vacuo. The crude material was purified by flash chromatography (silica gel, 30% to 80% ethyl acetate/hexanes) to afford methyl (2S,5S)-2-(6-(6-bromopyridin-3-yl)-1H-benzo[d]imidazol-2-yl)-4-oxo-1,2,4,5,6,7-hexahydroazepino[3,2,1-hi]indol-5-ylcarbamate (30 mgs, 64%). Calculated for C₂₆H₂₂BrN₅O₃ [M⁺] 531/533, found [M+H⁺] 532/534.

In a vial, methyl (2S,5S)-2-(6-(6-bromopyridin-3-yl)-1H-benzo[d]imidazol-2-yl)-4-oxo-1,2,4,5,6,7-hexahydroazepino[3,2,1-hi]indol-5-ylcarbamate (30 mg, 56.3 μmol), methyl (S)-3-methyl-1-oxo-1-((S)-2-(5-(4-(4,4,5,5-tetramethyl-1,3,2-dioxaborolan-2-yl)phenyl)-1H-imidazol-2-yl)pyrrolidin-1-yl)butan-2-ylcarbamate (28.0 mg, 56.3 μmol) and sodium bicarbonate (14.2 mg, 169 μmol) were combined with toluene (3.00 ml), ethanol (2.00 ml) and water (1 ml) to give a light yellow suspension, 1,1′-bis(diphenylphosphino)ferrocene-palladium(ii) dichloride dichloromethane complex (4.6 mg, 5.63 μmol) was added while the reaction mixture was degassed with argon. The reaction mixture was heated to 90° C. and stirred for 6 hours. The reaction mixture was poured into water and extracted with ethyl acetate; the organic layers were dried over sodium sulfate and concentrated in vacuo. The crude material was purified by preparative TLC (silica gel, 1.0 mm, 5% methanol/ethyl acetate once, 5% methanol/dichloromethane/ammonium hydroxide for twice) to afford ((2S,5S)-2-{6-[6-(4-{2-[(S)-1-((S)-2-Methoxycarbonylamino-3-methyl-butyryl)-pyrrolidin-2-yl]-3H-imidazol-4-yl}-phenyl)-pyridin-3-yl]-1H-benzoimidazol-2-yl}-4-oxo-1,2,4,5,6,7-hexahydro-azepino[3,2,1-hi]indol-5-yl)-carbamic acid methyl ester as pale yellow solid (26 mgs, 56%). Calculated for C₄₆H₄₇N₉O₆ [M⁺] 821.3, found [M+H⁺] 822; ¹H NMR (400 MHz, CHLOROFORM-d) δ ppm 0.83-0.97 (m, 6H) 1.01-1.13 (m, 1H) 1.87-2.50 (m, 6H) 2.90-3.09 (m, 2H) 3.17-3.35 (m, 1H) 3.51-3.62 (m, 1H) 3.63-3.80 (m, 7H) 3.82-3.95 (m, 1H) 4.16-4.49 (m, 3H) 5.30 (br. s., 1H) 5.58-5.69 (m, 1H) 6.01-6.25 (m, 2H) 6.98-7.14 (m, 2H) 7.19-7.34 (m, 2H) 7.47-8.36 (m, 9H) 8.90 (br. s., 1H) 10.53 (br. s., 1H).

### Example 13

### ((2S,5S)-2-{6-[6-(4-{2-[(S)-1-((S)-2-Methoxycarbonylamino-3-methyl-butyryl)-4,4-difluoro-pyrrolidin-2-yl]-3H-imidazol-4-yl}-phenyl)-pyridin-3-yl]-1H-benzoimidazol-2-yl}-4-oxo-1,2,4,5,6,7-hexahydro-azepino[3,2,1-hi]indol-5-yl)-carbamic acid methyl ester

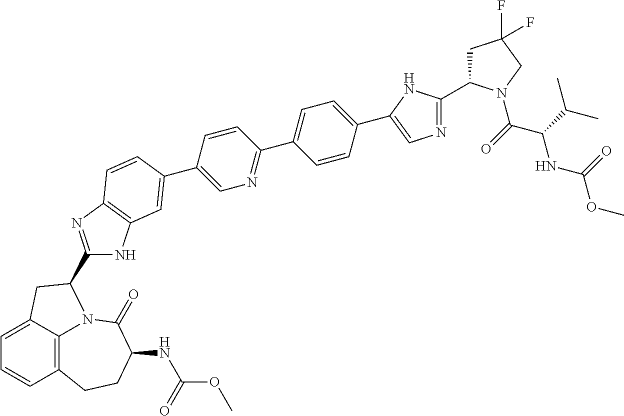

In a vial, methyl (2S,5S)-2-(6-(6-bromopyridin-3-yl)-1H-benzo[d]imidazol-2-yl)-4-oxo-1,2,4,5,6,7-hexahydroazepino[3,2,1-hi]indol-5-ylcarbamate (40 mg, 75.1 μmol) (see Example 13), methyl (S)-1-((S)-4,4-difluoro-2-(5-(4-(4,4,5,5-tetramethyl-1,3,2-dioxaborolan-2-yl)phenyl)-1H-imidazol-2-yl)pyrrolidin-1-yl)-3-methyl-1-oxobutan-2-ylcarbamate (40.0 mg, 75.1 μmol) (Intermediate 1) and sodium bicarbonate (18.9 mg, 225 μmol) were combined with toluene (4.5 ml), Ethanol (3.00 ml) and Water (1.5 ml) to give a light yellow suspension. 1,1′-Bis(diphenylphosphino)ferrocene-palladium(ii) dichloride dichloromethane complex (6.14 mg, 7.51 μmol) was added while the reaction mixture was degassed with Argon, The reaction mixture was heated to 90° C. and stirred for over night. The reaction mixture was poured into water and extracted with ethyl acetate; the organic layers were dried over sodium sulfate and concentrated in vacuo. The crude material was purified by flash chromatography (silica gel, 0% to 4% methanol/dichloromethane), the material was re-purified by preparative TLC (silica gel, 5% methanol/dichloromethane, eluted for 3 times) to afford ((2S,5S)-2-{6-[6-(4-{2-[(S)-1-((S)-2-methoxycarbonylamino-3-methyl-butyryl)-4,4-difluoro-pyrrolidin-2-yl]-3H-imidazol-4-yl}-phenyl)-pyridin-3-yl]-1H-benzoimidazol-2-yl}-4-oxo-1,2,4,5,6,7-hexahydro-azepino[3,2,1-hi]indol-5-yl)-carbamic acid methyl ester as pale yellow solid (47 mgs, 72%). Calculated for C₄₆H₄₅F₂N₉O₆ [M⁺] 857.3, found [M+H⁺] 858. ¹H NMR (300 MHz, DMSO-d₆) δ ppm 0.68-1.02 (m, 7H) 1.79-2.01 (m, 1H) 2.02-2.36 (m, 2H) 2.61-3.01 (m, 2H) 3.03-3.17 (m, 2H) 3.34-3.44 (m, 2H) 3.47-3.61 (m, 6H) 3.61-3.81 (m, 1H) 3.86-4.04 (m, 1H) 4.05-4.32 (m, 2H) 4.40-4.64 (m, 1H) 5.18-5.40 (m, 1H) 5.93 (m, J=11.00 Hz, 1H) 6.97-7.26 (m, 3H) 7.41-7.67 (m, 3H) 7.72-7.96 (m, 4H) 7.97-8.31 (m, 3H) 8.81-9.12 (m, 1H) 11.86-12.56 (m, 2H).

### Example 14

### {(2S,5S)-2-[6-(6-{2-[(S)-1-((S)-2-Methoxycarbonylamino-3-methyl-butyryl)-pyrrolidin-2-yl]-1H-imidazol-4-yl}-quinoxalin-2-yl)-1H-benzoimidazol-2-yl]-4-oxo-1,2,4,5,6,7-hexahydro-azepino[3,2,1-hi]indol-5-yl}-carbamic acid methyl ester

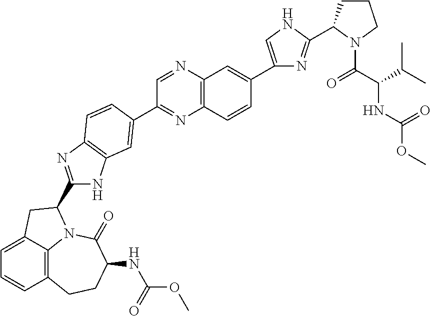

In a vial, methyl (2S,5S)-4-oxo-2-(6-(4,4,5,5-tetramethyl-1,3,2-dioxaborolan-2-yl)-1H-benzo[d]imidazol-2-yl)-1,2,4,5,6,7-hexahydroazepino[3,2,1-hi]indol-5-ylcarbamate (206 mg, 410 μmol) (see Example 13), 6-bromo-2-chloroquinoxaline (99.8 mg, 410 μmol) (Intermediate 13) and cesium carbonate (267 mg, 820 μmol) were combined with dioxane (5.00 ml), and water (1 ml) to give a light yellow solution. Tetrakis(triphenyl-phosphine)palladium (0) (47.4 mg, 41.0 mmol) was added while the reaction mixture was degassed with nitrogen. The reaction mixture was heated to 80° C. and stirred for over night. The reaction mixture was poured into saturated sodium chloride aqueous solution and extracted with ethyl acetate, the organic layers were dried over sodium sulfate and concentrated in vacuo. The crude material was purified by flash chromatography (silica gel, 50% to 100% ethyl acetate/hexanes). The impure material was re-purified by flash chromatography(silica gel, 0% to 3% methanol/dichloromethane) to afford methyl (2S,5S)-2-(6-(6-bromoquinoxalin-2-yl)-1H-benzo[d]imidazol-2-yl)-4-oxo-1,2,4,5,6,7-hexahydroazepino[3,2,1-hi]indol-5-ylcarbamate as pale yellow solid (135 mgs, 56%). Calculated for C₂₉H₂₃BrN₆O₃ [M⁺] 582/584, found [M+H⁺] 583/585.

In a flask, methyl (2S,5S)-2-(6-(6-bromoquinoxalin-2-yl)-1H-benzo[d]imidazol-2-yl)-4-oxo-1,2,4,5,6,7-hexahydroazepino[3,2,1-hi]indol-5-ylcarbamate (91 mg, 156 μmol), 4,4,4′,4′,5,5,5′,5′-octamethyl-2,2′-bi(1,3,2-dioxaborolane) (59.4 mg) and potassium acetate (45.9 mg, 468 μmol) were combined with dioxane (8.0 ml) to give a brown suspension. 1,1′-bis(diphenylphosphino)ferrocene-palladium(ii) dichloride dichloromethane complex (12.7 mg, 15.6 μmol) was added while degassed with nitrogen. The reaction mixture was heated to 90° C. and stirred for over night. The reaction mixture was poured into ethyl acetate, the organic layers were washed with brine once and dried over sodium sulfate and concentrated in vacuo. The crude material was purified by flash chromatography (silica gel, 5% methanol/dichloromethane). The impure material was re-purified by preparative TLC (silica gel, 5% methanol/dichloromethane once and ethyl acetate once) to afford 2-(2-((2S,5S)-5-(methoxycarbonylamino)-4-oxo-1,2,4,5,6,7-hexahydroazepino[3,2,1-hi]indol-2-yl)-1H-benzo[d]imidazol-6-yl)quinoxalin-6-ylboronic acid as pale yellow solid (26 mgs, 26%). Calculated for C₂₉H₂₅BN₆O₅ [M⁺] 548.2, found [M+H⁺] 549.

In a vial, 2-(2-((2S,5S)-5-(methoxycarbonylamino)-4-oxo-1,2,4,5,6,7-hexahydroazepino[3,2,1-hi]indol-2-yl)-1H-benzo[d]imidazol-6-yl)quinoxalin-6-ylboronic acid (26 mg), (S)-methyl 2-((S)-2-(4-iodo-1-((2-(trimethylsilyl)ethoxy)-methyl)-1H-imidazol-2-yl)pyrrolidine-1-carboxamido)-3-methylbutanoate (33 mg, 59.9 μmol) and sodium bicarbonate (11.9 mg, 142 μmol) were combined with toluene (3.00 ml), ethanol (2.00 ml) and water (1 ml) to give a light yellow suspension. 1,1′-bis(diphenyl-phosphino)ferrocene-palladium(ii) dichloride dichloromethane complex (3.87 mg, 4.74 μmol, Eq: 0.1) was added while the reaction mixture was degassed with argon. The reaction mixture was heated to 90° C. and stirred for 7 hours. The reaction mixture was poured into water and extracted with ethyl acetate, the organic layers were dried over sodium sulfate and concentrated in vacuo, used for the next step.

In a round-bottomed flask, {(2S,5S)-2-[6-(6-{2-[(S)-1-((S)-2-Methoxy-carbonylamino-3-methyl-butyryl)-pyrrolidin-2-yl]-1-(2-trimethylsilanyl-ethoxymethyl)-1H-imidazol-4-yl}-quinoxalin-2-yl)-1H-benzoimidazol-2-yl]-4-oxo-1,2,4,5,6,7-hexahydro-azepino[3,2,1-hi]indol-5-yl}-carbamic acid methyl ester (from previous step) was treated with hydrogen chloride in Dioxane (3 mL, 12.0 mmol) to give a dark brown solution. The mixture was stirred at room temperature for one day. The reaction mixture was poured into water and extracted with ethyl acetate, aqueous solution was adjusted PH>11 with 1N sodium hydroxide aqueous solution, extracted with ethyl acetate, the organic layers was dried over sodium sulfate and concentrated in vacuo. The crude material was purified by preparative TLC (silica gel, 5% methanol/dichloromethane/ammonium hydroxide for twice), the material was re-purified by preparative TLC (silica gel, (silica gel, 6% methanol/dichloromethane for twice) to afford {(2S,5S)-2-[6-(6-{2-[(S)-1-((S)-2-Methoxycarbonylamino-3-methyl-butyryl)-pyrrolidin-2-yl]-1H-imidazol-4-yl}-quinoxalin-2-yl)-1H-benzoimidazol-2-yl]-4-oxo-1,2,4,5,6,7-hexahydro-azepino[3,2,1-hi]indol-5-yl}-carbamic acid methyl esters yellow solid (5.9 mgs, 15% in two steps). Calculated for C₄₃H₄₄N₁₀O₆ [M⁺] 796.3, found [M+H⁺] 797.

¹H NMR (300 MHz, CHLOROFORM-d) δ ppm 0.73-0.93 (m, 6H) 1.55-1.68 (m, 2H) 1.92-1.99 (m, 2H) 2.06-2.19 (m, 1H) 2.22-2.46 (m, 2H) 2.85-3.07 (m, 2H) 3.09-3.32 (m, 1H) 3.43-3.60 (m, 1H) 3.59-3.71 (m, 6H) 3.74-3.89 (m, 1H) 4.10-4.51 (m, 3H) 5.10-5.58 (m, 2H) 5.91-6.11 (m, 2H) 6.86-7.10 (m, 2H) 7.24-8.55 (m, 8H) 9.22 (s, 1H) 10.11-10.79 (m, 2H).

### Example 15

### ((2S,5S)-2-{5-[2-(4-{2-[(S)-1-((8)-2-Methoxycarbonylamino-3-methyl-butyryl)-pyrrolidin-2-yl]-3H-imidazol-4-yl}-phenyl)-pyrimidin-5-yl]-1H-imidazol-2-yl}-4-oxo-1,2,4,5,6,7-hexahydro-azepino[3,2,1-hi]indol-5-yl)-carbamic acid methyl ester

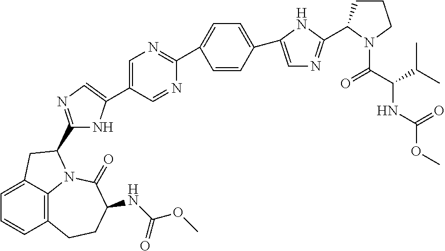

In a round-bottomed flask, 5-bromo-2-chloropyrimidine (13.4 g, 69.2 mmol), tributyl(1-ethoxyvinyl)tin (25 g, 69.2 mmol) and bis(triphenylphosphine)-palladium(ii) dichloride (3.4 g, 4.85 mmol) were combined with dimethylformate (200 ml) to give a yellow suspension. The reaction mixture was degassed and heated to 85° C. and stirred for 3 hours, cooled. A solution of potassium fluoride (35 g in 300 ml water) was added, stirred at room temperature about 30 minutes, ice water added (150 ml), yellow solid was obtained by filtration, which was washed with water several times, then the solid was washed with ethyl acetate, (white solid left and discarded), ethyl acetate solution was collected and dried over sodium sulfate and concentrated in vacuo. The crude material was purified by flash chromatography (silica gel, 7% to 10% ethyl acetate/hexanes) to afford 2-chloro-5-(1-ethoxy-vinyl)-pyrimidine as white solid (8.6 g, 67%). Calculated for C₈H₉ClN₂O [M⁺] 184, found [M+H⁺] 185.

In a 1 L round-bottomed flask, 2-chloro-5-(1-ethoxyvinyl)pyrimidine (8.6 g, 46.6 mmol) was combined with tetrahydrofuran (500 ml) and water (26 ml) to give a colorless solution. N-Bromosuccinimide (8.29 g, 46.6 mmol) was added by portions at 0° C., the reaction mixture was stirred at 0° C. for 2 hours. The reaction mixture was poured into water and extracted with ethyl acetate, the organic layers were washed by brine and dried over sodium sulfate and concentrated in vacuo. The crude material was purified by flash chromatography (silica gel, 10% to 20% ethyl acetate/hexanes) to afford 2-bromo-1-(2-chloropyrimidin-5-yl)ethanone as white solid (8.33 g, 75%). Calculated for C₆H₄BrClN₂O [M⁺] 234, found [2M+Na⁺-2] 489.

In a vial, 2-bromo-1-(2-chloropyrimidin-5-yl)ethanone (77.4 mg, 329 μmol,) was combined with Acetonitrile (3 ml) to give a colorless solution. (2S,5S)-5-(methoxycarbonylamino)-4-oxo-1,2,4,5,6,7-hexahydroazepino[3,2,1-hi]indole-2-carboxylic acid (0.1 g, 329 μmol) and N,N′-diisopropylethylamine (42.5 mg, 57.4 μA, 329 μmol) were added. the mixture was stirred at room temperature for 3 hours, The reaction mixture was poured into ethyl acetate and washed with 0.3N HCl aqueous solution, and brine, the organic layers were dried over sodium sulfate and concentrated in vacuo. The pale yellow foam obtained, used for the next step.

In a round-bottomed flask, (2S,5S)-2-(2-chloropyrimidin-5-yl)-2-oxoethyl 5-(methoxycarbonylamino)-4-oxo-1,2,4,5,6,7-hexahydroazepino[3,2,1-hi]indole-2-carboxylate (from previous step) was combined with xylene (5 ml) to give a light yellow solution. Ammonium acetate (180 mg, 2.34 mmol) was added. The reaction mixture was heated to 140° C. for 3 hours. Hexane was added into the reaction mixture, and the top solution was decanted, the result solid was purified by flash chromatography (silica gel, 5% methanol/dichloromethane). The impure material was re-purified by flash chromatography (silica gel, 30% to 100% ethyl acetate/hexanes, then 1:9 7% methanol/dichloromethane:ethyl acetate) to afford methyl (2S,5S)-2-(5-(2-chloropyrimidin-5-yl)-1H-imidazol-2-yl)-4-oxo-1,2,4,5,6,7-hexahydroazepino[3,2,1-hi]indol-5-ylcarbamate as light yellow solid (28 mgs, 23% in two steps). Calculated for C₂₁H₁₉ClN₆O₃ [M⁺] 438.1, found [M+H⁺] 439.

In a vial, methyl (2S,5S)-2-(5-(2-chloropyrimidin-5-yl)-1H-imidazol-2-yl)-4-oxo-1,2,4,5,6,7-hexahydroazepino[3,2,1-hi]indol-5-ylcarbamate (28 mg, 63.8 μmol), methyl (S)-3-methyl-1-oxo-1-((S)-2-(5-(4-(4,4,5,5-tetramethyl-1,3,2-dioxaborolan-2-yl)phenyl)-1H-imidazol-2-yl)pyrrolidin-1-yl)butan-2-ylcarbamate (38.0 mg, 76.6 μmol) and sodium bicarbonate (16.1 mg, 191 μmol) were combined with toluene (3.00 ml), ethanol (2.00 ml) and water (1 ml) to give a light yellow suspension. 1,1′-bis(diphenylphosphino)-ferrocene-palladium(ii) dichloride dichloromethane complex (5.21 mg, 6.38 μmol) was added while the reaction mixture was degassed with argon, the reaction mixture was heated to 90° C. and stirred for over night. The reaction mixture was poured into water and extracted with ethyl acetate, the organic layers were dried over sodium sulfate and concentrated in vacuo. The crude material was purified by flash chromatography (silica gel, 6% methanol/dichloromethane). The material was dissolved in 1N hydrogen chloride aqueous solution, washed with ethyl acetate, then aqueous solution was adjusted PH>11, extracted with ethyl acetate, the organic layers were dried over sodium sulfate and concentrated in vacuo. The crude material was purified by preparative TLC (silica gel, 6% methanol/dichloromethane, eluted for twice). Impure material was re-purified by preparative TLC (silica gel, 2% methanol/ethyl acetate, eluted for 3 times) to afford ((2S,5S)-2-{5-[2-(4-{2-[(S)-1-((S)-2-Methoxycarbonylamino-3-methyl-butyryl)-pyrrolidin-2-yl]-3H-imidazol-4-yl}-phenyl)-pyrimidin-5-yl]-1H-imidazol-2-yl}-4-oxo-1,2,4,5,6,7-hexahydro-azepino[3,2,1-hi]indol-5-yl)-carbamic acid methyl ester as light yellow solid (13 mgs, 26%). Calculated for C₄₁H₄₄N₁₀O₆ [M⁺] 772.3, found [M+H⁺] 773. ¹H NMR (300 MHz, CHLOROFORM-d) δ ppm 0.68-1.04 (m, 6H) 1.66-2.18 (m, 5H) 2.22-2.43 (m, 2H) 2.97 (d, J=17.37 Hz, 2H) 3.10-3.33 (m, 1H) 3.38-3.53 (m, 1H) 3.58-3.73 (m, 6H) 3.72-3.85 (m, 1H) 4.10-4.49 (m, 3H) 5.11-5.52 (m, 2H) 5.70-6.13 (m, 2H) 6.91-7.08 (m, 2H) 7.12-7.39 (m, 4H) 7.57-7.91 (m, 1H) 8.31 (d, J=7.93 Hz, 2H) 8.81-9.12 (m, 2H) 10.06-11.04 (m, 2H).

### Example 16

### ((2S,5S)-2-{5-[2-(4-{2-[(S)-1-((S)-2-Methoxycarbonylamino-3-methyl-butyryl)-4,4-difluoro-pyrrolidin-2-yl]-3H-imidazol-4-yl}-phenyl)-pyrimidin-5-yl]-1H-imidazol-2-yl}-4-oxo-1,2,4,5,6,7-hexahydro-azepino[3,2,1-hi]indol-5-yl)-carbamic acid methyl ester

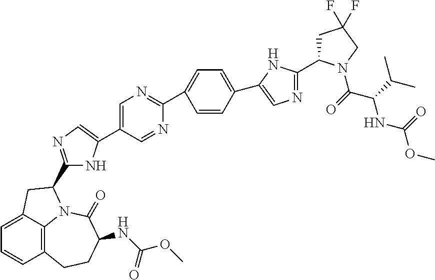

In a vial, methyl (2S,5S)-2-(5-(2-chloropyrimidin-5-yl)-1H-imidazol-2-yl)-4-oxo-1,2,4,5,6,7-hexahydroazepino[3,2,1-hi]indol-5-ylcarbamate (40 mg, 91.1 μmol) (see Example 16), methyl (S)-1-((S)-4,4-difluoro-2-(5-(4-(4,4,5,5-tetramethyl-1,3,2-dioxaborolan-2-yl)phenyl)-1H-imidazol-2-yl)pyrrolidin-1-yl)-3-methyl-1-oxobutan-2-ylcarbamate (48.5 mg, 91.1 μmol) (Intermediate 1) and sodium bicarbonate (23.0 mg, 273 μmol) were combined with toluene (4.5 ml), ethanol (3 ml) and water (1.5 ml) to give a light yellow suspension. 1,1′-Bis(diphenylphosphino)ferrocene-palladium(ii) dichloride dichloromethane complex (7.44 mg, 9.11 μmol) was added while the reaction mixture was degassed with Argon, The reaction mixture was heated to 90° C. and stirred for 5 hours. The reaction mixture was poured into 1M hydrogen chloride aqueous solution and extracted with ethyl acetate, the organic layers were discarded, the aqueous solution was adjusted PH>11, extracted with ethyl acetate, the organic layers were dried over sodium sulfate and concentrated in vacuo. The crude material was purified by preparative TLC (silica gel, 4% methanol/dichloromethane, eluted for 3 times), the impure material was re-purified by preparative TLC (silica gel, 3% methanol/ethyl acetate, eluted once and 5% methanol/dichloromethane, eluted for twice) to afford ((2s,5s)-2-{5-[2-(4-{2-[(s)-1-((s)-2-methoxycarbonylamino-3-methyl-butyryl)-pyrrolidin-2-yl]-3h-imidazol-4-yl}-phenyl)-pyrimidin-5-yl]-1h-imidazol-2-yl}-4-oxo-1,2,4,5,6,7-hexahydro-azepino[3,2,1-hi]indol-5-yl)-carbamic acid methyl ester as light yellow solid (27 mgs, 36%). Calculated for C₄₁H₄₂F₂N₁₀O₆[M⁺] 808.3, found [M+H⁺] 809. ¹H NMR (300 MHz, DMSO-d₆) δ ppm 0.83 (d, J=6.04 Hz, 6H) 1.77-2.37 (m, 3H) 2.67-3.18 (m, 4H) 3.36-3.42 (m, 1H) 3.46-3.72 (m, 7 H) 3.86-3.99 (m, 1H) 4.05-4.32 (m, 2H) 4.41-4.62 (m, 1H) 5.21-5.36 (m, 1H) 5.71-5.86 (m, 1H) 6.94-7.23 (m, 3H) 7.47 (d, J=7.93 Hz, 2H) 7.63 (br. s., 1H) 7.78 (br. s., 1H) 7.84 (d, J=7.93 Hz, 2H) 8.32 (d, J=7.60 Hz, 2H) 9.11 (s, 2H) 11.95-12.18 (m, 2H).

### Example 17

### ((2S,5S)-2-{5-[2-(6-{2-[(S)-1-((S)-2-Methoxycarbonylamino-3-methyl-butyryl)-pyrrolidin-2-yl]-3H-imidazol-4-yl}-naphthalen-2-yl)-pyrimidin-5-yl]-1H-imidazol-2-yl}-4-oxo-1,2,4,5,6,7-hexahydro-azepino[3,2,1-hi]indol-5-yl)-carbamic acid methyl ester

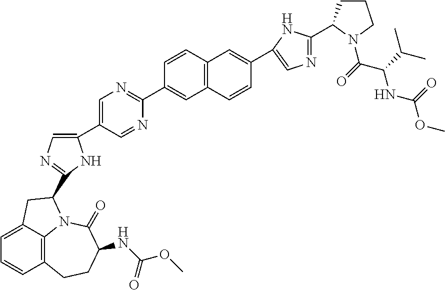

In a vial, methyl (2S,5S)-2-(5-(2-chloropyrimidin-5-yl)-1H-imidazol-2-yl)-4-oxo-1,2,4,5,6,7-hexahydroazepino[3,2,1-hi]indol-5-ylcarbamate (40 mg, 91.1 μmol) (see Example 13), methyl (S)-3-methyl-1-oxo-1-((S)-2-(5-(6-(4,4,5,5-tetramethyl-1,3,2-dioxaborolan-2-yl)naphthalen-2-yl)-4H-imidazol-2-yl)pyrrolidin-1-yl)butan-2-ylcarbamate (49.8 mg, 91.1 μmol) and sodium bicarbonate (23.0 mg, 273 μmol) were combined with toluene (4.5 ml), ethanol (3 ml) and water (1.5 ml) to give a light yellow suspension. 1,1′-Bis(diphenylphosphino)ferrocene-palladium(ii) dichloride dichloromethane complex (7.44 mg, 9.11 μmol) was added while the reaction mixture was degassed with argon. The reaction mixture was heated to 90° C. and stirred for 5 hours. The reaction mixture was poured into 1M hydrogen chloride aqueous solution and extracted with ethyl acetate, the organic layers were discarded, the aqueous solution was adjusted PH>11, extracted with ethyl acetate, the organic layers were dried over sodium sulfate and concentrated in vacuo. The crude material was purified by preparative TLC (silica gel, 5% methanol/dichloromethane, eluted for 3 times). The impure material was repurified by preparative TLC (silica gel, 3%, 5% methanol/ethyl acetate, eluted once and 5% methanol/dichloromethane, eluted for twice) to afford ((2S,5S)-2-{5-[2-(6-{2-[(S)-1-((S)-2-Methoxycarbonylamino-3-methyl-butyryl)-pyrrolidin-2-yl]-3H-imidazol-4-yl}-naphthalen-2-yl)-pyrimidin-5-yl]-1H-imidazol-2-yl}-4-oxo-1,2,4,5,6,7-hexahydro-azepino[3,2,1-hi]indol-5-yl)-carbamic acid methyl ester as light yellow solid (30 mgs, 40%). Calculated for C₄₅H₄₆N₁₀O₆ [M⁺] 822.3, found [M+H⁺] 823.

¹H NMR (300 MHz, DMSO-d₆) δ ppm 0.74-1.02 (m, 6H) 1.89-2.37 (m, 7H) 3.01-3.19 (m, 2H) 3.36-3.40 (m, 1H) 3.47-3.89 (m, 9H) 3.95-4.25 (m, 2H) 4.99-5.18 (m, 1H) 5.68-5.88 (m, 1H) 6.90-7.24 (m, 3H) 7.30 (d, J=8.69 Hz, 1H) 7.48 (d, J=8.31 Hz, 1H) 7.66 (s, 1H) 7.82 (s, 1H) 7.88-8.17 (m, 3H) 8.23 (s, 1H) 8.43 (d, J=8.69 Hz, 1H) 8.88 (s, 1H) 9.19 (s, 2H) 11.86 (br. s., 1H) 12.13 (br. s., 1H).

### Example 18

### ((2S,5S)-2-{5-[7-(4-{2-[(S)-1-((8)-2-Methoxycarbonylamino-3-methyl-butyryl)-pyrrolidin-2-yl]-3H-imidazol-4-yl}-phenyl)-quinolin-3-yl]-1H-imidazol-2-yl}-4-oxo-1,2,4,5,6,7-hexahydro-azepino[3,2,1-hi]indol-5-yl)-carbamic acid methyl ester trifluoacetic acid

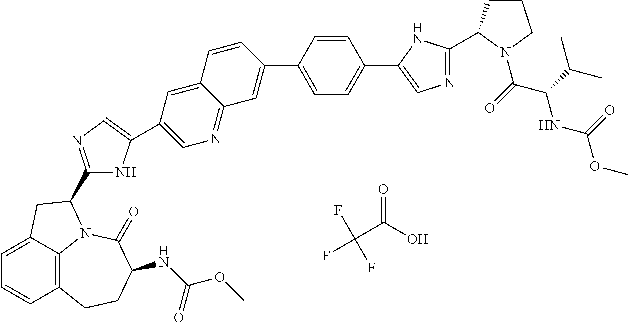

In a vial, 7-bromoquinoline-3-carboxylic acid (1 g, 3.97 mmol), N,O-dimethylhydroxylamine hydrochloride (580 mg, 5.95 mmol) and HATU (1.82 g, 4.79 mmol) were combined with dimethylformamide (20 ml) to give a off-white suspension. DIISOPROPYLETHYLAMINE (2.56 g, 3.46 ml, 19.8 mmol was added slowly (it turned into yellow solution); the mixture was stirred at room temperature about 3 hours. The reaction mixture was poured into water, and extracted with ethyl acetate, the organic layers were washed with brine, and dried over sodium sulfate and concentrated in vacuo. The crude material was purified by flash chromatography (silica gel, 40% to 50% ethyl acetate in hexanes) to afford 7-bromo-N-methoxy-N-methylquinoline-3-carboxamide as pale yellow oil (1.15 g, 98%). Calculated for C₁₂H₁₁BrN₂O₂[M⁺] 294.0, found [M+H⁺] 295.

To a solution of 7-bromo-N-methoxy-N-methylquinoline-3-carboxamide (1.15 g, 3.9 mmol) in tetrahydrofuran (50 ml), a solution of methylmagnesium bromide in diethyl ether (3M, 3.25 ml, 9.74 mmol) was added slowly at 0° C. under an atmosphere of nitrogen, stirred at 0° C. for one hour, at room temperature for 1 hour, a saturated ammonium chloride aqueous solution (2 ml) was add, the mixture was poured into 5% sodium bicarbonate aqueous solution, extracted by ethyl acetate. The organic layers were washed with brine, and dried over sodium sulfate and concentrated in vacuo. The crude material was purified by flash chromatography (silica gel, 10% to 30% ethyl acetate in hexanes) to afford 1-(7-bromoquinolin-3-yl)ethanone as light yellow solid (0.84 g, 86%). Calculated for CᵢᵢH₈BrNO [M⁺] 249.0, found [M+H⁺] 250.

In a round-bottomed flask, 1-(7-bromoquinolin-3-yl)ethanone (0.84 g, 3.36 mmol) and hydrogen bromide 48% aqueous solution (745 mg, 0.5 ml, 9.21 mmol) were combined with glacial acetic acid (20 ml) to give a yellow suspension, bromine (537 mg, 173 μl, 3.36 mmol) in HBr 48% aqueous solution (745 mg, 0.5 ml, 9.21 mmol)was added slowly, the mixture was stirred at room temperature for three days; the mixture was poured into water, the solid was collected by filtration, and washed with water, the solid was purified by flash chromatography (silica gel, 10% to 40% ethyl acetate in hexanes), then crystallized from ethyl acetate to afford 2-bromo-1-(7-bromoquinolin-3-yl)ethanone as off white solid (0.52 g, 47%). Calculated for C₁₁H₇Br₂NO [M⁺] 326.9, found [M+H⁺] 328/330.

In a round-bottomed flask, 2-bromo-1-(7-bromoquinolin-3-yl)ethanone (0.52 g, 1.58 mmol) and (2S,5S)-5-(methoxycarbonylamino)-4-oxo-1,2,4,5,6,7-hexahydroazepino[3,2,1-hi]indole-2-carboxylic acid (529 mg, 1.74 mmol) were combined with acetonitrile (20 ml) to give a off-white suspension. N,N′-diisopropylethylamine (409 mg, 552 μA, 3.16 mmol) was added. The reaction mixture was stirred at room temperature for 3 hours. The reaction mixture was poured into water and extracted with ethyl acetate. The organic layers were washed with brine, and dried over sodium sulfate and concentrated in vacuo to afford light brown foam, used for the next step.

To a 20 mL microwave vial was added the light brown foam from last step, ammonium acetate (523 mg, 6.79 mmol) and dioxane (13.0 ml). The vial was capped and heated in the microwave at 120° C. for 1 hour. The reaction mixture was poured into the saturated sodium bicarbonate aqueous solution, extracted with ethyl acetate, The organic layers were washed with brine, and dried over sodium sulfate and concentrated in vacuo; The crude material was purified by flash chromatography (silica gel, 50% to 100% ethyl acetate in hexanes) to afford methyl (2S,5S)-2-(5-(7-bromoquinolin-3-yl)-1H-imidazol-2-yl)-4-oxo-1,2,4,5,6,7-hexahydroazepino[3,2,1-hi]indol-5-ylcarbamate as yellow solid (0.37 g, 51%)

In a vial, methyl (2S,5S)-2-(5-(7-bromoquinolin-3-yl)-1H-imidazol-2-yl)-4-oxo-1,2,4,5,6,7-hexahydroazepino[3,2,1-hi]indol-5-ylcarbamate (62 mg, 116 μmol), methyl (S)-3-methyl-1-oxo-1-((S)-2-(5-(4-(4,4,5,5-tetramethyl-1,3,2-dioxaborolan-2-yl)phenyl)-1H-imidazol-2-yl)pyrrolidin-1-yl)butan-2-ylcarbamate (63.6 mg, 128 μmol) and sodium bicarbonate (29.3 mg, 349 μmol) were combined with Toluene (3.00 ml), Ethanol (2.00 ml) and Water (1 ml) to give a light yellow suspension. 1,1′-bis(diphenylphosphino)-ferrocene-palladium(ii)-dichloride dichloromethane complex (9.51 mg, 11.6 μmol) was added while the reaction mixture was degassed with Argon, The reaction mixture was heated to 85° C. and stirred for 7 hours. The reaction mixture was poured into 1M hydrogen chloride aqueous solution and extracted with ethyl acetate, the organic layers were discarded, the aqueous solution was adjusted PH>11, extracted with ethyl acetate, the organic layers were dried over sodium sulfate and concentrated in vacuo. The crude material was purified by HPLC to afford 42S,5S)-2-{5-[7-(4-{2-[(S)-1-((S)-2-Methoxy-carbonylamino-3-methyl-butyryl)-pyrrolidin-2-yl]-3H-imidazol-4-yl}-phenyl)-quinolin-3-yl]-1H-imidazol-2-yl}-4-oxo-1,2,4,5,6,7-hexahydro-azepino[3,2,1-hi]indol-5-yl)-carbamic acid methyl ester trifluoroacetic acid salt as yellow solid (65 mgs, 59%). Calculated for C₄₆H₄₇N₉O₆.C₂HF₃O₂ [M⁺]935.9, found [M+H⁺] 822 (free base).

¹H NMR (300 MHz, DMSO-d₆) δ ppm 0.72-0.89 (m, 6H) 1.94-2.21 (m, 5H) 2.23-2.43 (m, 2H) 3.04-3.20 (m, 2H) 3.36 (d, J=17.38 Hz, 1H) 3.53 (s, 6H) 3.68-4.31 (m, 5H) 5.06-5.22 (m, 1H) 5.83-5.97 (m, 1H) 6.87-7.60 (m, 7H) 7.82-8.25 (m, 8H) 8.40 (s, 1H) 8.69 (s, 1H) 9.36 (d, J=1.51 Hz, 1H).

### Example 19

### ((2S,5S)-2-{5-[7-(4-{2-[(S)-1-((S)-2-Methoxycarbonylamino-3-methyl-butyryl)-4,4-difluoro-pyrrolidin-2-yl]-3H-imidazol-4-yl}-phenyl)-quinolin-3-yl]-1H-imidazol-2-yl}-4-oxo-1,2,4,5,6,7-hexahydro-azepino[3,2,1-hi]indol-5-yl)-carbamic acid methyl ester trifluoroacetic acid

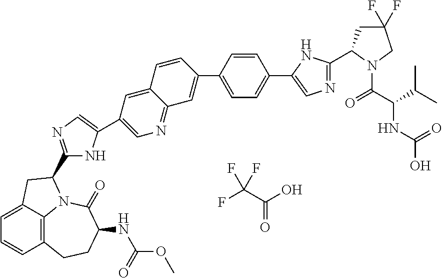

In a vial, methyl (2S,5S)-2-(5-(7-bromoquinolin-3-yl)-1H-imidazol-2-yl)-4-oxo-1,2,4,5,6,7-hexahydroazepino[3,2,1-hi]indol-5-ylcarbamate (62 mg, 116 μmol) (see Example 19), methyl (S)-1-((S)-4,4-difluoro-2-(5-(4-(4,4,5,5-tetramethyl-1,3,2-dioxaborolan-2-yl)phenyl)-1H-imidazol-2-yl)pyrrolidin-1-yl)-3-methyl-1-oxobutan-2-ylcarbamate (68.2 mg, 128 μmol) (Intermediate 1) and sodium bicarbonate (29.3 mg, 349 μmol) were combined with Toluene (3.00 ml), Ethanol (2.00 ml) and Water (1.00 ml) to give a light yellow suspension. 1,1′-bis(diphenylphosphino)ferrocene-palladium(ii) dichloride dichloromethane complex (9.51 mg, 11.6 μmol) was added while the reaction mixture was degassed with Argon, The reaction mixture was heated to 85° C. and stirred for 7 hours. The reaction mixture was poured into 1M hydrogen chloride aqueous solution and extracted with ethyl acetate, the organic layers were discarded, the aqueous solution was adjusted PH>11, extracted with ethyl acetate, the organic layers were dried over sodium sulfate and concentrated in vacuo. The crude material was purified by HPLC to afford 42S,5S)-2-{5-[7-(4-{2-[(S)-1-((S)-2-Methoxy-carbonylamino-3-methyl-butyryl)-4,4-difluoro-pyrrolidin-2-yl]-3H-imidazol-4-yl}-phenyl)-quinolin-3-yl]-1H-imidazol-2-yl}-4-oxo-1,2,4,5,6,7-hexahydroazepino-[3,2,1-hi]indol-5-yl)-carbamic acid methyl ester trifluoroacetic acid as yellow solid (53 mgs, 46%). Calculated for C₄₆H₄₅F₂N₉O₆.C₂HF₃O₂ [M⁺] 971.9; found [M+H⁺] 858 (free base). ¹H NMR (300 MHz, DMSO-d₆) δ ppm 0.75-0.85 (m, 6H) 1.86-1.99 (m, 1H) 2.05-2.18 (m, 1H) 2.23-2.38 (m, 1H) 2.64-3.22 (m, 4H) 3.32-3.41 (m, 1H) 3.50-3.57 (m, 6H) 3.60-4.62 (m, 5H) 5.25-5.39 (m, 1H) 5.79-5.96 (m, 1H) 6.76-7.37 (m, 5H) 7.45-7.59 (m, 2H) 7.86-7.97 (m, 2H) 8.01-8.15 (m, 6H) 8.38 (s, 1H) 8.68 (s, 1H) 9.35 (d, J=1.51 Hz, 1H).

### Example 20

### ((2S,5S)-2-{5-[7-(4-[5-Chloro-2-[(S)-1-((S)-2-methoxycarbonylamino-3-m ethyl-butyryl)-pyrrolidin-2-yl]-3H-imidazol-4-yl}-phenyl)-quinolin-3-yl]-1H-imidazol-2-yl}-4-oxo-1,2,4,5,6,7-hexahydro-azepino[3,2,1-hi]indol-5-yl)-carbamic acid methyl ester trifluoroacetic acid salt

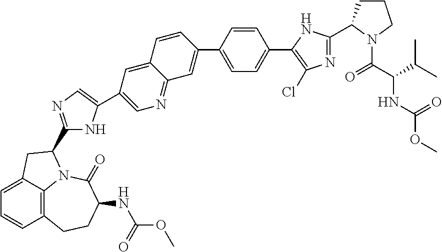

In a vial, methyl (2S,5S)-2-(5-(7-bromoquinolin-3-yl)-1H-imidazol-2-yl)-4-oxo-1,2,4,5,6,7-hexahydroazepino[3,2,1-hi]indol-5-ylcarbamate (66 mg, 124 μmol) (see Example 19), methyl (S)-1-((S)-2-(4-chloro-5-(4-(4,4,5,5-tetramethyl-1,3,2-dioxaborolan-2-yl)phenyl)-1H-imidazol-2-yl)pyrrolidin-1-yl)-3-methyl-1-oxobutan-2-ylcarbamate (85.0 mg, 160 μmol) and sodium bicarbonate (31.2 mg, 372 μmol) were combined with toluene (3.00 ml), ethanol (2.00 ml) and water (1.00 ml) to give a light yellow suspension. 1,1′-bis(diphenylphosphino)ferrocene-palladium(ii) dichloride dichloromethane complex (10.1 mg, 12.4 μmol) was added while the reaction mixture was degassed with argon. The reaction mixture was heated to 85° C. and stirred for 3 hours. The reaction mixture was poured into 1M hydrogen chloride aqueous solution and extracted with ethyl acetate, the organic layers were discarded, the aqueous solution was adjusted PH>11, extracted with ethyl acetate, the organic layers were dried over sodium sulfate and concentrated in vacuo. The crude material was purified by flash chromatography (silica gel, 5% methanol/dichloromethane/ammonium hydroxide). The impure material was re-purified by preparative TLC (silica gel, 1.0 mm, 3% methanol/dichloromethane/ammonium hydroxide, eluted for 3 times) to afford ((2S,5S)-2-{5-[7-(4-{5-Chloro-2-[(S)-1-((S)-2-methoxy-carbonylamino-3-methyl-butyryl)-pyrrolidin-2-yl]-3H-imidazol-4-yl}-phenyl)-quinolin-3-yl]-1H-imidazol-2-yl}-4-oxo-1,2,4,5,6,7-hexahydro-azepino[3,2,1-hi]indol-5-yl)-carbamic acid methyl ester as yellow solid, (67 mgs, 63%). Calculated for C₄₆H₄₆ClN₉O₆ [M⁺] 856.3, found [M+H⁺] 857. ¹H NMR (300 MHz, DMSO-d₆) δ ppm 0.78-1.02 (m, 6H) 1.83-2.39 (m, 7H) 3.10 (d, J=4.53 Hz, 2H) 3.40-3.60 (m, 7H) 3.60-3.90 (m, 3H) 3.96-4.24 (m, 2H) 4.96-5.12 (m, 1H) 5.77-5.88 (m, 1H) 6.97-7.07 (m, 1H) 7.08-7.14 (m, 1H) 7.16-7.24 (m, 1H) 7.26-7.36 (m, 1H) 7.44-7.56 (m, 1H) 7.78-8.13 (m, 7H) 8.29 (s, 1H) 8.53 (s, 1H) 9.33 (d, J=1.51 Hz, 1H) 12.06 (br. s., 1H) 12.62 (br. s., 1H).

### Example 21

### ((2S,5S)-2-{4-[4-(6-{2-[(S)-1-((S)-2-Methoxycarbonylamino-3-methyl-butyryl)-pyrrolidin-2-yl]-1H-imidazol-4-yl}-quinolin-2-yl)-phenyl]-1H-imidazol-2-yl}-4-oxo-1,2,4,5,6,7-hexahydro-azepino[3,2,1-hi]indol-5-yl)-carbamic acid methyl ester trifluoroacetic acid salt

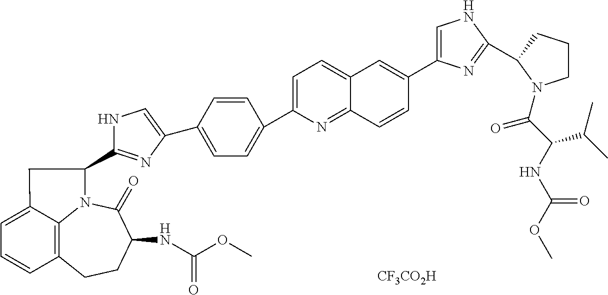

In a round-bottomed flask, 6-bromo-2-chloroquinoline (2.278 g, 9.39 mmol), 4,4,4′,4′,5,5,5′,5′-octamethyl-2,2′-bi(1,3,2-dioxaborolane) (2.45 g, 9.65 mmol) and potassium acetate (3.69 g, 37.6 mmol) were combined with dioxane (10 ml) to give a light brown suspension. While the mixture was degassed with argon, 1,1′-bis(diphenylphosphino)ferrocene-palladium(ii) dichloride dichloromethane complex (767 mgs, 939 μmol) was added. The reaction mixture was heated at 90° C. over night, cooled. The reaction mixture was poured into water and extracted with ethyl acetate. The organic layers were washed with brine, dried over sodium sulfate and concentrated in vacuo. The crude material was purified by flash chromatography (silica gel, 20%, and 100% ethyl acetate in hexanes) to afford 2-chloro-6-(4,4,5,5-tetramethyl-1,3,2-dioxaborolan-2-yl)-quinoline as brown solid (1.3 g, 47%). Calculated for C₁₅H₁₇BClNO₂ [M⁺] 289.1, found [M+H⁺] 290.

In a round-bottomed flask, 2-chloro-6-(4,4,5,5-tetramethyl-1,3,2-dioxaborolan-2-yl)quinoline (770 mg, 2.66 mmol), (S)-tert-butyl 2-(4-iodo-1H-imidazol-2-yl)pyrrolidine-1-carboxylate (966 mg, 2.66 mmol) and sodium bicarbonate (670 mg, 7.98 mmol) were combined with toluene (15.0 ml), ethanol (10.0 ml) and water (5 ml) to give a light brown suspension, 1,1′-bis(diphenylphosphino)ferrocene-palladium(ii) dichloride dichloromethane complex (221 mg, 271 μmol) was added while degassed with argon. The reaction mixture was heated to 85° C. for 7 hours. The reaction mixture was poured into water and extracted with ethyl acetate. The organic layers were washed with brine and dried over sodium sulfate and concentrated in vacuo. The crude material was purified by flash chromatography (silica gel, 10% to 70% ethyl acetate in hexanes), then crystallized from ethyl acetate/hexane to afford (S)-tert-butyl 2-(4-(2-chloroquinolin-6-yl)-1H-imidazol-2-yl)pyrrolidine-1-carboxylate as brown solid (0.48 g, 45%). Calculated for C₂₁H₂₃ClN₄O₂ [M⁺]398.1, found [M+H⁺] 399.

In a round-bottomed flask, methyl (2S,5S)-4-oxo-2-(5-(4-(4,4,5,5-tetramethyl-1,3,2-dioxaborolan-2-yl)phenyl)-1H-imidazol-2-yl)-1,2,4,5,6,7-hexahydroazepino[3,2,1-hi]indol-5-ylcarbamate (0.34 g, 579 μmol), (S)-tert-butyl 2-(4-(2-chloroquinolin-6-yl)-1H-imidazol-2-yl)pyrrolidine-1-carboxylate (0.183 g, 459 μmol) and sodium bicarbonate (116 mg, 1.38 mmol) were combined with toluene (6.00 ml), ethanol (4.00 ml) and water (2 ml) to give a light brown suspension. 1,1′-Bis(diphenylphosphino)ferrocene-palladium(ii) dichloride dichloromethane complex (37.5 mg, 45.9 μmol) was added while degassed with argon. The reaction mixture was heated to 90° C. for over night (LC-Mass showed only small amount of product formed). The reaction mixture was transferred into a microwave vial, more catalyst and dimethoxyethane (4 ml) were added. The vial was capped and heated in the microwave at 120° C. for 60 minutes and 130° C. for 30 minutes. The reaction mixture was poured into water and extracted with ethyl acetate, the organic layers were dried over sodium sulfate and concentrated in vacuo. The crude material was purified by flash chromatography (silica gel, 20%, 50%, 80%, 100% ethyl acetate in hexanes, and 3% methanol/dichloromethane) to afford (S)-tert-butyl 2-(4-(2-(4-(2-((2S,5S)-5-(methoxycarbonylamino)-4-oxo-1,2,4,5,6,7-hexahydroazepino[3,2,1-hi]indol-2-yl)-1H-imidazol-5-yl)phenyl)quinolin-6-yl)-1H-imidazol-2-yl)pyrrolidine-1-carboxylate as light brown foam (0.24 g, 68%). Calculated for C₄₄H₄₄N₈O₅ [M⁺]764.3, found [M+H⁺] 765.

In a round-bottomed flask, (S)-tert-butyl 2-(4-(2-(4-(2-((2S,5S)-5-(methoxycarbonylamino)-4-oxo-1,2,4,5,6,7-hexahydroazepino[3,2,1-hi]indol-2-yl)-1H-imidazol-5-yl)phenyl)quinolin-6-yl)-1H-imidazol-2-yl)pyrrolidine-1-carboxylate (0.24 g, 314 μmol) was combined with a solution of hydrogen chloride in dioxane (5 mL, 20.0 mmol) to give a yellow suspension. The mixture was stirred at room temperature over night. The reaction mixture was concentrated in vacuo to give a yellow solid, which was further washed with 20% ethyl acetate/hexane, then used for the next step.

In a round-bottomed flask, (2s)-2-[(methoxycarbonyl)amino]-3-methylbutanoic acid (22.5 mg, 128 μmol), HATU (48.8 mg, 128 μmol) and were combined with N,N-dimethylformamide (3 ml) to give a yellow solution. Methyl (2S,5S)-4-oxo-2-(5-(4-(6-(2-((S)-pyrrolidin-2-yl)-1H-imidazol-4-yl)quinolin-2-yl)phenyl)-1H-imidazol-2-yl)-1,2,4,5,6,7-hexahydroazepino[3,2,1-hi]indol-5-ylcarbamate hydrochloride (60 mg, 85.6 μmol and N,N′-diisopropylethylamine (55.3 mg, 74.7 μA, 428 μmol) were added, the reaction mixture was stirred at room temperature for 4 hours. The mixture was poured into 0.5N hydrogen chloride aqueous solution, washed with ethyl acetate; the aqueous solution was adjusted PH>11 with 1 N sodium hydroxide aqueous solution, extracted with ethyl acetate, the organic layers were washed with brine, and dried over sodium sulfate and concentrated in vacuo. The crude material was purified by HPLC to afford ((2S,5S)-2-{4-[4-(6-{2-[(S)-1-((S)-2-Methoxycarbonylamino-3-methyl-butyryl)-pyrrolidin-2-yl]-1H-imidazol-4-yl}-quinolin-2-yl)-phenyl]-1H-imidazol-2-yl}-4-oxo-1,2,4,5,6,7-hexahydro-azepino[3,2,1-hi]indol-5-yl)-carbamic acid methyl ester trifluoroacetic acid as yellow solid (28 mgs, 32%). Calculated for C₄₆H₄₇N₉O₆′ 2 C₂HF₃O₂ [M⁺] 1049.97, found [M+H⁺] 822 (free base). ¹H NMR (300 MHz, DMSO-d₆) δ ppm 0.67-0.97 (m, 6H) 1.92-2.23 (m, 5H) 2.22-2.40 (m, 2H) 3.08-3.19 (m, 2H) 3.29-3.36 (m, 1H) 3.51-3.54 (m, 6H) 3.83-3.92 (m, 3H) 4.02-4.27 (m, 2H) 5.07-5.24 (m, 1H) 5.82-5.99 (m, 1H) 6.93-7.26 (m, 3H) 7.34 (d, J=8.31 Hz, 1H) 7.52 (d, J=7.93 Hz, 1H) 7.84-8.62 (m, 11H).

### Example 22

### ((2S,5S)-2-{5-[2-(4-{2-[(S)-1-((S)-2-Methoxycarbonylamino-3-methyl-butyryl)-pyrrolidin-2-yl]-1H-imidazol-4-yl}-phenyl)-quinolin-6-yl]-1H-imidazol-2-yl}-4-oxo-1,2,4,5,6,7-hexahydro-azepino[3,2,1-hi]indol-5-yl)-carbamic acid methyl ester trifluoroacetic acid salt

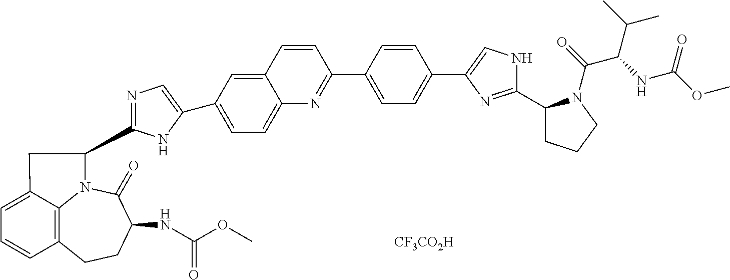

In a round-bottomed flask, (2S,5S)-5-(methoxycarbonylamino)-4-oxo-1,2,4,5,6,7-hexahydroazepino[3,2,1-hi]indole-2-carboxylic acid (0.5 g, 1.64 mmol), 2-(3H-[1,2,3]triazolo[4,5-b]pyridin-3-yl)-1,1,3,3-tetramethylisouronium hexafluorophosphate(V) (656 mg, 1.73 mmol) and aminoacetaldehyde dimethyl acetal (173 mg, 1.64 mmol) were combined with dimethylformamide (10 ml) to give a light yellow solution, N,N′-diisopropylethylamine (849 mg, 1.15 ml, 6.57 mmol, was added. The mixture was stirred at room temperature for 4 hours. The reaction mixture was poured into water and extracted with ethyl acetate. The organic layers were washed with brine, dried over sodium sulfate and concentrated in vacuo to afford light brown oil (0.642 g). Used for the next step.

In a round-bottomed flask, methyl (2S,5S)-2-(2,2-dimethoxyethyl-carbamoyl)-4-oxo-1,2,4,5,6,7-hexahydroazepino[3,2,1-hi]indol-5-ylcarbamate (642 mg, 1.64 mmol) and ammonium acetate (680 mg, 8.82 mmol) were combined with xylene (6 ml) and dioxane (2 ml) to give a light brown suspension. The reaction mixture was heated to 140° C. for over night. (LC-Mass showed the major was SM). The mixture was transferred into a microwave vial, acetic acid (5 ml) was added, and the vial was capped and heated in the microwave at 150° C. for 2 hour. The reaction mixture was poured into water, the dark solid was filtered off. The aqueous solution was adjusted PH>10, extracted with ethyl acetate, the organic layers were dried over sodium sulfate and concentrated in vacuo. The crude material was purified by flash chromatography (silica gel, 0% to 5% methanol/ethyl acetate) to afford a clear gum (100 mgs), LC-MS showed it contains both desired product [M+H] 327 and a by-product [M+H] 304 by 1:1 ratio, which was used for the next step.

To a solution of mixture from previous step (100 mg, 306 μmol) in dichloromethane (5 ml) was added N-iodosuccinimide (145 mg, 643 μmol) in portions at 0° C., the mixture was stirred for one hour, and poured into water, extracted with dichloromethane, the organic layers were dried over sodium sulfate and concentrated in vacuo to afford a yellow solid [M+H] 579, which was used for the next step.

In a round-bottomed flask, the mixture from previous step and sodium sulfite (328 mg, 2.6 mmol) were combined with ethanol (1.5 ml) and Water (3.5 ml) to give a white suspension. The mixture was heated at 110° C. for one day. Most of the ethanol was evaporated, solid was isolated from aqueous solution by filtration, and washed with more water, a light yellow solid obtained (56 mg), [M+H] 453, which was used for the next step.

In a round-bottomed flask, 2-chloro-6-(4,4,5,5-tetramethyl-1,3,2-dioxaborolan-2-yl)quinoline (40 mg, 138 μmol), methyl (2S,5S)-2-(4-iodo-1H-imidazol-2-yl)-4-oxo-1,2,4,5,6,7-hexahydroazepino[3,2,1-hi]indol-5-ylcarbamate (56 mg, 124 μmol) and sodium bicarbonate (31.2 mg, 371 μmol) were combined with toluene (1.5 ml), ethanol (1.00 ml), and water (0.5 ml) to give a light brown suspension, 1,1′-bis(diphenyl-phosphino)ferrocene-palladium(ii) dichloride dichloromethane complex (10.1 mg, 12.4 μmol) was added while degassed with argon. The reaction mixture was heated to 85° C. for 7 hours. The reaction mixture was poured into water, extracted with ethyl acetate. The organic layers were washed with brine, dried over sodium sulfate and concentrated in vacuo. The crude material was purified by flash chromatography (silica gel, 20%, 50%, 80% and 100% EtOAc in hexanes) to afford methyl (2S,5S)-2-(4-(2-chloroquinolin-6-yl)-1H-imidazol-2-yl)-4-oxo-1,2,4,5,6,7-hexahydroazepino[3,2,1-hi]indol-5-ylcarbamate as a yellow solid (29 mgs, 48%). Calculated for C₂₆H₂₂ClN₅O₃ [M⁺] 487.1, found [M+H⁺] 488.

To a 2 mL microwave vial was added methyl (2S,5S)-2-(4-(2-chloroquinolin-6-yl)-1H-imidazol-2-yl)-4-oxo-1,2,4,5,6,7-hexahydroazepino[3,2,1-hi]indol-5-ylcarbamate (29 mg, 59.4 mmol), methyl (S)-3-methyl-1-oxo-1-((S)-2-(4-(4-(4,4,5,5-tetramethyl-1,3,2-dioxaborolan-2-yl)phenyl)-1H-imidazol-2-yl)pyrrolidin-1-yl)butan-2-ylcarbamate (29.5 mg, 59.4 μmol) and sodium bicarbonate (25.0 mg, 297 μmol) in 1,2-Dimethoxyethane (1.5 ml) and Water (0.1 ml). 1,1′-bis(diphenylphosphino)ferrocene-palladium(ii) dichloride dichloromethane complex (4.85 mg, 5.94 μmol) was added while degassed with argon. The vial was capped and heated in the microwave at 140° C. for 30 min. The mixture was poured into 0.5N HCl aqueous solution, washed with ethyl acetate; the aqueous solution was adjusted PH>11 with 1 N sodium hydroxide aqueous solution, extracted with ethyl acetate, the organic layers were washed with brine, and dried over sodium sulfate and concentrated in vacuo. The crude material was purified by HPLC to afford ((2S,5S)-2-{5-[2-(4-{2-[(S)-1-((S)-2-Methoxycarbonylamino-3-methyl-butyryl)-pyrrolidin-2-yl]-1H-imidazol-4-yl}-phenyl)-quinolin-6-yl]-1H-imidazol-2-yl}-4-oxo-1,2,4,5,6,7-hexahydro-azepino[3,2,1-hi]indol-5-yl)-carbamic acid methyl ester trifluoroacetic acid as yellow solid (5 mgs, 8%). Calculated for C₄₆H₄₇N₉O₆.2C₂HF₃O₂ [M⁺] 1049.97, found [M+H⁺] 822 (free base). ¹H NMR (300 MHz, DMSO-d₆) δ ppm 0.58-1.00 (m, 6H) 1.95-2.28 (m, 6H) 2.69-2.74 (m, 1H) 3.00-3.19 (m, 2H) 3.48-3.60 (m, 6H) 3.63-4.22 (m, 6H) 5.05-5.19 (m, 1H) 5.73-5.95 (m, 1H) 6.91-7.23 (m, 3H) 7.34 (d, J=8.31 Hz, 1H) 7.50 (d, J=8.31 Hz, 1H) 7.87-8.54 (m, 11H).

### Example 23

### ((2S,5S)-2-{4-[6-(6-{2-[(S)-1-((S)-2-Methoxycarbonylamino-3-methyl-butyryl)pyrrolidin-2-yl]-3H-imidazol-4-yl}-naphthalen-2-yl)-pyridin-3-yl]-1Himidazol-2-yl}-4-oxo-1,2,4,5,6,7-hexahydro-azepino[3,2,1-hi]indol-5-yl)-carbamic acid methyl ester di-trifluoroacetic acid salt

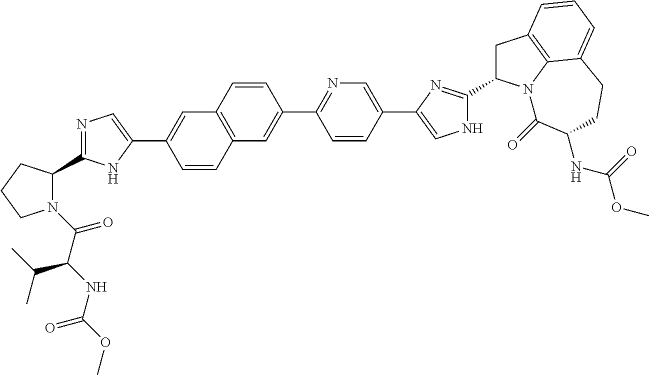

Diisopropylethylamine (1.27 g, 1.72 ml, 9.86 mmol, Eq: 1.5) was added to a flask containing 2-bromo-1-(6-bromopyridin-3-yl)ethanone (1.83 g, 6.57 mmol) and (2S,5S)-5-(methoxycarbonylamino)-4-oxo-1,2,4,5,6,7-hexahydroazepino[3,2,1-hi]indole-2-carboxylic acid (2 g, 6.57 mmol) in Acetonitrile (50.0 mL). Left to stir at room temperature for 2 h. Evaporated to dryness, dissolved in EtOAc, washed with brine, dried (MgSO₄) and evaporated to dryness to give (2S,5S)-2-(6-bromopyridin-3-yl)-2-oxoethyl 5-(methoxycarbonylamino)-4-oxo-1,2,4,5,6,7-hexahydroazepino[3,2,1-hi]indole-2-carboxylate. The residue was dissolved in dioxane (15 mL) and placed in a sealed tube containing ammonium acetate (1.52 g, 19.7 mmol). The mixture was heated at 140° C. in a microwave for 60 min. The cooled mixture was then evaporated to dryness. The residue was dissolved in EtOAc, washed with brine, dried (MgSO₄) and evaporated to dryness under reduced pressure. The residue was crystallized from a mixture of EtOAc and MeOH. The crystals were collected by filtration, washed with Et₂O and dried under vacuum. Chromatography (80 g, SiO₂; 30-100% EtOAc in Hexanes) provided methyl (2S,5S)-2-(4-(6-bromopyridin-3-yl)-1H-imidazol-2-yl)-4-oxo-1,2,4,5,6,7-hexahydroazepino[3,2,1-hi]indol-5-ylcarbamate as a yellow solid (0.47 g, 13.8%): ESI-LRMS m/z calculated for C₂₂H₂₀BrN₅O₃ [M⁺] 482, found 483 [M+H⁺].

Bromine (3.32 g, 1.07 ml, 20.8 mmol) was added to a heated solution of 1-(6-bromonaphthalen-2-yl)ethanone (5.18 g, 20.8 mmol) in acetic acid (100 mL). Left to cool to rt overnight. The precipitate was filtered, washed with Et₂O and dried under vacuum to give 2-bromo-1-(6-bromonaphthalen-2-yl)ethanone (5.02 g, 73%).

Diisopropylethylamine (962 μL, 5.51 mmol) was added to a flask containing 2-bromo-1-(6-bromonaphthalen-2-yl)ethanone (1.2 g, 3.67 mmol) and (S)-1-((S)-2-(methoxycarbonylamino)-3-methylbutanoyl)pyrrolidine-2-carboxylic acid (1 g, 3.67 mmol) in Acetonitrile (50 mL). After stirring for 24 h, the reaction mixture was evaporated to dryness, diluted with EtOAc, washed with brine, dried (MgSO₄) and evaporated to dryness to give (S)-2-(6-bromonaphthalen-2-yl)-2-oxoethyl 1-((S)-2-(methoxycarbonylamino)-3-methylbutanoyl)pyrrolidine-2-carboxylate (1.59 g, 71%) as a light grey solid: ESI-LRMS m/z calculated for C₂₄H₂₇BrN₂O₆ [M⁺] 519, found 521 [M+2H⁺].

A sealed flask containing (S)-2-(6-bromonaphthalen-2-yl)-2-oxoethyl 1-((S)-2-(methoxycarbonylamino)-3-methylbutanoyl)pyrrolidine-2-carboxylate (1.58 g, 3.04 mmol) and ammonium acetate (703 mg, 9.13 mmol) in dioxane (15 mL) was heated at 140° C. for 1 h in a microwave. The cooled solution was evaporated to dryness under reduced pressure, dissolved in EtOAc, washed with brine, dried (MgSO₄) and evaporated to dryness under reduced pressure. Chromatography (80 g SiO₂; 50-100% EtOAc in Hexanes 40 min) provided methyl (S)-1-((S)-2-(5-(6-bromonaphthalen-2-yl)-1H-imidazol-2-yl)pyrrolidin-1-yl)-3-methyl-1-oxobutan-2-ylcarbamate (0.93 g, 61%) as a light yellow foam: ESI-LRMS m/z calculated for C₂₄H₂₇BrN₂O₆

[M⁺] 499, found 501 [M+2]-11.

A sealed tube containing methyl (S)-1-((S)-2-(5-(6-bromonaphthalen-2-yl)-1H-imidazol-2-yl)pyrrolidin-1-yl)-3-methyl-1-oxobutan-2-ylcarbamate (0.88 g, 1.76 mmol), bis(pinacolato)diboron (895 mg, 3.52 mmol), tetrakis (102 mg, 88.1 μmol) and potassium acetate (380 mg, 3.88 mmol) in dioxane (26.4 mL) was heated for 5 h at 110° C. The cooled reaction mixture was evaporated to dryness under reduced pressure, diluted in EtOAc, washed with brine, dried (MgSO₄) and evaporated to dryness. Chromatography (40 g SiO₂, 30-100% EtOAc in Hexanes) provided [(S)-2-Methyl-1-((S)-2-{5-[6-(4,4,5,5-tetramethyl-[1,3,2]dioxaborolan-2-yl)-naphthalen-2-yl]-1H-imidazol-2-yl}-pyrrolidine-1-carbonyl)-propyl]-carbamic acid methyl ester (800 mg of material that was 85% pure by LCMS, 70%) as a yellow foam: ESI-LRMS m/z calculated for C₃₀H₃₀BN₄O₅ [M⁺] 546, found 547 [M+H⁺].

A sealed tube containing methyl (S)-3-methyl-1-oxo-1-((S)-2-(5-(6-(4,4,5,5-tetramethyl-1,3,2-dioxaborolan-2-yl)naphthalen-2-yl)-1H-imidazol-2-yl)pyrrolidin-1-yl)butan-2-ylcarbamate (271 mg, 421 μmol), methyl (2S,5S)-2-(4-(6-bromopyridin-3-yl)-1H-imidazol-2-yl)-4-oxo-1,2,4,5,6,7-hexahydroazepino[3,2,1-hi]indol-5-ylcarbamate (0.203 g, 421 μmol), sodium bicarbonate (106 mg, 1.26 mmol, Eq: 3) and tetrakis (48.6 mg, 42.1 μmol) in Toluene (2.5 mL), EtOH (1.5 mL), Water (500 μL) was heated in a microwave for 2 h at 100° C. Chromatographic purification (12 g SiO₂; 40-100% EtOAc in hexanes, then 5% MeOH in EtOAc) yielded a crude product, which was further purified by reverse phase HPLC to afford ((2S,5S)-2-{4-[6-(6-{2-[(S)-1-((S)-2-Methoxycarbonylamino-3-methyl-butyryl)pyrrolidin-2-yl]-3H-imidazol-4-yl}-naphthalen-2-yl)-pyridin-3-yl]-1Himidazol-2-yl}-4-oxo-1,2,4,5,6,7-hexahydro-azepino[3,2,1-hi]indol-5-yl)-carbamic acid methyl ester di-trifluoroacetic acid salt (134 mg, 30%) as a yellow solid: ESI-LRMS m/z calcd for C₄₆H₄₇N₉O₆ [M⁺] 822, found 823 [M+H⁺]. ¹H NMR (300 MHz, DMSO-d₆) δ ppm 0.81 (d, J=15 Hz, 3H), 0.83 (d, J=15 Hz, 3H), 1.90-2.54, 2.95-3.15, 3.25-4.20 (each m, 20H), 4.35-4.48 (m, 2H), 5.15 (br t, 1H), 5.87 (br q, 1H), 6.99-7.19 (m, 3H), 7.35 (d, J=8 Hz, 1H), 7.54 (d, J=8 Hz, 1H), 7.91-8.39 (m, 10H), 8.75 (s, 1H) and 9.11 (s, 1 H).

### Example 24

### ((2S,5S)-2-{4-[6-(5-{2-[(S)-1-((S)-2-Methoxycarbonylamino-3-methyl-butyryl)-pyrrolidin-2-yl]-3H-imidazol-4-yl}-pyridin-2-yl)-naphthalen-2-yl ]-1H-imidazol-2-yl}-4-oxo-1,2,4,5,6,7-hexahydro-azepino[3,2,1-hi]indol-5-yl)-carbamic acid methyl ester di-trifluoroacetic acid salt

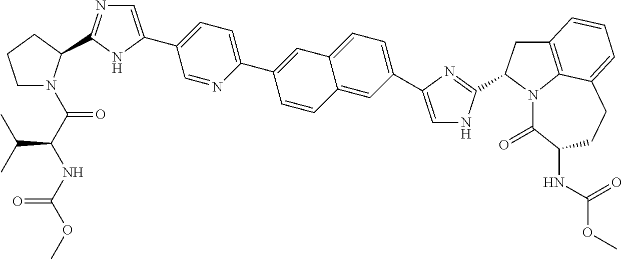

Diisopropylethylamine (952 μL, 5.45 mmol) was added to a flask containing 2-bromo-1-(6-bromopyridin-3-yl)ethanone (1.01 g, 3.63 mmol) and (S)-1-((S)-2-(methoxy-carbonylamino)-3-methylbutanoyl)pyrrolidine-2-carboxylic acid (0.989 g, 3.63 mmol) in acetonitrile (50.0 mL). The reaction mixture was evaporated to dryness, dissolved in EtOAc, washed with brine, dried (MgSO₄), evaporated to dryness and then triturated with EtOAc, filtered, to remove inorganics, and evaporated to dryness under reduced pressure. The crude (S)-2-(6-bromopyridin-3-yl)-2-oxoethyl 1-((S)-2-(methoxycarbonylamino)-3-methylbutanoyl)pyrrolidine-2-carboxylate (1.71 g, 3.64 mmol) was dissolved in dioxane (15.0 mL) and added to a 20 mL tube containing ammonium acetate (841 mg, 10.9 mmol). The tube was sealed and then heated at 140° C. for 1 h. The cooled mixture was evaporated to dryness under reduced pressure, dissolved in EtOAc, washed with brine, dried (MgSO₄) and evaporated under reduced pressure. Chromatography (40 g SiO₂; 30-100% EtOAc in hexanes) afforded methyl (S)-1-((S)-2-(5-(6-bromopyridin-3-yl)-1H-imidazol-2-yl)pyrrolidin-1-yl)-3-methyl-1-oxobutan-2-ylcarbamate as a yellow foam (0.79 g, 48%): ESI-LRMS m/z calcd for C₁₉H₂₄BrN₅O₃ [M⁺] 450, found 449 [M−H⁺].

Diisopropylethylamine (1.72 mL, 9.86 mmol) was added to a flask containing 2-bromo-1-(6-bromonaphthalen-2-yl)ethanone (2.16 g, 6.57 mmol) and (2S,5S)-5-(methoxycarbonylamino)-4-oxo-1,2,4,5,6,7-hexahydroazepino[3,2,1-hi]indole-2-carboxylic acid (2 g, 6.57 mmol) in acetonitrile (50 mL). It was left to stir at room temperature. After stirring for 24 h, the reaction mixture was evaporated to dryness, diluted with EtOAc, washed with brine, dried (MgSO₄) and evaporated to dryness. The residue was dissolved in dioxane (15 mL) and placed in a 20 mL tube containing ammonium acetate (839 mg, 10.9 mmol). The tube was sealed and then heated at 140° C. in a microwave for 60 min. The cooled mixture was evaporated to dryness under reduced pressure and the residue dissolved in EtOAc, washed with brine, dried (MgSO₄) and evaporated to dryness. The residue was crystallized from EtOAc and MeOH, filtered, washed with Et₂O and dried under vacuum to afford methyl (2S,5S)-2-(4-(6-bromonaphthalen-2-yl)-1H-imidazol-2-yl)-4-oxo-1,2,4,5,6,7-hexahydroazepino[3,2,1-hi]indol-5-ylcarbamate as a white solid (0.86 g, 45%): ESI-LRMS m/z calcd for C₂₇H₂₃BrN₄O₃ [M⁺]531, found 532 [M+H⁺].

A sealed tube containing methyl (2S,5S)-2-(4-(6-bromonaphthalen-2-yl)-1H-imidazol-2-yl)-4-oxo-1,2,4,5,6,7-hexahydroazepino[3,2,1-hi]indol-5-ylcarbamate (0.50 g, 941 μmol), bis(pinacolato)diboron (478 mg, 1.88 mmol), tetrakis (54.4 mg, 47.0 μmol) and potassium acetate (203 mg, 2.07 mmol) in dioxane (15 mL) was heated for 5 h at 110° C. The cooled reaction mixture was evaporated to dryness under reduced pressure. The residue was dissolved in EtOAc, washed with brine, dried (MgSO₄) and evaporated under reduced pressure. Chromatography (40 g SiO₂; 20-100% EtOAc in Hexanes) provided methyl (2S,5S)-4-oxo-2-(4-(6-(4,4,5,5-tetramethyl-1,3,2-dioxaborolan-2-yl)naphthalen-2-yl)-1H-imidazol-2-yl)-1,2,4,5,6,7-hexahydroazepino[3,2,1-hi]indol-5-ylcarbamate [0.454 g (88% purity) 73% yield) as a white foam: ESI-LRMS m/z calcd for C₃₃H₃₅BrN₄O₅ [M⁺]579, found 580 [M+2]-11.

A sealed tube containing methyl (S)-1-((S)-2-(5-(6-bromopyridin-3-yl)-1H-imidazol-2-yl)pyrrolidin-1-yl)-3-methyl-1-oxobutan-2-ylcarbamate (156 mg, 346 μmol), methyl (2S,5S)-4-oxo-2-(4-(6-(4,4,5,5-tetramethyl-1,3,2-dioxaborolan-2-yl)naphthalen-2-yl)-1H-imidazol-2-yl)-1,2,4,5,6,7-hexahydroazepino[3,2,1-hi]indol-5-ylcarbamate (0.2 g, 346 μmol), tetrakis (40.0 mg, 34.6 μmol, Eq: 0.1) and sodium bicarbonate (87.1 mg, 1.04 mmol,) in toluene (2.5 mL), ethanol (1.5 mL) and water (500 μL) was heated in a microwave at 100° C. for 1 h. The cooled mixture was diluted with EtOAc and water. The organic layer was separated, dried (MgSO₄) and evaporated to dryness under reduced pressure. Flash chromatography (12 g SiO₂; 40-100% EtOAc in hexanes then 5% MeOH in EtOAc). The crude product was further purified by reverse phase HPLC to afford ((2S,5S)-2-{4-[6-(5-{2-[(S)-1-((S)-2-Methoxycarbonylamino-3-methyl-butyryl)-pyrrolidin-2-yl]-3H-imidazol-4-yl}-pyridin-2-yl)-naphthalen-2-yl]-1H-imidazol-2-yl}-4-oxo-1,2,4,5,6,7-hexahydro-azepino[3,2,1-hi]indol-5-yl)-carbamic acid methyl ester di-trifluoroacetic acid (0.125 g, 35%) as a yellow solid: ESI-LRMS m/z calcd for C₄₆H₄₇N₉O₆ [M⁺] 822, found 823 [M+H⁺]. ¹H NMR (300 MHz, DMSO-d₆) δ ppm 0.81 (d, J=14 Hz, 3H), 0.83 (d, J=14 Hz, 3H), 1.86-2.60 (m, 8H), 3.14 (br t, 2H), 3.29-4.26 (m, 12H), 5.14 (br t, 1H), 5.92 (dd, J=4 and 10.5 Hz, 1H), 7.03-7.39 (m, 5H), 7.33 (d, J=8 Hz, 1H), 7.90-8.40 (m, 9 H), 8.75 (s, 1H) and 9.11 (s, 1H).

### Example 25

### {(2S,5S)-9-Bromo-2-[5-(4′-{2-[(R)-1-((R)-2-methoxycarbonylamino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester

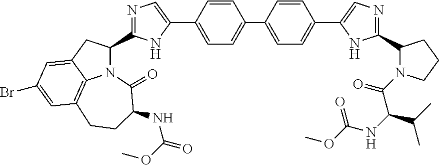

To a 0° C. solution of (2S,5S)-ethyl 5-(methoxycarbonylamino)-4-oxo-1,2,4,5,6,7-hexahydroazepino[3,2,1-hi]indole-2-carboxylate (1.5 g, 4.5 mmol) in 25 mL of N,N-dimethylformamide was added N-bromosuccinimide (2.8 g, 15.7 mmol). The reaction mixture was slowly warmed to ambient temperature, and stirred for 3 h. The reaction mixture was partitioned between water and ethyl acetate, the organic layer was washed with aqueous sodium thiosulfate, water, and then dried over sodium sulfate. The mixture was filtered, concentrated, and the residue purified by flash chromatography (1:4 ethyl acetate/hexanes) to afford (2S,5S)-ethyl 9-bromo-5-(methoxycarbonylamino)-4-oxo-1,2,4,5,6,7-hexahydroazepino[3,2,1-hi]indo le-2-carboxylate (1.47 g, 79%) as a colorless foam: ESI-LRMS m/e calc'd for C₁₇H₂₀BrN₂O₅ [M]⁺412, found [M+H]⁺ 413.

To a solution of (2S,5S)-ethyl 9-bromo-5-(methoxycarbonylamino)-4-oxo-1,2,4,5,6,7-hexahydroazepino[3,2,1-hi]indole-2-carboxylate (1.12 g, 2.72 mmol) in tetrahydrofuran (20 ml) at 0° C. was added a solution of lithium hydroxide (326 mg, 13.6 mmol) in water (10 ml) dropwise via syringe over 5 minutes. The resulting yellow solution was stirred at 0° C. for 4 hours before being made acidic with a 1 N hydrochloric acid solution (pH 4). The resulting solution was extracted with ethyl acetate (4×30 mL). The organic phases were combined, washed with brine (1×50 ml) and dried over sodium sulfate. The mixture was filtered and evaporated and the solid dried under high vac over night to afford (2S,5S)-9-bromo-5-methoxycarbonylamino-4-oxo-1,2,4,5,6,7-hexahydro-azepino[3,2,1-hi]indole-2-carboxylic acid (0.98 g, 94%) as a tan solid: ESI-LRMS m/e calc'd for C₁₅H₁₆BrN₂O₅ [M]⁺] 383, found [M+H]⁺ 384.

To a heterogeneous mixture of methyl (S)-1-((S)-2-(5-(4′-(2-aminoacetyl)biphenyl-4-yl)-1H-imidazol-2-yl)pyrrolidin-1-yl)-3-methyl-1-oxobutan-2-ylcarbamate (984 mg, 1.95 mmol), (2S,5S)-9-bromo-5-(methoxycarbonylamino)-4-oxo-1,2,4,5,6,7-hexahydroazepino[3,2,1-hi]indole-2-carboxylic acid (898 mg, 2.34 mmol), 2-(7-aza-1H-benzotriazole-1-yl)-1,1,3,3-tetramethyluronium hexafluorophosphate (743 mg, 1.95 mmol) in N,N-dimethylformamide (40.0 ml) at room temperature was added N,N-DIISOPROPYLETHYLAMINE (505 mg, 683 μA, 3.91 mmol) dropwise via syringe. The resulting dark yellow solution was allowed to stir at room temperature for 5 hr, after which the reaction mixture was poured into water (100 ml) and extracted with ethyl acetate (3×100 mL). The organic phases were combined and washed with 1 N hydrochloric acid solution (1×100 ml), saturated sodium bicarbonate (1×100 ml) and brine (1×100 ml) and dried over anhydrous magnesium sulfate. The mixture was filtered and evaporated to afford a yellowish film which was purified by automated flash chromatography (Analogix 270, Analogix Superflash SF40-80 g, 0% to 10% methanol/dichloromethane) to afford {(2S,5S)-9-bromo-2-[2-(4′-{2-[(R)-1-((R)-2-methoxycarbonylamino-3-methyl-butyryl)-pyrrolidin-2-yl]-3H-imidazol-4-yl}-biphenyl-4-yl)-2-oxo-ethylcarbamoyl]-4-oxo-1,2,4,5,6,7-hexahydro-azepino[3,2,1-hi]indol-5-yl}-carbamic acid methyl ester (1.07 g, 63%) as a yellow solid: ESI-LRMS m/e calc'd for C₄₃H₄₆BrN₇O₈ [M]⁺869, found [M+H]⁺ 870.

A mixture of {(2S,5S)-9-bromo-2-[2-(4′-{2-[(R)-1-((R)-2-methoxycarbonylamino-3-methyl-butyryl)-pyrrolidin-2-yl]-3H-imidazol-4-yl}-biphenyl-4-yl)-2-oxo-ethylcarbamoyl]-4-oxo-1,2,4,5,6,7-hexahydro-azepino[3,2,1-hi]indol-5-yl}-carbamic acid methyl ester (965 mg, 1.11 mmol), ammonium acetate (428 mg, 5.55 mmol) and xylene (100 mL) was sealed in a tube and heated to 140° C. for 4 h. Solution was cooled to room temperature and let stand overnight. The solution was diluted with ethyl acetate (250 ml) and washed successively with a saturated sodium bicarbonate solution (250 ml), brine (250 ml) and then dried over anhydrous magnesium sulfate. The mixture was filtered and evaporated to afford a yellow film which was triturated with dichloromethane and hexane to afford a gummy solid which was purified by automated flash chromatography (Analogix 270, Analogix SuperFlash SF25-40 g, 0 to 2% methanol/ethyl acetate) to afford {(2S,5S)-9-bromo-2-[5-(4′-{2-[(R)-1-((R)-2-methoxycarbonylamino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester (239 mg, 25%) as a yellow solid: ESI-LRMS m/e calc'd for C₄₃H₄₅BrN₈O₆ [M]⁺850, found [M+H]⁺ 851; ¹H NMR (DMSO-d₆, 300 MHz) δ ppm: 7.02-7.98 (m, 15H), 5.78 (d, J=9.8 Hz, 1H), 5.08 (br. s., 1H), 4.01-4.14 (m, 1H), 3.72-3.92 (m, 1H), 3.43-3.65 (m, 8H), 3.39 (s, 1H), 3.08 (br. s., 2H), 1.65-2.21 (m, 2H), 1.00-1.35 (m, 2H), 0.65-1.02 (m, 8H).

### Example 26

### {(2S,5S)-9-Cyano-2-[5-(4′-{2-[(R)-1-((R)-2-methoxycarbonylamino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester

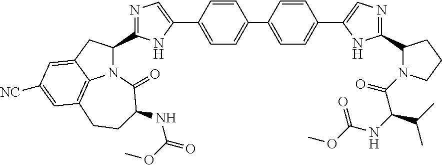

A solution of N,N,N′,N′-tetramethylethylenediamine (133 mg, 1.14 mmol), zinc cyanide (14.2 mg, 120 μmol), tris(dibenzylideneacetone)dipalladium(0) (14.2 mg, 15.5 μmol), 4,5-bis(diphenylphosphino)-9,9-dimethylxanthene (32.6 mg, 56.3 μmol), {(2S,5S)-9-bromo-2-[5-(4′-{2-[(R)-1-((R)-2-methoxycarbonylamino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester (141.5 mg, 167 μmol) in N,N-dimethylformamide (5 ml) was degassed with nitrogen, sealed in a tube and heated in a microwave reactor to 160° C. for 5 minutes. After cooling, the reaction mixture was filtered through a pad of Celite and silica gel, and the filter cake washed with N,N-dimethylformamide (1 mL). The resulting filtrate was purified by reverse phase HPLC using a 50 g Polaris C18A column eluting with acetonitrile/water (30% to 100%) to afford, after combining like fractions, {(2S,5S)-9-cyano-2-[5-(4′-{2-[(R)-1-((R)-2-methoxycarbonylamino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester (55 mg, 41%) as a light yellow solid: ESI-LRMS m/e calc'd for C₄₄H₄₆N₉O₆ [M]⁺796, found [M]⁺ 796; ¹H NMR (DMSO-d₆, 300 MHz) δ ppm: 7.16-8.00 (m, 11H), 5.81 (d, J=9.0 Hz, 1H), 5.08 (br. s., 1H), 3.49-4.36 (m, 10H), 3.12 (br. s., 2H), 1.79-2.40 (m, 5H), 1.10-1.36 (m, 1H), 0.88 (d, J=15.3 Hz, 5H).

### Example 27

### {(2S,5S)-9-Carbamoyl-2-[5-(4′-{2-[(R)-1-((R)-2-methoxycarbonylamino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester

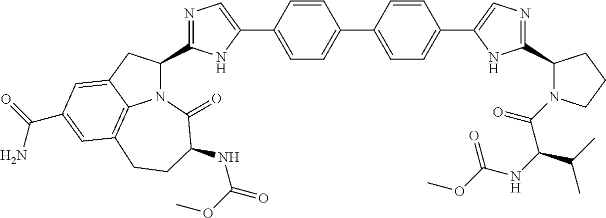

To a solution of {(2S,5S)-9-cyano-2-[5-(4′-{2-[(R)-1-((R)-2-methoxycarbonylamino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester (11.5 mg, 14.4 μmol), potassium carbonate (3.99 mg, 28.9 μmol) in a mixture of dimethylsulfoxide and d₆-dimethylsulfoxide (5:1, 5 ml total) was added a 30% solution of hydrogen peroxide (1.46 μA, 14.4 μmol). The resulting solution was stirred for 1 h at room temperature after which an additional drop of hydrogen peroxide was added and the resulting solution allowed to stir for an addition 2H. The reaction mixture was filtered, diluted with 1 ml of dimethylsulfoxide and purified by reverse phase HPLC using a 50 g Polaris C18A column eluting with acetonitrile/water (30% to 100%) to afford {(2S,5S)-9-carbamoyl-2-[5-(4′-{2-[(R)-1-((R)-2-methoxycarbonylamino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester (2.8 mg, 24%) as an off white gummy solid: ESI-LRMS m/e calc'd for C₄₄H₄₇N₉O₇ [M]⁺813, found [M+H]⁺ 814; ¹H NMR (DMSO-d₆, 300 MHz) δ ppm: 11.41-12.09 (m, 2H), 6.94-8.18 (m, 15H), 5.81 (d, J=10.0 Hz, 1H), 5.08 (br. s., 1H), 3.42-4.39 (m, 13H), 1.57-2.42 (m, 7H), 0.66-1.43 (m, 8H).

### Example 28

### {(2S,5S)-2-[5-(4′-{2-[(S)-1-((8)-2-Methoxycarbonylamino-3-methyl-butyryl)-pyrrolidin-2-yl]-3H-imidazol-4-yl}-biphenyl-4-yl)-1H-imidazol-2-yl]-4,7-dioxo-1,2,4,5,6,7-hexahydro-azepino[3,2,1-hi]indol-5-yl}-carbamic acid methyl ester

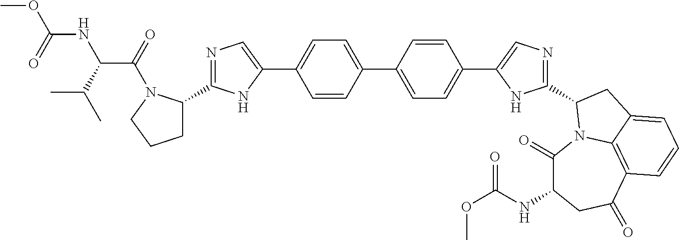

(2S,5S)-5-Methoxycarbonylamino-4,7-dioxo-1,2,4,5,6,7-hexahydro-azepino[3,2,1-hi]indole-2-carboxylic acid methyl ester (5.5 g, 16 mmol) was combined with methanol (125 ml) and cooled to 5° C. Sodium hydroxide (4.5 g, 113 mmol) dissolved in 70 mL of cold water, was added over 0.25 hr by syringe. After 4 hr at 5° C., the cold basic solution was made acidic with cold 3 M HCl (pH 1-2) and most of the solvent was removed under vacuum. The residue was dissolved in 20% THF/dichloromethane and washed with brine to give a bright yellow solution. Drying (MgSO₄) and filtration gave a solution which was reduced under vacuum to give a foam that became a solid powder when triturated with hexane to give a mixture of two diasteriomers with differing retention times. These were separated by silica gel chromatography to give (2S,5S)-5-methoxycarbonylamino-4,7-dioxo-1,2,4,5,6,7-hexahydro-azepino[3,2,1-hi]indole-2-carboxylic acid. ESI-LRMS m/e [M+] 391.

(2S,5S)-5-Methoxycarbonylamino-4,7-dioxo-1,2,4,5,6,7-hexahydro-azepino[3,2,1-hi]indole-2-carboxylic acid (4.5 g, 14.1 mmol), HATU (5.38 g, 14.1 mmol), Hunig's base (9.14 g, 12.3 mL, 70.7 mmol) were combined in DMF (150 mL). To this was added 1-(4-bromophenyl)-2-(bromoamino)ethanone (4.43 g, 14.1 mmol) as the HCl salt. This was stirred at room temperature for 48 hr. The crude reaction mixture was concentrated under vacuum. The residue was dissolved in ethyl acetate and washed with 10% brine/water (2×). The aqueous portion was back extracted with ethyl acetate. The combined organic extracts were washed with water and brine. After drying (MgSO₄), the solvent was removed in vacuo and the crude material was purified by silica gel chromatography (220 g column, 60-100% ethyl acetate in hexanes). This gave 2.8 g (35% yield) of (2S,5S)-5-methoxycarbonylamino-4,7-dioxo-1,2,4,5,6,7-hexahydro-azepino[3,2,1-hi]indole-2-carboxylic acid 2-(4-bromo-phenyl)-2-oxo-ethyl ester. ESI-LRMS m/e [M+] 515.

In a 25 mL Parr pressure vessel, (2S,5S)-5-methoxycarbonylamino-4,7-dioxo-1,2,4,5,6,7-hexahydro-azepino[3,2,1-hi]indole-2-carboxylic acid 2-(4-bromo-phenyl)-2-oxo-ethyl ester (1.05 g, 2.04 mmol) and ammonium acetate (1.57 g, 20.4 mmol) were combined with dioxane (15 ml) and sparged for 5 minutes with nitrogen. The vessel was sealed and the reaction was heated to 110-115° C. for 18 hr. The vessel was cooled and the contents diluted with ethyl acetate and filtered through Celite. The crude material was purified by silica gel chromatography (23 g column, 60-80% ethyl acetate in hexanes). This gave 450 mg (36% yield) of {(2S,5S)-2-[5-(4-bromo-phenyl)-1H-imidazol-2-yl]-4,7-dioxo-1,2,4,5,6,7-hexahydro-azepino[3,2,1-hi]indol-5-yl}-carbamic acid methyl ester which was taken to the next step.

In a sealed tube a mixture of [(S)-2-methyl-1-((S)-2-{5-[4-(4,4,5,5-tetramethyl-[1,3,2]dioxaborolan-2-yl)-phenyl]-1H-imidazol-2-yl}-pyrrolidine-1-carbonyl)-propyl]-carbamic acid methyl ester (265 mg, 0.496 mmol) (prepared by Chembiotek), {(2S,5S)-2-[5-(4-bromo-phenyl)-1H-imidazol-2-yl]-4,7-dioxo-1,2,4,5,6,7-hexahydro-azepino[3,2,1-hi]indol-5-yl}-carbamic acid methyl ester (220 mg, 0.444 mmol), aqueous sodium bicarbonate (224 mg, 2.66 mmol) in water (3 mL) and t-butanol (15 mL) was sparged for 10 minutes with nitrogen. PdCl₂(dppf) (42 mg, 0.08 mmol) was added and the sealed mixture heated at 80° C. for 4 hr. The cooled mixture was decanted into water and the solids filtered. The crude solid was purified by silica gel chromatography (40 g column, ethyl acetate then 40-50% tetrahydrofuran/ethyl acetate) to afford {(2S,5S)-2-[5-(4′-{2-[(S)-1-((S)-2-methoxycarbonylamino-3-methyl-butyryl)-pyrrolidin-2-yl]-3H-imidazol-4-yl}-biphenyl-4-yl)-1H-imidazol-2-yl]-4,7-dioxo-1,2,4,5,6,7-hexahydro-azepino[3,2,1-hi]indol-5-yl}-carbamic acid methyl ester as a solid (90 mg, 24%): ESI-LRMS m/e [M+]: 496.0. ¹H NMR (400 MHz), DMSO-_{d6}) δ 12.00 (s, 1H) 11.79 (s, 1H) 7.58-7.85 (m, 10H) 7.55 (d, J=2, 1H) 7.52 (d, J=2, 1H) 7.31 (d, J=8.5, 1H) 5.93 (m, 1H) 5.08 (dd, J=6.9, 2.9, 1H) 4.61 (m, 1H) 4.03 (m, 1H) 3.74-3.81 (m, 6H) 3.57 (s, 3H) 3.54 (s, 3H) 3.42 (m, 2H) 2.78 (m, 1H) 1.99-2.07 (m, 3H) 2.15-2.11 (m, 1H) 0.90 (d, J=6.8, 3H) 0.85 (d, J=6.7, 3H).

### Example 29

### {(2S,5S)-7-Hydroxy-2-[5-(4′-{2-[(S)-1-((S)-2-methoxycarbonylamino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester

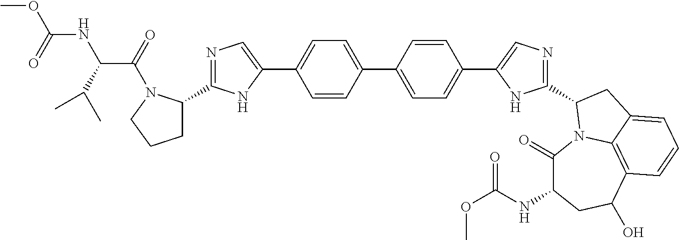

A solution of {(2S,5S)-2-[5-(4′-{2-[(S)-1-((S)-2-methoxycarbonylamino-3-methyl-butyryl)-pyrrolidin-2-yl]-3H-imidazol-4-yl}-biphenyl-4-yl)-1H-imidazol-2-yl]-4,7-dioxo-1,2,4,5,6,7-hexahydro-azepino[3,2,1-hi]indol-5-yl}-carbamic acid methyl ester (26 mg, 33 μmol) was suspended in a mixture of THF (2 mL) and ethanol (10 mL) and treated at 5° C. with sodium borohydride (12 mg, 318 μmol). The mixture was allowed to come to room temperature over 0.5 hr. The solution was diluted with 20% brine/water and extracted into dichloromethane (2×). After drying (MgSO₄), the solvent was removed in vacuo and the crude material was purified by silica gel chromatography (40-60% THF/ethyl acetate) to give 10 mg of {(2S,5S)-7-hydroxy-2-[5-(4′-{2-[(S)-1-((S)-2-methoxycarbonylamino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester (29%). LC/MS [M+]: 787.8. ¹H NMR (400 MHz), DMSO-d₆) δ 11.82 (s, 1H) 11.77 (s, 1H) 7.48-7.84 (m, 10H) 7.41-7.19 (m, 3H) 7.10 (t, 1H) 5.67-5.83 (m, 2H) 5.08 (m, 1H) 4.92 (m, 1H) 4.42 (m, 1H) 4.06 (m, 1H) 3.81 (m, 1H) 3.64 (m, 1H) 3.51-3.57 (m, 6H) 3.38 (m, 1H) 2.14 (m, 4H) 1.95 (m, 4H) 0.90 (d, J=6.8, 3H) 0.85 (d, J=6.8, 3H).

### Example 30

### {(2S,5S)-2-[5-(4′-{5-Chloro-2-[(S)-1-((S)-2-methoxycarbonyl amino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester

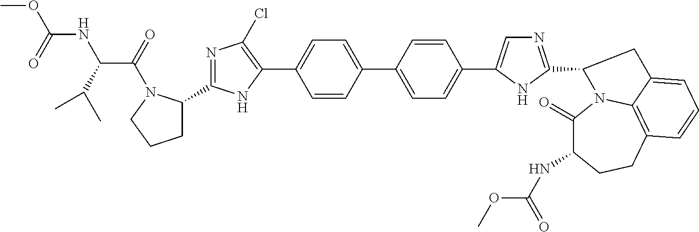

To a stirred solution of 2-amino-1-(4-bromophenyl)ethanone hydrochloride (2.0 g, 7.98 mmol) in THF (20 ml) was added N,N-diisopropylethylamine (1.24 g, 9.58 mmol) and di-tert-butyl dicarbonate (1.74 g, 7.98 mmol). Stir the reaction at room temperature for 2 h. Concentrate the reaction in vacuo. The crude mixture was diluted with ethyl acetate and washed with a 2N HCl solution, a saturated sodium bicarbonate solution a saturated sodium chloride solution and dried over magnesium sulfate, filtered and concentrated. The crude product obtained was purified by ISCO flash chromatography (Teledyne Isco RediSep Flash Column 80 g; (0% to 100% ethyl acetate/hexane) to afford, [2-(4-bromo-phenyl)-2-oxo-ethyl]-carbamic acid tert-butyl ester, as a white solid, (2.07 g, 83%): ESI-LRMS m/e calcd for C₁₃H₁₆BrNO₃ [M⁺] 313, found 314 [M+H⁺].

In a sealed tube 1,1′-bis(diphenylphosphino)ferrocene-palladium(II)dichloride (147 mg, 201 μmol) was added to a mixture of methyl (S)-3-methyl-1-oxo-1-((S)-2-(5-(4-(4,4,5,5-tetramethyl-1,3,2-dioxaborolan-2-yl)phenyl)-1H-imidazol-2-yl)pyrrolidin-1-yl)butan-2-ylcarbamate (1.0 g, 2.01 mmol), [2-(4-bromophenyl)-2-oxo-ethyl]-carbamic acid tert-butyl (633 mg, 2.01 mmol) and sodium bicarbonate (508 mg, 6.04 mmol) in 1,2-dimethoxyethane (10 ml) and water (2 ml). The reaction mixture was flushed with nitrogen, capped and heated in an oil bath (80° C.) for 16 h. The reaction mixture was concentrated and partitioned between 20% methanol/methylene chloride and water and the aqueous phase extracted with 20% methanol/methylene chloride. The combined organic phases were washed with a saturated sodium chloride solution and dried over magnesium sulfate, filtered and concentrated. The crude product obtained was purified by ISCO flash chromatography (Teledyne Isco RediSep Flash Column 80 g; (0% to 100% ethyl acetate/hexane) to afford, [(S)-1-((S)-2-{5-[4′-(2-tert-butoxycarbonylamino-acetyl)-biphenyl-4-yl]-1H-imidazol-2-yl}-pyrrolidine-1-carbonyl)-2-methyl-propyl]-carbamic acid methyl ester as a yellow oil, (715 mg, 59%): ESI-LRMS m/e calcd for C₃₃H₄₁N₅O₆ [M⁺] 603, found 604 [M+H⁺].

To a solution of [(S)-1-((S)-2-{544′-(2-tert-Butoxycarbonylamino-acetyl)-biphenyl-4-yl]-1H-imidazol-2-yl}-pyrrolidine-1-carbonyl)-2-methyl-propyl]-carbamic acid methyl ester (230 mg, 381 μmol) in DMF (5.0 ml) was added N-chlorosuccinimide (61.0 mg, 457 μmol). The reaction mixture was heated at 50° C. for 16 h. After the reaction was complete by TLC it was cooled to room temperature. The crude mixture was diluted with ethyl acetate and washed with water and a saturated sodium chloride solution and dried over magnesium sulfate, filtered and concentrated. The crude product obtained was purified by ISCO flash chromatography (Teledyne Isco RediSep Flash Column 40 g; (50% to 100% ethyl acetate/hexane) to afford, [(S)-1-((S)-2-{5-[4′-(2-tert-Butoxycarbonylamino-acetyl)-biphenyl-4-yl]-4-chloro-1H-imidazol-2-yl}-pyrrolidine-1-carbonyl)-2-methyl-propyl]-carbamic acid methyl ester as a yellow oil, (220 mg, 91%): ESI-LRMS m/e calcd for C₃₃H₄₀ClN₅O₆ [M⁺] 637, found 638 [M+H⁺].

A mixture of [(S)-1-((S)-2-{5-[4′-(2-tert-butoxycarbonylamino-acetyl)-biphenyl-4-yl]-4-chloro-1H-imidazol-2-yl}-pyrrolidine-1-carbonyl)-2-methyl-propyl]-carbamic acid methyl ester (370 mg, 580 μmol) and 4.0M HCl in dioxane solution (10 ml) in methanol (10 ml) was stirred at room temperature for 4 h. Concentrate the reaction in vacuo to afford, methyl (S)-1-((S)-2-(5-(4′-(2-amino acetyl)biphenyl-4-yl)-4-chloro-1H-imidazol-2-yl)pyrrolidin-1-yl)-3-methyl-1-oxobutan-2-ylcarbamate hydrochloride as a brown solid, (317 mg, 95%): ESI-LRMS m/e calcd for C₂₈H₃₂ClN₅O₄ HCl [M⁺] 537, found 538 [M+H⁺] (free base).

To a heterogeneous mixture of methyl (S)-1-((S)-2-(5-(4′-(2-aminoacetyl)biphenyl-4-yl)-4-chloro-1H-imidazol-2-yl)pyrrolidin-1-yl)-3-methyl-1-oxobutan-2-ylcarbamate hydrochloride (315 mg, 548 μmol), (2S,5S)-5-(methoxycarbonylamino)-4-oxo-1,2,4,5,6,7-hexahydroazepino[3,2,1-hi]indole-2-carboxylic acid (200 mg, 657 μmol) and HATU (208 mg, 548 μmol) in DMF (10.0 ml) was added N,N-diisopropylethylamine (213 mg, 1.64 mmol) dropwise at room temperature. Stir the clear dark yellow reaction at room temperature for 4 h. The reaction mixture was diluted with ethyl acetate and washed with water, 1N hydrochloric acid, a saturated sodium bicarbonate solution, a saturated sodium chloride solution and dried over magnesium sulfate, filtered and concentrated to afford, {(2S,5S)-2-[2-(4′-{5-Chloro-2-[(S)-1-((S)-2-methoxycarbonylamino-3-methyl-butyryl)-pyrrolidin-2-yl]-3H-imidazol-4-yl}-biphenyl-4-yl)-2-oxo-ethylcarbamoyl]-4-oxo-1,2,4,5,6,7-hexahydro-azepino[3,2,1-hi]indol-5-yl}-carbamic acid methyl ester as a brown solid, (440 mg, 97%): ESI-LRMS m/e calcd for C₄₃H₄₆ClN₇O₈ [M⁺] 823, found 824 [M+H⁺].

A mixture of {(2S,5S)-2-[2-(4′-{5-Chloro-2-[(S)-1-((S)-2-methoxycarbonylamino-3-methyl-butyryl)-pyrrolidin-2-yl]-3H-imidazol-4-yl}-biphenyl-4-yl)-2-oxo-ethylcarbamoyl]-4-oxo-1,2,4,5,6,7-hexahydro-azepino[3,2,1-hi]indol-5-yl (440 mg, 534 μmol) and ammonium acetate (206 mg, 2.67 mmol) in xylene (10 ml) was heated in a sealed tube at 140° C. for 4 h. The reaction mixture was diluted with methylene chloride and washed with a saturated sodium bicarbonate solution, a saturated sodium chloride solution and dried over magnesium sulfate, filtered and concentrated. The crude product obtained was purified by reverse phase HPLC using a 50 g Polaris C18A column eluting with acetonitrile/water (30% to 100%) to afford an off-white powder, (59 mg, 14%):

ESI-LRMS m/e calcd for C₄₆H₄₂N₈O₆ [M⁺] 805, found 806 [M+H⁺]; ¹H NMR (400 MHz, DMSO-d₆) δ ppm 0.72-1.00 (m, 6H) 1.70-2.35 (m, 6H) 2.75-3.95 (m, 2H) 3.28-3.44 (m, 2 H) 3.65 (d, J=6.27 Hz, 6H) 3.75-3.85 (m, 1H) 4.01-4.20 (m, 1H) 4.45-4.60 (m, 1H) 4.85-4.95 (m, 1H) 5.75-5.86 (m, 1H) 6.87-7.05 (m, 3H) 7.20 (s, 1H) 7.55-780 (m, 9H) 11.56-11.92 (m, 2H).

### Example 31

### {(2S,5S)-2-[5-(4-(6-{2-[(S)-1-((S)-2-Methoxycarbonylamino-3-methyl-butyryl)-pyrrolidin-2-yl]-3H-imidazol-4-yl}-naphthalen-2-yl)-phenyl)-1H-imidazol-2-yl]-4-oxo-1,2,4,5,6,7-hexahydro-azepino[3,2,1-hi]indol-5-yl}-carbamic acid methyl ester

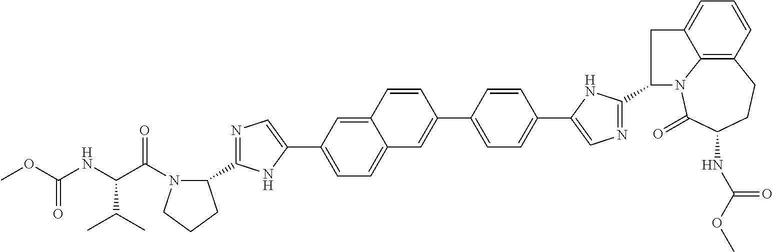

A mixture of (S)-tert-butyl 2-(5-(6-bromonaphthalen-2-yl)-1H-imidazol-2-yl)pyrrolidine-1-carboxylate (500 mg, 1.13 mmol) and 4.0M HCl in dioxane solution (15 ml) in methanol (20 ml) was stirred at room temperature for 3 h. Concentrate the reaction in vacuo to afford, (S)-5-(6-bromonaphthalen-2-yl)-2-(pyrrolidin-2-yl)-1H-imidazole hydrochloride as a yellow solid, (425 mg, 99%): ESI-LRMS m/e calcd for C₁₆H₁₆BrN₃HCl [M⁺] 342, found 343 [M+H⁺] (free base).

N,N-Diisopropyethylamine (461 mg, 3.56 mmol) was added dropwise at room temperature to a heterogeneous mixture of (S)-5-(6-bromonaphthalen-2-yl)-2-(pyrrolidin-2-yl)-1H-imidazole hydrochloride (450 mg, 1.19 mmol), (S)-2-(methoxycarbonylamino)-3-methylbutanoic acid (208 mg, 1.19 mmol), HATU (452 mg, 1.19 mmol) and DMF (10 ml). After the addition was complete the reaction mixture was stirred at room temperature for 4 h. The reaction mixture was diluted with ethyl acetate and washed with water, 1N hydrochloric acid, a saturated sodium bicarbonate solution, a saturated sodium chloride solution and dried over magnesium sulfate, filtered and concentrated. The crude product obtained was purified by ISCO flash chromatography (Teledyne Isco RediSep Flash Column 40 g; (0% to 100% ethyl acetate/hexane) to afford, methyl (S)-1-((S)-2-(5-(6-bromonaphthalen-2-yl)-1H-imidazol-2-yl)pyrrolidin-1-yl)-3-methyl-1-oxobutan-2-ylcarbamate as a white solid, (392 mg, 66%): ESI-LRMS m/e calcd for C₂₄H₂₇BrN₄O₃ [M⁺] 499, found 500 [M+H⁺].

1,1′-Bis(diphenylphosphino)ferrocene-palladium(II)dichloride dichloromethane complex (39 mg, 48 μmol) was added to a sealed tube containing a mixture of methyl (S)-1-((S)-2-(5-(6-bromonaphthalen-2-yl)-1H-imidazol-2-yl)pyrrolidin-1-yl)-3-methyl-1-oxobutan-2-ylcarbamate (240 mg, 481 μmol), 4,4,4′,4′,5,5,5′,5′-octamethyl-2,2′-bi(1,3,2-dioxaborolane) (244 mg, 961 mop, potassium acetate (141 mg, 1.44 mmol) and DMSO (10 ml). The vessel was purged with nitrogen, capped and heated with an oil bath at 80° C. for 4 h. Cool the reaction to room temperature and filter through celite. The crude mixture was diluted with ethyl acetate and washed with water, a saturated sodium chloride solution and dried over magnesium sulfate, filtered and concentrated. The crude product obtained was purified by ISCO flash chromatography (Teledyne Isco RediSep Flash Column 40 g; (20% to 100% ethyl acetate/hexane) to afford, methyl (S)-3-methyl-1-oxo-1-((S)-2-(5-(6-(4,4,5,5-tetramethyl-1,3,2-dioxaborolan-2-yl)naphthalen-2-yl)-1H-imidazol-2-yl)pyrrolidin-1-yl)butan-2-ylcarbamate as a white solid, (174 mg, 66%): ESI-LRMS m/e calcd for C₃₀H₃₉BN₄O₅ [M⁺] 546, found 547 [M+H⁺].

In a sealed tube 1,1′-bis(diphenylphosphino)ferrocene-palladium(II)dichloride (23 mg, 31 mmol) was added to a mixture of methyl (S)-3-methyl-1-oxo-1-((S)-2-(5-(6-(4,4,5,5-tetramethyl-1,3,2-dioxaborolan-2-yl)naphthalen-2-yl)-1H-imidazol-2-yl)pyrrolidin-1-yl)butan-2-ylcarbamate (170 mg, 311 μmol), methyl (2S,5S)-2-(5-(4-bromophenyl)-1H-imidazol-2-yl)-4-oxo-1,2,4,5,6,7-hexahydroazepino[3,2,1-hi]indol-5-ylcarbamate (150 mg, 311 mop and sodium bicarbonate (78 mg, 933 mol) in 1,2-dimethoxyethane (6 ml) and water (1 ml). The reaction mixture was flushed with nitrogen, capped and heated in an oil bath (80° C.) for 16 h. The reaction mixture was concentrated and partitioned between 20% methanol/methylene chloride and water and the aqueous phase extracted with 20% methanol/methylene chloride. The combined organic phases were washed with a saturated sodium chloride solution and dried over magnesium sulfate, filtered and concentrated. The crude product obtained was purified by reverse phase HPLC using a 50 g Polaris C18A column eluting with acetonitrile/water (30% to 100%) to afford, {(2S,5S)-2-[5-(4-(6-{2-[(S)-1-((S)-2-Methoxycarbonylamino-3-methyl-butyryl)-pyrrolidin-2-yl]-3H-imidazol-4-yl}-naphthalen-2-yl)-phenyl)-1H-imidazol-2-yl]-4-oxo-1,2,4,5,6,7-hexahydro-azepino[3,2,1-hi]indol-5-yl}-carbamic acid methyl ester as a white solid, (42 mg, 16%): ESI-LRMS m/e calcd for C₄₇H₄₈N₈O₆ [M⁺] 820, found 821 [M+H⁺]; ¹H NMR (400 MHz, DMSO-d₆) δ PPM 0.72-1.00 (m, 6H) 1.80-2.35 (m, 8H) 2.99-3.14 (m, 2H) 3.33 (s, 1H) 3.35-3.44 (m, 2H) 3.55 (d, J=6.27 Hz, 6H) 3.81 (br. s., 1H) 4.01-4.20 (m, 1H) 5.08 (br. s., 1H) 5.77 (d, J=9.79 Hz, 1H) 6.96-7.37 (m, 8H) 7.40-7.89 (m, 9H) 11.56-11.92 (m, 2H).

### Example 32

### {(2S,5S)-2-[5-(4′-{5-Chloro-2-[(S)-4,4-difluoro-1-((S)-2-methoxycarbonyl amino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester

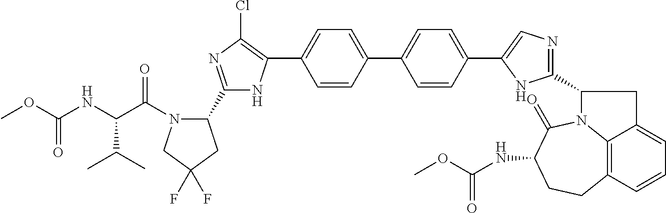

N,N-Diisopropyethylamine (1.55 g, 12.00 mmol) was added dropwise at room temperature to a heterogeneous mixture of 2-amino-1-(4-bromophenyl)ethanone hydrochloride (1.00 g, 3.99 mmol), (2S,5S)-5-(methoxycarbonylamino)-4-oxo-1,2,4,5,6,7-hexahydroazepino[3,2,1-hi]indole-2-carboxylic acid (1.21 g, 3.99 mmol), HATU (1.52 mg, 3.99 mmol) and DMF (10 ml). After the addition was complete the reaction mixture was stirred at room temperature for 4 h. The reaction mixture was diluted with ethyl acetate and washed with water, 1N hydrochloric acid, a saturated sodium bicarbonate solution, a saturated sodium chloride solution and dried over magnesium sulfate, filtered and concentrated. The crude product obtained was purified by ISCO flash chromatography (Teledyne Isco RediSep Flash Column 120 g; (0% to 100% ethyl acetate/hexane) to afford, methyl (2S,5S)-2-(2-(4-bromophenyl)-2-oxoethylcarbamoyl)-4-oxo-1,2,4,5,6,7-hexahydroazepino[3,2,1-hi]indol-5-ylcarbamate as a white solid, (1.25 g, 63%): ESI-LRMS m/e calcd for C₂₃H₂₂BrN₃O₅ [M⁺]500, found 501 [M+H⁺].

In a sealed tube 1,1′-bis(diphenylphosphino)ferrocene-palladium(II)dichloride (77 mg, 105 μmol) was added to a mixture of (S)-tert-butyl 4,4-difluoro-2-(5-(4-(4,4,5,5-tetramethyl-1,3,2-dioxaborolan-2-yl)phenyl)-1H-imidazol-2-yl)pyrrolidine-1-carboxylate (500 mg, 1.05 mmol), methyl (2S,5S)-2-(2-(4-bromophenyl)-2-oxoethylcarbamoyl)-4-oxo-1,2,4,5,6,7-hexahydroazepino[3,2,1-hi]indol-5-ylcarbamate (526 mg, 1.05 mmol) and sodium bicarbonate (265 mg, 3.16 mmol) in 1,2-dimethoxyethane (6 ml) and water (1 ml). The reaction mixture was flushed with nitrogen, capped and heated in an oil bath (80° C.) for 16 h. The reaction mixture was concentrated and partitioned between 20% methanol/methylene chloride and water and the aqueous phase extracted with 20% methanol/methylene chloride. The combined organic phases were washed with a saturated sodium chloride solution and dried over magnesium sulfate, filtered and concentrated. The crude product obtained was purified by ISCO flash chromatography (Teledyne Isco RediSep Flash Column 40 g; (0% to 100% ethyl acetate/hexane) to afford, (S)-tert-butyl 4,4-difluoro-2-(5-(4′-(2-42S,5S)-5-(methoxycarbonylamino)-4-oxo-1,2,4,5,6,7-hexahydroazepino[3,2,1-hi]indole-2-carboxamido)acetyl)biphenyl-4-yl)-1H-imidazol-2-yl)pyrrolidine-1-carboxylate as an orange solid, (250 mg, 31%): ESI-LRMS m/e calcd for C₄₁H₄₂F₂N₆O₇ [M⁺] 768, found 769 [M+H⁺].

To a solution of (S)-tert-butyl 4,4-difluoro-2-(5-(4′-(2-((2S,5S)-5-(methoxy-carbonylamino)-4-oxo-1,2,4,5,6,7-hexahydroazepino[3,2,1-hi]indole-2-carboxamido)acetyl)biphenyl-4-yl)-1H-imidazol-2-yl)pyrrolidine-1-carboxylate (250 mg, 325 mmol) in DMF (5 ml) was added N-chlorosuccinimide (52.0 mg, 390 μmol). The reaction mixture was heated at 50° C. for 16 h. After the reaction was complete by TLC it was cooled to room temperature. The crude mixture was diluted with ethyl acetate and washed with water and a saturated sodium chloride solution and dried over magnesium sulfate, filtered and concentrated. The crude product obtained was purified by ISCO flash chromatography (Teledyne Isco RediSep Flash Column 40 g; (50% to 100% ethyl acetate/hexane) to afford, (S)-tert-butyl 2-(4-chloro-5-(4′-(2-((2S,5S)-5-(methoxycarbonylamino)-4-oxo-1,2,4,5,6,7-hexahydroazepino[3,2,1-hi]indole-2-carboxamido)acetyl)biphenyl-4-yl)-1H-imidazol-2-yl)-4,4-difluoropyrrolidine-1-carboxylate as a white solid, (100 mg, 38%): ESI-LRMS m/e calcd for C₄₁H₄₁ClF₂N₆O₇ [M⁺] 803, found 804 [M+H⁺].

A mixture of (S)-tert-butyl 2-(4-chloro-5-(4′-(2-((2S,5S)-5-(methoxycarbonylamino)-4-oxo-1,2,4,5,6,7-hexahydroazepino[3,2,1-hi]indole-2-carboxamido)acetyl)biphenyl-4-yl)-1H-imidazol-2-yl)-4,4-difluoropyrrolidine-1-carboxylate (100 mg, 124 μmol) and ammonium acetate (48 mg, 622 mol) in xylenes (10 ml) was heated in a sealed tube at 140° C. for 4 h. The reaction mixture was diluted with ethyl acetate and washed with a saturated sodium bicarbonate solution, a saturated sodium chloride solution and dried over magnesium sulfate, filtered and concentrated. The crude product obtained was purified by ISCO flash chromatography (Teledyne Isco RediSep Flash Column 40 g; (30% to 100% ethyl acetate/hexane) to afford, (S)-tert-butyl 2-(4-chloro-5-(4′-(2-((2S,5S)-5-(methoxycarbonylamino)-4-oxo-1,2,4,5,6,7-hexahydroazepino[3,2,1-hi]indol-2-yl)-1H-imidazol-5-yl)biphenyl-4-yl)-1H-imidazol-2-yl)-4,4-difluoropyrrolidine-1-carboxylate as a light brown oil, (31 mg, 32%): ESI-LRMS m/e calcd for C₄₁H₄₀ClF₂N₇O₅ [M⁺] 784, found 785 [M+H⁺].

A mixture of (S)-tert-butyl 2-(4-chloro-5-(4′-(2-((2S,5S)-5-(methoxycarbonylamino)-4-oxo-1,2,4,5,6,7-hexahydroazepino[3,2,1-hi]indol-2-yl)-1H-imidazol-5-yl)biphenyl-4-yl)-1H-imidazol-2-yl)-4,4-difluoropyrrolidine-1-carboxylate (30 mg, 38 μmol) and 4.0M HCl in dioxane solution (10 ml) in methanol (10 ml) was stirred at room temperature for 4 h. Concentrate the reaction in vacuo to afford, methyl (2S,5S)-2-(5-(4′-(4-chloro-2-((S)-4,4-difluoropyrrolidin-2-yl)-1H-imidazol-5-yl)biphenyl-4-yl)-1H-imidazol-2-yl)-4-oxo-1,2,4,5,6,7-hexahydroazepino[3,2,1-hi]indol-5-ylcarbamate hydrochloride as a yellow solid, (25 mg, 91%): ESI-LRMS m/e calcd for C₃₆H₃₂ClF₂N₇O₃ HCl [M⁺] 684, found 685 [M+H⁺] (free base).

N,N-Diisopropyethylamine (13.5 mg, 104 mol) was added dropwise at room temperature to a heterogeneous mixture of methyl (2S,5S)-2-(5-(4′-(4-chloro-2-((S)-4,4-difluoropyrrolidin-2-yl)-1H-imidazol-5-yl)biphenyl-4-yl)-1H-imidazol-2-yl)-4-oxo-1,2,4,5,6,7-hexahydroazepino[3,2,1-hi]indol-5-ylcarbamate hydrochloride (25 mg, 35 mop, (S)-2-(methoxycarbonylamino)-3-methylbutanoic acid (6 mg, 35 mol), HATU (13 mg, 35 mol) and DMF (5 ml). After the addition was complete the reaction mixture was stirred at room temperature for 4 h. The reaction mixture was diluted with ethyl acetate and washed with water, 1N hydrochloric acid, a saturated sodium bicarbonate solution, a saturated sodium chloride solution and dried over magnesium sulfate, filtered and concentrated. The crude product obtained was purified by reverse phase HPLC using a 50 g Polaris C18A column eluting with acetonitrile/water (30% to 100%) to afford, {(2S,5S)-2-[5-(4′-{5-Chloro-2-[(S)-4,4-difluoro-1-((S)-2-methoxycarbonyl amino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester as a white solid, (12 mg, 41%): ESI-LRMS m/e calcd for C₄₃H₄₃ClF₂N₈O₆ [M⁺]841, found 842 [M+H⁺]; ¹H NMR (300 MHz, DMSO-d₆) δ ppm 1.21-1.32 (m, 2H) 1.59-1.70 (m, 1H) 1.80-2.38 (m, 8H) 2.82-2.94 (m, 1H) 3.03-3.13 (m, 2H) 3.34-3.37 (m, 1H) 3.40-3.44 (m, 1H) 3.56 (s, 6H) 3.60-3.69 (m, 1H) 4.06-4.21 (m, 1H) 4.39-4.52 (m, 2H) 5.77-5.83 (m, 1H) 6.96-7.23 (m, 4H) 7.43-7.57 (m, 2H) 7.60-7.83 (m, 8H) 11.84 (br. s., 1 H) 12.22 (br. s., 1H).

### Example 33

### {(2S,5S)-2-[5-(4′-{2-[(28,48)-4-Hydroxy-1-((8)-2-methoxycarbonylamino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester

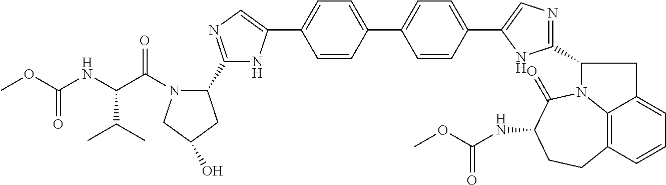

N,N-Diisopropyethylamine (1.55 g, 12.00 mmol) was added dropwise at room temperature to a heterogeneous mixture of 2-amino-1-(4-bromophenyl)ethanone hydrochloride (1.00 g, 3.99 mmol), (2S,5S)-5-(methoxycarbonylamino)-4-oxo-1,2,4,5,6,7-hexahydroazepino[3,2,1-hi]indole-2-carboxylic acid (1.21 g, 3.99 mmol), HATU (1.52 g, 3.99 mmol) and DMF (10 ml). After the addition was complete the reaction mixture was stirred at room temperature for 4 h. The reaction mixture was diluted with ethyl acetate and washed with water, 1N hydrochloric acid, a saturated sodium bicarbonate solution, a saturated sodium chloride solution and dried over magnesium sulfate, filtered and concentrated. The crude product obtained was purified by ISCO flash chromatography (Teledyne Isco RediSep Flash Column 120 g (0% to 100%) to afford, methyl (2S,5S)-2-(2-(4-bromophenyl)-2-oxoethylcarbamoyl)-4-oxo-1,2,4,5,6,7-hexahydroazepino[3,2,1-hi]indol-5-ylcarbamate as a white solid, (1.25 g, 63%): ESI-LRMS m/e calcd for C₂₃H₂₂BrN₃O₅ [M⁺]500, found 501 [M+H⁺].

A mixture of methyl (2S,5S)-2-(2-(4-bromophenyl)-2-oxoethylcarbamoyl)-4-oxo-1,2,4,5,6,7-hexahydroazepino[3,2,1-hi]indol-5-ylcarbamate (1.25 g, 2.50 mmol) and ammonium acetate (963 mg, 12.50 mmol) in xylenes (10 ml) was heated in a sealed tube at 140° C. for 4 h. The reaction mixture was diluted with ethyl acetate and washed with a saturated sodium bicarbonate solution, a saturated sodium chloride solution and dried over magnesium sulfate, filtered and concentrated. The crude product obtained was purified by ISCO flash chromatography (Teledyne Isco RediSep Flash Column 40 g; (0% to 100% ethyl acetate/hexane) to afford, methyl (2S,5S)-2-(5-(4-bromophenyl)-1H-imidazol-2-yl)-4-oxo-1,2,4,5,6,7-hexahydroazepino[3,2,1-hi]indol-5-ylcarbamate as a brown solid, (800 mg, 67%): ESI-LRMS m/e calcd for C₂₃H₂₁BrN₄O₃ [M⁺] 481, found 482 [M+H⁺].

In a sealed tube 1,1′-bis(diphenylphosphino)ferrocene-palladium(II)dichloride (76 mg, 104 μmol) was added to a mixture of tert-butyl 2-oxo-2-(4-(4,4,5,5-tetramethyl-1,3,2-dioxaborolan-2-yl)phenyl)ethylcarbamate (375 mg, 1.04 mmol), methyl (2S,5S)-2-(5-(4-bromophenyl)-1H-imidazol-2-yl)-4-oxo-1,2,4,5,6,7-hexahydroazepino[3,2,1-hi]indol-5-ylcarbamate (500 mg, 1.04 mmol) and sodium bicarbonate (262 mg, 3.12 mmol) in 1,2-dimethoxyethane (6 ml) and water (1 ml). The reaction mixture was flushed with nitrogen, capped and heated in an oil bath (80° C.) for 16 h. The reaction mixture was concentrated and partitioned between 20% methanol/methylene chloride and water and the aqueous phase extracted with 20% methanol/methylene chloride. The combined organic phases were washed with a saturated sodium chloride solution and dried over magnesium sulfate, filtered and concentrated. The crude product obtained was purified by ISCO flash chromatography (Teledyne Isco RediSep Flash Column 40 g; (0% to 100% ethyl acetate/hexane) to afford, ((2S,5S)-2-{5-[4′-(2-tert-butoxycarbonylamino-acetyl)-biphenyl-4-yl]-1H-imidazol-2-yl}-4-oxo-1,2,4,5,6,7-hexahydro-azepino[3,2,1-hi]indol-5-yl)-carbamic acid methyl ester as an off-white solid, (120 mg, 18%): ESI-LRMS m/e calcd for C₃₆H₃₇N₅O₆ [M⁺] 635, found 636 [M+H⁺].

A mixture of ((2S,5S)-2-{5-[4′-(2-tert-butoxycarbonylamino-acetyl)-biphenyl-4-yl]-1H-imidazol-2-yl}-4-oxo-1,2,4,5,6,7-hexahydro-azepino[3,2,1-hi]indol-5-yl)-carb amic acid methyl ester (120 mg, 189 μmol) and 4.0M HCl in dioxane solution (10 ml) in methanol (10 ml) was stirred at room temperature for 3 h. Concentrate the reaction in vacuo to afford, methyl (2S,5S)-2-(5-(4′-(2-amino acetyl)biphenyl-4-yl)-1H-imidazol-2-yl)-4-oxo-1,2,4,5,6,7-hexahydroazepino[3,2,1-hi]indol-5-ylcarbamate hydrochloride as a yellow solid, (106 mg, 99%): ESI-LRMS m/e calcd for C₃₁H₂₉N₅O₄ HCl [M⁺] 535, found 536 [M+H⁺] (free base).

N,N-Diisopropyethylamine (85 mg, 656 mop was added dropwise at room temperature to a heterogeneous mixture of, methyl (2S,5S)-2-(5-(4′-(2-aminoacetyl)biphenyl-4-yl)-1H-imidazol-2-yl)-4-oxo-1,2,4,5,6,7-hexahydroazepino[3,2,1-hi]indol-5-ylcarbamate hydrochloride (125 mg, 219 mol), (2S,4S)-1-(tert-butoxycarbonyl)-4-hydroxypyrrolidine-2-carboxylic acid (51 mg, 219 mol), HATU (83 mg, 219 mol) and DMF (10 ml). After the addition was complete the reaction mixture was stirred at room temperature for 4 h. The reaction mixture was diluted with ethyl acetate and washed with water, 1N hydrochloric acid, a saturated sodium bicarbonate solution, a saturated sodium chloride solution and dried over magnesium sulfate, filtered and concentrated. The crude product obtained was purified by ISCO flash chromatography (Teledyne Isco RediSep Flash Column 40 g (0% to 100%) to afford, (2S,4S)-tert-butyl 4-hydroxy-2-(2-(4′-(2-((2S,5S)-5-(methoxycarbonylamino)-4-oxo-1,2,4,5,6,7-hexahydroazepino[3,2,1-hi]indol-2-yl)-1H-imidazol-5-yl)biphenyl-4-yl)-2-oxoethylcarbamoyl)pyrrolidine-1-carboxylate as a yellow solid, (100 mg, 61%): ESI-LRMS m/e calcd for C₄₁H₄₄N₆O₈ [M⁺] 748, found 749 [M+H⁺].

A mixture of (2S,4S)-tert-butyl 4-hydroxy-2-(2-(4′-(2-((2S,5S)-5-(methoxycarbonylamino)-4-oxo-1,2,4,5,6,7-hexahydroazepino[3,2,1-hi]indol-2-yl)-1H-imidazol-5-yl)biphenyl-4-yl)-2-oxoethylcarbamoyl)pyrrolidine-1-carboxylate (100 mg, 134 mol) and ammonium acetate (52 mg, 668 mol) in xylenes (10 ml) was heated in a sealed tube at 140° C. for 4 h. The reaction mixture was diluted with ethyl acetate and washed with a saturated sodium bicarbonate solution, a saturated sodium chloride solution and dried over magnesium sulfate, filtered and concentrated. The crude product obtained was purified by ISCO flash chromatography (Teledyne Isco RediSep Flash Column 40 g; (0% to 100% ethyl acetate/hexane) to afford, (2S,4S)-tert-butyl 4-hydroxy-2-(5-(4′-(2-((2S,5S)-5-(methoxycarbonylamino)-4-oxo-1,2,4,5,6,7-hexahydroazepino[3,2,1-hi]indol-2-yl)-1H-imidazol-5-yl)biphenyl-4-yl)-1H-imidazol-2-yl)pyrrolidine-1-carboxylate as a brown solid, (60 mg, 62%): ESI-LRMS m/e calcd for C₄₁H₄₃N₇O₆ [M⁺] 729, found 730 [M+H⁺].

A mixture of(2S,4S)-tert-butyl 4-hydroxy-2-(5-(4′-(2-((2S,5S)-5-(methoxycarbonylamino)-4-oxo-1,2,4,5,6,7-hexahydroazepino[3,2,1-hi]indol-2-yl)-1H-imidazol-5-yl)biphenyl-4-yl)-1H-imidazol-2-yl)pyrrolidine-1-carboxylate (60 mg, 82 μmol) and 4.0M HCl in dioxane solution (10 ml) in methanol (10 ml) was stirred at room temperature for 3 h. Concentrate the reaction in vacuo to afford, methyl (2S,5S)-2-(5-(4′-(2-((2S,4S)-4-hydroxypyrrolidin-2-yl)-1H-imidazol-5-yl)biphenyl-4-yl)-1H-imidazol-2-yl)-4-oxo-1,2,4,5,6,7-hexahydroazepino[3,2,1-hi]indol-5-ylcarbamate hydrochloride as a brown solid, (53 mg, 99%): ESI-LRMS m/e calcd for C₃₆H₃₅N₇O₄ HCl [M⁺] 629, found 630 [M+H⁺] (free base).

N,N-Diisopropyethylamine (36 mg, 279 mop was added dropwise at room temperature to a heterogeneous mixture of methyl (2S,5S)-2-(5-(4′-(2-((2S,4S)-4-hydroxypyrrolidin-2-yl)-1H-imidazol-5-yl)biphenyl-4-yl)-1H-imidazol-2-yl)-4-oxo-1,2,4,5,6,7-hexahydroazepino[3,2,1-hi]indol-5-ylcarbamate hydrochloride (60 mg, 90 mop, (S)-2-(methoxycarbonylamino)-3-methylbutanoic acid (16 mg, 90 mop, HATU (34 mg, 90 mol) and DMF (10 ml). After the addition was complete the reaction mixture was stirred at room temperature for 3 h. The reaction mixture was diluted with ethyl acetate and washed with water, 1N hydrochloric acid, a saturated sodium bicarbonate solution, a saturated sodium chloride solution and dried over magnesium sulfate, filtered and concentrated. The crude product obtained was purified by reverse phase HPLC using a 50 g Polaris C18A column eluting with acetonitrile/water (30% to 100%) to afford, {(2S,5S)-2-[5-(4′-{2-[(2S,4S)-4-Hydroxy-1-((S)-2-methoxycarbonylamino-3-methyl-butyryl)-pyrrolidin-2-yl]-3H-imidazol-4-yl}biphenyl-4-yl)-1H-imidazol-2-yl]-4-oxo-1,2,4,5,6,7-hexahydroazepino[3,2,1hi]indol-5-yl}-carbamic acid methyl ester as a light yellow solid, (8 mg, 11%): ESI-LRMS m/e calcd for C₄₃H₄₆N₈O₇ [M⁺] 786, found 787 [M+H⁺]; ¹H NMR (300 MHz, DMSO-d₆) δ ppm 1.21-1.32 (m, 2H) 1.59-1.70 (m, 1H) 1.80-2.38 (m, 9H) 2.82-2.94 (m, 1 H) 3.03-3.13 (m, 2H) 3.34-3.37 (m, 1H) 3.40-3.44 (m, 1H) 3.56 (s, 6H) 3.60-3.69 (m, 1H) 4.06-4.21 (m, 1H) 4.39-4.52 (m, 2H) 5.77-5.83 (m, 1H) 6.96-7.23 (m, 3H) 7.43-7.57 (m, 3H) 7.60-7.83 (m, 9H) 8.90 (s, 1H) 11.84 (br. s., 1H) 12.22 (br. s., 1H).

### Example 34

### ((2S,5S)-2-{5-[4-(5-{2-[(S)-1-((8)-2-Methoxycarbonylamino-3-methyl-butyryl)-pyrrolidin-2-yl]-3H-imidazol-4-yl}-pyridin-2-yl)-phenyl]-1H-imidazol-2-yl}-4-oxo-1,2,4,5,6,7-hexahydro-azepino[3,2,1-hi]indol-5-yl)-carbamic acid methyl ester

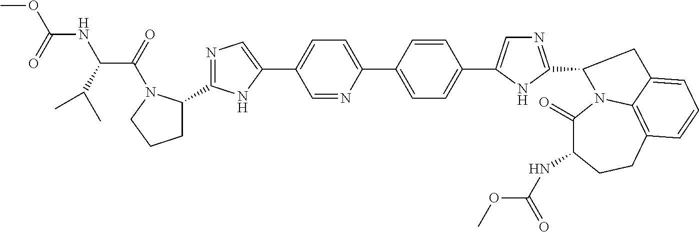

A suspension of 6-chloronicotinic acid (1.58 g, 10.0 mmol) in methylene chloride (50 ml) and DMF (4 drops) was treated with oxalyl chloride (1.53 g, 12.0 mmol). Stir the reaction mixture for 2.5 h and add trimethylsilyldiazomethane (25 ml, 2.0M solution, 50.1 mmol). Stir the reaction for 2 h and pass HCl gas through the mixture for 10 min. The reaction mixture was washed with a saturated sodium bicarbonate solution, a saturated sodium chloride solution and dried over magnesium sulfate, filtered and concentrated to afford, 2-chloro-1-(6-chloropyridin-3-yl)ethanone as a light brown solid, (1.85 g, 97%): ESI-LRMS m/e calcd for C₇H₅Cl₂NO [M⁺] 190, found 191 [M+H']

To a stirred solution of 2-chloro-1-(6-chloropyridin-3-yl)ethanone (1.85 g, 9.74 mmol) in toluene (30 ml) was added hexamethylenetetramine (1.36 g, 9.74 mmol). The mixture was stirred at 40° C. for 16 h. The resulting solid was filtered and washed with toluene and ether to afford a brown solid. The solid was added to ethanol (40 ml) and concentrated HCl (15 ml) and the mixture was stirred at room temperature for 20 h. Concentrate the mixture to afford, 2-amino-1-(6-chloropyridin-3-yl)ethanone hydrochloride as an orange solid (2.00 g, 99%): ESI-LRMS m/e calcd for C₇H₇ClN₂O HCl [M⁺] 170, found 171 [M+H⁺] (free base)

N,N-Diisopropyethylamine (3.87 mg, 29.90 mmol) was added dropwise at room temperature to a heterogeneous mixture of 2-amino-1-(6-chloropyridin-3-yl)ethanone hydrochloride (2.00 g, 9.66 mmol), (S)-1-(tert-butoxycarbonyl)pyrrolidine-2-carboxylic acid (2.08 g, 9.66 mmol), HATU (3.67 g, 9.66 mmol) and DMF (20 ml). After the addition was complete the reaction mixture was stirred at room temperature for 2 h. The reaction mixture was diluted with ethyl acetate and washed with water, a saturated sodium bicarbonate solution, a saturated sodium chloride solution and dried over magnesium sulfate, filtered and concentrated. The crude product obtained was purified by ISCO flash chromatography (Teledyne Isco RediSep Flash Column 40 g (0% to 100%) to afford, (S)-tert-butyl 2-(2-(6-chloropyridin-3-yl)-2-oxoethylcarbamoyl)pyrrolidine-1-carboxylate as an orange solid, (1.00 g, 28%): ESI-LRMS m/e calcd for C₁₇H₂₂ClN₃O₄ [M⁺] 367, found 368 [M+H⁺].

A mixture of (S)-tert-butyl 2-(2-(6-chloropyridin-3-yl)-2-oxoethylcarbamoyl)pyrrolidine-1-carboxylate (1.00 g, 2.72 mmol) and ammonium acetate (1.05 g, 13.60 mmol) in xylenes (20 ml) was heated in a sealed tube at 140° C. for 4 h. The reaction mixture was diluted with ethyl acetate and washed with a saturated sodium bicarbonate solution, a saturated sodium chloride solution and dried over magnesium sulfate, filtered and concentrated to afford, (S)-tert-butyl 2-(5-(6-chloropyridin-3-yl)-1H-imidazol-2-yl)pyrrolidine-1-carboxylate as a red oil, (600 mg, 63%): ESI-LRMS m/e calcd for C₁₇H₂₁ClN₄O₂ [M⁺] 348, found 349 [M+H⁺].

A mixture of (S)-tert-butyl 2-(5-(6-chloropyridin-3-yl)-1H-imidazol-2-yl)pyrrolidine-1-carboxylate (600 mg, 1.72 mmol) and 4.0M HCl in dioxane solution (15 ml) in methanol (15 ml) was stirred at room temperature for 3 h. Concentrate the reaction in vacuo to afford, (S)-2-chloro-5-(2-(pyrrolidin-2-yl)-1H-imidazol-5-yl)pyridine hydrochloride as an orange solid, (480 mg, 98%): ESI-LRMS m/e calcd for C₁₂H₁₃ClN₄ HCl [M⁺] 248, found 249 [M+H⁺] (free base).

N,N-Diisopropyethylamine (653 mg, 5.05 mmol) was added dropwise at room temperature to a heterogeneous mixture of (S)-2-chloro-5-(2-(pyrrolidin-2-yl)-1H-imidazol-5-yl)pyridine hydrochloride (480 mg, 1.68 mmol), (S)-2-(methoxycarbonylamino)-3-methylbutanoic acid (295 mg, 1.68 mmol), HATU (640 mg, 1.68 mmol) and DMF (10 ml). After the addition was complete the reaction mixture was stirred at room temperature for 5 h. The reaction mixture was diluted with ethyl acetate and washed with water and a saturated sodium chloride solution and dried over magnesium sulfate, filtered and concentrated. The crude product obtained was purified by ISCO flash chromatography (Teledyne Isco RediSep Flash Column 40 g (30% to 100% ethyl acetate/hexane) to afford, methyl (S)-1-((S)-2-(5-(6-chloropyridin-3-yl)-1H-imidazol-2-yl)pyrrolidin-1-yl)-3-methyl-1-oxobutan-2-ylcarbamate as an orange solid, (240 mg, 35%): ESI-LRMS m/e calcd for C₁₉H₂₄ClN₅O₃ [M⁺]405, found 406 [M+H⁺].

In a sealed tube tetrakis(triphenylphosphine)palladium (0) (68 mg, 59 μmol) was added to a mixture of tert-butyl 2-oxo-2-(4-(4,4,5,5-tetramethyl-1,3,2-dioxaborolan-2-yl)phenyl)ethylcarbamate (214 mg, 591 mol), methyl (S)-1-((S)-2-(5-(6-chloropyridin-3-yl)-1H-imidazol-2-yl)pyrrolidin-1-yl)-3-methyl-1-oxobutan-2-ylcarbamate (240 mg, 591 mol) and cesium carbonate (385 mg, 1.18 mmol) in 1,4-dioxane (6 ml) and water (1 ml). The reaction mixture was flushed with nitrogen, capped and heated in an oil bath (80° C.) for 16 h. The reaction mixture was concentrated and partitioned between 20% methanol/methylene chloride and water and the aqueous phase extracted with 20% methanol/methylene chloride. The combined organic phases were washed with a saturated sodium chloride solution and dried over magnesium sulfate, filtered and concentrated. The crude product obtained was purified by ISCO flash chromatography (Teledyne Isco RediSep Flash Column 40 g; (0% to 10% methanol/methylene chloride) to afford, {(S)-1-[(S)-2-(5-{6-[4-(2-tert-Butoxycarbonylamino-acetyl)-phenyl]-pyridin-3-yl}-1H-imidazol-2-yl)-pyrrolidine-1-carbonyl]-2-methyl-propyl}-carbamic acid methyl ester as an orange solid, (161 mg, 45%): ESI-LRMS m/e calcd for C₃₂H₄₀N₆O₆ [M⁺]604, found 605 [M+H⁺].

A mixture of {(S)-1-[(S)-2-(5-{6-[4-(2-tert-Butoxycarbonylamino-acetyl)-phenyl]-pyridin-3-yl}-1H-imidazol-2-yl)-pyrrolidine-1-carbonyl]-2-methyl-propyl}-carbamic acid methyl ester (160 mg, 265 mol) and 4.0M HCl in dioxane solution (10 ml) in methanol (10 ml) was stirred at room temperature for 3 h. Concentrate the reaction in vacuo to afford, methyl (S)-1-((S)-2-(5-(6-(4-(2-amino ac etyl)phenyl)pyridin-3-yl)-1H-imidazol-2-yl)pyrrolidin-1-yl)-3-methyl-1-oxobutan-2-ylcarbamate hydrochloride as a yellow solid, (146 mg, 99%): ESI-LRMS m/e calcd for C₂₇H₃₃N₆O₄₆ HCl [M⁺]504, found 505 [M+H⁺] (free base).

N,N-Diisopropyethylamine (140 mg, 1.08 mmol) was added dropwise at room temperature to a heterogeneous mixture of methyl (S)-1-((S)-2-(5-(6-(4-(2-amino acetyl)phenyl)pyridin-3-yl)-1H-imidazol-2-yl)pyrrolidin-1-yl)-3-methyl-1-oxobutan-2-ylcarbamate hydrochloride (195 mg, 360 mol), (2S,5S)-5-(methoxycarbonylamino)-4-oxo-1,2,4,5,6,7-hexahydroazepino[3,2,1-hi]indole-2-carboxylic acid (110 mg, 360 μmol), HATU (137 mg, 360 μmol) and DMF (10 ml). After the addition was complete the reaction mixture was stirred at room temperature for 4 h. The reaction mixture was diluted with ethyl acetate and washed with water, a saturated sodium bicarbonate solution and a saturated sodium chloride solution and dried over magnesium sulfate, filtered and concentrated. The crude product obtained was purified by ISCO flash chromatography (Teledyne Isco RediSep Flash Column 40 g (0% to 10% methanol/methylene chloride) to afford, ((2S,5S)-2-{2-[4-(5-{2-[(S)-1-((S)-2-methoxycarbonylamino-3-methyl-butyryl)-pyrrolidin-2-yl]-3H-imidazol-4-yl}-pyridin-2-yl)-phenyl]-2-oxo-ethylcarbamoyl}-4-oxo-1,2,4,5,6,7-hexahydro-azepino[3,3,1-hi]indol-5-yl)-carbamic acid methyl ester as a brown oil, (218 mg, 77%): ESI-LRMS m/e calcd for C₄₂H₄₆N₈O₈ [M⁺] 790, found 791 [M+H⁺].

A mixture of ((2S,5S)-2-{2-[4-(5-{2-[(S)-1-((S)-2-methoxycarbonylamino-3-methyl-butyryl)-pyrrolidin-2-yl]-3H-imidazol-4-yl}-pyridin-2-yl)-phenyl]-2-oxo-ethylcarbamoyl}-4-oxo-1,2,4,5,6,7-hexahydro-azepino[3,3,1-hi]indol-5-yl)-carbamic acid methyl ester (218 mg, 276 μmol) and ammonium acetate (106 mg, 1.38 mmol) in xylenes (10 ml) was heated in a sealed tube at 140° C. for 4 h. The reaction mixture was diluted with ethyl acetate and washed with a saturated sodium bicarbonate solution, a saturated sodium chloride solution and dried over magnesium sulfate, filtered and concentrated. The crude product obtained was purified by reverse phase HPLC using a 50 g Polaris C18A column eluting with acetonitrile/water (30% to 100%) to afford, ((2S,5S)-2-{5-[4-(5-{2-[(S)-1-((S)-2-Methoxycarbonylamino-3-methyl-butyryl)-pyrrolidin-2-yl]-3H-imidazol-4-yl}-pyridin-2-yl)-phenyl]-1H-imidazol-2-yl}-4-oxo-1,2,4,5,6,7-hexahydro-azepino[3,2,1-hi]indol-5-yl)-carbamic acid methyl ester as a white solid, (10 mg, 5%): ESI-LRMS m/e calcd for C₄₂H₄₅N₉O₆ [M⁺] 771, found 772 [M+H⁺]; ¹H NMR (300 MHz, DMSO-d₆) δ 0.72-1.00 (m, 6H) 1.80-2.35 (m, 6H) 2.99-3.14 (m, 2H) 3.33 (s, 1 H) 3.35-3.44 (m, 2H) 3.55 (d, J=6.27 Hz, 6H) 3.81 (br. s., 1H) 4.01-4.20 (m, 1H) 5.08 (br. s., 1H) 5.77 (d, J=9.79 Hz, 1H) 6.96-7.37 (m, 4H) 6.97-7.05 (m, 1H) 7.21-7.36 (m, 1H) 7.40-7.89 (m, 10H) 11.56-11.92 (m, 2H).

### Example 35

### {(2S,5S)-2-[5-(2-Fluoro-4′-{2-[(S)-1-((S)-2-methoxycarbonylamino-3-methyl-butyryl)-pyrrolidin-2-yl]-1H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester

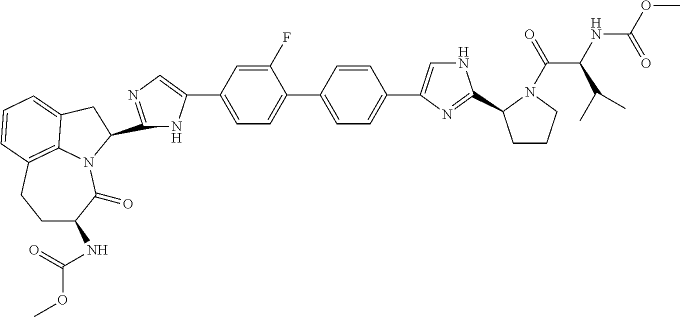

4-Bromo-3-fluorobenzoic acid (2.46 g, 11.2 mmol) was dissolved in MeOH (9 ml) and toluene (4 ml) and then was cooled in an ice-water bath to 0° C. (Trimethylsilyl)-diazamethane (11 ml, 2 M in hexane, 22 mmol) was added dropwise to the reaction mixture at 0° C. The reaction mixture was raised to room temperature and stirred for 40 min at RT and was concentrated in vacuo. The resulting residue was added toluene and further concentrated under high vacuum to obtain 2.5 g (95% yield) of Methyl 4-bromo-3-fluorobenzoate as white solid. ¹H NMR (DMSO-d₆) δ: 7.75-7.83 (m, 1H), 7.72 (d, J=9.0 Hz, 1H), 7.61 (d, J=8.3 Hz, 1H), 3.20 (s, 1H).

To a solution of Methyl 4-bromo-3-fluorobenzoate (1 g, 4.29 mmol) and chloroiodomethane (1.87 ml, 25.75 mmol) in THF(dry, 45 ml) at −78° C. in acetone-dry-ice bath under N₂ atmosphere, LDA (2 M in THF/Heptane/EtPh, 25.74 ml, 51.48 mmol) was added dropwise. The reaction mixture was stirred for 2 hours at −78° C. and then was quenched by dropwise addition of AcOH/THF (10 ml, 1/1 v/v) at −78° C. The resulting mixture was warmed to room temperature and stirred for 20 min at RT. Then the reaction mixture was partitioned in EtOAC and saturated NaHCO₃ to make the aqueous phase neutral. The organic phase was washed with water and dried over Na₂SO₄. The residue was purified by flash silica gel column separation (EtOAc/Hexane 0 to 5% gradient, then 5%) and was further purified by C-18 HPLC(MeCN/Water 10-100%) to obtain 304 mg (28% yield) of 1-(4-bromo-3-fluoro-phenyl)-2-chloro-ethanone as off-white waxy solid. ¹H NMR(CHLOROFORM-d) δ: 7.50-7.87 (m, 3H), 4.33 (s, 2H).

To the 15 ml 20% Piperidine/DMF solution(piperidine 3 ml, 30.4 mmol; DMF 12 ml) was added (2S,5S)-5-(((9H-fluoren-9-yl)methoxy)carbonylamino)-4-oxo-1,2,4,5,6,7-hexahydroazepino[3,2,1-hi]indole-2-carboxylic acid (1500 mg, 3.20 mmol). The resulting solution was stirred at room temperature for 1 hr. White precipitation was generated during this time period. The reaction mixture was filtered and the resulting solid was washed with hexane (3×) and diethyl ether (3×). The solid was further dried under high vacuum to obtain 650 mg (82% yield) of (2S,5S)-5-Amino-4-oxo-1,2,4,5,6,7-hexahydro-azepino[3,2,1-hi]indole-2-carboxylic acid as white solid. ESI-LCMS m/e calcd. for C₁₃H₁₄N₂O₃ 246 [M⁺], found 247 [M+H⁺].

Sodium carbonate (168 mg, 1.58 mol) was added to a mixture of (2S,5S)-5-amino-4-oxo-1,2,4,5,6,7-hexahydroazepino[3,2,1-hi]indole-2-carboxylic acid (650 mg, 2.64 mol) in NaOH aq. solution (0.6 M aq. solution, 4.4 ml, 2.64 mol). The mixture was cooled with ice-water bath. Then Methyl chloroformate (223 ul, 2.90 mol) was added drop-wise to the above mixture. The ice-water bath was removed and the reaction mixture was stirred at room temperature for 3 hours. The reaction mixture was washed with diethyl ether (3×). The resulting aqueous phase was then acidified to pH of 1-2 by addition of con. HCl. The above acidic aqueous phase was extracted with dichloromethane (3×) and the combined organic phase was concentrated in vacuo to obtain 614 mg (76% yield) of (2S,5S)-5-(methoxycarbonylamino)-4-oxo-1,2,4,5,6,7-hexahydroazepino[3,2,1-hi]indole-2-carboxylic acid as white solid. ESI-LCMS m/e calcd. for C₁₅H₁₆N₂O₅ 304 [M⁺], found 305 [M+H⁺].

To a mixture of 1-(4-bromo-3-fluorophenyl)-2-chloroethanone (147 mg, 0.58 mmol) and (2S,5S)-5-(methoxycarbonylamino)-4-oxo-1,2,4,5,6,7-hexahydroazepino[3,2,1-hi]indole-2-carboxylic acid, 196 mg, 0.64 mol) and KI (124 mg, 0.75 mol) in Acetonitrile (10 ml), N,N′-diisopropylethylamine (131 μl, 0.75 mol) was added. The reaction mixture was then heated to 50° C. in an oil bath for 3 hours. The reaction mixture was evaporated under vacuum. The residue was partitioned with dichloromethane and water. The aqueous phase was extracted by methylenechloride (2×). The combined organic phase was evaporated under vacuum and the residue was purified by flash silica gel column separation(EtOAc/hexane 0 to 40% gradient, then 40%) to provide 189 mg(63% yield) of (2S,5S)-5-Methoxycarbonylamino-4-oxo-1,2,4,5,6,7-hexahydro-azepino[3,2,1-hi]indole-2-carboxylic acid 2-(4-bromo-3-fluoro-phenyl)-2-oxo-ethylester as solid. ESI-LCMS m/e calcd. for C₂₃H₂₀BrFN₂O₆ 518.05 [M⁺], found 519 [M+H⁺].

A stream of nitrogen was bubbled through the mixture of (2S,5S)-2-(4-bromo-3-fluorophenyl)-2-oxoethyl 5-(methoxycarbonylamino)-4-oxo-1,2,4,5,6,7-hexahydroazepino[3,2,1-hi]indole-2-carboxylate (139 mg, 0.268 mol) in Xylene (4 ml) in a sealable reaction tube for 5 min. NH₄OAc (413 mg, 5.35 mol) was then added to the reaction mixture. The reaction tube was sealed and stirred in a 140° C. oil bath for 3 h. The crude mixture was partitioned between DICHLOROMETHANE and 1M Na₂CO₃ to make the aqueous phase slightly basic (pH=8). The aqueous phase was extracted by DICHLOROMETHANE twice. The combined DICHLOROMETHANE phases were evaporated. The residue was purified by flash silica gel column separation (EtOAc/Hexane 0 to 60% gradient, then 60%) to provide 112 mg (83% yield) of {(2S,5S)-2-[5-(4-Bromo-3-fluoro-phenyl)-1H-imidazol-2-yl]-4-oxo-1,2,4,5,6,7-hexahydro-azepino[3,2,1-hi]indol-5-yl}-carbamic acid methyl ester as white sticky solid. ESI-LCMS m/e calcd. for C₂₃H₂₀BrFN₄O₃ 498.07 [M⁺], found 498.9 [M+H⁺]

To a mixture of methyl (2S,5S)-2-(5-(4-bromo-3-fluorophenyl)-1H-imidazol-2-yl)-4-oxo-1,2,4,5,6,7-hexahydroazepino[3,2,1-hi]indol-5-ylcarbamate (112 mg, 0.224 mol) and methyl (S)-3-methyl-1-oxo-1-((S)-2-(4-(4-(4,4,5,5-tetramethyl-1,3,2-dioxaborolan-2-yl)phenyl)-1H-imidazol-2-yl)pyrrolidin-1-yl)butan-2-ylcarbamate (122 mg, 0.248 mol) in 4 ml t-BuOH in a sealable tube, PdCl₂(dppf) (41 mg, 0.056 mol) and NaHCO₃ (56 mg, 0.672 mmol) were added. Then 0.4 ml water was added. The resulting suspension was bubbled with a stream of nitrogen for 3 min. Then the tube was sealed and heated in a 90° C. oil-bath for 2 h with stirring. The reaction mixture was cooled to room temperature and was filter through celite and washed with dichloromethane. The filtrate was evaporated under vacuum. The residue was partitioned between dichloromethane and water. The aqueous phase was extracted with dichloromethane (2×). The combined organic phases were evaporated. The residue was dissolved by MeCN/MeOH (1/1) and was subjected to C-18 prep-HPLC separation (MeCN/Water 10-90%). The desired fractions were extracted by dichloromethane (3×) and evaporated. The resulting solid was further purified by silica gel flash column separation (MeOH/dichloromethane 0-4% gradient, then 4%). The desired fractions were collected and concentrated under vacuum to obtain 43 mg (25% yield) {(2S,5S)-2-[5-(2-Fluoro-4′-{2-[(S)-1-((S)-2-methoxycarbonylamino-3-methyl-butyryl)-pyrrolidin-2-yl]-1H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester as white powder. ESI-LCMS m/e calcd. for C₄₃H₄₅FN₈O₆ 788.3 [M⁺], found 789.2 [M+H⁺]; ¹H NMR (DMSO-d₆) δ: 11.69-12.55 (m, 2H), 6.94-7.90 (m, 14H), 5.77 (d, J=9.0 Hz, 1H), 5.08 (br. s., 1H), 3.95-4.25 (m, 3H), 3.80 (br. s., 2H), 3.54 (d, J=4.1 Hz, 6H), 3.01-3.24 (m, 3H), 1.72-2.40 (m, 7H), 0.87 (dd, J=14.9, 6.6 Hz, 6H).

### Example 36

### {(2S,5S)-2-[5-(3-Fluoro-4′-{2-[(S)-1-((S)-2-methoxycarbonylamino-3-methyl-butyryl)-pyrrolidin-2-yl]-1H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester

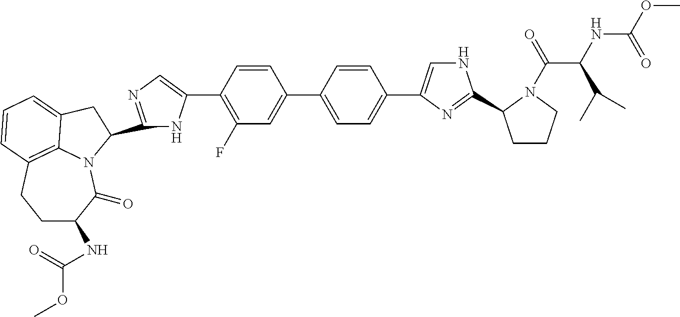

To a mixture of 2-bromo-1-(4-bromo-2-fluorophenyl)ethanone (purchased from BetaPharma, 254 mg, 0.86 mmol) and (2S,5S)-5-(methoxycarbonylamino)-4-oxo-1,2,4,5,6,7-hexahydroazepino[3,2,1-hi]indole-2-carboxylic acid (289 mg, 0.95 mmol) in acetonitrile (10 ml), diisopropylethylamine(195 μl, 1.12 mmol) was added. The reaction mixture was then stirred at RT for 4 h. The reaction mixture was evaporated under vacuum. The residue was partitioned with dichloromethane and water. The aqueous phase was extracted by dichloromethane (2×). The combined organic phase was evaporated under vacuum and the residue was purified by flash silica gel column separation (EtOAc/Hexane 0-to 30% gradient, then 30%) to provide 405 mg (87% yield) of (2S,5S)-5-methoxycarbonylamino-4-oxo-1,2,4,5,6,7-hexahydro-azepino[3,2,1-hi]indole-2-carboxylic acid 2-(4-bromo-2-fluoro-phenyl)-2-oxo-ethylester as solid.

A stream of nitrogen was bubbled through the mixture of ((2S,5S)-2-(4-bromo-2-fluorophenyl)-2-oxoethyl 5-(methoxycarbonylamino)-4-oxo-1,2,4,5,6,7-hexahydroazepino[3,2,1-hi]indole-2-carboxylate (355 mg, 0.684 mmol) in Xylene (7 ml) in a sealable reaction tube for 5 min. NH₄OAc (1.05 g, 13.7 mmol) was then added to the reaction mixture. The reaction tube was sealed and stirred in a 140° C. oil bath for 3 h. The crude mixture was partitioned between dichloromethane and 1M Na₂CO₃ to make the aqueous phase slightly basic (pH=8). The aqueous phase was extracted by dichloromethane twice. The combined organic phases were evaporated. The residue was purified by flash silica gel column (EtOAc/Hexane 0 to 40% gradient, then 40%) to provide 274 mg (80% yield) of {(2S,5S)-2-[5-(4-Bromo-2-fluoro-phenyl)-1H-imidazol-2-yl]-4-oxo-1,2,4,5,6,7-hexahydro azepino[3,2,1-hi]indol-5-yl}-carbamic acid methyl ester as white solid. ESI-LCMS m/e calcd. for C₂₃H₂₀BrFN₄O₃ 498.07 [M⁺], found 498.9 [M+H⁺].

To a mixture of Methyl (2S,5S)-2-(5-(4-bromo-2-fluorophenyl)-1H-imidazol-2-yl)-4-oxo-1,2,4,5,6,7-hexahydroazepino[3,2,1-hi]indol-5-ylcarbamate (120 mg, 0.24 mmol) and Methyl (S)-3-methyl-1-oxo-1-((S)-2-(4-(4-(4,4,5,5-tetramethyl-1,3,2-dioxaborolan-2-yl)phenyl)-1H-imidazol-2-yl)pyrrolidin-1-yl)butan-2-ylcarbamate (155 mg, 0.31 mmol) in 4 ml t-butanol in a sealable tube., PdCl₂(dppf) (44 mg, 0.06 mmol) and NaHCO₃ (60 mg, 0.72 mmol) were added. Then 0.4 ml water was added. The resulting suspension was bubbled with a stream of nitrogen for 3 min. Then, the tube was sealed and heated in a 90° C. oil-bath for 3 h with stirring. The reaction mixture was cooled to room temperature and was filter through celite and was washed with dichloromethane. The filtrate was evaporated under vacuum. The residue was partitioned between dichloromethane and water. The aqueous phase was extracted with dichloromethane (2×). The combined organic phases were evaporated. The residue was dissolved by MeCN/MeOH(1/1) and was subjected to C-18 HPLC separation(MeCN/Water 10-90% gradient) and was further purified by silica gel flash column separation(MeOH/dichloromethane 0-4% gradient, then 4%). The fractions were checked with both TLC and LC/MS. The desired fractions were collected and concentrated under vacuum to obtain 84 mg (44% yield) of {(2S,5S)-2-[5-(3-Fluoro-4′-{2-[(S)-1-((S)-2-methoxycarbonylamino-3-methyl-butyryl)-pyrrolidin-2-yl]-1H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester as white powder. ESI-LCMS m/e calcd. for C₄₃H₄₅FN₈O₆ 788.3 [M⁺], found 789.3 [M+H⁺]; ¹H NMR (DMSO-d₆) δ ppm 11.74-12.45 (m, 2H), 6.98-8.07 (m, 14H), 5.72-5.93 (m, 1H), 5.10 (br. s., 1H), 3.99-4.29 (m, 2H), 3.83 (br. s., 2H), 3.57 (d, J=4.7 Hz, 6H), 3.44 (br. s., 2H), 3.11 (br. s., 2H), 1.84-2.37 (m, 7H), 0.90 (dd, J=15.6, 6.4 Hz, 6H).

### Example 37

### {(2S,5S)-2-[5-(2′-Fluoro-4′-{2-[(S)-1-((S)-2-methoxycarbonylamino-3-methyl-butyryl)-pyrrolidin-2-yl]-1H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester

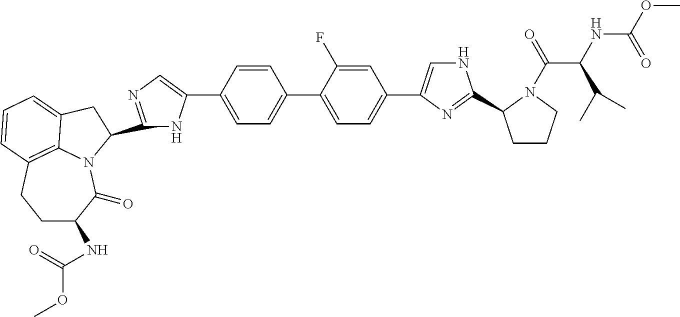

To the mixture of 2-bromo-1-(4-bromo-2-fluorophenyl)ethanone (1 g, 3.98 mmol) and (S)-1-((S)-2-(methoxycarbonylamino)-3-methylbutanoyl)pyrrolidine-2-carboxylic acid (1.19 g, 4.38 mmol) and KI (858 mg, 5.17 mmol) in acetonitrile (40 ml), diisopropylethylamine (901 μl, 5.17 mmol) was added. The reaction mixture was then heated to 50° C. in an oil bath for 3 hours. The reaction mixture was evaporated under vacuum. The residue was partitioned with dichloromethane and water. The aqueous phase was extracted by dichloromethane (2×). The combined organic phase was evaporated under vacuum and the residue was purified by flash silica gel column separation (EtOAc/Hexane 0-to 35% gradient, then 35%) to provide 319 mg (16% yield) of (S)-1-((S)-2-Methoxycarbonylamino-3-methyl-butyryl)-pyrrolidine-2-carboxylic acid 2-(4-bromo-3-fluoro-phenyl)-2-oxo-ethyl ester as sticky solid. ESI-LCMS m/e calcd. for C₂₀H₂₄BrFN₂O₆ 486.1 [M⁺], found 487.8 [M+H⁺].

A stream of nitrogen was bubbled through the mixture of (S)-2-(4-bromo-3-fluorophenyl)-2-oxoethyl 145)-2-(methoxycarbonylamino)-3-methylbutanoyl)-pyrrolidine-2-carboxylate (319 mg, 0.0654 mmol) in xylene (6 ml) in a sealable reaction tube for 5 min. NH₄OAc (1009 mg, 13.09 mmol) was then added to the reaction mixture. The reaction tube was sealed and stirred in a 140° C. oil bath for 3 h. The crude mixture was partitioned between dichloromethane and 1M Na₂CO₃ to make the aqueous phase slightly basic (pH=8). The aqueous phase was extracted with dichloromethane, twice. The combined organic phases were evaporated. The residue was purified by flash silica gel column separation (EtOAc/Hexane 0-70% gradient, then 70%) to provide 152 mg(49% yield) of ((S)-1-{(S)-2-[5-(4-bromo-3-fluoro-phenyl)-1H-imidazol-2-yl]-pyrrolidine-1-carbonyl}-2-methyl-propyl)-carbamic acid methyl ester as off-white solid. ESI-LCMS m/e calcd. for C₂₀H₂₄BrFN₄O₃ 466.1 [M⁺], found 466.9 [M+H⁺].

To a mixture of 2-Bromo-1-(4-bromo-2-fluorophenyl)ethanone (792 mg, 2.85 mmol) and (2S,5S)-5-(methoxycarbonylamino)-4-oxo-1,2,4,5,6,7-hexahydroazepino[3,2,1-hi]indole-2-carboxylic acid (954 mg, 3.13 mmol) in acetonitrile (20 ml), diisopropylethylamine (645 μA, 3.71 mmol) was added. The reaction mixture was then stirred at RT for 4 h. The reaction mixture was evaporated under vacuum. The residue was partitioned with dichlormethane and water. The aqueous phase was extracted by methylenechloride (2×). The combined organic phase was evaporated under vacuum and the residue was purified by flash silica gel column separation (EtOAc/Hexane 0-to 40% gradient, then 40%) to provide 1.285 g (89% yield) of (2S,5S)-5-Methoxycarbonylamino-4-oxo-1,2,4,5,6,7-hexahydro-azepino[3,2,1-hi]indole-2-carboxylic acid 2-(4-bromo-phenyl)-2-oxo-ethyl ester as white powder. ESI-LCMS m/e calcd. for C₂₃H₂₁BrN₄O₃ 500 [M⁺], found 501 [M+H⁺].

A stream of nitrogen was bubbled through the mixture of (2S,5S)-2-(4-bromophenyl)-2-oxoethyl 5-(methoxycarbonylamino)-4-oxo-1,2,4,5,6,7-hexahydroazepino[3,2,1-hi]indole-2-carboxylate (1285 mg, 0.080 mmol) in Xylene (10 ml) in a sealable reaction tube for 5 min. NH₄OAc (3.95 g, 51.3 mmol) was then added to the reaction mixture. The reaction tube was sealed and stirred in a 140° C. oil bath for 3 h. The crude mixture was partitioned between methylene chloride and 1M Na₂CO₃ to make the aqueous phase slightly basic (pH=8). The aqueous phase was extracted by dichloromethane, twice. The combined organic phases were evaporated. The residue was purified by flash silica gel column separation (EtOAc/Hexane 0-40% gradient, then 40%) to provide 679 mg (55% yield) of {(2S,5S)-2-[5-(4-Bromo-phenyl)-1H-imidazol-2-yl]-4-oxo-1,2,4,5,6,7-hexahydro-azepino[3,2,1-hi]indol-5-yl}-carbamic acid methyl ester as white solid. ESI-LCMS m/e calcd. for C₂₃H₂₁BrN₄O₃ 480 [M⁺], found 481 [M+H⁺].

In a pressure tube, potassium acetate (408 mg, 4.16 mmol), methyl (2S,5S)-2-(5-(4-bromophenyl)-1H-imidazol-2-yl)-4-oxo-1,2,4,5,6,7-hexahydroazepino[3,2,1-hi]indol-5-ylcarbamate (400 mg, 0.83 1 mmol) and (Bis(pinacolato)diboron, 633 mg, 2.49 mmol) were combined with 1,4-dioxane (anhydrous, 6 ml). The reaction mixture was bubbled with nitrogen stream for 4 min. Then PdCl₂(dppf) (61 mg, 0.083 mmol) was added to the reaction mixture. The reaction mixture was bubbled with nitrogen stream for 4 min before the sealed tube was capped. The sealed tube was then heated with an oil bath at 110° C. for overnight. The reaction mixture was filtered through Celite and washed with dichloromethane. The filtrate was partitioned in dichloromethane and an aqueous solution of saturated NaHCO₃ solution/water (⅕). The resulting aqueous phase was extracted twice with dichloromethane The combined dichlormethane phases were concentrated in vacuo. The residue was purified by flash silica gel flash column separation (EtOAc/Hexane 0 to 60% gradient, then 60%) to obtain 425 mg(97% yield) of as white solid. ESI-LCMS m/e calcd. for C₂₉H₃₃BN₄O₅ 528.2 [M⁺], found 529.1 [M+H⁺].

To a mixture of methyl (S)-1-((S)-2-(5-(4-bromo-3-fluorophenyl)-1H-imidazol-2-yl)pyrrolidin-1-yl)-3-methyl-1-oxobutan-2-ylcarbamate (60 mg, 0.128 mmol) and Methyl (2S,5S)-4-oxo-2-(5-(4-(4,4,5,5-tetramethyl-1,3,2-dioxaborolan-2-yl)phenyl)-1H-imidazol-2-yl)-1,2,4,5,6,7-hexahydroazepino[3,2,1-hi]indol-5-ylcarbamate (75 mg, 0.141 mmol) in a sealable tube, K₃PO₄ (218 mg, 1.024 mmol, Pd(OAc)₂ (7.2 mg, 0.032 mmol) and RuPhos (30 mg, 0.064 mmol) were added. Then, n-BuOH/H₂O(6 ml, v/v:3/1) was added. The resulting suspension was bubbled with a stream of nitrogen for 4 min. Then the tube was sealed and heated in a 90° C. oil-bath for 3 h with stirring. The reaction mixture was cooled to room temperature and was filtered through Celite and washed with dichloromethane. The filtrate was evaporated under vacuum. The residue was partitioned between dichloromethane and water. The aqueous phase was extracted with methylenechloride (2×). The combined methylenechloride phases were evaporated. The residue was subjected to flash silica gel column separation (MeOH/dichloromethane 0 to 4% gradient, then 4% to obtain 31 mg (31% yield) of {(2S,5S)-2-[5-(2′-Fluoro-4′-{2-[(S)-1-((S)-2-methoxycarbonylamino-3-methyl-butyryl)-pyrrolidin-2-yl]-1H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester as solid. ESI-LCMS m/e calcd. for C₄₃H₄₅FN₈O₆ 788.3 [M⁺], found 789.3 [M+H⁺]; ¹H NMR (DMSO-d₆) δ ppm 5.69-5.83 (m, 1H), 5.07 (d, J=3.8 Hz, 1H), 3.97-4.25 (m, 2H), 3.80 (br. s., 2H), 3.54 (d, J=4.7 Hz, 6H), 3.39 (d, J=11.5 Hz, 2H), 3.08 (br. s., 2H), 1.82-2.34 (m, 7H), 0.88 (dd, J=16.4, 6.6 Hz, 6H).

### Example 38

### {(2S,5S)-2-[5-(2′-Cyano-4′-{2-[(S)-1-((8)-2-methoxycarbonylamino-3-methyl-butyryl)-pyrrolidin-2-yl]-1H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester

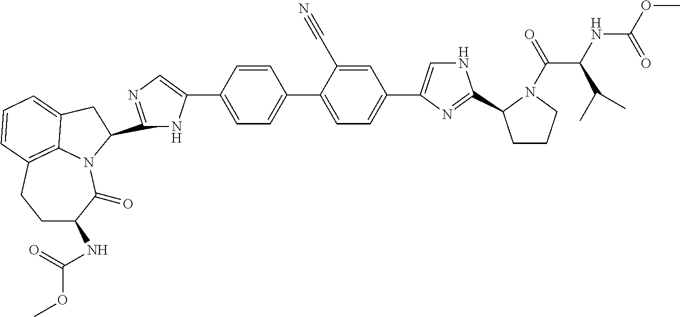

To a solution of N-Boc-L-proline (10 g, 50.2 mmol) in MeOH (100 ml), glyoxal (40% w/w in water, 25 g, 170.6 mmol) and NH₄OH (5N aq. solution 191 ml, 954 mmol) were added. The reaction mixture was stirred at RT overnight. The reaction mixture was partitioned between dichloromethane and water. The aqueous phase was extracted twice more with dichloromethane. The combined organic phases were evaporated under vacuum and the residue was purified by silica gel flash column (MeOH/dichloromethane 0 to 3% gradient, then 3%) to afford 9.87 g (82% yield) of (S)-2-(1H-Imidazol-2-yl)-pyrrolidine-1-carboxylic acid tert-butyl ester as off-white solid.

ESI-LCMS m/e calcd. for C₁₂H₁₉N₃O₂ 237.1 [M⁺], found 237.9 [M+H⁺].

To the solution of (S)-tert-butyl 2-(1H-imidazol-2-yl)pyrrolidine-1-carboxylate (9 g, 37.9 mmol) in methylenechloride (250 ml) at 0° C. (ice-water bath) was added NIS (17.9 g, 79.6 mmol) slowly. The reaction mixture was stirred at 0° C. (ice-water bath) for 1 h. Then the reaction mixture was partitioned with water and more dichloromethane. The organic phase was washed with water twice and then was evaporated. The residue was purified by silica gel flash column separation (EtOAc/Hexane 0 to 25%, then 25%) to obtain 13.9 g (75% yield) of (S)-2-(4,5-Diiodo-1H-imidazol-2-yl)-pyrrolidine-1-carboxylic acid tert-butyl ester as yellow solid. ESI-LCMS m/e calcd. for C₁₂H₁₇H₂N₃O₂ 489 [M⁺], found 490 [M+H⁺].

To the suspension of (S)-tert-butyl 2-(4,5-diiodo-1H-imidazol-2-yl)pyrrolidine-1-carboxylate (7 g, 14.3 mmol) in 310 ml EtOH/H₂O (3/7 v/v) was added Na₂SO₃ (15.3 g, 12.2 mmol). The reaction mixture was refluxed (with oil bath of 110° C.) for 24 hours. EtOH was evaporated under vacuum. Then the reaction mixture was partitioned between water and dichloromethane. The organic phase was washed with Brine and concentrated under vacuum. The residue was purified by silica gel flash column separation (EtOAc/hexane 0-25% gradient, then 25%) to obtain 4.4 g (84% yield) of (S)-2-(4-Iodo-1H-imidazol-2-yl)-pyrrolidine-1-carboxylic acid tert-butyl ester as white solid. ESI-LCMS m/e calcd. for C₁₂H₁₈IN₃O₂ 363 [M⁺], found 364 [M+H⁺].

To the solution of (S)-tert-butyl 2-(4-iodo-1H-imidazol-2-yl)pyrrolidine-1-carboxylate (286 mg, 0.787 mmol) in methylenechloride (6 ml), HCl (4M in dioxane, 3.94 ml, 15.75 mmol) was added. The reaction mixture was stirred at RT for 1 h. The mixture was evaporated under vacuum. Toluene was added to the residue and was evaporated under high vacuum to obtain (S)-4-iodo-2-(pyrrolidin-2-yl)-1H-imidazole hydrochloride as solid which was used directly for next reaction. ESI-LCMS m/e calcd. for C₇H₁₀IN₃ 263 [M⁺], found 264 [M+H⁺].

To the mixture of (S)-4-iodo-2-(pyrrolidin-2-yl)-1H-imidazole hydrochloride (0.787 mmol, crude from last step), (S)-2-(methoxycarbonylamino)-3-methylbutanoic acid (165 mg, 0.944 mmol), HOBt (149 mg, 1.102 mmol), EDCI (211 mg, 1.102 mmol) in DMF (anhydrous, 4 ml) was added N,N′-diisopropylethylamine (823 ul, 4.722 mmol). The reaction mixture was stirred at room temperature for overnight. The solvent was evaporated under high vacuum. The resulting residue was partitioned with water and dichloromethane. The aq phase was extracted by dichloromethane, twice. The combined organic phases were evaporated under vacuum. The resulting residue was purified by flash silica gel column separation (EtOAc/Hexane 0 to 70% gradient, then 70%) to obtain 215 mg (65% yield) of {(S)-1-[(S)-2-(5-Iodo-1H-imidazol-2-yl)-pyrrolidine-1-carbonyl]-2-methyl-propyl}-carbamic acid methyl ester as off-white solid. ESI-LCMS m/e calcd. for C₂₁H₂₄ClN₅O₃ 420.1 [M⁺], found 421.0 [M+H⁺].

Methyl (S)-1-((S)-2-(5-iodo-1H-imidazol-2-yl)pyrrolidin-1-yl)-3-methyl-1-oxobutan-2-ylcarbamate (215 mg, 0.512 mmol), 4-Chloro-3-cyanophenylboronic acid (purchased from Combiblock, 102 mg, 0.563 mmol), PdCl₂(DPPF) (94 mg, 0.128 mmol) and NaHCO₃ (129 mg, 1.536 mmol) were mixed in a sealable tube. Then 4 ml t-BuOH/H₂O(v/v 10:1)) was added to the mixture. The resulting suspension was bubbled with a stream of nitrogen for 3 min. Then the tube was sealed and heated in a 90° C. oil-bath for 4 h with stirring. The reaction mixture was cooled to room temperature and was filtered through Celite and washed with dichloromethane. The filtrate was evaporated under vacuum. The residue was partitioned between dichloromethane and water. The aqueous phase was extracted with dichloromethane (2×). The combined organic phases were evaporated. The residue was purified by flash silica gel column separation (EtOAc/Hexane 0-70% gradient, then 70%) to obtain 89 mg(40% yield) of ((S)-1-{(S)-2-[5-(4-Chloro-3-cyano-phenyl)-1H-imidazol-2-yl]-pyrrolidine-1-carbonyl}-2-methyl-propyl)-carbamic acid methyl ester as light brown powder. ESI-LCMS m/e calcd. for C₂₁H₂₄ClN₅O₃ 429.2 [M⁺], found 430.0 [M+H⁺].

To the mixture of Methyl (S)-1-((S)-2-(5-(4-chloro-3-cyanophenyl)-1H-imidazol-2-yl)pyrrolidin-1-yl)-3-methyl-1-oxobutan-2-ylcarbamate (80 mg, 0.186 mmol) and Methyl (2S,5S)-4-oxo-2-(5-(4-(4,4,5,5-tetramethyl-1,3,2-dioxaborolan-2-yl)phenyl)-1H-imidazol-2-yl)-1,2,4,5,6,7-hexahydroazepino[3,2,1-hi]indol-5-ylcarbamate (108 mg, 0.205 mmol) in a sealable tube, K₃PO₄ (316 mg, 1.49 mmol), Pd(OAc)₂ (10.4 mg, 0.0465 mmol) and Ru-Phos (43 mg, 0.093 mmol) were added. Then, nBuOH/H₂O(6 ml, v/v:3/1) was added. The resulting suspension was bubbled with a stream of nitrogen for 4 min. The tube was sealed and heated in a 90° C. oil-bath for 3 h with stirring. The reaction mixture was cooled to room temperature and was filtered through Celite and washed with dichloromethane. The filtrate was evaporated under vacuum. The residue was partitioned between dichloromethane and water. The aqueous phase was extracted with methylenechloride (2×). The combined organic phases were evaporated. The residue was purified by flash silica gel column separation (MeOH/dichloromethane 0 to 5% gradient, then 5%) and then further purified by C-18 prep HPLC separation (MeCN/Water 10 to 80% gradient). The desired product fractions were combined, extracted with dichloromethane (3×). The combined organic phases were evaporated under vacuum to provide 36 mg(24% yield) of {(2S,5S)-2-[5-(2′-Cyano-4′-{2-[(S)-1-((S)-2-methoxycarbonylamino-3-methyl-butyryl)-pyrrolidin-2-yl]-1H-imidazol-4-yl}-biphenyl-4-yl)-1Himidazol-2-yl]-4-oxo-1,2,4,5,6,7-hexahydro-azepino[3,2,1-hi]indol-5-yl}-carbamic acid methyl ester as off-white powder. ESI-LCMS m/e calcd. for C₄₄H₄₅N₉O₆ 795.3 [M⁺], found 796.2 [M+H⁺]; ¹H NMR (DMSO-d₆) δ: 11.78-12.50 (m, 2H), 6.95-8.33 (m, 14H), 5.71-5.84 (m, 1H), 5.08 (br. s., 1H), 3.98-4.23 (m, 2H), 3.81 (br. s., 2H), 3.55 (d, J=4.7 Hz, 6H), 3.17 (d, J=5.3 Hz, 2H), 3.09 (br. s., 2H), 1.72-2.38 (m, 7H), 0.89 (dd, J=17.1, 6.6 Hz, 6H).

### Example 39

### {(2S,5S)-2-[5-(4′-{2-[(2S,4R)-4-Cyano-1-((S)-2-methoxycarbonylamino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester

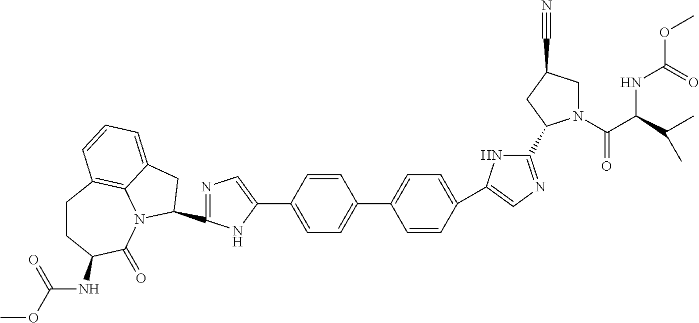

To the mixture of 2-bromo-1-(4-bromo-2-fluorophenyl)ethanone (2.1 g, 7.56 mmol), (2S,4R)-1-(tert-butoxycarbonyl)-4-cyanopyrrolidine-2-carboxylic acid (2 g, 8.32 mmol) in acetonitrile (38 ml), N,N′-diisopropylethylamine (1.71 ml, 9.83 mmol) was added. The reaction mixture was then stirred at RT for 3 h. The reaction mixture was evaporated under vacuum. The residue was partitioned with dichloromethane and water. The aqueous phase was extracted by dichloromethane (2×). The combined organic phase was evaporated under vacuum and the residue was purified by flash silica gel column separation (EtOAc/Hexane 0 to 30% gradient, then 30%) to provide 2.67 g (81% yield) of (2S,4R)-4-cyano-pyrrolidine-1,2-dicarboxylic acid 2-[2-(4-bromo-phenyl)-2-oxo-ethyl]ester 1-tert-butyl ester as sticky white solid. ESI-LCMS m/e calcd. for C₁₉H₂₁BrN₂O₅ 436.1 [M⁺], found 336.9 [M-100+H⁺].

A stream of nitrogen was bubbled through the mixture of (2S,4R)-2-(2-(4-bromophenyl)-2-oxoethyl) 1-tert-butyl 4-cyanopyrrolidine-1,2-dicarboxylate (1 g, 2.287 mmol) in Xylene (10 ml) in a sealable reaction tube for 5 min. NH₄OAc (3.53 g, 45.74 mmol) was then added to the reaction mixture. The reaction tube was sealed and stirred in the 140° C. oil bath for 3 h. The crude mixture was partitioned between dichloromethane and 1M Na₂CO₃ to make the aqueous phase neutral. The aqueous phase was extracted by dichloromethane, twice. The combined organic phases were evaporated. The residue was purified by flash silica gel column separation (EtOAc/Hexane 0 to 40% gradient, then 40%) to obtain 704 mg (74% yield) of (2S,4R)-2-[5-(4-Bromo-phenyl)-1H-imidazol-2-yl]-4-cyano-pyrrolidine-1-carboxylic acid tert-butyl ester as white solid. ESI-LCMS m/e calcd. for C₁₉H₂₁BrN₄O₂ 416.1 [M⁺], found 417.9 [M+H⁺].

To the solution of (2S,4R)-tert-butyl 2-(5-(4-bromophenyl)-1H-imidazol-2-yl)-4-cyanopyrrolidine-1-carboxylate (400 mg, 0.958 mmol) in methylenechloride (10 ml), HCl (4M in dioxane, 4.79 ml, 19.17 mmol) was added. The reaction mixture was stirred at RT for overnight. The mixture was evaporated under vacuum. Toluene was added to the residue and was evaporated under high vacuum. The resulting (3R,5S)-5-(5-(4-bromophenyl)-1H-imidazol-2-yl)pyrrolidine-3-carbonitrile hydrochloride as solid was used directly for next reaction. ESI-LCMS m/e calcd. for C₁₄H₁₃BrN₄ 316.0 [M⁺], found 316.9 [M+H⁺].

To the mixture of (3R,5S)-5-(5-(4-bromophenyl)-1H-imidazol-2-yl)pyrrolidine-3-carbonitrile hydrochloride (0.958 mmol, crude from last step), (S)-2-(methoxycarbonylamino)-3-methylbutanoic acid (201 mg, 1.15 mmol), HOBt (1181 mg, 1.34 mmol) and EDCI (256 mg, 1.34 mmol) in DMF(anhydrous, 5 ml) was added N,N′-diisopropylethylamine (1 ml, 5.748 mmol). The reaction mixture was stirred at room temperature for overnight. The solvent was evaporated under high vacuum.

The resulting residue was partitioned with water and dichloromethane. The aqueous phase was extracted by dichloromethane, twice. The combined organic phases were evaporated under vacuum. The resulting residue was purified by flash silica gel column separation (EtOAc/Hexane 0-70% gradient, then 70%) to obtain 384 mg (84% yield) of ((S)-1-{(2S,4R)-2-[5-(4-Bromo-phenyl)-1H-imidazol-2-yl]-4-cyano-pyrrolidine-1-carbonyl}-2-methyl-propyl)-carbamic acid methyl ester as the sticky oil. ESI-LCMS m/e calcd. for C₂₁H₂₄BrN₅O₃ 473.1 [M⁺], found 473.9 [M+H⁺].

To the mixture of Methyl (S)-1-((2S,4R)-2-(5-(4-bromophenyl)-1H-imidazol-2-yl)-4-cyanopyrrolidin-1-yl)-3-methyl-1-oxobutan-2-ylcarbamate, 82 mg, 0.172 mmol) and Methyl (2S,5S)-4-oxo-2-(5-(4-(4,4,5,5-tetramethyl-1,3,2-dioxaborolan-2-yl)phenyl)-1H-imidazol-2-yl)-1,2,4,5,6,7-hexahydroazepino[3,2,1-hi]indol-5-ylcarbamate (100 mg, 0.189 mmol) in a sealable tube, K₃PO₄ (294 mg, 1.376 mmol, Pd(OAc)₂ (9.7 mg, 0.043 mmol) and RuPhos (40 mg, 0.086 mmol) were added. Then n-BuOH/H₂O (6 ml, v/v:5/1) were added. The resulting suspension was bubbled with a stream of nitrogen for 4 min. Then the tube was sealed and heated in a 90° C. oil-bath for 3 h with stirring. The reaction mixture was cooled to room temperature and was filtered through Celite and washed with dichloromethane. The filtrate was evaporated under vacuum. The residue was partitioned between dichloromethane and water. The aqueous phase was extracted with dichloromethane (2×). The combined organic phases were evaporated under vacuum. The crude mixture was dissolved in MeCN/MeOH (1/1 v/v) and subject to C-18 HPLC separation (MeCN/Water 0 to 90% gradient) and was further purified by flash silica gel column separation (EtOAc/Hexane 0-80% gradient, then EtOAc/Hexane 80%, then (5%-MeOH/dichloromethane) to obtain 5 mg (4% yield) of {(2S,5S)-2-[5-(4′-{2-[(2S,4R)-4-Cyano-1-((S)-2-methoxycarbonylamino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester as the off-white solid.

ESI-LCMS m/e calcd. for C₄₄H₄₅N₉O₆ 795.3 [M⁺], found 796.3 [M+H⁺]; ¹H NMR (DMSO-d₆) δ: 11.68-12.48 (m, 2H), 6.52-7.97 (m, 15H), 5.55-5.96 (m, 1H), 5.11-5.42 (m, 1H), 3.92-4.29 (m, 4H), 3.73-3.91 (m, 1H), 3.55 (s, 6H), 2.93-3.25 (m, 4H), 1.82-2.31 (m, 5H), 0.84 (d, J=5.5 Hz, 6H).

### Example 40

### {(2S,5S)-2-[5-(4′-{2-[(28,48)-4-Cyano-1-((8)-2-methoxycarbonylamino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester

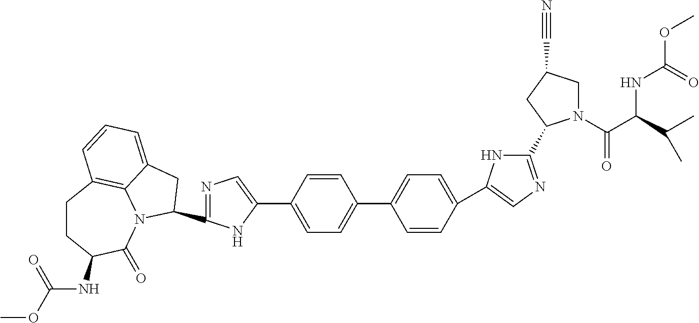

The mixture of (2S,4S)-1-tert-butyl 2-methyl 4-cyanopyrrolidine-1,2-dicarboxylate (2 g, 7.865 mmol), LiOH(188 mg, 7.865 mmol) and water (40 ml) was stirred at 40° C. in an oil bath for 3 h. The reaction solution was cooled to RT and NaHSO₄ (1 g, 8.337 mmol) was added in several portions to make pH as 2. The mixture was extracted with EtOAc three times. The combined EtOAc phases were evaporated under vacuum to provide 1.88 g (99% yield) of (2S,4S)-1-tert-Butoxycarbonyl-4-cyano-pyrrolidine-2-carboxylic acid as white solid. ES-negative-LCMS m/e calcd. for CᵢᵢH₁₅N₂O₄ 239.1 [M⁺], found 238 [M−H⁺].

To the mixture of 2-bromo-1-(4-bromo-2-fluorophenyl)ethanone, 2.1 g, 7.56 mmol), (2S,4S)-1-(tert-butoxycarbonyl)-4-cyanopyrrolidine-2-carboxylic acid (7.86 mmol, crude from last step) in acetonitrile (40 ml), N,N′-diisopropylethylamine (1.62 ml, 9.29 mmol) was added. The reaction mixture was then stirred at RT for 3 h. The reaction mixture was evaporated under vacuum. The residue was partitioned with dichloromethane and water. The aqueous phase was extracted by dichloromethane (2×). The combined organic phase was evaporated under vacuum to yield, 3.403 g crude product of (2S,4S)-4-cyano-pyrrolidine-1,2-dicarboxylic acid 24244-bromo-phenyl)-2-oxo-ethyl]ester 1-tert-butyl ester as oil was obtained and was then used for next step without further purification. ESI-LCMS m/e calcd. for C₁₉H₂₁BrN₂O₅ 436.1 [M⁺], found 337 [M-100+H⁺].

A stream of nitrogen was bubbled through the mixture of (2S,4R)-2-(2-(4-bromophenyl)-2-oxoethyl) 1-tert-butyl 4-cyanopyrrolidine-1,2-dicarboxylate (3.55 mmol, crude from last step) in Xylene (10 ml) in a sealable reaction tube for 5 min. NH₄OAc (5.47 g, 71 mmol) was then added to the reaction mixture. The reaction tube was sealed and stirred in a 140° C. oil bath for 3 h. The crude mixture was partitioned between dichloromethane and 1M Na₂CO₃ to make the aqueous phase neutral. The aqueous phase was extracted by dichloromethane, twice. The combined organic phases were evaporated. The residue was purified by flash silica gel column (EtOAc/Hexane 0 to 60% gradient, then 60%) to obtain 945 mg (64% yield) of (2S,4S)-2-[5-(4-bromo-phenyl)-1H-imidazol-2-yl]-4-cyano-pyrrolidine-1-carboxylic acid tert-butyl ester as white solid. ESI-LCMS m/e calcd. for C₁₉H₂₁BrN₄O₂ 416.1 [M⁺], found 416.9 [M+H⁺].

In a pressure tube, Potassium acetate (588 mg, 5.99 mmol), (2S,4S)-tert-butyl 24544-bromophenyl)-1H-imidazol-2-yl)-4-cyanopyrrolidine-1-carboxylate (500 mg, 1.198 mmol), Bis(pinacolato)diboron (913 mg, 3.594 mmol) were combined with 1,4-dioxane (anhydrous, 8 ml). The reaction mixture was bubbled with nitrogen stream for 4 min. Then PdCl₂(dppf) (88 mg, 0.120 mmol) was added to the reaction mixture. The reaction mixture was bubbled with nitrogen stream for 4 min. The sealed tube was capped and heated with an oil bath at 110° C. for 3 h. The reaction mixture was filtered through Celite and washed with dichloromethane. The filtrate was partitioned in dichloromethane and aqueous saturated NaHCO₃ solution/water(1:5). The resulting aqueous phase was extracted twice with DICHLOROMETHANE. The combined organic phases were concentrated in vacuo. The residue was purified by flash silica gel column separation (EtOAC/Hexane 0 to 60% gradient, then 60%) to provide 405 mg(73% yield) of (2S,4S)-4-cyano-2-{5-[4-(4,4,5,5-tetramethyl-[1,3,2]dioxaborolan-2-yl)-phenyl]-1H-imidazol-2-yl}-pyrrolidine-1-carboxylic acid tert-butyl ester. ESI-LCMS m/e calcd. for C₂₅H₃₃BN₄O₄ 464.2 [M⁺], found 465.1 [M+H⁺].

To the mixture of (Methyl (2S,5S)-2-(5-(4-bromophenyl)-1H-imidazol-2-yl)-4-oxo-1,2,4,5,6,7-hexahydroazepino[3,2,1-hi]indol-5-ylcarbamate (159 mg, 0.331 mmol) and (2S,4S)-tert-butyl 4-cyano-2-(5-(4-(4,4,5,5-tetramethyl-1,3,2-dioxaborolan-2-yl)phenyl)-1H-imidazol-2-yl)pyrrolidine-1-carboxylate (200 mg, 0.431 mmol) in a sealable tube, K₃PO₄ (562 mg, 2.648 mmol, Pd(OAc)₂ (18 mg, 0.083 mmol) and RuPhos (77 mg, 0.166 mmol) were added. Then n-BuOH/H₂O(6 ml, v/v:5/1) was added. The resulting suspension was bubbled with a stream of nitrogen for 4 min. Then the tube was sealed and heated in a 90° C. oil-bath for 3 h with stirring. The reaction mixture was cooled to room temperature and was filtered through Celite and washed with dichloromethane. The filtrate was evaporated under vacuum. The residue was partitioned between dichloromethane and water. The aqueous phase was extracted with methylenechloride (2×). The combined organic phases were evaporated under vacuum. The crude mixture was purified by flash silica gel column separation(0 to 5% MeOH/dichloromethane gradient) to obtain 120 mg (49% yield) of (2S,4S)-4-Cyano-2-(5-{4′-[2-((2S,5S)-5-methoxycarbonylamino-4-oxo-1,2,4,5,6,7-hexahydro-azepino[3,2,1-hi]indol-2-yl)-3H-imidazol-4-yl]-biphenyl-4-yl}-1H-imidazol-2-yl)-pyrrolidine-1-carboxylic acid tert-butyl ester. ESI-LCMS m/e calcd. for C₄₂H₄₂N₈O₅ 738.3 [M⁺], found 739.2 [M+H⁺].

To the solution of (2S,4S)-tert-butyl 4-cyano-2-(5-(4′-(2-((2S,5S)-5-(methoxycarbonylamino)-4-oxo-1,2,4,5,6,7-hexahydroazepino[3,2,1-hi]indol-2-yl)-1H-imidazol-5-yl)biphenyl-4-yl)-1H-imidazol-2-yl)pyrrolidine-1-carboxylate (30 mg, 0.0406 mmol) in methylenechloride (1 ml), HCl (4M in dioxane, 0.203 ml, 0.812 mmol) was added. The mixture was evaporated under vacuum. Toluene was added to the residue and was evaporated under high vacuum to obtain [(2S,5S)-2-(5-{4′-[2-((2S,4S)-4-cyano-pyrrolidin-2-yl)-3H-imidazol-4-yl]-biphenyl-4-yl}-1H-imidazol-2-yl)-4-oxo-1,2,4,5,6,7-hexahydro-azepino[3,2,1-hi]indol-5-yl]-carbamic acid methyl ester hydrochloride as solid which was used directly for next reaction. ESI-LCMS m/e calcd. for C₃₇H₃₄N₈O₃ 638.3 [M⁺], found 639.2 [M+H⁺].

To the mixture of Methyl (2S,5S)-2-(5-(4′-(2-((2S,4S)-4-cyanopyrrolidin-2-yl)-1H-imidazol-5-yl)biphenyl-4-yl)-1H-imidazol-2-yl)-4-oxo-1,2,4,5,6,7-hexahydroazepino[3,2,1-hi]indol-5-ylcarbamate hydrochloride (0.0406 mmol, crude from last step), (S)-2-(methoxycarbonylamino)-3-methylbutanoic acid, 8.5 mg, 0.0487 mmol), Propylphosphonic Anhydride solution(50% in DMF, 59 μl, 0.101 mmol) in DMF(anhydrous, 1 ml) was added N,N′-diisopropylethylamine (42 μl, 0.244 mmol). The reaction mixture was stirred at room temperature for 30 min. The resulting residue was partitioned with water and dichloromethane. The aqueous phase was extracted by dichloromethane, twice. The combined organic phases were evaporated under vacuum. The resulting residue was purified by C-18 prep-HPLC (MeCN/Water 20-75% gradient) to obtain 15 mg of {(2S,5S)-2-[5-(4′-{2-[(2S,4S)-4-cyano-1-((S)-2-methoxycarbonylamino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester as solid. ESI-LCMS m/e calcd. for C₄₄H₄₅N₉O₆ 795.3 [M⁺], found 796.3 [M+H⁺]; ¹H NMR (DMSO-d₆) δ: 11.72-12.47 (m, 2H), 6.90-7.94 (m, 15H), 5.77 (d, J=8.9 Hz, 1H), 5.02-5.26 (m, 1H), 4.46 (br. s., 1H), 3.77-4.28 (m, 5H), 3.54 (br. s., 6H), 2.63-3.20 (m, 4H), 1.74-2.44 (m, 5H), 0.82 (d, J=7.0 Hz, 5H).

### Example 41

### {(2S,5S)-2-{5-[4-(6-{2-[(2S,4S)-4-Cyano-1-((S)-2-methoxycarbonylamino-3-methyl-butyryl)-pyrrolidin-2-yl]-3H-imidazol-4-yl}-naphthalen-2-yl)-phenyl]-1H-imidazol-2-yl}-4-oxo-1,2,4,5,6,7-hexahydro-azepino[3,2,1-hi]indol-5-yl}-carbamic acid methyl ester

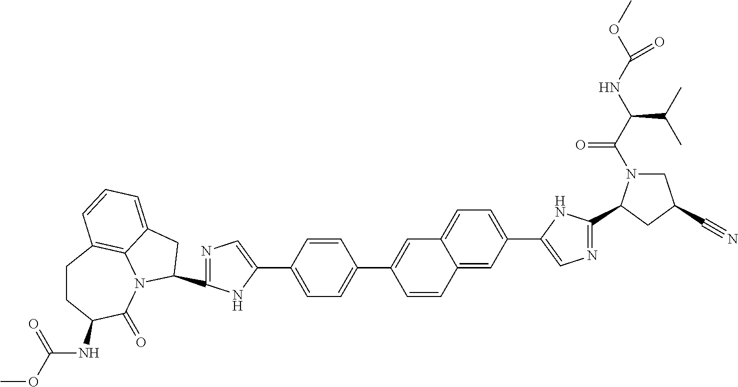

A solution of (2-Bromonaphthalene, 6.21 g, 30 mmol) in dichloromethane (100 ml), which was cooled to 0° C. with ice-water bath, was added A1Cl₃ (4.4 g, 33 mmol) and (2-chloroacetyl chloride, 2.63 ml, 33 mmol). The reaction mixture was stirred at 0° C. for 1 h. The reaction was raised to room temperature and water (50 ml) was added. The organic phase was washed with water twice and evaporated under vacuum. The obtained solid was washed with EtOAc/hexane (100 ml, v/v 1/9) and filtered to obtain 5.2 g crude product as solid. 520 ml EtOAc/hexane (9/1, v/v) was used to totally dissolve the above crude product under refluxing condition in 90° C. oil bath. The solution was cooled to room temperature in 3 hours and crystal appeared. After filtration and washing with hexane, 2.08 g (24% yield) of 1-(6-Bromo-naphthalen-2-yl)-2-chloro-ethanone was obtained. ESI-LCMS m/e calcd. for C₁₂H₈BrClO 281.9 [M⁺], found 282.8 [M-100+H⁺]; ¹H NMR(CHLOROFORM-d) d: 8.47 (s, 1H), 8.02-8.12 (m, 2H), 7.87 (d, J=8.5 Hz, 2H), 7.68 (d, J=7.5 Hz, 1H), 4.83 (s, 2H).

To the mixture of 1-(6-bromonaphthalen-2-yl)-2-chloroethanone (1 g, 3.53 mmol), and (2S,4S)-1-(tert-butoxycarbonyl)-4-cyanopyrrolidine-2-carboxylic acid (932 mg, 3.88 mmol) and KI (761 mg, 4.58 mmol) in Acetonitrile (40 ml), N,N′-diisopropylethylamine (797 μA, 4.58 mmol) was added. The reaction mixture was then heated to 50° C. in an oil bath for 3 hours. The crude mixture was cooled to room temperature and was partitioned between water and dichloromethane. The aqueous phase was extracted with dichloromethane (3×). The combined organic phases were concentrated under vacuum to obtain (2S,4S)-4-Cyano-pyrrolidine-1,2-dicarboxylic acid 2-[2-(6-bromo-naphthalen-2-yl)-2-oxo-ethyl]ester 1-tert-butyl ester as the light brown solid which was used for next step directly. ESI-LCMS m/e calcd. for C₂₃H₂₃BrN₂O₅ 486.1 [M⁺], found 387 [M-100+H⁺].

A stream of nitrogen was bubbled through the mixture of (2S,4S)-2-(2-(6-bromonaphthalen-2-yl)-2-oxoethyl) 1-tert-butyl 4-cyanopyrrolidine-1,2-dicarboxylate(crude from last step, 3.53 mmol) in Xylene (20 ml) in a sealable reaction tube for 5 min. NH₄OAc (5.43 g, 70.54 mmol) was then added to the reaction mixture. The reaction tube was sealed and stirred in a 140° C. oil bath for 3 h. The crude mixture was partitioned between dichloromethane and 1M Na₂CO₃ to make the aqueous phase neutral. The aqueous phase was extracted by dichloromethane, twice. The combined organic phases were evaporated. The residue was purified by flash silica gel column (EtOAc/Hexane 0 to 60% gradient, then 60%) to obtain 449 mg (27% yield) of (2S,4S)-2-[5-(6-Bromo-naphthalen-2-yl)-1H-imidazol-2-yl]-4-cyano-pyrrolidine-1-carboxylic acid tert-butyl ester as light brown solid. ESI-LCMS m/e calcd. for C₂₃H₂₃BN₄O₂ 466.1 [M⁺], found 467 [M+H⁺].

(2S,4S)-tert-butyl 2-(5-(6-bromonaphthalen-2-yl)-1H-imidazol-2-yl)-4-cyanopyrrolidine-1-carboxylate (195 mg, 0.418 mmol) and (methyl (2S,5S)-4-oxo-2-(5-(4-(4,4,5,5-tetramethyl-1,3,2-dioxaborolan-2-yl)phenyl)-1H-imidazol-2-yl)-1,2,4,5,6,7-hexahydroazepino[3,2,1-hi]indol-5-ylcarbamate (287 mg, 0.543 mmol) and K₃PO₄ (710 mg, 3.344 mmol) were mixed in a sealable tube. Then, n-BuOH/H₂O (6 ml, v/v:5/1) were added. The resulting suspension was bubbled with a stream of Argon for 4 min and then was vacuumed with house vacuum for 2 min. The above degassing and vacuuming process were repeated twice. Then the Pd(OAc)₂ (18 mg, 0.083 mmol) and RuPhos (77 mg, 0.166 mmol) were added. The above degassing and vacuuming process were repeated twice. Then the tube was sealed and heated in a 90° C. oil-bath for 3 h with stirring. The reaction mixture was cooled to room temperature and was filtered through Celite and washed with dichloromethane. The filtrate was evaporated under vacuum. The residue was partitioned between dichloromethane and water. The aqueous phase was extracted with methylenechloride (2×). The combined organic phases were evaporated under vacuum. The resulting mixture was purified by silica gel flash column separation (MeOH/dichloromethane 0 to 5% gradient, then 5%) and further purified by the C-18 prep-HPLC (MeCN/Water 10 to 100% gradient) to obtain 101 mg (31% yield) of (2S,4S)-4-Cyano-2-[5-(6-{4-[2-((2S,5S)-5-methoxycarbonylamino-4-oxo-1,2,4,5,6,7-hexahydro-azepino[3,2,1-hi]indol-2-yl)-3H-imidazol-4-yl]-phenyl}-naphthalen-2-yl)-1H-imidazol-2-yl]-pyrrolidine-1-carboxylic acid tert-butyl ester. ESI-LCMS m/e calcd. for C₄₆H₄₄N₈O₅ 788.3 [M⁺], found 789.4 [M+H⁺].

To the solution of 2S,4S)-tert-butyl 4-cyano-2-(5-(6-(4-(2-((2S,5S)-5-(methoxycarbonylamino)-4-oxo-1,2,4,5,6,7-hexahydroazepino[3,2,1-hi]indol-2-yl)-1H-imidazol-5-yl)phenyl)naphthalen-2-yl)-1H-imidazol-2-yl)pyrrolidine-1-carboxylate (100 mg, 0.127 mmol) in methylenechloride (2 ml), HCl (4M in dioxane, 0.634 ml, 2.54 mmol) was added. The reaction mixture was stirred at RT for 30 min. The mixture was evaporated under vacuum. Toluene was added to the residue and was evaporated under high vacuum. The resulting {(2S,5S)-2-[5-(4-{6-[2-((2S,4S)-4-Cyano-pyrrolidin-2-yl)-3H-imidazol-4-yl]-naphthalen-2-yl}-phenyl)-1H-imidazol-2-yl]-4-oxo-1,2,4,5,6,7-hexahydro-azepino[3,2,1-hi]indol-5-yl}-carbamic acid methyl ester hydrochloride as solid was used directly for next reaction. ESI-LCMS m/e calcd. for C₄₁H₃₆N₈O₃ 688.3 [M⁺], found 689.2 [M+H⁺].

To a stirred solution of (S)-2-(methoxycarbonylamino)-3-methylbutanoic acid (27 mg, 0.152 mmol) in DMF (dry, 1 ml) was added HATU (120 mg, 0.318 mmol) and N,N′-diisopropylethylamine (22 ul, 0.127 mmol). After stirring at room temperature for 30 min, a solution of Methyl (2S,5S)-2-(5-(4-(6-(2-((2S,4S)-4-cyanopyrrolidin-2-yl)-1H-imidazol-5-yl)naphthalen-2-yl)phenyl)-1H-imidazol-2-yl)-4-oxo-1,2,4,5,6,7-hexahydroazepino[3,2,1-hi]indol-5-ylcarbamate hydrochloride (0.127 mmol, crude from last step) and N,N′-diisopropylethylamine (111 μA, 0.635 mmol) in DMF(dry, 1 ml) was added. The reaction mixture was stirred at room temperature for 1 h. The reaction mixture was diluted with dichloromethane and then sat. NH₄Cl aq. solution was carefully added to the mixture with stirring to make the pH=7. The organic phase was separated and concentrated under vacuum. The resulting residue was purified by C-18 HPLC separation(MeCN/H₂O 10-100%) and then was further purified by flash silica gel column separation (MeOH/dichloromethane 0-5% gradient, then 5%) to provide 16 mg (15% yield) of {(2S,5S)-2-{5-[4-(6-{2-[(2S,4S)-4-cyano-1-((S)-2-methoxycarbonylamino-3-methyl-butyryl)-pyrrolidin-2-yl]-3H-imidazol-4-yl}-naphthalen-2-yl)-phenyl]-1H-imidazol-2-yl}-4-oxo-1,2,4,5,6,7-hexahydro-azepino[3,2,1-hi]indol-5-yl}-carbamic acid methyl ester as white solid. ESI-LCMS m/e calcd. for C₄₈H₄₇N₉O₆ 845.4 [M⁺], found 846.4 [M+H⁺]; ¹H NMR (DMSO-d₆) δ ppm 11.67-12.68 (m, 2H), 6.43-8.44 (m, 17H), 5.80 (d, J=9.3 Hz, 1H), 5.04-5.32 (m, 1H), 3.78-4.59 (m, 5H), 3.57 (br. s, 6H), 2.62-3.21 (m, 4H), 1.87-2.38 (m, 5H), 0.75-0.99 (m, 6H).

### Example 42

### {(2S,5S)-2-{5-[4-(6-{2-[(S)-4,4-Difluoro-1-((S)-2-methoxycarbonylamino-3-methyl-butyryl)-pyrrolidin-2-yl]-3H-imidazol-4-yl}-naphthalen-2-yl)-phenyl]-1H-imidazol-2-yl}-4-oxo-1,2,4,5,6,7-hexahydro-azepino[3,2,1-hi]indol-5-yl}-carbamic acid methyl ester

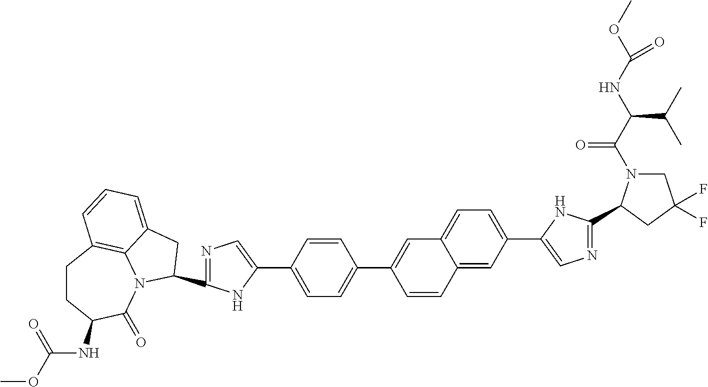

To the mixture of (1-(6-bromonaphthalen-2-yl)-2-chloroethanone (676 mg, 2.384 mmol), (S)-1-(tert-butoxycarbonyl)-4,4-difluoropyrrolidine-2-carboxylic acid, 659 mg, 2.622 mmol) and KI (514 mg, 3.099 mmol) in acetonitrile (30 ml), N,N′-diisopropylethylamine (540 μA, 3.099 mmol) was added. The reaction mixture was then heated to 50° C. in an oil bath for 3 hours. The crude was cooled to room temperature and was partitioned between water and dichloromethane. The aqueous phase was extracted with dichloromethane (3×). The combined organic phases were concentrated under vacuum to obtain (S)-4,4-Difluoro-pyrrolidine-1,2-dicarboxylic acid 24246-bromo-naphthalen-2-yl)-2-oxo-ethyl]ester 1-tert-butyl ester as the light brown colored to use for next step directly. ESI-LCMS m/e calcd. for C₂₂H₂₂BrF₂NO₅ 497.1 [M⁺], found 397.9 [M-100+H⁺].

A stream of nitrogen was bubbled through the mixture of (S)-2-(2-(6-bromonaphthalen-2-yl)-2-oxoethyl) 1-tert-butyl 4,4-difluoropyrrolidine-1,2-dicarboxylate (crude from last step, 2.384 mmol) in xylene (20 ml) in a sealable reaction tube for 5 min. NH₄OAc (3.67 g, 47.68 mmol) was then added to the reaction mixture. The reaction tube was sealed and stirred in the 140° C. oil bath for 3 h. The crude mixture was partitioned between dichloromethane and 1M Na₂CO₃ to make the aqueous phase neutral. The aqueous phase was extracted by dichloromethane, twice. The combined organic phases were evaporated. The residue was purified by flash silica gel column separation (EtOAc/Hexane 0 to 30% gradient, then 30%) to obtain 837 mg (73% yield) of (S)-2-[5-(6-bromo-naphthalen-2-yl)-1H-imidazol-2-yl]-4,4-difluoro-pyrrolidine-1-carboxylic acid tert-butyl ester as light brown solid. ESI-LCMS m/e calcd. for C₂₂H₂₂BrF₂N₃O₂ 477.1 [M⁺], found 477.9 [M+H⁺].

Two parallel reactions with the same scale were conducted as following: (S)-tert-butyl 2-(5-(6-bromonaphthalen-2-yl)-1H-imidazol-2-yl)-4,4-difluoropyrrolidine-1-carboxylate, 200 mg, 0.418 mmol), Methyl (2S,5S)-4-oxo-2-(5-(4-(4,4,5,5-tetramethyl-1,3,2-dioxaborolan-2-yl)phenyl)-1H-imidazol-2-yl)-1,2,4,5,6,7-hexahydroazepino[3,2,1-hi]indol-5-ylcarbamate (287 mg, 0.543 mmol) and K₃PO₄ (710 mg, 3.344 mmol) were mixed in a sealable tube. Then, n-BuOH/H₂O (6 ml, v/v:5/1) was added. The resulting suspension was bubbled with a stream of argon for 4 min and then, was degassed with house vacuum for 2 min. The above degassing and vacuuming process were repeated twice. Then the Pd(OAc)₂ (18 mg, 0.083 mmol) and RuPhos (77 mg, 0.166 mmol) were added. The above degassing and vacuuming process were repeated twice. Then the tube was sealed and heated in a 90° C. oil-bath for 3 h with stirring. The reaction mixture was cooled to room temperature. The crude mixture of two batches was combined, filtered through Celite, and washed with dichloromethane. The filtrate was evaporated under vacuum. The residue was partitioned between dichloromethane and water. The aqueous phase was extracted with dichloromethane (2×). The combined organic phases were evaporated under vacuum. The resulting mixture was purified by silica gel flash column separation (MeOH/dichloromethane 0 to 5% gradient, then 5%) and was further purified by the C-18 prep-HPLC(MeCN/water 10 to 100%) and 284 mg (42% yield) of (S)-4,4-difluoro-2-[5-(6-{4-[2-((2S,5S)-5-methoxycarbonylamino-4-oxo-1,2,4,5,6,7-hexahydro-azepino[3,2,1-hi]indol-2-yl)-3H-imidazol-4-yl]-phenyl}-naphthalen-2-yl)-1H-imidazol-2-yl]-pyrrolidine-1-carboxylic acid tert-butyl ester was obtained. ESI-LCMS m/e calcd. for C₄₅H₄₃F₂N₇O₅ 799.3 [M⁺], found 800.3 [M+H⁺].

To the solution of (S)-tert-butyl 4,4-difluoro-2-(5-(6-(4-(2-((2S,5S)-5-(methoxycarbonyl-amino)-4-oxo-1,2,4,5,6,7-hexahydroazepino[3,2,1-hi]indol-2-yl)-1H-imidazol-5-yl)phenyl)naphthalen-2-yl)-1H-imidazol-2-yl)pyrrolidine-1-carboxylate (100 mg, 0.125 mmol) in methylenechloride (2 ml), HCl (4M in dioxane, 0.625 ml, 2.50 mmol) was added. The reaction mixture was stirred at RT for 30 min. The mixture was evaporated under vacuum to remove solvent and HCl. Toluene was added to the residue and was evaporated under high vacuum. The resulting {(2S,5S)-2-[5-(4-{6-[2-((S)-4,4-Difluoro-pyrrolidin-2-yl)-3H-imidazol-4-yl]-naphthalen-2-yl}-phenyl)-1H-imidazol-2-yl]-4-oxo-1,2,4,5,6,7-hexahydro-azepino[3,2,1-hi]indol-5-yl}-carbamic acid methyl ester hydrochloride as white solid was used directly for next reaction. ESI-LCMS m/e calcd. for C₄₀H₃₅F₂N₇O₃ 699.3 [M⁺], found 700.3 [M+H⁺].

To the mixture of methyl (2S,5S)-2-(5-(4-(6-(2-((S)-4,4-difluoropyrrolidin-2-yl)-1H-imidazol-5-yl)naphthalen-2-yl)phenyl)-1H-imidazol-2-yl)-4-oxo-1,2,4,5,6,7-hexahydroazepino[3,2,1-hi]indol-5-ylcarbamate hydrochloride (0.125 mmol, crude from last step), (S)-2-(methoxycarbonylamino)-3-methylbutanoic acid, 175 mg, 1 mmol), and Propylphosphonic Anhydride solution(50% in EtOAc, 1.27 g solution, 2 mmol) in THF(anhydrous, 2 ml) was added N,N′-diisopropylethylamine (523 ul, 3 mmol) dropwise. The reaction mixture was stirred at 70° C. in an oil bath for overnight. The reaction mixture was concentrated under vacuum. Then the saturated NaHCO₃ aqueous solution was added to the residue to have the pH to weak basic (pH=8). The resulting muddy mixture was partitioned with dichloromethane and water. The aqueous phase was extracted with dichloromethane (2×). The combined organic phases were evaporated and was purified by the flash silica gel column separation (EtOAc/Hexane 0 to 80% gradient, then 80%), and was further purified by C-18 prep-HPLC separation (MeCN/H₂O 10-100% gradient) to obtain 26 mg (24% yield) of {(2S,5S)-2-{5-[4-(6-{2-[(S)-4,4-difluoro-1-((S)-2-methoxycarbonylamino-3-methyl-butyryl)-pyrrolidin-2-yl]-3H-imidazol-4-yl}-naphthalen-2-yl)-phenyl]-1H-imidazol-2-yl}-4-oxo-1,2,4,5,6,7-hexahydro-azepino[3,2,1-hi]indol-5-yl}-carbamic acid methyl ester as solid. ESI-LCMS m/e calcd. for C₄₇H₄₆F₂N₈O₆ 856.3 [M⁺], found 857.3 [M+H⁺]; ¹H NMR (DMSO-d₆) δ: 11.71-12.61 (m, 2H), 6.91-8.34 (m, 17H), 5.78 (d, J=9.2 Hz, 1H), 5.32 (t, J=7.3 Hz, 1H), 3.64-4.74 (m, 5H), 3.48-3.64 (m, 6H), 2.65-3.20 (m, 4H), 1.76-2.38 (m, 5H), 0.86 (d, J=5.8 Hz, 6H).

### Example 43

### {(2S,5S)-2-[5-(4-{2-[(S)-1-((S)-3-Methoxy-2-methoxycarbonylamino-propionyl)-pyrrolidin-2-yl]-3H-imidizol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester

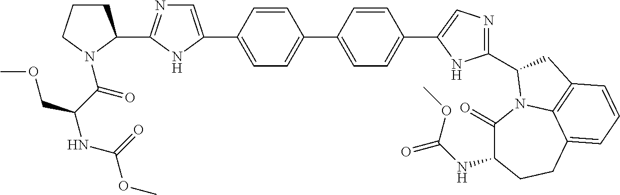

A mixture of (S)-3-methoxy-2-(methoxycarbonylamino)propanoic acid (28.9 mg, 163 mmol, Eq: 1.00), HATU (68.2 mg, 179 μmol, Eq: 1.1) and N,N′-diisopropylethylamine (84.2 mg, 114 μl, 652 μmol, Eq: 4) in DMF (5.00 ml) was stirred at rt for 30 minutes. Then, added ((2S,5S)-4-oxo-2-{5-[4-((S)-2-pyrrolidin-2-yl-)-biphenyl-4-yl]-1H-imidazol-2-yl}-1,2,4,5,6,7-hexahydro-azepino[3,2,1-hi]indol-5-yl)-carbamic acid methyl ester (28.9 mg, 0.163 mmol, Eq:1.0) (Intermediate 18). Reaction was stirred at room temperature for 3 hours. The reaction mixture was then concentrated in vacuo and purified by reverse phase chromatography to afford {(2S,5S)-2-[5-(4-{2-[(S)-1-((S)-3-methoxy-2-methoxycarbonylamino-propionyl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester as a white solid (7.0 mg, 5.56%). LCMS calcd for C₄₃H₄₄N₈O₆ (m/e) 772, obsd 773 (M 4H). ¹H NMR (400 MHz, DMSO-d₆) δ ppm 1.82-3.92 (m, 27H) 4.14 (t, J=7.91 Hz, 1H) 4.56 (d, J=4.52 Hz, 1H) 5.07 (d, J=6.53 Hz, 1H) 5.77 (d, J=9.54 Hz, 1H) 6.89-7.91 (m, 12H) 11.55-11.93 (m, 1H).

### Example 44

### (2S,5S)-2-[5-(4-{2-[(S)-1-((S)-2-Cyclopropyl-2-methoxycarbonylamino-acetyl)-pyrrolidin-2-yl]-3H-imidizol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester

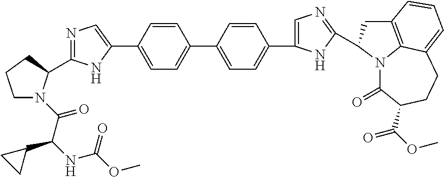

The compound was prepared using same procedure described in Example 44, to afford 7 mg, 8% yield. LCMS calcd for C₄₃H₄₄N₈O₆ (m/e) 768, obsd 769 (M 4H). ¹H NMR (400 MHz, DMSO-d₆) δ ppm 0.01-0.78 (m, 4H) 1.78-2.41 (m, 5H) 2.87-3.20 (m, 3H) 3.21-3.47 (m, 2 H) 3.41-3.84 (m, 8H) 3.96-4.27 (m, 2H) 5.07 (d, J=3.76 Hz, 1H) 5.77 (d, J=9.29 Hz, 1H) 6.91-7.88 (m, 13H) 11.63-11.90 (m, 2H).

### Example 45

### {(2S,5S)-2-[5-(4-{2-[(S)-1-((S)-2-Methoxycarbonylamino-butyryl)-pyrrolidin-2-yl]-3H-imidizol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester

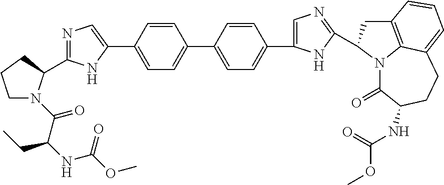

In a 50 mL round-bottomed flask, (S)-2-(5-(4′-(2-((2S,5S)-5-(methoxycarbonylamino)-4-oxo-1,2,4,5,6,7-hexahydroazepino[3,2,1-hi]indol-2-yl)-1H-imidazol-5-yl)biphenyl-4-yl)-1H-imidazol-2-yl)pyrrolidinium chloride (40 mg, 61.5 μmol, Eq: 1.00) (Intermediate 18), 1-propanephosphonic acid cyclic anhydride 50% ethyl acetate (97.9 mg, 90.6 μA, 154 μmol, Eq: 2.5) (S)-2(methoxycarbonylamino)butanoic acid (9.91 mg, 61.5 μmol, Eq: 1.00) and N,N′-diisopropylethylamine (4 Eq) were combined with THF (3 ml). Reaction was heated at 70 C for 3 hrs. The reaction mixture was then concentrated in vacuo. To the crude residue was added, aqueous saturated sodium bicarbonate. The solid was filtered and washed with water to afford {(2S,5S)-2-[5-(4-{2-[(S)-1-((S)-2-methoxycarbonylamino-butyryl)-pyrrolidin-2-yl]-3H-imidizol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester (35 mg, 75% yield) as a white solid. LCMS calcd for C₄₂H₄₄N₈O₆ (m/e) 756, obsd 757 (M 4H). ¹H NMR (400 MHz, DMSO-d₆) δ ppm 0.64-4.45 (m, 29H), 5.14 (br. s., 1H), 5.63-6.04 (m, 1H), 6.94-8.08 (m, 12H), 11.62-12.01 (m, 1H).

### Example 46

### {(2S,5S)-2-[5-(4-{2-[(S)-1-((8)-4-Methoxy-2-Methoxycarbonylamino-butyryl)-pyrrolidin-2-yl]-3H-imidizol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester

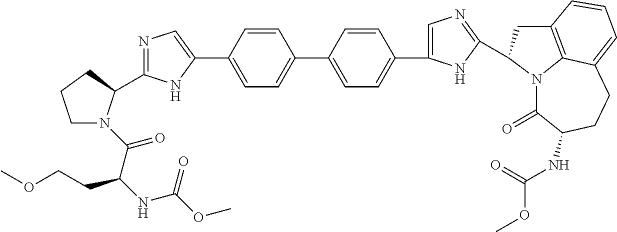

The compound was prepared using 1-propanephosphonic acid cyclic anhydride in 50% ethyl acetate method as describe in Example 46 to afford (12 mg, 47% yield) as a white solid. LCMS calcd for C₄₃H₄₆N₈O₇ (m/e) 786, obsd 787 (M 4H). ¹H NMR (400 MHz, DMSO-d₆) δ ppm 0.71-3.77 (m, 29H) 4.05-4.30 (m, 1H) 4.47 (d, J=6.02 Hz, 1H) 5.15 (br. s., 1H) 5.83 (d, J=9.79 Hz, 1H) 6.95-8.08 (m, 12H) 11.70-12.02 (m, 1H).

### Example 47

### {(2S,5S)-2-[5-(4-{2-[(S)-1-(3-Methoxycarbonylamino-oxetane-3-carbonyl)-pyrrolidin-2-yl]-3H-imidizol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester

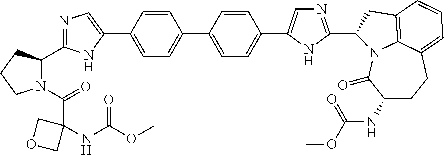

The compound was prepared using 1-propanephosphonic acid cyclic anhydride in 50% ethyl acetate method as described in Example 46 to afford (43 mg, 91% yield) as a white solid. LCMS calcd for C₄₂H₄₂N₈O₇ (m/e) 770, obsd 771 (M⁺+H). ¹H NMR (400 MHz, DMSO-d₆) δ ppm 0.64-2.45 (m, 18H) 3.62 (s, 6H) 4.06-5.22 (m, 5H) 5.83 (d, J=9.54 Hz, 1H) 6.97-7.99 (m, 12H) 11.91 (br. s., 1H).

### Example 48

### ((2S,5S)-2-{5-[4-(2-{(8)-1-[2-Methoxycarbonylamino-2-(tetrahydro-pyran-4-yl)-pyrrolidin-2-yl]-3H-imidizol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester

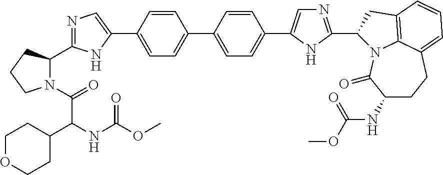

The compound was prepared using 1-propanephosphonic acid cyclic anhydride in 50% ethyl acetate method as described in Example 46 to afford (31 mg, 59% yield) as a white solid. LCMS calcd for C₄₅H₄₈N₈O₇ (m/e) 812, obsd 813 (M 4H). ¹H NMR (400 MHz, DMSO-d₆) δ ppm 0.68-2.16 (m, 14H) 2.91-3.48 (m, 12H) 3.80-4.37 (m, 4H) 5.14 (br. s., 1H) 5.84 (d, J=9.29 Hz, 1H) 6.92-6.97 (m, 1H) 6.95-8.06 (m, 12H) 11.91 (br. s., 1H).

### Example 49

### {(2S,5S)-2-[5-(4-{2-[(S)-1-((8)-2-Methoxycarbonylamino-propionyl)-pyrrolidin-2-yl]-3H-imidizol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester

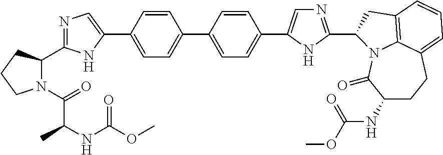

The compound was prepared using 1-propanephosphonic acid cyclic anhydride in 50% ethyl acetate method as mentioned above to afford (31 mg, 59% yield) as a white solid. LCMS calcd for C₄ᵢH₄₂N₈O₆ (m/e) 742, obsd 743 (M⁺+H). ¹H NMR (400 MHz, DMSO-d₆) δ ppm 0.73-4.39 (m, 25H) 5.14 (br. s., 1H) 5.67-5.76 (m, 1H) 6.84-6.95 (m, 1H) 6.91-7.98 (m, 12H) 6.92-6.97 (m, 1H) 11.75-12.02 (m, 1H).

### Example 50

### {(2S,5S)-2-[5-(4-{2-[(S)-1-((S)-3-Hydroxy-2-methoxycarbonylamino-butyl)-pyrrolidin-2-yl]-3H-imidizol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester

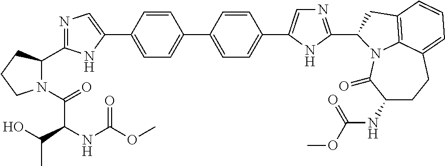

The compound was prepared using 1-propanephosphonic acid cyclic anhydride in 50% ethyl acetate method as described in Example 46 to afford (17 mg, 68% yield) as a white solid. LCMS calcd for C₄₂H₄₄N₈O₇ (m/e) 772, obsd 773 (M 4H). ¹H NMR (400 MHz, DMSO-d₆) δ ppm 0.72-3.85 (m, 27H) 4.05-4.51 (m, 2H) 5.01-5.31 (m, 1H) 5.84 (d, J=9.54 Hz, 1H) 6.91-8.07 (m, 12H) 11.66-12.05 (m, 1H).

### Example 51

### {(2S,5S)-2-[5-(4-{2-[(S)-1-((S)-4-Methanesulfonyl-2-methoxycarbonylamino-butyl)-pyrrolidin-2-yl]-3H-imidizol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester

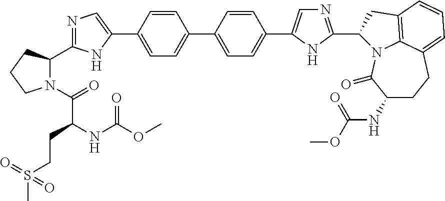

The compound was prepared using 1-propanephosphonic acid cyclic anhydride in 50% ethyl acetate method as described in Example 46, to afford (17 mg, 68% yield) as a white solid. LCMS calcd for C₄₃H₄₆N₈O₈S (m/e) 834, obsd 835 (M 4H). ¹H NMR (400 MHz, DMSO-d₆) ppm 0.18-1.08 (m, 4H) 1.29-1.95 (m, 6H) 1.40-1.91 (m, 6H) 2.28-2.56 (m, 14H) 2.50 (s, 1H) 3.12 (s, 6H) 3.59-3.80 (m, 1H) 4.04 (d, J=5.52 Hz, 1H) 4.63 (d, J=6.78 Hz, 1H) 5.33 (d, J=9.54 Hz, 1H) 6.17-7.64 (m, 12H) 11.40 (br. s., 1H).

### Example 52

### ((2S,5S)-2-{5-[4-(6-{2-[(S)-1-((S)-2-Methoxycarbonylamino-3-methyl-butyryl)-pyrrolidin-2-yl]-3H-imidazol-4-yl}-quinoxalin-2-yl)-phenyl]-1H-imidazol-2-yl}-4-oxo-1,2,4,5,6,7-hexahydro-azepino[3,2,1-hi]indol-5-yl)-carbamic acid methyl ester

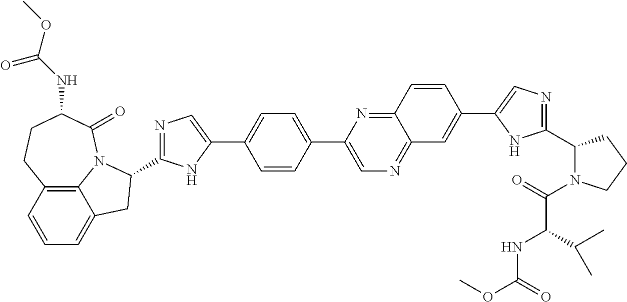

To a stirred solution of (S)-2-(methoxycarbonylamino)-3-methylbutanoic acid (7.89 mg, 45.1 μmol, Eq: 1.00) in DMF (0.5 ml), was added HATU (18.8 mg, 49.6 μmol, Eq: 1.1) and Hunig's base (23.3 mg, 31.5 μA, 180 μmol, Eq: 4). After stirring for 30 min, methyl (2S,5S)-4-oxo-2-(5-(4-(6-(2-((S)-pyrrolidin-2-yl)-1H-imidazol-5-yl)quinoxalin-2-yl)phenyl)-1H-imidazol-2-yl)-1,2,4,5,6,7-hexahydroazepino[3,2,1-hi]indol-5-ylcarbamate hydrochloride (31.6 mg, 45.1 mmol, Eq: 1.00) was added in DMF (0.5 ml) (Intermediate 27) and the reaction mixture was stirred at rt. for 4 hrs. The volatiles were removed in vacuo, the residue was chromatographed (silica, gradient 0-10% (1% NH₄OH in MeOH)— dichloromethane) to obtain ((2S,5S)-2-{5-[4-(6-{2-[(S)-1-((S)-2-Methoxycarbonylamino-3-methyl-butyryl)-pyrrolidin-2-yl]-3H-imidazol-4-yl}-quinoxalin-2-yl)-phenyl]-1H-imidazol-2-yl}-4-oxo-1,2,4,5,6,7-hexahydro-azepino[3,2,1-hi]indol-5-yl)-carbamic acid methyl ester (20.5 mg, 24.9 μmol, 55.3% yield), as a yellow powder. LC/MS (M⁺+H)=823, ¹H NMR (400 MHz, DMSO-d₆) ppm 0.75-1.04 (m, 7H) 1.89-2.37 (m, 7H) 2.98-3.21 (m, 2H) 3.47-3.71 (m, 8H) 3.84 (br. s., 2H) 4.01-4.24 (m, 2 H) 5.11 (d, J=4.02 Hz, 1H) 5.79 (d, J=9.54 Hz, 1H) 6.98-7.36 (m, 4H) 7.50 (d, J=8.28 Hz, 1 H) 7.62-7.72 (m, 1H) 7.79-7.95 (m, 3H) 8.00-8.12 (m, 1H) 8.20-8.51 (m, 5H) 9.38-9.63 (m, 1H) 11.88-12.10 (m, 2H) 12.17-12.62 (m, 1H).

### Example 53

### ((2S,5S)-2-{5-[4-(6-{2-[(S)-1-((2S,3S)-2-Methoxycarbonylamino-3-methyl-pentanoyl)-pyrrolidin-2-yl]-3H-imidazol-4-yl}-quinoxalin-2-yl)-phenyl]-1H-imidazol-2-yl}-4-oxo-1,2,4,5,6,7-hexahydroazepino[3,2,1-hi]indol-5-yl)-carbamic acid methyl ester

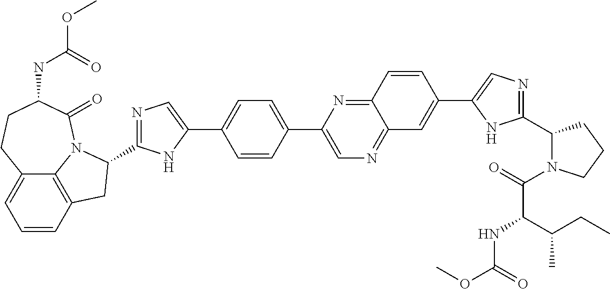

To a stirred solution of (2S,3S)-2-(methoxycarbonylamino)-3-methylpentanoic acid (9.38 mg, 49.6 μmol, Eq: 1.1) in DMF (0.5 ml) at rt. was added HATU (18.8 mg, 49.6 μmol, Eq: 1.1) and Hunig's base (23.3 mg, 31.5 μl, 180 μmol, Eq: 4). After stirring for 30 min, methyl (2S,5S)-4-oxo-2-(5-(4-(6-(2-((S)-pyrrolidin-2-yl)-1H-imidazol-5-yl)quinoxalin-2-yl)phenyl)-1H-imidazol-2-yl)-1,2,4,5,6,7-hexahydroazepino[3,2,1-hi]indol-5-ylcarbamate hydrochloride (31.6 mg, 45.1 μmol, Eq: 1.00) (Intermediate 27) was added in DMF (0.5 ml) and the reaction mixture was stirred at rt. for 3 hrs. The volatiles were evaporated and the residue was chromatographed (silica gel, gradient 0-10% MeOH (1% NH₄OH in MeOH-dichloromethane) to obtain ((2S,5S)-2-{5-[4-(6-{2-[(S)-1-((2S,3S)-2-methoxycarbonylamino-3-methyl-pentanoyl)-pyrrolidin-2-yl]-3H-imidazol-4-yl}-quinoxalin-2-yl)-phenyl]-1H-imidazol-2-yl}-4-oxo-1,2,4,5,6,7-hexahydroazepino[3,2,1-hi]indol-5-yl)-carbamic acid methyl ester (23.6 mg, 28.2 μmol, 62.6% yield) as a yellow powder. LC/MS (M⁺+H)=837. ¹H NMR (400 MHz, DMSO-d₆) δ ppm 0.72-0.98 (m, 9H) 1.23 (br. s., 10H) 1.39-1.81 (m, 3H) 1.89-2.36 (m, 7H) 3.09 (d, J=6.02 Hz, 3H) 3.45-3.69 (m, 10H) 3.85 (br. s., 2H) 4.14 (br. s., 3H) 5.11 (d, J=3.51 Hz, 1H) 5.79 (d, J=9.79 Hz, 1H) 6.94-7.58 (m, 6H) 7.62-8.13 (m, 5H) 8.17-8.50 (m, 5H) 9.36-9.67 (m, 1H) 11.86-12.13 (m, 2H) 12.19-12.60 (m, 1H).

### Example 54

### ((2S,5S)-2-{5-[4-(6-{2-[(S)-1-((S)-2-Methoxycarbonylamino-3,3-dimethyl-butyryl)-pyrrolidin-2-yl]-3H-imidazol-4-yl}-quinoxalin-2-yl)-phenyl]-1H-imidazol-2-yl}-4-oxo-1,2,4,5,6,7-hexahydro-azepino[3,2,1-hi]indol-5-yl)-carbamic acid methyl ester

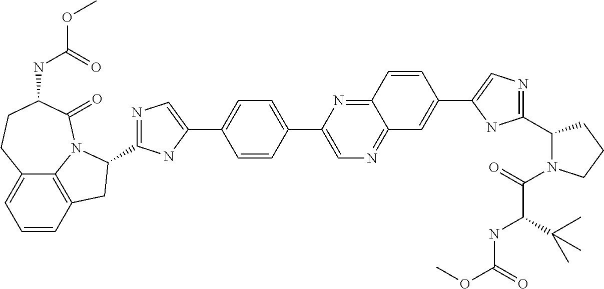

To a stirred solution of (S)—N-(methoxycarbonyl)-tent-leucine (6.25 mg, 33.0 μmol, Eq: 1.1) in DMF (0.5 ml) was added HATU (12.6 mg, 33.0 μmol, Eq: 1.1) and Hunig's base (15.5 mg, 21.0 μA, 120 μmol, Eq: 4). After stirring for 30 min. a solution of methyl (2S,5S)-4-oxo-2-(5-(4-(6-(2-((S)-pyrrolidin-2-yl)-1H-imidazol-5-yl)quinoxalin-2-yl)phenyl)-1H-imidazol-2-yl)-1,2,4,5,6,7-hexahydroazepino[3,2,1-hi]indol-5-ylcarbamate hydrochloride (21.1 mg, 30.0 μmol, Eq: 1.00) (Intermediate 27) in DMF (0.5 ml) was added and reaction mixture was stirred at room temperature for overnight. The volatiles were removed in vacuo, the residue was chromatographed (silica gel, gradient 0-10% MeOH (1% NH₄OH in MeOH)— dichloromethane) then it was further purified by Preparative TLC to obtain ((2S,5S)-2-{5-[4-(6-{2-[(S)-1-((S)-2-methoxycarbonylamino-3,3-dimethyl-butyryl)-pyrrolidin-2-yl]-3H-imidazol-4-yl}-quinoxalin-2-yl)-phenyl]-1H-imidazol-2-yl}-4-oxo-1,2,4,5,6,7-hexahydro-azepino[3,2,1-hi]indol-5-yl)-carbamic acid methyl ester (11.5 mg, 45.7% yield) as a yellow powder. LC/MS (M⁺+H)=837.

¹H NMR (400 MHz, CHLOROFORM-d) δ ppm 0.77-1.35 (m, 22H) 1.74 (br. s., 1H) 1.91-2.49 (m, 8H) 2.70-3.43 (m, 5H) 3.45-3.80 (m, 11H) 3.84-4.01 (m, 2H) 4.38 (d, J=9.29 Hz, 3H) 5.20-5.82 (m, 4H) 5.85-6.14 (m, 2H) 6.96-7.48 (m, 12H) 7.63 (d, J=6.78 Hz, 1H) 7.85-8.34 (m, 6H) 8.47 (s, 1H) 9.29 (s, 1H) 10.21 (br. s., 1H) 10.40-10.62 (m, 1H) 10.97-11.10 (m, 1H).

### Example 55

### ((2S,5S)-2-{5-[4-(6-{2-[(S)-1-((R)-2-Methoxycarbonylamino-2-phenyl-acetyl)-pyrrolidin-2-yl]-3H-imidazol-4-yl}-quinoxalin-2-yl)-phenyl]-1H-imidazol-2-yl}-4-oxo-1,2,4,5,6,7-hexahydro-azepino[3,2,1-hi]indol-5-yl)-carbamic acid methyl ester

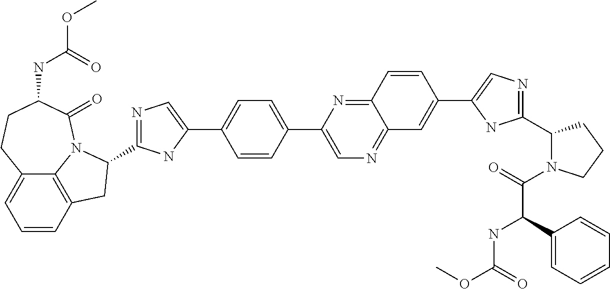

To a stirred solution of (R)-2-(methoxycarbonylamino)-2-phenylacetic acid (6.91 mg, 33.0 μmol, Eq: 1.1) in DMF (0.5 ml) at rt. was added HATU (12.6 mg, 33.0 μmol, Eq: 1.1) and Hunig's base (15.5 mg, 21.0 μA, 120 μmol, Eq: 4). After stirring for 30 min. a solution of methyl (2S,5S)-4-oxo-2-(5-(4-(6-(2-((S)-pyrrolidin-2-yl)-1H-imidazol-5-yl)quinoxalin-2-yl)phenyl)-1H-imidazol-2-yl)-1,2,4,5,6,7-hexahydroazepino[3,2,1-hi]indol-5-ylcarbamate hydrochloride (21.1 mg, 30.0 μmol, Eq: 1.00) (Intermediate 27) in DMF (0.5 ml) was added and reaction mixture was stirred at rt. for 18 hrs, TLC and LC/MS shows only starting material, reaction mixture heated at 50° C. for 6 hrs. LC/MS shows only product. The volatiles were removed in vacuo, the residue chromatographed (silica, 0-10% MeOH (1% NH₄OH in MeOH)— dichloromethane) to obtain ((2S,5S)-2-{5-[4-(6-{2-[(S)-1-((R)-2-methoxycarbonylamino-2-phenyl-acetyl)-pyrrolidin-2-yl]-3H-imidazol-4-yl}-quinoxalin-2-yl)-phenyl]-1H-imidazol-2-yl}-4-oxo-1,2,4,5,6,7-hexahydro-azepino[3,2,1-hi]indol-5-yl)-carbamic acid methyl ester (12.5 mg, 14.6 μmol, 48.6% yield) as a yellow powder. LC/MS (M⁺+H)=857. ¹H NMR (400 MHz, CHLOROFORM-d) δ ppm 0.87 (d, J=6.53 Hz, 2H) 1.11-1.36 (m, 3H) 1.50-1.81 (m, 2H) 1.85-2.49 (m, 8H) 2.84 (br. s., 1H) 2.95-3.37 (m, 5H) 3.43-3.88 (m, 12H) 4.05-4.57 (m, 3H) 5.18-5.59 (m, 3H) 5.62-6.28 (m, 5H) 6.89-7.55 (m, 18H) 7.77 (d, J=6.27 Hz, 2H) 7.97-8.22 (m, 4H) 8.35 (br. s., 1H) 9.23 (br. s., 1H).

### Example 56

### ((2S,5S)-2-{5-[2-(4-{2-[(S)-1-((28,38)-2-Methoxycarbonylamino-3 methyl-pentanoyl)-pyrrolidin-2-yl]-3H-imidazol-4-yl}-phenyl) quinoxalin-6-yl]-1H-imidazol-2-yl}-4-oxo-1,2,4,5,6,7-hexahydro-azepino[3,2,1-hi]indol-5-yl)-carbamic acid methyl ester

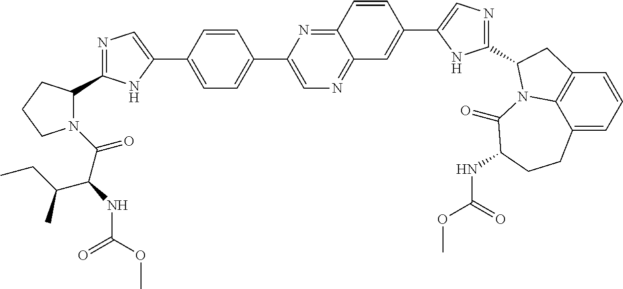

The compound was prepared using 1-propanephosphonic acid cyclic anhydride in 50% ethyl acetate method as mentioned above (Example 56) to afford 14 mg, (21% yield) as a white solid. ESI-LRMS m/e calcd for C₄₆H₄₈N_{1006 [}M⁺] 836.0, found 837.1 [M+H]. ¹H NMR (400 MHz, DMSO-d₆) δ ppm 0.80 (br. s., 6H) 0.87 (br. s., 11H) 1.22-1.29 (m, 3H) 2.17 (br. s., 1H) 2.33 (br. s., 1H) 2.67 (br. s., 1H) 3.11 (br. s., 4H) 3.42 (br. s., 2H) 3.55 (d, J=7.28 Hz, 21H) 4.14 (br. s., 2H) 5.81 (d, J=9.29 Hz, 3H) 7.10 (br. s., 2H) 7.20 (d, J=6.78 Hz, 2H) 7.51 (d, J=6.78 Hz, 3H) 7.85 (br. s., 1H) 8.04 (br. s., 2H) 8.22-8.33 (m, 5H) 9.52 (br. s., 1H) 12.08 (br. s., 2H).

### Example 57

### ((2S,5S)-2-{4-[4-(5-{2-[(S)-4,4-Difluoro-1-(2-methoxycarbonylamino-3-methyl-butyryl)-pyrrolidin-2-yl]-3H-imidazol-4-yl}-pyrimidin-2-yl)-phenyl]-1H-imidazol-2-yl}-4-oxo-1,2,4,5,6,7-hexahydro-azepino[3,2,1-hi]indol-5-yl)-carbamic acid methyl ester

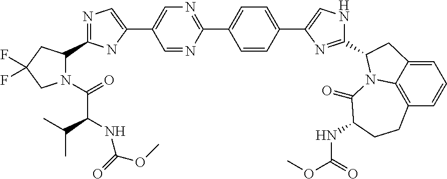

To a stirred solution of (S)-2-(methoxycarbonylamino)-3-methylbutanoic acid (4.2 mg, 24.0 μmol, Eq: 1.1) in DMF (0.5 ml) was added HATU (9.12 mg, 24.0 μmol, Eq: 1.1) and Hunig's base (11.3 mg, 15.2 μA, 87.2 μmol, Eq: 4). After stirring for 30 min. methyl (2S,5S)-2-(4-(4-(5-(2-((S)-4,4-difluoropyrrolidin-2-yl)-1H-imidazol-5-yl)pyrimidin-2-yl)phenyl)-1H-imidazol-2-yl)-4-oxo-1,2,4,5,6,7-hexahydroazepino[3,2,1-hi]indol-5-ylcarbamate hydrochloride (15 mg, 21.8 μmol, Eq: 1.00) (Intermediate 29) was added in DMF (0.5 ml) and the reaction mixture was stirred at rt. for 4 hrs. The volatiles were removed in vacuo, the residue chromatographed (silica, 0-10% MeOH (1% NH₄OH in MeOH)—dichloromethane to obtain ((2S,5S)-2-{4-[4-(5-{2-[(S)-4,4-Difluoro-1-(2-methoxycarbonylamino-3-methyl-butyryl)-pyrrolidin-2-yl]-3H-imidazol-4-yl}-pyrimidin-2-yl)-phenyl]-1H-imidazol-2-yl}-4-oxo-1,2,4,5,6,7-hexahydro-azepino[3,2,1-hi]indol-5-yl)-carbamic acid methyl ester (11.8 mg, 14.6 mmol, 66.9% yield) as an off-white powder. LC/MS (M⁺+H)=809. ¹H NMR (400 MHz, DMSO-d₆) δ ppm 1.77-2.37 (m, 9H) 3.01-3.16 (m, 4H) 3.47-3.69 (m, 10H) 3.96 (t, J=8.16 Hz, 2H) 4.05-4.35 (m, 3H) 4.52 (br. s., 1H) 5.19-5.45 (m, 1H) 5.77 (d, J=12.05 Hz, 1H) 6.96-7.33 (m, 5H) 7.62 (s, 4H) 7.73-7.98 (m, 4H) 8.33 (d, J=8.28 Hz, 3H) 9.05-9.26 (m, 3 H) 11.91 (br. s., 1H) 12.25 (br. s., 1H).

### Example 58

### ((1S,2S)-1-{(8)-4,4-Difluoro-2-[5-(2-{4-[2-((2S,5S)-5-methoxycarbonylamino-4-oxo-1,2,4,5,6,7-hexahydro-azepino[3,2,1-hi]indol-2-yl)-1H-imidazol-4-yl]-phenyl}-pyrimidin-5-yl)-1H-imidazol-2-yl]-pyrrolidine-1-carbonyl}-2-methyl-butyl)-carbamic acid methyl ester

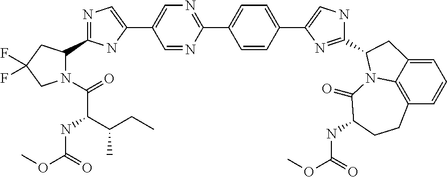

To a stirred solution of (2S,3S)-2-(methoxycarbonylamino)-3-methylpentanoic acid (3.93 mg, 20.8 μmol, Eq: 1.1) in DMF (0.5 ml) was added HATU (7.9 mg, 20.8 μmol, Eq: 1.1) and Hunig's base (9.77 mg, 13.2 μA, 75.6 μmol, Eq: 4). After stirring for 30 min. methyl (2S,5S)-2-(4-(4-(5-(2-((S)-4,4-difluoropyrrolidin-2-yl)-1H-imidazol-5-yl)pyrimidin-2-yl)phenyl)-1H-imidazol-2-yl)-4-oxo-1,2,4,5,6,7-hexahydroazepino[3,2,1-hi]indol-5-ylcarbamate hydrochloride (13 mg, 18.9 μmol, Eq: 1.00) (Intermediate 29) was added in DMF (0.5 ml) and the reaction mixture was stirred at rt. for 4 hrs. The volatiles were removed in vacuo, the residue chromatographed (silica, 0-10% MeOH (1% NH₄OH in MeOH)—dichloromethane to obtain ((1S,2S)-1-{(S)-4,4-Difluoro-2-[5-(2-{4-[2-((2S,5S)-5-methoxycarbonylamino-4-oxo-1,2,4,5,6,7hexahydro-azepino[3,2,1-hi]indol-2-yl)-1H-imidazol-4-yl]-phenyl}-pyrimidin-5-yl)-1H-imidazol-2-yl]-pyrrolidine-1-carbonyl}-2-methyl-butyl)-carbamic acid methyl ester (12.5 mg, 15.2 μmol, 80.4% yield) as a white powder. LC/MS (M⁺+H)=823. ¹H NMR (400 MHz, DMSO-d₆) δ ppm 0.61-0.93 (m, 9H) 0.99-1.83 (m, 6H) 1.98-2.37 (m, 4H) 2.60-3.18 (m, 7 H) 3.43-3.74 (m, 10H) 3.89-4.36 (m, 5H) 4.53 (br. s., 1H) 5.31 (t, J=7.15 Hz, 1H) 5.77 (d, J=10.04 Hz, 1H) 6.90-7.33 (m, 4H) 7.42-7.67 (m, 3H) 7.72-8.02 (m, 4H) 8.21-8.54 (m, 3 H) 9.06-9.29 (m, 3H) 11.92 (br. s., 1H) 12.27 (br. s., 1H).

### Example 59

### ((2S,5S)-2-{5-[2-(4-{2-[(S)-1-((S)-2-Methoxycarbonylamino-3-methyl-butyryl)-pyrrolidin-2-yl]-3H-imidazol-4-yl}-phenyl)-quinoxalin-6-yl]-1H-imidazol-2-yl}-4-oxo-1,2,4,5,6,7-hexahydro-azepino[3,2,1-hi]indol-5-yl)-carbamic acid methyl ester

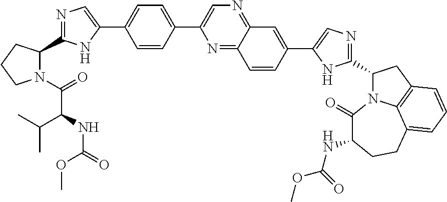

A solution of methyl (S)-1-((S)-2-(5-(4-(6-bromoquinoxalin-2-yl)phenyl)-1H-imidazol-2-yl)pyrrolidin-1-yl)-3-methyl-1-oxobutan-2-ylcarbamate (4.47 g, 7.74 mmol) (Intermediate 30) and tributyl(1-ethoxyvinyl)stannane (4.19 g, 11.6 mmol) in dioxane (14 mL) was purged with nitrogen for ten minutes followed by addition of PdCl₂(dppf)-CH₂Cl₂ adduct. The mixture was purged again with nitrogen for ten minutes followed by heating at 80° C. for 6 hours. Solvent was then removed by evaporation under reduced pressure. The crude product was purified by column (0-5% MeOH in DCM) to give methyl (S)-1-((S)-2-(5-(4-(6-(1-ethoxyvinyl)quinoxalin-2-yl)phenyl)-1H-imidazol-2-yl)pyrrolidin-1-yl)-3-methyl-1-oxobutan-2-ylcarbamate (quantitative): ESI-LRMS m/e calcd for C₃₂H₃₆N₆O₄ [M⁺] 568, found 569 [M+H⁺].

To a stirred solution of methyl (S)-1-((S)-2-(5-(4-(6-(1-ethoxyvinyl) quinoxalin-2-yl)phenyl)-1H-imidazol-2-yl)pyrrolidin-1-yl)-3-methyl-1-oxobutan-2-ylcarbamate (4.4 g, 7.74 mmol) in THF and water was added NBS (1.38 g, 7.74 mmol) at 0° C. in four portions over 15 minutes. The mixture was then stirred at 0° C. for 45 minutes, followed by concentration in vacuo. The crude product was purified by column (0-5% MeOH in dichloromethane) to give methyl (S)-1-((S)-2-(5-(4-(6-(2-bromoacetyl) quinoxalin-2-yl)phenyl)-1H-imidazol-2-yl)pyrrolidin-1-yl)-3-methyl-1-oxobutan-2-ylcarbamate (988 mg, 21%): ESI-LRMS m/e calcd for C₃₀H₃₁BrN₆O₄ [M⁺] 620, found 521 [M+H⁺].

To a solution of methyl (S)-1-((S)-2-(5-(4-(6-(2-bromoacetyl)quinoxalin-2-yl)phenyl)-1H-imidazol-2-yl)pyrrolidin-1-yl)-3-methyl-1-oxobutan-2-ylcarbamate (950 mg, 1.53 mmol) and (2S,5S)-5-(methoxycarbonylamino)-4-oxo-1,2,4,5,6,7-hexahydroazepino[3,2,1-hi]indole-2-carboxylic acid (490 mg, 1.61 mmol) in dichloromethane (50 mL) was added N,N′-diisopropylethylamine (0.67 mL, 3.83 mmol) at 0° C. The resulting solution was stirred room temperature overnight. LC-MS showed bromide starting material consumed and major peak gave [M+1]⁺ of desired product. The solvent was removed by evaporation and the crude product was purified by column (0-10% MeOH in DCM) to (2S,5S)-2-(2-(4-(2-((S)-1-((S)-2-(methoxycarbonylamino)-3-methylbutanoyl)pyrrolidin-2-yl)-1H-imidazol-5-yl)phenyl)quinoxalin-6-yl)-2-oxoethyl-5-(methoxycarbonylamino)-4-oxo-1,2,4,5,6,7-hexahydroazepino[3,2,1-hi]indole-2-carboxylate (400 mg, 31%): ESI-LRMS m/e calcd for C₄₅H₄₆N₈O₉ [M⁺] 842, found 843 [M+H⁺].

A mixture of (2S,5S)-2-(2-(4-(2-((S)-1-((S)-2-(methoxycarbonylamino)-3-methyl butanoyl)pyrrolidin-2-yl)-1H-imidazol-5-yl)phenyl)quinoxalin-6-yl)-2-oxoethyl 5-(methoxy carbonyl amino)-4-oxo-1,2,4,5,6,7-hexahydroazepino[3,2,1-hi]indole-2-carboxylate (400 mg, 0.475 mmol), ammonium acetate (183 mg, 2.37 mmol) and dioxane (30 mL) in a sealed vial was purged with nitrogen and heated at 110° C. for 4 hours. LC/MS showed only around −30-40% conversion to product. Another portion of ammonium acetate (183 mg, 2.37 mmol) was added to the mixture. The resulting mixture was purged with nitrogen and heated at 110° C. for another 6 hours. The solvent was then removed by evaporation in vacuo. The residue was stirred with dichloromethane (300 ml) for 1 hour followed by filtration. The filtrate was evaporated in vacuo. The crude product was purified by column (0-10% MeOH in dichloromethane) to give ((2S,5S)-2-{5-[2-(4-{2-[(S)-1-((S)-2-Methoxycarbonylamino-3-methyl-butyryl)-pyrrolidin-2-yl]-3H-imidazol-4-yl}-phenyl)-quinoxalin-6-yl]-1H-imidazol-2-yl}-4-oxo-1,2,4,5,6,7-hexahydro-azepino[3,2,1-hi]indol-5-yl)-carbamic acid methyl ester (166 mg, 42.5%): ESI-LRMS m/e calcd for C₄₅H₄₆N₁₀O₆ [M⁺] 822, found 823. ¹H NMR (400 MHz, DMSO-d₆) δ ppm 0.86, 0.92 (2d, 6 H) 1.88-2.07 (m, 2H) 2.08-2.22 (m, 2H) 2.23-2.36 (m, 1H) 3.03-3.14 (m, 2H) 3.17 (d, 2 H) 3.40 (d, 1H) 3.55 (d, 6H) 3.60-3.71 (m, 1H) 3.82 (t, 1H) 4.03-4.21 (m, 2H) 5.05-5.13 (m, 1H) 5.81 (d, 2H) 7.03 (t, 1H) 7.11 (d, 1H) 7.20 (d, 1H) 7.31 (d, 1H) 7.51 (d, 1H) 7.65 (s, 1H) 7.85 (s, 1H) 7.92 (d, 2H) 8.04 (d, 1H) 8.20-8.38 (m, 3H) 9.52 (s, 1H) 11.90 (s, 1H) 12.08 (s, 1H).

### Example 60

### {(2S,5S)-2-[5-(4′-{5-[(S)-1-((R)-2-Methoxycarbonylamino-2-phenyl-acetyl)-pyrrolidin-2-yl]-4H-[1,2,4]triazol-3-yl}-biphenyl-4-yl)-1H-imidazol-2-yl]-4-oxo-1,2,4,5,6,7-hexahydro-azepino[3,2,1-hi]indol-5-yl}-carbamic acid methyl ester

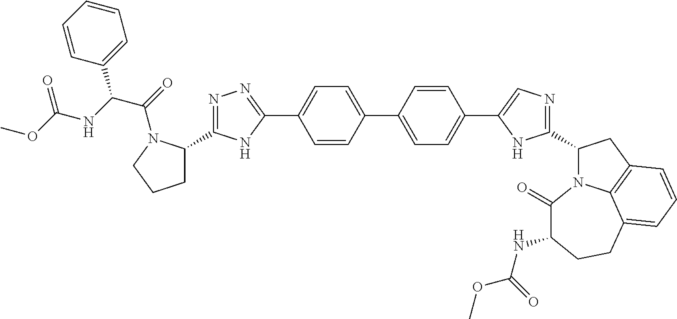

### (R)-2-(methoxycarbonylamino)-2-phenylacetic acid

To a mixture of (R)-2-amino-2-phenylacetic acid (9.2 g, 60.9 mmol), sodium hydroxide (4.87 g, 122 mmol), water (100 mL), and toluene (100 mL) cooled in ice-water bath was added dropwise methyl chloroformate (6.33 g, 66.9 mmol). The mixture was then warmed to room temperature and stirred for 12 h. The organic phase was separated. The aqueous phase was acidified with 6 N HCl at 0° C., and extracted with EtOAc. The EtOAc solution was washed with water and brine, dried over sodium sulfate, filtered and evaporated to give a white solid product. The product was recrystallized from ethyl acetate to give pure crystalline product, (R)-2-(methoxycarbonylamino)-2-phenylacetic acid (7.9 g, 62%): ESI-LRMS m/e calcd for C₁₀H₁₁NO₄ [M⁺] 209, found 208 [M−H⁺].

A solution of methyl (2S,5S)-4-oxo-2-(5-(4′-(5-((S)-pyrrolidin-2-yl)-4H-1,2,4-triazol-3-yl)biphenyl-4-yl)-1H-imidazol-2-yl)-1,2,4,5,6,7-hexahydroazepino[3,2,1-hi]indol-5-yl carbamate (7 mg, 0.011 mmol) (Intermediate 31), (R)-2-(methoxycarbonylamino)-2-phenylacetic acid (4.76 mg, 0.023 mmol), N,N′-diisopropylethylamine (7.4 mg, 0.057 mmol), and HATU (8.7 mg, 0.023 mmol) in DMF (1 mL) stayed at room temperature for 12 hours. The solution was then separated by basic preparative HPLC (5-100% acetonitrile with 0.1% TEA in water with 0.1% ammonia) to give {(2S,5S)-2-[5-(4′-{5-[(S)-1-(methylenechloride-2-Methoxycarbonylamino-2-phenyl-acetyl)-pyrrolidin-2-yl]-4H-[1,2,4]triazol-3-yl}-biphenyl-4-yl)-1H-imidazol-2-yl]-4-oxo-1,2,4,5,6,7-hexahydro-azepino[3,2,1-hi]indol-5-yl}-carbamic acid methyl ester (4.2 mg, 45.8%): ESI-LRMS m/e calcd for C₄₅H₄₃N₉O₆ [M⁺] 805, found 806. ¹H NMR (400 MHz, DMSO-d₆) δ ppm 0.98 (m, 2H) 1.17 (m, 2H) 2.08 (m, 1H) 2.19 (m, 1H) 2.26 (m, 1H) 2.38 (m, 1H) 2.60 (m, 1H) 3.02 (m, 2H) 3.09 (m, 1H) 3.45 (s, 3H) 3.59 (s, 3H) 3.65 (m, 1H) 3.84 (m, 1H) 4.08 (m, 1H) 5.07 (m, 1H) 5.45 (d, 1H), 5.68-5.74 (m, 2H) 6.92-6.98 (m, 2H) 7.03 (d, 1H) 7.12 (d, 1H) 7.25-7.37 (m, 4H) 7.40 (d, 1H) 7.48-7.51 (m, 1H) 7.60-7.66 (m, 2H) 7.69-7.77 (m, 3H) 7.87 (d, 1H) 7.98 (dd, 1H) 11.78 (s, 1H).

### Example 61

### {(2S,5S)-2-[5-(4′-{5-[(S)-1-((S)-2-Methoxycarbonylamino-3-methyl-butyryl)-pyrrolidin-2-yl]-4H-[1,2,4] triazol-3-yl}-biphenyl-4-yl)-1H-imidazol-2-yl]-4-oxo-1,2,4,5,6,7-hexahydro-azepino[3,2,1-hi]indol-5-yl}-carbamic acid methyl ester

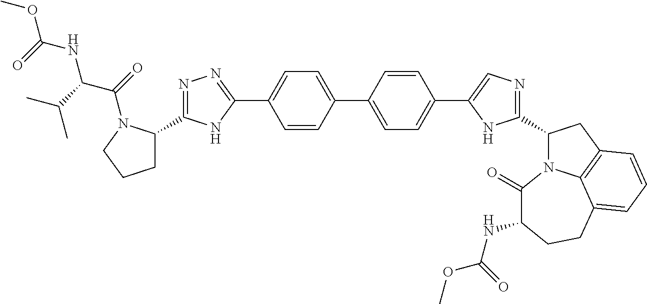

A mixture of methyl (2S,5S)-4-oxo-2-(5-(4′-(5-((S)-pyrrolidin-2-yl)-4H-1,2,4-triazol-3-yl)biphenyl-4-yl)-1H-imidazol-2-yl)-1,2,4,5,6,7-hexahydroazepino[3,2,1-hi]indol-5-ylcarbamate (128 mg (impure), 0.21 mmol) (Intermediate 30), (S)-2-(methoxycarbonylamino)-3-methylbutanoic acid (72.9 mg, 0.42 mmol), N,N′-diisopropylethylamine (0.36 mL, 2.1 mmol), and HATU (198 mg, 0.52 mmol) in anhydrous DMF (2 mL) was stayed at room temperature overnight. The solution was separated by prep HPLC using 10-100% acetonitrile containing 0.1% TFA in water containing 0.1% TFA as elute. The desired peak was concentrated and purified again via basic prep HPLC (20-100% ACN containing 0.1% TEA in water containing 0.1% ammonia) to give {(2S,5S)-2-[5-(4′-{5-[(S)-1-((S)-2-Methoxycarbonylamino-3-methyl-butyryl)-pyrrolidin-2-yl]-4H-[1,2,4]triazol-3-yl}-biphenyl-4-yl)-1H-imidazol-2-yl]-4-oxo-1,2,4,5,6,7-hexahydro-azepino[3,2,1-hi]indol-5-yl}-carbamic acid methyl ester (16.6 mg, 10.3%): ESI-LRMS m/e calcd for C₄₂H₄₅N₉O₆ [M⁺] 771, found 772. ¹H NMR (400 MHz, DMSO-d₆) δ ppm 0.79, 0.86 (2 d, 6H) 0.91-1.18 (m, 3H) 1.75-1.98 (m, 4H) 2.00-2.28 (m, 4H) 2.95-3.08 (m, 2H) 3.43-3.50 (m, 6H) 3.51-3.60 (m, 1H) 3.71-3.82 (m, 2H) 3.93-4.13 (M, 2H) 5.05-5.11 (m, 1H) 5.68-5.75 (m, 1H) 6.95 (t, 1H) 7.03 (d, 1H) 7.13 (t, 1H) 7.22 (d, 1H) 7.40 (d, 1H) 7.50 (s, 1H) 7.62 (d, 2H), 7.73 (m, 3H) 7.93 (d, 2H) 11.78 (s, 1H).

### Example 62

### {(2S,5S)-2-[5-(4′-{5-[(S)-1-((8)-2-Methoxycarbonylamino-3-methyl-butyryl)-pyrrolidin-2-yl]-1H-imidazol-2-yl}-biphenyl-4-yl)-[1,3,4]oxadiazol-2-yl]-4-oxo-1,2,4,5,6,7-hexahydro-azepino[3,2,1-hi]indol-5-yl}-carbamic acid methyl ester

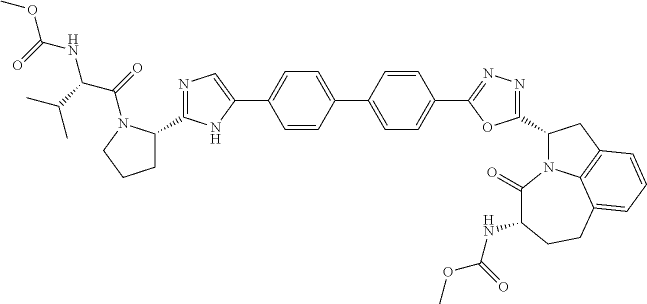

A mixture of (2S,5S)-5-(methoxycarbonylamino)-4-oxo-1,2,4,5,6,7-hexahydroazepino[3,2,1-hi]indole-2-carboxylic acid (1 g, 3.29 mmol), 4-bromobenzohydrazide (707 mg, 3.29 mmol), N,N′-diisopropylethylamine (1.15 mL, 6.57 mmol), and HATU (1.87 g, 4.93 mmol) in DMF (12 mL) was stayed at room temperature for 12 hours. The solution was diluted with EtOAc, washed with water and brine, dried over sodium sulfate, filtered, and evaporated to give methyl (2S,5S)-2-(2-(4-bromobenzoyl)hydrazinecarbonyl)-4-oxo-1,2,4,5,6,7-hexahydroazepino[3,2,1-hi]indol-5-ylcarbamate as a light yellow solid (quantitative): ESI-LRMS m/e calcd for C₂₂H₂₁BrN₄O₅ [M⁺] 502, found 503 [M+H⁺].

To a suspension of methyl (2S,5S)-2-(2-(4-bromobenzoyl)hydrazinecarbonyl)-4-oxo-1,2,4,5,6,7-hexahydroazepino[3,2,1-hi]indol-5-yl carbamate (1.65 g, 3.29 mmol), PPh₃ (1.29 g, 4.94 mmol) and N,N′-diisopropylethylamine (1.72 mL, 9.87 mmol) in acetonitrile (25 mL) at room temperature was added hexachloroethane (1.09 g, 4.61 mmol). The mixture was stirred at room temperature overnight. Solvent was then removed by evaporation. The residue was purified by column chromatography (0-60% EtOAc in hexane) to give methyl (25,55)-2-(5-(4-bromophenyl)-1,3,4-oxadiazol-2-yl)-4-oxo-1,2,4,5,6,7-hexahydroazepino[3,2,1-hi]indol-5-ylcarbamate (1.02 g, 64%): ESI-LRMS m/e calcd for C₂₂H₁₉BrN₄O₄ [M⁺]484, found 485 [M+H⁺].

To a microwave reactor vessel was added methyl (2S,5S)-2-(5-(4-bromophenyl)-1,3,4-oxadiazol-2-yl)-4-oxo-1,2,4,5,6,7-hexahydroazepino[3,2,1-hi]indol-5-ylcarbamate (200 mg, 0.41 mmol), methyl (S)-3-methyl-1-oxo-1-((S)-2-(2-(4-(4,4,5,5-tetramethyl-1,3,2-dioxaborolan-2-yl)phenyl)-1H-imidazol-5-yl)pyrrolidin-1-yl)butan-2-ylcarbamate (411 mg, 0.82 mmol), cesium carbonate (270 mg, 0.82 mmol), PdCl₂(dppf)-CH₂Cl₂ adduct (15.1 mg, 0.021 mmol), DMF (4 mL), and water (0.3 mL). The vessel was then sealed, de-gassed, flashed with nitrogen three times, and heated to 135° C. for 15 min. The mixture was filtered and the solution was purified by prep. HPLC (40-100% ACN containing 0.1% TEA in water containing 0.1% ammonia) to give {(2S,5S)-2-[5-(4′-{5-[(S)-1-((S)-2-Methoxycarbonylamino-3-methyl-butyryl)-pyrrolidin-2-yl]-1H-imidazol-2-yl}-biphenyl-4-yl)-[1,3,4]oxadiazol-2-yl]-4-oxo-1,2,4,5,6,7-hexahydro-azepino[3,2,1-hi]indol-5-yl}-carbamic acid methyl ester (8 mg, 2.5%): ESI-LRMS m/e calcd for C₄₂H₄₄N₈O₇ [M⁺]772, found 773.

### Example 63

### {(2S,5S)-2-[5-(4′-{2-[(R)-4-((R)-2-Methoxycarbonylamino-2-phenyl-acetyl)-morpholin-3-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester

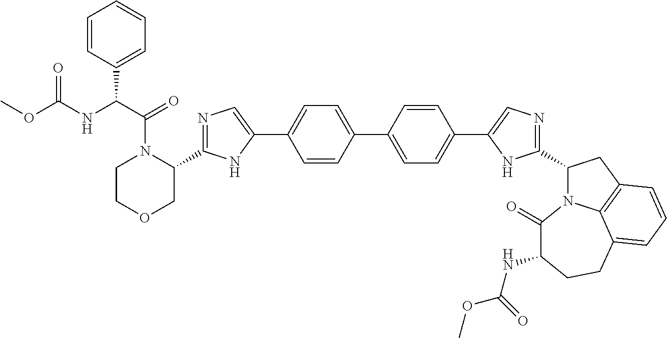

A mixture of (S)-4-(tert-butoxycarbonyl)morpholine-3-carboxylic acid (1 g, 4.36 mmol), HATU (2.49 g, 6.54 mmol), N,N′-diisopropylethylamine (2.29 mL, 13.1 mmol) in DMF (15 mL) was stirred at room temperature for 30 minutes, followed by addition of 2-amino-1-(4-bromophenyl)ethanone hydrochloride (1.1 g, 4.41 mmol). The mixture was stirred at room temperature over the weekend. The solution was diluted with EtOAc (200 mL), washed with water and brine, dried over sodium sulfate, filtered, and evaporated to offer an oily product, (S)-tert-butyl 3-(2-(4-bromophenyl)-2-oxoethylcarbamoyl)morpholine-4-carboxylate (quantitative): ESI-LRMS m/e calcd for C₁₈H₂₃BrN₂O₅ [M⁺] 424, found 425.

A mixture of (S)-tert-butyl 3-(2-(4-bromophenyl)-2-oxoethylcarbamoyl) morpholine-4-carboxylate (1.42 g, 3.32 mmol), ammonium acetate (5.12 g, 66.4 mmol), and xylene (100 mL) was heated to reflux overnight. The solvent was then removed by evaporation under reduced pressure. The residue was treated with EtOAc (250 mL). The mixture was filtered and evaporated. The residue was purified by column (0-50% EtOAc in hexane) to give (R)-tert-butyl 3-(5-(4-bromophenyl)-1H-imidazol-2-yl) morpholine-4-carboxylate (1.2 g, 88.5%): ESI-LRMS m/e calcd for C₁₈H₂₂BrN₃O₃ [M⁺] 409, found 410.

To a 20-mL microwave reaction vial was added (R)-tert-butyl 3-(5-(4-bromophenyl)-1H-imidazol-2-yl)morpholine-4-carboxylate (1.2 g, 2.94 mmol), 4,4,4′,4′,5,5,5′,5′-octamethyl-2,2′-bi(1,3,2-dioxaborolane) (2.24 g, 8.82 mmol), PdCl₂(dppf)-CH₂Cl₂ adduct (215 mg, 0.294 mmol), potassium acetate (865 mg, 8.82 mmol) and dioxane (14 mL). The vial was sealed, degassed, flashed with nitrogen three times and heated to 150° C. for 2 hours. The mixture was treated with EtOAc (300 mL) and water. The biphasic mixture was stirred with charcoal for an hour, followed by filtration through celite. The organic phase was separated, washed with brine, dried over sodium sulfate, filtered, and evaporated in vacuo. The residue was purified by column (0-10% MeOH in methylene chloride) to give (R)-tert-butyl 3-(5-(4-(4,4,5,5-tetramethyl-1,3,2-dioxaborolan-2-yl)phenyl)-1H-imidazol-2-yl) morpholine-4-carboxylate as a yellow solid (671 mg, 50.1%): ESI-LRMS m/e calcd for C₂₄H₃₄BN₃O₅ [M⁺] 455, found 456.

To a microwave reactor vessel was added methyl (2S,5S)-2-(5-(4-bromophenyl)-1H-imidazol-2-yl)-4-oxo-1,2,4,5,6,7-hexahydroazepino[3,2,1-hi]indol-5-ylcarbamate (85 mg, 0.18 mmol), (R)-tert-butyl 3-(5-(4-(4,4,5,5-tetramethyl-1,3,2-dioxaborolan-2-yl)phenyl)-1H-imidazol-2-yl)morpholine-4-carboxylate (96.5 mg, 0.21 mmol), sodium carbonate (37.4 mg, 0.35 mmol), PdCl2(dppf)-CH₂Cl₂ adduct (12.9 mg, 0.018 mmol), DMF (5 mL), and water (0.5 mL). The vessel was sealed, degassed, flashed with nitrogen, and heated at 135° C. for 15 min. The mixture was then treated with EtOAc (150 mL) and water (20 mL) followed by stirring with charcoal for 1 hour and filtration through celite. The organic layer was separated, washed with brine, dried over sodium sulfate, filtered, and evaporated. The residue was purified by prep HPLC (20-100% acetonitrile containing 0.1% TFA in water containing 0.1% TFA) to give (R)-tert-butyl 3-(5-(4′-(2-((2S,5S)-5-(methoxycarbonylamino)-4-oxo-1,2,4,5,6,7-hexahydroazepino[3,2,1-hi]indol-2-yl)-1H-imidazol-5-yl)biphenyl-4-yl)-1H-imidazol-2-yl)morpholine-4-carboxylate: %): ESI-LRMS m/e calcd for %): ESI-LRMS m/e calcd for C₄ᵢH₄₃N₇O₆ [M⁺] 729, found 730.

A solution of (R)-tert-butyl 3-(5-(4′-(2-((2S,5S)-5-(methoxycarbonylamino)-4-oxo-1,2,4,5,6,7-hexahydroazepino[3,2,1-hi]indol-2-yl)-1H-imidazol-5-yl)biphenyl-4-yl)-1H-imidazol-2-yl)morpholine-4-carboxylate (129 mg, 0.18 mmol) and 50% TFA in dichloromethane (10 mL) was stayed at room temperature for 2 hours. LC-MS showed starting material was completely converted to product. The solution was evaporated and the residue was purified by preparative HPLC (5-100% acetonitrile (with 0.1% TFA) in water (with 0.1% TFA)) to give methyl (2S,5S)-2-(5-(4′-(2-((R)-morpholin-3-yl)-1H-imidazol-5-yl)biphenyl-4-yl)-1H-imidazol-2-yl)-4-oxo-1,2,4,5,6,7-hexahydroazepino[3,2,1-hi]indol-5-ylcarbamate (16.7 mg, 15%): ESI-LRMS m/e calcd for %): ESI-LRMS m/e calcd for C₃₆H₃₅N₇O₄ [M⁺] 629, found 630.

A mixture of methyl (2S,5S)-2-(5-(4′-(2-((R)-morpholin-3-yl)-1H-imidazol-5-yl)biphenyl-4-yl)-1H-imidazol-2-yl)-4-oxo-1,2,4,5,6,7-hexahydroazepino[3,2,1-hi]indol-5-ylcarbamate (16 mg, 0.025 mmol), (R)-2-(methoxycarbonylamino)-2-phenylacetic acid (10.6 mg, 0.05 mmol), N,N′-diisopropylethylamine (16.4 mg, 0.13 mmol), HATU (19.3 mg, 0.51 mmol), and DMF (1 mL) was stayed at room temperature for 12 hrs. The solution was then separated by basic preparative HPLC (5-100% acetonitrile containing 0.1% TEA in water containing 0.1% ammonia) to give {(2S,5S)-2-[5-(4′-{2-[(R)-4-((R)-2-Methoxycarbonylamino-2-phenyl-acetyl)-morpholin-3-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester (10.1 mg, 48.4%): ESI-LRMS m/e calcd for C₄₆H₄₄N₈O₇ [M⁺] 820, found 821. ¹H NMR (400 MHz, DMSO-d₆) δ ppm 0.89 (m, 1H) 1.08 (m, 1H) 2.03-2.12 (m, 1H) 2.14-2.24 (m, 1H) 2.26 (m, 1H) 2.38 (m, 1H) 2.60 (m, 1H) 2.93-3.06 (m, 4H) 3.49 (s, 3H) 3.50 (s, 3H) 4.03-4.12 (m, 1H) 4.36 (d, 1H) 5.43 (d, 1H) 5.59-5.67 (m, 1H) 5.67-5.75 (m, 1H) 6.95 (t, 1H) 7.03 (d, 1H) 7.12 (d, 1H) 7.23-7.43 (m, 3H) 7.45-7.50 (m, 1H) 7.52-7.63 (m, 3H) 7.67-7.73 (m, 2H) 7.78 (d, 1H) 11.76 (s, 1H).

### Example 64

### {(2S,5S)-2-[5-(4′-{2-[(R)-4-((S)-2-Methoxycarbonylamino-3-methyl-butyryl)-morpholin-3-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester

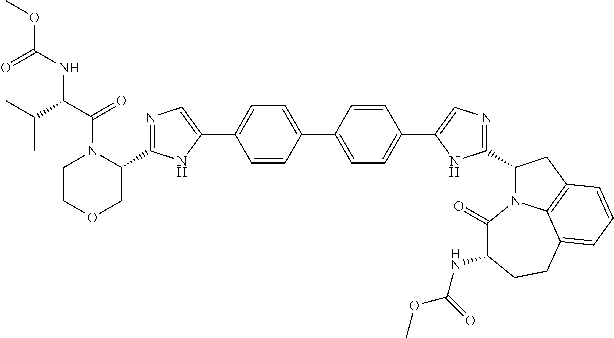

A mixture of methyl (2S,5S)-2-(5-(4′-(2-((R)-morpholin-3-yl)-1H-imidazol-5-yl)biphenyl-4-yl)-1H-imidazol-2-yl)-4-oxo-1,2,4,5,6,7-hexahydroazepino[3,2,1-hi]indol-5-ylcarbamate (131 mg, 0.21 mmol) (see Example 63), (S)-2-(methoxycarbonylamino)-3-methylbutanoic acid (73 mg, 0.42 mmol), N,N′-diisopropylethylamine (269 mg, 2.1 mmol), HATU (198 mg, 0.52 mmol) and DMF (2 mL) stayed at room temperature overnight. The solution was separated by prep HPLC using 10-100% acetonitrile containing 0.1% TFA in water containing 0.1% TFA as elute. The desired peak was concentrated and purified again via basic prep HPLC (20-100% acetonitrile containing 0.1% TEA in water containing 0.1% ammonia) to give {(2S,5S)-2-[5-(4′-{2-[(R)-4-((S)-2-Methoxycarbonylamino-3-methyl-butyryl)-morpholin-3-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester (20 mg, 12.2%): ESI-LRMS m/e calcd for C₄₃H₄₆N₈O₇ [M⁺] 786, found 787.

### Example 65

### {(2S,5S)-2-[5-(4′-{2-[(S)-3-((R)-2-Methoxycarbonylamino-2-phenyl-acetyl)-3-aza-bicyclo[3.1.0]hex-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester

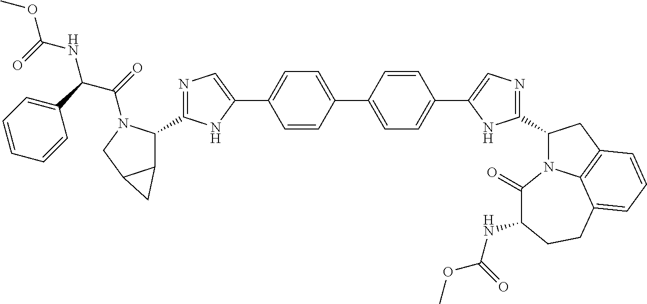

To a microwave reactor vessel was added methyl (2S,5S)-2-(5-(4-bromophenyl)-1H-imidazol-2-yl)-4-oxo-1,2,4,5,6,7-hexahydroazepino[3,2,1-hi]indol-5-ylcarbamate (305 mg, 0.63 mmol), tert-butyl 2-oxo-2-(4-(4,4,5,5-tetramethyl-1,3,2-dioxaborolan-2-yl)phenyl)ethylcarbamate (687 mg, 1.9 mmol), sodium carbonate (134 mg, 1.27 mmol), PdCl₂(dppf)-CH₂Cl₂ adduct (46.4 mg, 0.063 mmol), DMF (12 mL), and water (2 mL). The vessel was then sealed, degassed, flashed with nitrogen, and heated at 135° C. for 15 minutes via microwave. The mixture was then treated with EtOAc (150 mL) and water (20 mL) followed by stirring with charcoal for 1 hour and filtration through celite. The organic layer was separated, washed with brine, dried over sodium sulfate, filtered, and evaporated. The residue was purified by column (0-70% EtOAc in hexane) to give ((2S,5S)-2-{544′-(2-tert-Butoxycarbonylamino-acetyl)-biphenyl-4-yl]-1H-imidazol-2-yl}-4-oxo-1,2,4,5,6,7-hexahydro-azepino[3,2,1-hi]indol-5-yl)-carbamic acid methyl ester: ESI-LRMS m/e calcd for C₃₆H₃₇N₅O₆ [M⁺] 635, found 636.

((2S,5S)-2-{5-[4′-(2-tert-Butoxycarbonylamino-acetyl)-biphenyl-4-yl]-1H-imidazol-2-yl}-4-oxo-1,2,4,5,6,7-hexahydro-azepino[3,2,1-hi]indol-5-yl)-carbamic acid methyl ester (403 mg, 0.63 mmol) was treated with 50% TFA in dichloromethane (10 mL) at room temperature. The resulting solution was stayed at room temperature for 2 hours. LC-MS showed starting material was completely converted to product. The solution was evaporated and the residue was purified by preparative HPLC (5-100% ACN (with 0.1% TFA) in water (with 0.1% TFA)) to give methyl (2S,5S)-2-(5-(4′-(2-aminoacetyl)biphenyl-4-yl)-1H-imidazol-2-yl)-4-oxo-1,2,4,5,6,7-hexahydroazepino[3,2,1-hi]indol-5-ylcarbamate (33.8 mg. 10%): ESI-LRMS m/e calcd for C₃₁H₂₉N₅O₄ [M⁺] 535, found 536.

A solution of (S)-3-Aza-bicyclo [3.1.0] hexane-2,3-dicarboxylic acid 3-tert-butyl ester (28.7 mg, 0.13 mmol), methyl (2S,5S)-2-(5-(4′-(2-aminoacetyl)biphenyl-4-yl)-1H-imidazol-2-yl)-4-oxo-1,2,4,5,6,7-hexahydroazepino[3,2,1-hi]indol-5-ylcarbamate (33.8 mg, 0.063 mmol), N,N′-diisopropylethylamine (41 mg, 0.32 mmol), HATU (24 mg, 0.063 mmol) and DMF (3 mL) was stayed at room temperature overnight. The solution was diluted with EtOAc (100 mL), followed by washing with water (×2) and brine (×2), drying over sodium sulfate, filtration, and evaporation to give (S)-2-(2-{4′-[2-((2S,5S)-5-Methoxycarbonylamino-4-oxo-1,2,4,5,6,7-hexahydro-azepino[3,2,1-hi]indol-2-yl)-3H-imidazol-4-yl]-biphenyl-4-yl}-2-oxo-ethylcarbamoyl)-3-aza-bicyclo[3.1.0]hexane-3-carboxylic acid tert-butyl ester (29 mg, 61.7%): ESI-LRMS m/e calcd for C₄₂H₄₄N₆O₇ [M⁺] 744, found 745.

A mixture of (S)-2-(2-{4′-[2-((2S,5S)-5-Methoxycarbonylamino-4-oxo-1,2,4,5,6,7-hexahydro-azepino[3,2,1-hi]indol-2-yl)-3H-imidazol-4-yl]-biphenyl-4-yl}-2-oxo-ethylcarbamoyl)-3-aza-bicyclo[3.1.0]hexane-3-carboxylic acid tert-butyl ester (46.9 mg, 0.063 mmol), ammonium acetate (48.6 mg, 0.63 mmol), and xylene (10 mL) was heated at reflux for 24 hours. The solvent was then removed by evaporation in vacuo. The residue was treated with TFA (1 mL) for 1 hour, followed by evaporation in vacuo to remove excess TFA. The residue was purified by acidic prep. HPLC (5-100% acetonitrile containing 0.1% TFA in water containing 0.1% TFA) to give [(2S,5S)-2-(5-{4′-[(S)-2-(3-Aza-bicyclo[3.1.0]hex-2-yl)-3H-imidazol-4-yl]-biphenyl-4-yl}-1H-imidazol-2-yl)-4-oxo-1,2,4,5,6,7-hexahydro-azepino[3,2,1-hi]indol-5-yl]-carbamic acid methyl ester (5.1 mg, 12.9%): ESI-LRMS m/e calcd for C₃₇H₃₅N₇O₃ [M⁺] 625, found 626.

A mixture of [(2S,5S)-2-(5-{4′-[(S)-2-(3-Aza-bicyclo[3.1.0]hex-2-yl)-3H-imidazol-4-yl]-biphenyl-4-yl}-1H-imidazol-2-yl)-4-oxo-1,2,4,5,6,7-hexahydro-azepino[3,2,1-hi]indol-5-yl]-carbamic acid methyl ester (5.1 mg, 0.008 mmol), (R)-2-(methoxycarbonylamino)-2-phenylacetic acid (3.31 mg, 0.016 mmol), N,N′-diisopropylethylamine (10 mg, 0.08 mmol), HATU (7.5 mg, 0.02 mmol) and DMF (2 mL) was stirred at room temperature overnight. The solution was purified directly through HPLC (0-100 acetonitrile containing 0.1% TFA in water containing 0.1% TFA). The desired peak was collected and lyophilyzed to offer {(2S,5S)-2-[5-(4′-{2-[(S)-3-((R)-2-Methoxycarbonylamino-2-phenyl-acetyl)-3-aza-bicyclo[3.1.0]hex-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester (3 mg, 46.4%): ESI-LRMS m/e calcd for C₄₇H₄₄N₈O₆ [M⁺] 816, found 817.

### Intermediate 32

### (S)-3-methyl-1-oxo-1-((S)-2-(5-(4-(4,4,5,5-tetramethyl-1,3,2-dioxaborolan-2-yl)phenyl)-1H-imidazol-2-yl)pyrrolidin-1-yl)butan-2-yl-carbamate

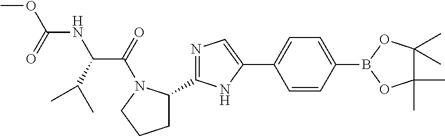

N,N-Diisopropyethylamine (3.2 g, 24.7 mmol) was added dropwise at room temperature to a heterogeneous mixture of 2-amino-1-(4-bromophenyl)ethanone hydrochloride (2.0 g, 7.98 mmol), (S)-1-(tert-butoxycarbonyl)pyrrolidine-2-carboxylic acid (1.72 g, 7.98 mmol), HATU (3.04 g, 7.98 mmol) and DMF (20 mL). After the addition was complete the reaction mixture was stirred at room temperature for 2 h. The reaction mixture was diluted with ethyl acetate and washed with water, 1N hydrochloric acid, a saturated sodium bicarbonate solution, a saturated sodium chloride solution and dried over magnesium sulfate, filtered and concentrated. The crude product obtained was purified by ISCO flash chromatography (Teledyne Isco RediSep Flash Column 40 g; (0% to 100% ethyl acetate/hexane) to afford, (S)-tert-butyl 2-(2-(4-bromophenyl)-2-oxoethylcarbamoyl)pyrrolidine-1-carboxylate as a white solid, (2.50 g, 76%): ESI-LRMS m/e calcd for C₁₈H₂₃BrN₂O₄ [M⁺] 410, found 411 [M+H⁺].

A mixture of (S)-tert-butyl 2-(2-(4-bromophenyl)-2-oxoethylcarbamoyl)pyrrolidine-1-carboxylate (2.50 g, 6.08 mmol) and ammonium acetate (2.34 g, 30.4 mmol) in xylenes (10 mL) was heated in a sealed tube at 140° C. for 4 h. The reaction was then cooled to room temperature and diluted with ethyl acetate. The organic fraction was washed with a saturated sodium bicarbonate solution, a saturated sodium chloride solution and dried over magnesium sulfate, filtered and concentrated. The crude product obtained was purified by ISCO flash chromatography (Teledyne Isco RediSep Flash Column 40 g; (00% to 100% ethyl acetate/hexane) to afford, (S)-tert-butyl 2-(5-(4-bromophenyl)-1H-imidazol-2-yl)pyrrolidine-1-carboxylate as a yellow solid, (1.77 g, 74%): ESI-LRMS m/e calcd for C₁₈H₂₂BrN₃O₂ [M⁺] 392, found 393 [M+H⁺].

A mixture of (S)-tert-butyl 2-(5-(4-bromophenyl)-1H-imidazol-2-yl)pyrrolidine-1-carboxylate (2.45 g, 6.25 mmol,) and 4.0 M HCl/dioxane solution (15 mL) in methanol (30 mL) was stirred at room temperature for 4 h. Concentrate the reaction in vacuo. The crude mixture was made basic with saturated sodium bicarbonate solution and was extracted from the aqueous layer with ethyl acetate. The combined organic fractions were washed with a saturated sodium chloride solution and dried over magnesium sulfate, filtered and concentrated to afford, (S)-5-(4-bromophenyl)-2-(pyrrolidin-2-yl)-1H-imidazole hydrochloride as an orange solid, (1.80 g, 98%): ESI-LRMS m/e calcd for C₁₃H₁₄BrN₃ HCl [M⁺]328.5, found 293 [M+H⁺] (free base).

N,N-Diisopropyethylamine (2.92 g, 22.6 mmol) was added dropwise at room temperature to a heterogeneous mixture of (S)-5-(4-bromophenyl)-2-(pyrrolidin-2-yl)-1H-imidazole hydrochloride (2.2 g, 7.53 mmol), (S)-2-(methoxycarbonylamino)-3-methylbutanoic acid (1.32 g, 7.53 mmol), HATU (2.86 g, 7.53 mmol) and DMF (20 mL). After the addition was complete the reaction mixture was stirred at room temperature for 5 h. The reaction mixture was diluted with ethyl acetate and washed with water, 1N hydrochloric acid, a saturated sodium bicarbonate solution, a saturated sodium chloride solution and dried over magnesium sulfate, filtered and concentrated. The crude product obtained was purified by ISCO flash chromatography (Teledyne Isco RediSep Flash Column 80 g; (0% to 100% ethyl acetate/hexane) to afford, ((S)-1-{(S)-2-[5-(4-bromo-phenyl)-1H-imidazol-2-yl]-pyrrolidine-1-carbonyl}-2-methyl-propyl)-carbamic acid methyl ester as a yellow solid, (2.20 g, 765%): ESI-LRMS m/e calcd for C₂₀H₂₅BrN₄O₃ [M⁺]449, found 450 [M+H⁺].

1,1′-bis(diphenylphosphino)ferrocene-palladium(II)dichloride dichloromethane complex (436 mg, 0.53 mmol) was added to a sealed tube containing a mixture of ((S)-1-{(S)-2-[5-(4-bromo-phenyl)-1H-imidazol-2-yl]-pyrrolidine-1-carbonyl}-2-methyl-propyl)-carbamic acid methyl ester (2.40 mg, 5.34 mmol), 4,4,4′,4′,5,5,5′,5′-octamethyl-2,2′-bi(1,3,2-dioxaborolane) (4.07 g, 16.0 mmol), potassium acetate (2.62 mg, 26.7 mmol) and 1,4-dioxane (40 ml). The vessel was purged with nitrogen, capped and heated with an oil bath at 80° C. overnight. Cool the reaction to room temperature and filter through celite. Concentrate the reaction in vacuo. The crude mixture was diluted with methylene chloride and washed with water, a saturated sodium chloride solution and dried over magnesium sulfate, filtered and concentrated. The crude product obtained was purified by ISCO flash chromatography (Teledyne Isco RediSep Flash Column 40 g; (50% to 100% ethyl acetate/hexane) to afford, methyl (S)-3-methyl-1-oxo-1-((S)-2-(5-(4-(4,4,5,5-tetramethyl-1,3,2-dioxaborolan-2-yl)phenyl)-1H-imidazol-2-yl)pyrrolidin-1-yl)butan-2-yl-carbamate as a brown solid, (1.78 g, 67%): ESI-LRMS m/e calcd for C₂₆H₃₇BN₄O₅ [M⁺] 496, found 497 [M+H⁺].

### Intermediate 33

### [(2S,5S)-2-(6-Bromo-1H-benzoimidazol-2-yl)-4-oxo-1,2,4,5,6,7-hexahydro-azepino[3,2,1-hi]indol-5-yl]-carbamic acid methyl ester

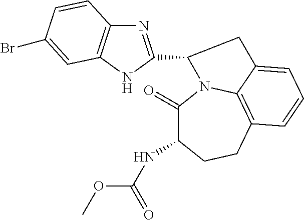

(2S,5S)-Fmoc-5-amino-1,2,4,5,6,7-hexahydro-azepino[3,2,1-HI]indole-4-one-2-carboxylic acid (from a commercial source, 0.8 g, 1.71 mmol), HATU (714 mg, 1.88 mmol) and 4-bromo-1,2-benzenediamine (319 mg, 1.71 mmol) were combined with DMF (20 ml) in a round bottomed flask. N,N-diisopropylethylamine (441 mg, 585 μA, 3.42 mmol, Eq: 2) was added, the resulting mixture was stirred at room temperature for 2 h. The reaction mixture was poured into water and extracted with EtOAc, The organic layers were washed with brine and dried over sodium sulfate and concentrated in vacuo. The crude material was purified by flash chromatography (silica gel, 50% to 80% EtOAc in Hexane) to give a brown solid as compound 2 (a mixture of regioisomers by LCMS) which is used in the next step.

A mixture of (9H-fluoren-9-yl)methyl (2S,5S)-2-(2-amino-4-bromophenylcarbamoyl)-4-oxo-1,2,4,5,6,7-hexahydroazepino[3,2,1-hi]indol-5-yl-carbamate and its regioisomer (1.09 g, 1.71 mmol) was combined with acetic acid (15 ml) to give a brown suspension. The reaction mixture was heated at 90° C. for one hour, then poured into ice water. The pH was adjusted to 6-7 with 3 N NaOH aqueous solution (about 90 ml), collected the solid by filtration, washed with water, the solid was dissolved in dichloromethane, the organic solution were dried over Na₂SO₄ and concentrated in vacuo, brown solid obtained. The crude material was purified by flash chromatography (silica gel, 20% to 50% EtOAc in hexanes) to give compound 3 as yellow foam (0.877 g, 82% in 2 steps). M+1=619/621.

In a round-bottomed flask, a compound of (9H-fluoren-9-yl)methyl (2S,5S)-2-(6-bromo-1H-benzo[d]imidazol-2-yl)-4-oxo-1,2,4,5,6,7-hexahydroazepino[3,2,1-hi]indol-5-yl-carbamate (0.875 g, 1.41 mmol, Eq: 1.00), were combined with 20% piperidine in DMF (10 ml) to give a yellow solution. The reaction mixture was stirred at RT for 3 hours. The reaction mixture was poured into water, and extracted with EtOAc, The organic layers were washed with brine and dried over Na₂SO₄ and concentrated in vacuo. The crude material was purified by silica gel plug (silica gel, 7% MeOH in DCM/conc. NH₄OH) to give compound 4 as yellow foam (0.57 g, 100%). M+1=397/399.

In a round-bottomed flask, a compound of (2S,5S)-5-amino-2-(6-bromo-1H-benzo[d]imidazol-2-yl)-1,2,6,7-tetrahydroazepino[3,2,1-hi]indol-4(5H)-one (0.57 g, 1.43 mmol, Eq: 1.00), was combined with dichloromethane (20 ml) to give a yellow solution. Pyridine (227 mg, 232 μl, 2.87 mmol, Eq: 2) and methyl chloroformate (155 mg, 1.65 mmol, Eq: 1.15) were added at 0° C. The mixture was stirred in ice bath about 2 hours, TLC showed there was still some SM left, extra 0.1 ml of methyl chloroformate added, stirred in ice bath about one hour, LC-Mass showed the formation of compound 5, the reaction mixture was poured into water, and extracted with dichloromethane. The organic layers were washed with brine and dried over Na₂SO₄ and concentrated in vacuo, the residue was used for the next step.

The crude material was dissolved in the mixture of THF (10 ml) and conc. NH₄OH aq solution (2 ml), and stirred at RT for 2 hours. The reaction mixture was poured into water and extracted with EtOAc, the organic layers were washed with brine and dried over Na₂SO₄ and concentrated in vacuo. The crude material was purified by silica gel plug (5% MeOH in DCM/Conc. NH₄OH) to give compound 6 as pale yellow foam (0.65 g, 99% in 2 steps). M+1=455/457.

### Intermediate 34

### Methyl (S)-1-((S)-4,4-difluoro-2-(5-(4-(4,4,5,5-tetramethyl-1,3,2-dioxaborolan-2-yl)phenyl)-1H-imidazol-2-yl)pyrrolidin-1-yl)-3-methyl-1-oxobutan-2-yl-carbamate

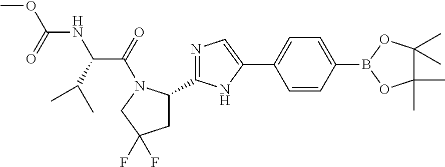

In a 50 mL pear-shaped flask, (S)-1-(tert-butoxycarbonyl)-4,4-difluoropyrrolidine-2-carboxylic acid (1 g, 3.98 mmol, Eq: 1.00) and HATU (1.51 g, 3.98 mmol, Eq: 1.00) were combined with DMF (15 ml) to give a colorless solution and stirred at rt for 10 min. 2-amino-1-(4-bromophenyl)ethanone HCl (995 mg, 3.98 mmol, Eq: 1.00) was added followed by drop wise addition of DIPEA (1.54 g, 2.09 ml, 11.9 mmol, Eq: 3). The suspension became a orange solution once the addition of the amine was completed. It was stirred at RT for 1 hr and diluted with brine (100 ml) and H₂O (50 ml). The ppt was filtered and washed with H₂O and dried to afford (S)-tert-butyl 2-(2-(4-bromophenyl)-2-oxoethylcarbamoyl)-4,4-difluoropyrrolidine-1-carboxylate as a light yellow solid. (1.8 g, >96%): ESI-LRMS m/e calcd for C₁₈H₂₁BrF₂N₂O₄ [M⁺] 447, found 448 [M+H⁺].

In a 50 mL seal tube, (S)-tert-butyl 2-(2-(4-bromophenyl)-2-oxoethylcarbamoyl)-4,4-difluoropyrrolidine-1-carboxylate (1.8 g, 4.02 mmol, Eq: 1.00) and acetic acid, ammonia salt (1.55 g, 20.1 mmol, Eq: 5.00) were combined with xylene (16 ml). The reaction mixture was heated to 140° C. and stirred for 4 hr. The reaction mixture was cooled to RT and diluted with EtOAc (50 ml). It was washed with water and brine, dried with MgSO₄ concentrated and purified on column. CH₂Cl₂, 30%, 50%, 80% EtOAc/CH₂Cl₂ to afford (S)-tert-butyl 24544-bromophenyl)-1H-imidazol-2-yl)-4,4-difluoropyrrolidine-1-carboxylate as a light yellow solid. 2-yl)pyrrolidine-1-carboxylate as a yellow solid, (1.77 g, 74%): ESI-LRMS m/e calcd for C₁₈H₂₂BrF₂N₃O₂ [M⁺] 428, found 429 [M+H⁺].

In a 10 mL pear-shaped flask, (S)-tert-butyl 2-(5-(4-bromophenyl)-1H-imidazol-2-yl)-4,4-difluoropyrrolidine-1-carboxylate (1.0 g, 2.33 mmol, Eq: 1.00) was combined with CH₂Cl₂ (6 ml) to give a light yellow solution. TFA (2.96 g, 2 mL, 26.0 mmol, Eq: 11.1) was added and stirred at RT for 2 hr. It was concentrated in vacuo to afford (S)-5-(4-bromophenyl)-2-(4,4-difluoropyrrolidin-2-yl)-1H-imidazole as a viscous oil and used without further purification.

In a 20 mL pear-shaped flask, (S)-2-(methoxycarbonylamino)-3-methylbutanoic acid (416 mg, 2.38 mmol, Eq: 1.3) and HATU (695 mg, 1.83 mmol, Eq: 1.00) were combined with DMF (10 ml) to give a colorless solution. (S)-5-(4-bromophenyl)-2-(4,4-difluoropyrrolidin-2-yl)-1H-imidazole (600 mg, 1.83 mmol, Eq: 1.00) in 2 ml of DMF was added and followed by drop wise addition of .DIPEA (1.18 g, 1.6 ml, 9.14 mmol, Eq: 5). I was stirred at RT for 1 hr then poured into ice/water. It was extracted with EtOAc (2×30 ml), washed with brine and water. The organic was dried over MgSO₄, concentrated and purified on column. CH₂Cl₂, 1%, 2%, 5% MeOH/CH₂Cl₂ to afford methyl (S)-1-((S)-2-(5-(4-bromophenyl)-1H-imidazol-2-yl)-4,4-difluoropyrrolidin-1-yl)-3-methyl-1-oxobutan-2-yl-carbamate as an orange foaming solid. (650 mg, 73%): ESI-LRMS m/e calcd for C₂₀H₂₅BrF₂N₄O₃ [M⁺] 485, found 486 [M+H⁺].

In a 20 mL seal tube, methyl (S)-1-((S)-2-(5-(4-bromophenyl)-1H-imidazol-2-yl)-4,4-difluoropyrrolidin-1-yl)-3-methyl-1-oxobutan-2-yl-carbamate (300 mg, 618 μmol, Eq: 1.00), 4,4,4′,4′,5,5,5′,5′-octamethyl-2,2′-bi(1,3,2-dioxaborolane) (471 mg, 1.85 mmol, Eq: 3.0) and potassium acetate (303 mg, 3.09 mmol, Eq: 5.0) were combined with 1,4-dioxane (6 ml) to give a light yellow suspension. It was degassed for 20 min and 1,1′-BIS(DIPHENYLPHOSPHINO)FERROCENE-PALLADIUM(II)DICHLORIDE DICHLOROMETHANE COMPLEX (50.5 mg, 61.8 μmol, Eq: 0.10) was added, flushed with N₂, sealed and stirred at 80° C. for 16 hr. It was cooled to rt and diluted with EtOAc (2×20 ml). The organics was washed with brine and water, dried with MgSO₄ and purified on column. CH₂Cl₂, 1%, 2%, 3% to 5% MeOH/CH₂Cl₂ to afford Methyl (S)-1-((S)-4,4-difluoro-2-(5-(4-(4,4,5,5-tetramethyl-1,3,2-dioxaborolan-2-yl)phenyl)-1H-imidazol-2-yl)pyrrolidin-1-yl)-3-methyl-1-oxobutan-2-yl-carbamate as an orange solid. (300 mg, 87%): ESI-LRMS m/e calcd for C₂₆H₃₇BF₂N₄O₅ [M⁺] 532, found 533 [M+H⁺].

### Example 66

### {(2S,5S)-2-[5-(4′-{2-[(S)-1-((S)-2-Methoxycarbonylamino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester

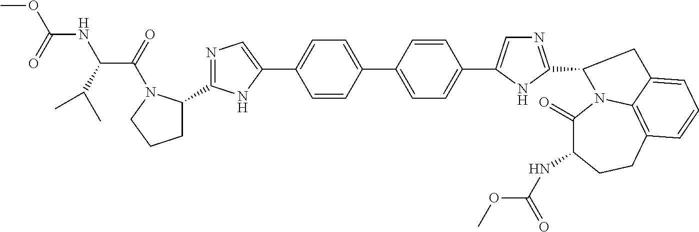

N,N′ diisopropylethylamine (414 mg, 3.20 mmol) was added dropwise at room temperature to a heterogeneous mixture of (2S,5S)-FMOC-5-amino-1,2,4,5,6,7-hexahydroazepino[3,2,1-Hi]indol-4-one-2-carboxylic acid (500 mg, 1.07 mmol), 2-amino-1-(4-bromo-phenyl)-ethanone hydrochloride (267 mg, 1.07 mmol), HATU (406 mg, 1.07 mmol) and DMF (17 mL). After addition was complete the reaction was stirred at room temperature overnight. The reaction mixture was diluted with ethyl acetate and washed with water, 1N hydrochloric acid, a saturated sodium bicarbonate solution, a saturated sodium chloride solution and dried over magnesium sulfate, filtered and concentrated to afford, {(2S,5S)-2-[2-(4-bromo-phenyl)-2-oxo-ethylcarbamoyl]-4-oxo-1,2,4,5,6,7-hexahydro-azepino[3,2,1-hi]indol-5-yl]carbamic acid (9H-fluoren-9-yl)methyl ester as a light yellow solid, (602 mg, 85%): ESI-LRMS m/e calcd for C₃₆H₃₀BrN₃O₅ [M⁺] 664, found 665 [M+H⁺].

To a stirred mixture of {(2S,5S)-2-[2-(4-bromo-phenyl)-2-oxo-ethylcarbamoyl]-4-oxo-1,2,4,5,6,7-hexahydro-azepino[3,2,1-hi]indol-5-yl]carbamic acid (9H-fluoren-9-yl)methyl ester (600 mg, 0.90 mmol) dissolved in DMF (10 mL) was added piperidine (2 mL). The mixture was stirred at room temperature for 1 h and then concentrated in vacuo to afford, (2S,5S)-5-amino-4-oxo-1,2,4,5,6,7-hexahydro-azepino[3,2,1-hi]indo le-2-carboxylic acid [2-(4-bromo-phenyl)-2-oxo-ethyl]-amide as a yellow powder, (400 mg, 100%): ESI-LRMS m/e calcd for C₂₁H₂₀BrN₃O₃ [M⁺] 442, found 443 [M+H⁺].

To an iced cooled solution of (2S,5S)-5-amino-4-oxo-1,2,4,5,6,7-hexahydro-azepino[3,2,1-hi]indole-2-carboxylic acid [2-(4-bromo-phenyl)-2-oxo-ethyl]-amide (400 mg, 0.90 mmol) dissolved in DMF (12 mL) was added sodium carbonate (115 mg, 1.09 mmol) and methyl chloroformate (94 mg, 1.00 mmol). After the addition was complete the ice bath was removed and the reaction stirred at room temperature for 1 h. The reaction mixture was diluted with ethyl acetate and washed with water, a saturated sodium chloride solution and dried over magnesium sulfate, filtered and concentrated. The crude product obtained was purified by ISCO flash chromatography (Teledyne Isco RediSep Flash Column 40 g; (0% to 100% ethyl acetate/hexane) to afford, {(2S,5S)-2-[2-(4-bromo-phenyl)-2-oxo-ethylcarbamoyl]-4-oxo-1,2,4,5,6,7-hexahydro-azepino[3,2,1-hi]indol-5-yl}-carbamic acid methyl ester as a white solid, (175 mg, 39%): ESI-LRMS m/e calcd for C₂₃H₂₃BrN₃O₅ [M⁺] 500, found 501 [M+H⁺].

A mixture of {(2S,5S)-2-[2-(4-bromo-phenyl)-2-oxo-ethylcarbamoyl]-4-oxo-1,2,4,5,6,7-hexahydro-azepino[3,2,1-hi]indol-5-yl}-carbamic acid methyl ester (174 mg, 0.35 mmol) and ammonium acetate (134 mg, 1.74 mmol) in xylenes (10 mL) was heated in a sealed tube at 140° C. for 4 h. The reaction was then cooled to room temperature and diluted with ethyl acetate. The organic fraction was washed with a saturated sodium bicarbonate solution, a saturated sodium chloride solution and dried over magnesium sulfate, filtered and concentrated. The crude product obtained was purified by ISCO flash chromatography (Teledyne Isco RediSep Flash Column 40 g; (30% to 100% ethyl acetate/hexane) to afford, {(2S,5S)-2-[5-(4-bromo-phenyl)-1H-imidazol-2-yl]-4-oxo-1,2,4,5,6,7-hexahydro-azepino[3,2,1-hi]indol-5-yl}-carbamic acid methyl ester as a white solid, (175 mg, 39%): ESI-LRMS m/e calcd for C₂₃H₂₁BrN₄O₃ [M⁺] 481, found 482 [M+H⁺].

In a sealed tube tetrakis(triphenylphosphine)palladium(0) (16 mg, 0.02 mmol) was added to a mixture of methyl (S)-3-methyl-1-oxo-1-((S)-2-(5-(4-(4,4,5,5-tetramethyl-1,3,2-dioxaborolan-2-yl)phenyl)-1H-imidazol-2-yl)pyrrolidin-1-yl)butan-2-yl-carbamate (69 mg, 0.14 mmol), {(2S,5S)-2-[5-(4-bromo-phenyl)-1H-imidazol-2-yl]-4-oxo-1,2,4,5,6,7-hexahydro-azepino[3,2,1-hi]indol-5-yl}-carbamic acid methyl ester (Intermediate 32) (67 mg, 0.14 mmol) and sodium bicarbonate (35 mg, 0.41 mmol) in 1,2-dimethoxyethane (6 mL) and water (1 mL). The reaction mixture was flushed with nitrogen, capped and heated to 80° C. for 16 h. The reaction mixture was concentrated and partitioned between 20% methanol/methylene chloride and water and the aqueous phase extracted with 20% methanol/methylene chloride. The combined organic phases were washed with a saturated sodium chloride solution and dried over magnesium sulfate, filtered and concentrated. The crude product obtained was purified by reverse phase HPLC using a 50 g Polaris C18A column eluting with acetonitrile/water (30% to 100%) to afford, {(2S,5S)-2-[5-(4′-{2-[(S)-1-((S)-2-methoxycarbonylamino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester acid as a white solid, (31 mg, 29%):

ESI-LRMS m/e calcd for C₄₃H₄₆N₈O₆ [M⁺] 770, found 771 [M+H⁺]; ¹H NMR (400 MHz, DMSO-d₆) d ppm 0.72-1.00 (m, 6H) 1.24 (br. s., 1H) 1.80-2.35 (m, 6H) 2.99-3.14 (m, 2H) 3.33 (s, 1H) 3.35-3.44 (m, 1H) 3.55 (d, J=6.27 Hz, 6H) 3.81 (br. s., 1H) 4.01-4.20 (m, 1H) 5.08 (br. s., 1H) 5.77 (d, J=9.79 Hz, 1H) 6.96-7.37 (m, 4H) 6.97-7.05 (m, 1H) 7.21-7.36 (m, 1H) 7.40-7.89 (m, 9H) 11.56-11.92 (m, 2H)

### Example 67

### {(2S,5S)-2-[5-(4′-{2-[(1S,9S)-9-methoxycarbonylamino-6,10-dioxo-octahydro-pyridazino[1,2-a][1,2]diazepin-1-yl])-3H-imidazol-4-yl]-biphenyl-4-yl)-1H-imidazol-2-yl]-4-oxo-1,2,4,5,6,7-hexahydro-azepino[3,2,1-hi]indol-5-yl}-carbamic acid methyl ester

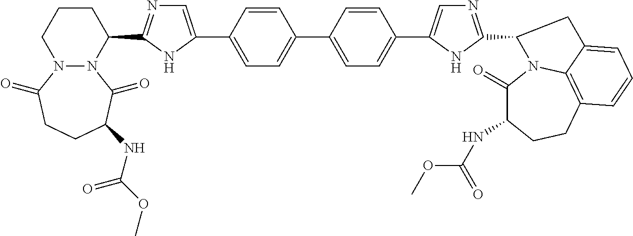

To a solution of (1S,9S)-tert-butyl 9-(1,3-dioxoisoindolin-2-yl)-6,10-dioxooctahydro-1H-pyridazino[1,2-a][1,2]diazepine-1-carboxylate (2.00 g, 4.68 mmol) in ethanol (10 mL) was added hydrazine (180 mg, 5.61 mmol). The reaction was stirred at room temperature for 3 h. The ethanol and excess hydrazine were concentrated in vacuo and the residue co-evaporated with ethanol to afford, (1S,9S)-tert-butyl 9-amino-6,10-dioxooctahydro-1H-pyridazino[1,2-a][1,2]diazepine-1-carboxylate as a white powder, (1.63 g, 100%): ESI-LRMS m/e calcd for C₁₄H₂₃N₃O₄[M⁺] 297, found 298 [M+H⁺].

To an ice-cooled solution of (1S,9S)-tert-butyl 9-amino-6,10-dioxooctahydro-1H-pyridazino[1,2-a][1,2]diazepine-1-carboxylate (1.50 g, 5.04 mmol) in DMF (15 mL) was added sodium carbonate (642 mg, 6.05 mmol) followed by methyl chloroformate (524 mg, 5.55 mmol). After the addition was complete the ice bath was removed and the reaction stirred at room temperature for 2 h. The reaction mixture was diluted with ethyl acetate and washed with water 2N hydrochloric acid, a saturated sodium chloride solution and dried over magnesium sulfate, filtered and concentrated to afford, (1S,9S)-tert-butyl 9-(methoxycarbonylamino)-6,10-dioxooctahydro-1H-pyridazino[1,2-a][1,2]diazepine-1-carboxylate as a white solid, (1.28 g, 71%): ESI-LRMS m/e calcd for C₁₆H₂₅N₃O₆[M⁺] 355, found 356 [M+H⁺].

To a solution of (1S,9S)-tert-butyl 9-(methoxycarbonylamino)-6,10-dioxooctahydro-1H-pyridazino[1,2-a][1,2]diazepine-1-carboxylate (1.25 g, 3.52 mmol) dissolved into methylene chloride (10 mL) was added trifluoroacetic acid (10 mL). The reaction was stirred at room temperature for 1 h and concentrated in vacuo. Toluene (5 mL) was added and the reaction concentrated in vacuo to afford (1S,9S)-9-(methoxycarbonylamino)-6,10-dioxooctahydro-1H-pyridazino[1,2-a][1,2]diazepine-1-carboxylic acid as a white solid, (587 mg, 68%): ESI-LRMS m/e calcd for C₁₂H₁₇N₃O₆[M⁺] 299, found 300 [M+H⁺].

N,N′ diisopropylethylamine (680 mg, 5.26 mmol) was added dropwise at room temperature to a heterogeneous mixture of (1S,9S)-9-(methoxycarbonylamino)-6,10-dioxooctahydro-1H-pyridazino[1,2-a][1,2]diazepine-1-carboxylic acid (525 mg, 1.75 mmol), 2-amino-1-(4-bromo-phenyl)-ethanone hydrochloride (439 mg, 1.75 mmol), HATU (667 mg, 1.75 mmol) and DMF (10 mL). After addition was complete the reaction was stirred at room temperature for 4 h. The reaction mixture was diluted with ethyl acetate and washed with water, 1N hydrochloric acid, a saturated sodium bicarbonate solution, a saturated sodium chloride solution and dried over magnesium sulfate, filtered and concentrated. The crude product obtained was purified by ISCO flash chromatography (Teledyne Isco RediSep Flash Column 40 g; (30% to 100% ethyl acetate/hexane) to afford, methyl (4S,7S)-4-(2-(4-bromophenyl)-2-oxoethylcarbamoyl)-6,10-dioxooctahydro-1H-pyridazino[1,2-a][1,2]diazepin-7-yl-carbamate as a light yellow solid, (587 mg, 68%): ESI-LRMS m/e calcd for C₂₀H₂₃BrN₄O₆ [M⁺] 495, found 496 [M+H⁺].

A mixture of methyl (4S,7S)-4-(2-(4-bromophenyl)-2-oxoethylcarbamoyl)-6,10-dioxooctahydro-1H-pyridazino[1,2-a][1,2]diazepin-7-yl-carbamate (500 mg, 1.01 mmol) and ammonium acetate (389 mg, 5.05 mmol) in xylenes (10 mL) was heated in a sealed tube at 140° C. for 4 h. The reaction was then cooled to room temperature and diluted with ethyl acetate. The organic fraction was washed with a saturated sodium bicarbonate solution, a saturated sodium chloride solution and dried over magnesium sulfate, filtered and concentrated. The crude product obtained was purified by ISCO flash chromatography (Teledyne Isco RediSep Flash Column 40 g; (30% to 100% ethyl acetate/hexane) to afford, methyl (4S,7S)-4-(5-(4-bromophenyl)-1H-imidazol-2-yl)-6,10-dioxooctahydro-1H-pyridazino[1,2-a][1,2]diazepin-7-yl-carbamate as a yellow solid, (435 mg, 91%): ESI-LRMS m/e calcd for C₂₀H₂₂BrN₅O₄ [M⁺] 476, found 477 [M+H⁺].

1,1′-bis(diphenylphosphino)ferrocene-palladium(II)dichloride dichloromethane complex (159 mg, 0.20 mmol) was added to a sealed tube containing a mixture of methyl (4S,7S)-4-(5-(4-bromophenyl)-1H-imidazol-2-yl)-6,10-dioxooctahydro-1H-pyridazino[1,2-a][1,2]diazepin-7-yl-carbamate (Intermediate 32) (925 mg, 1.94 mmol), 4,4,4′,4′,5,5,5′,5′-octamethyl-2,2′-bi(1,3,2-dioxaborolane) (1.48 g, 5.83 mmol), potassium acetate (953 mg, 9.71 mmol) and 1,4-dioxane (40 ml). The vessel was purged with nitrogen, capped and heated with an oil bath at 80° C. overnight. Cool the reaction to room temperature and filter through celite. Concentrate the reaction in vacuo. The crude mixture was diluted with methylene chloride and washed with water, a saturated sodium chloride solution and dried over magnesium sulfate, filtered and concentrated. The crude product obtained was purified by ISCO flash chromatography (Teledyne Isco RediSep Flash Column 40 g; (50% to 100% ethyl acetate/hexane) to afford, methyl (4S,7S)-6,10-dioxo-4-(5-(4-(4,4,5,5-tetramethyl-1,3,2-dioxaborolan-2-yl)phenyl)-1H-imidazol-2-yl)octahydro-1H-pyridazino[1,2-a][1,2]diazepin-7-yl-carbamate as a brown solid, (358 mg, 35%): ESI-LRMS m/e calcd for C₂₆H₃₄BN₅O₆ [M⁺] 523, found 524 [M+H⁺].

In a sealed tube 1,1′-bis(diphenylphosphino)ferrocene-palladium(II)dichloride (27 mg, 0.37 mmol) was added to a mixture of methyl (4S,7S)-6,10-dioxo-4-(5-(4-(4,4,5,5-tetramethyl-1,3,2-dioxaborolan-2-yl)phenyl)-1H-imidazol-2-yl)octahydro-1H-pyridazino[1,2-a][1,2]diazepin-7-yl-carbamate (192 mg, 0.37 mmol), {(2S,5S)-2-[5-(4-bromo-phenyl)-1H-imidazol-2-yl]-4-oxo-1,2,4,5,6,7-hexahydro-azepino[3,2,1-hi]indol-5-yl}-carbamic acid methyl ester (177 mg, 0.37 mmol) and sodium bicarbonate (93 mg, 0.1.10 mmol) in 1,2-dimethoxyethane (6 mL) and water (1 mL). The reaction mixture was flushed with nitrogen, capped and heated to 80° C. for 16 h. The reaction mixture was concentrated and partitioned between 20% methanol/methylene chloride and water and the aqueous phase extracted with 20% methanol/methylene chloride. The combined organic phases were washed with a saturated sodium chloride solution and dried over magnesium sulfate, filtered and concentrated. The crude product obtained was purified by reverse phase HPLC using a 50 g Polaris C18A column eluting with acetonitrile/water (30% to 100%) to afford, {(2S,5S)-2-[5-(4′-{2-[(1S,9S)-9-methoxycarbonylamino-6,10-dioxo-octahydro-pyridazino[1,2-a][1,2]diazepin-1-yl])-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester as an off-white solid, (61 mg, 21%): ESI-LRMS m/e calcd for C₄₃H₄₃N₉O₇ [M⁺] 797, found 798 [M+H⁺]; ¹H NMR (300 MHz, DMSO-d₆) δ ppm 1.21-1.32 (m, 2H) 1.59-1.70 (m, 1H) 1.80-2.38 (m, 8H) 2.82-2.94 (m, 1H) 3.03-3.13 (m, 2H) 3.34-3.37 (m, 1H) 3.40-3.44 (m, 1H) 3.56 (s, 6H) 3.60-3.69 (m, 1H) 4.06-4.21 (m, 1H) 4.39-4.52 (m, 2H) 5.77-5.83 (m, 1H) 6.96-7.23 (m, 3H) 7.43-7.57 (m, 3H) 7.60-7.83 (m, 8H) 11.84 (br. s., 1H) 12.22 (br. s., 1H)

### Example 68

### {(2S,5S)-2-[6-(6-{2-[(S)-1-((8)-2-Methoxycarbonylamino-3-methyl-butyryl)-pyrrolidin-2-yl]-3H-imidazol-4-yl}-naphthalen-2-yl)-1H-benzoimidazol-2-yl]-4-oxo-1,2,4,5,6,7-hexahydro-azepino[3,2,1-hi]indol-5-yl}-carbamic acid methyl ester

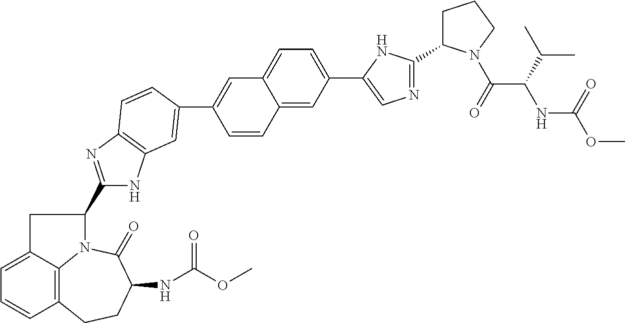

Methyl (2S,5S)-2-(6-bromo-1H-benzo[d]imidazol-2-yl)-4-oxo-1,2,4,5,6,7-hexahydroazepino[3,2,1-hi]indol-5-yl-carbamate (Intermediate 33) (45 mg, 98.8 μmol, Eq: 1.00), methyl (S)-3-methyl-1-oxo-1-((S)-2-(5-(6-(4,4,5,5-tetramethyl-1,3,2-dioxaborolan-2-yl)naphthalen-2-yl)-1H-imidazol-2-yl)pyrrolidin-1-yl)butan-2-yl-carbamate (prepared according to US 2010/0316607A1, 54.0 mg, 98.8 μmol, Eq: 1.) and sodium bicarbonate (25.2 mg, 297 mmol, Eq: 3) were combined with toluene (1.5 ml), ethanol (1.00 ml) and water (500 μA) to give a light yellow suspension, Tetrakis(triphenylphosphine)palladium (0) (11.4 mg, 9.88 μmol, Eq: 0.1) was added while the reaction mixture was degassed with nitrogen, The reaction mixture was stirred overnight at 90° C. The reaction mixture was poured into water and extracted with EtOAc. The combined organic layers were dried over Na₂SO₄ and concentrated in vacuo. The crude material was purified by flash chromatography (silica gel, 2% to 3% MeOH/EtOAc). 6-(6-{2-[(S)-1-((S)-2-Methoxycarbonylamino-3-methyl-butyryl)-pyrrolidin-2-yl]-1H-imidazol-4-yl}-naphthalen-2-yl)-2-((2S,5S)-5-methoxycarbonylamino-4-oxo-1,2,4,5,6,7-hexahydro-azepino[3,2,1-hi]indol-2-yl)-benzoimidazole-1-carboxylic acid methyl ester was obtained as a white solid (22 mgs, 28%). MS: 795⁺ (M+1). ¹H NMR (400 MHz, DMSO-d₆) δ ppm 12.22-12.30 (m, 1H) 11.79 (br. s., 1H) 8.21 (d, 1H) 8.10 (s, 1H) 7.74-8.03 (m, 5H) 7.53-7.65 (m, 2H) 7.46 (d, 1H) 7.26 (d, 1H) 7.19 (d, 1H) 7.12 (d, 1H) 7.04 (t, 1H) 5.95 (d, 1H) 5.11 (d, 1H) 4.18 (t, 1H) 4.04-4.13 (m, 1H) 3.83 (br. s., 2H) 3.71 (dd, 2H) 3.56 (s, 3H) 3.55 (s, 3H) 3.34-3.45 (m, 1H) 3.11 (t, 2H) 1.85-2.29 (m, 7H) 0.95 (d, 3H) 0.87 (d, 3H).

### Example 69

### N,N′-[1,4-phenylenebis[1H-benzimidazole-6,2-diyl-(2S,5S)-1,2,4,5,6,7-hexahydro-4-oxoazepino[3,2,1-hi]indole-5,2-diyl]]biscarbamic acid C,C′ dimethyl ester

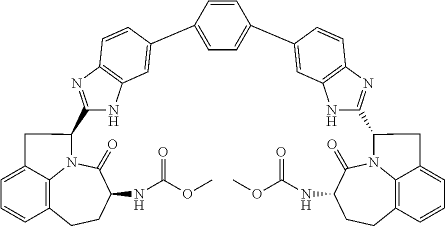

Methyl (2S,5S)-2-(6-bromo-1H-benzo[d]imidazol-2-yl)-4-oxo-1,2,4,5,6,7-hexahydroazepino[3,2,1-hi]indol-5-yl-carbamate (Intermediate 33) (50 mg, 110 μmol, Eq: 2.08), 1,4-benzenediboronic acid (9 mg, 52.7 μmol, Eq: 1.00) and sodium bicarbonate (26.5 mg, 316 mmol, Eq: 6) were mixed in a vial with toluene (1.5 ml), ethanol (1.00 ml), water (500 μA) to give a light yellow suspension. The mixture was degassed with nitrogen before tetrakis(triphenylphosphine)palladium (0) (6.09 mg, 5.27 μmol, Eq: 0.1) was added, the resulting suspension was heated at 90° C. and stirred overnight.

The reaction mixture was poured into water and extracted with EtOAc. The organic layers were dried over Na₂SO₄ and concentrated in vacuo. The crude material was purified by silica gel plug (silica gel, 5% MeOH in EtOAc). The early fractions was re-purified twice by preparative TLC plate (90% EtOAc/Hex) to give a light yellow solid as compound 1 (3 mgs, 11%). MS: 453 (M+H)'. The more polar portion was re-purified twice by preparative TLC plate (6% MeOH/DCM) to give a off-white solid as compound 2 (6 mgs, 12%). MS: 827⁺ (M+1). ¹H NMR (300 MHz, CHLOROFORM-d) δ ppm 10.40 (br. s, 2H) 7.35-8.11 (m, 10H) 7.23-7.30 (m, 2H) 6.98-7.13 (m, 4H) 5.91-6.18 (m, 4H) 4.22-4.51 (m, 4H) 3.75 (s, 6H) 3.58 (dd, 2H) 3.28 (br. s., 2H) 2.88-3.09 (m, 2H) 2.24-2.53 (m, 2H) 1.91-2.14 (m, 2H).

### Example 70

### {(2S,5S)-2-[5-(4′-{2-[(S)-4,4-Difluoro-1-((S)-2-Methoxycarbonylamino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester

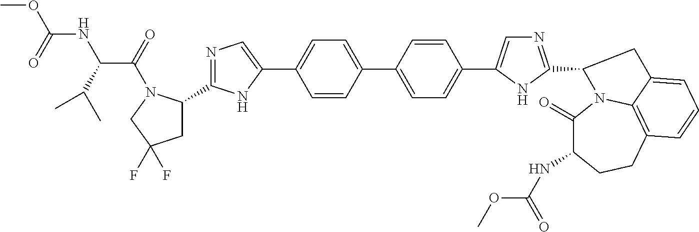

In a 10 mL seal tube, methyl (S)-1-((S)-4,4-difluoro-2-(5-(4-(4,4,5,5-tetramethyl-1,3,2-dioxaborolan-2-yl)phenyl)-1H-imidazol-2-yl)pyrrolidin-1-yl)-3-methyl-1-oxobutan-2-yl-carbamate (79.1 mg, 149 μmol, Eq: 1.3) (intermediate 34), methyl (2S,5S)-2-(5-(4-bromophenyl)-1H-imidazol-2-yl)-4-oxo-1,2,4,5,6,7-hexahydroazepino[3,2,1-hi]indol-5-yl-carbamate (55 mg, 114 μmol, Eq: 1.00) and sodium bicarbonate (0.4 ml, saturated solution, excess) were combined with tert-butanol (2 ml) to give a light brown solution and degassed for 5 min. PdCl₂(DPPF) (8.36 mg, 11.4 μmol, Eq: 0.1) was added and flushed with nitrogen and sealed heating at 90° C. for 5 hr. LC-MS shows the 100% conversion and the reaction is clean. It was diluted with EtOAc (30 ml), washed with H₂O, dried with MgSO₄ and concentrated, purified on column. CH₂Cl₂, 2%, 3%, 5% MeOH/CH₂Cl₂ to afford {(2S,5S)-2-[5-(4′-}2-[(S)-4,4-Difluoro-1-((S)-2-Methoxycarbonylamino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester as an light yellow solid. (50 mg, 51%). ESI-LRMS m/e calcd for C₄₃H₄₄F₂N₈O₆, [M⁺] 806, found 807 [M+H⁺]. ¹H NMR (400 MHz, DMSO-d₆) d ppm 0.72-0.96 (m, 14H) 1.75-2.35 (m, 6H) 3.08 (d, J=5.52 Hz, 9H) 3.47-3.71 (m, 15H) 3.93 (s, 3H) 4.16 (d, J=8.28 Hz, 4H) 4.41-4.74 (m, 2H) 5.29 (t, J=7.53 Hz, 2H) 5.66-5.93 (m, 3H) 6.95-7.25 (m, 7H) 7.38-7.95 (m, 24H) 11.68-12.14 (m, 3H)

### BIOLOGICAL EXAMPLES

Determination of compounds HCV GT1b inhibitory replicon activity using the replicon luciferase reporter assay

The 2209-23 cell line was developed at Roche by stable transfection of the hepatoma cell line Huh-7 with a GT-1b Con1 subgenomic bicistronic replicon as previously described. Subgenomic replicon cell line was established in cured Huh7 cells, obtained from R. Bartenschlager (J. Virol. 2003 March; 77 (5):3007-19) The GT-1a H77 subgenomic replicon vector pRLuc H771b 75 S/I, was created by replacing the non structural region of the GT-1b Con1 subgenomic replicon by the one of the H77 strain, except for the first 75 amino acids of the NS3 protein that are from GT-1b Con1 strain. (J. Virol. 2001 77:5352-59) The GT-1a pRLuc H771b 75 S/I subgenomic replicon cell line was established in cured Huh7 cells, obtained from R. Bartenschlager. (J. Virol. 2003 March; 77 (5):3007-19)

All the subgenomic replicon cell lines were cultured in Dulbecco's Modified Eagle Medium (DMEM-Glutamax™-I; Invitrogen Cat #10569-010). The medium was supplemented with 10% Fetal Bovine Serum (Invitrogen Cat #10082-147), 1% penicillin/streptomycin (Mediatech Cat #30-002-CI) and 500 μg/ml of G418 (Mediatech Cat #30-234-CI). Cells were maintained at 37° C. in a humidified 5% CO₂ atmosphere.

2209-23 cells were plated at a cell density of 5000 cells per well in 96 well plates (Becton Dickinson, Cat #35 3296). Cells were plated in 90 μl of Dulbecco's Modified Eagle Medium (DMEM-Glutamax™-I), (Invitrogen Cat #10569-010) medium was supplemented with 5% Fetal Bovine Serum (Invitrogen Cat #10082-147), 1% penicillin/streptomycin (Mediatech Cat #30-002-CI). The pRluc H771b 75 S/I cells were plated in 96-well plate at 3000 cells/well in DMEM-Glutamax™-I containing 5% FBS and 1% penicillin/streptomycin in 90 μl final volume. Cells were allowed to equilibrate for 24 hours at 37° C. and 5% CO₂ at which time compounds were added. Compounds (or medium as a control) were added 24 hours post-plating in 3 fold dilutions at a final DMSO concentration of 1% in 10 ul volume. Renilla luciferase reporter signal was read 72 hours after addition of compounds using the Renilla Luciferase Assay System (Promega, cat #E2820). EC50 values were defined as the compound concentration at which a 50% reduction in the levels of renilla luciferase reporter was observed as compared to control samples in the absence of compound and was determined by non-linear fitting of compound dose-response data. The EC50 was approximated if maximum percentage inhibition was less than 90% and more than 70%.

Determination of compounds cytotoxicity using the HCV GT1b replicon cell line measuring WST1.

2209-23 cells were plated at a cell density of 5000 cells per well in clear flat-bottom 96 well plate (Becton Dickinson, Cat #35 3075) for cell viability studies. The WST-1 cell proliferation assay (Roche Diagnostic, Cat#11644807001) was used to determine cell viability. Assay plates were set up in the same format as in the replicon assay. After 3 days of compound incubation 10 μl of WST-1 reagent was added to each well for 2 hours at 37° C. and 5% CO₂, following manufacturer's instructions. Absorption reading at 450 nm (reference filter at 650 nm) was determined using MRX Revelation microtiter plate reader (Lab System). CC₅₀ values were defined as the compound concentration required for reducing cell viability by 50% as compared to the untreated control in absence of compound and was determined by non-linear fitting of compound dose-response data. Representative assay data can be found in Table II below:

TABLE II

| Compound | GT-1a (IC₅₀) | GT-1b (IC₅₀) | GT-1b - EC₅₀ |
|---|---|---|---|
| # | (nM) | (nM) | (nM) |
| I-1 | 6.328 | | |
| I-2 | 18.495 | | |
| I-3 | 660 | | |
| I-4 | 4.112 | | |
| I-5 | 49.84 | 0.093 | |
| I-6 | 17.545 | 0.2 | |
| I-7 | 32.16 | | |
| I-8 | 2.54 | | |
| I-9 | 3.357 | 0.03 | |
| I-10 | 43.255 | 0.1 | |
| I-11 | 19.218 | 2.82 | |
| I-12 | 18.55 | | |
| I-13 | 41.75 | | |
| I-14 | nd | | |
| I-15 | 13.4 | | |
| I-16 | 29.2 | | |
| I-17 | 3.2 | | |
| I-18 | 21.6 | | |
| I-19 | 2.89 | 0.16 | |
| I-20 | 12 | | |
| I-21 | 0.395 | | |
| I-22 | 1.445 | | |
| I-23 | 0.767 | | |
| I-24 | 1.157 | | |
| I-25 | 31.25 | 1.21 | |
| I-26 | 7.715 | | |
| I-27 | 5.082 | | |
| I-28 | 1.814 | 0.01 | |
| I-29 | 1.025 | 0.01 | |
| I-30 | 1.004 | | |
| I-31 | 1.132 | 0.41 | |
| I-32 | 5.775 | | |
| I-33 | 46.1 | | |
| I-34 | 86.65 | | |
| I-35 | 5.92 | 0.01 | |
| I-36 | 13.81 | | |
| I-37 | 5.225 | | |
| I-38 | 22.4 | | |
| I-39 | 5.345 | | |
| I-40 | 7.85 | | |
| I-41 | 2.488 | | |
| I-42 | 1.168 | | |
| I-43 | 5.64 | | |
| I-44 | 11.4 | | |
| I-45 | 17.15 | | |
| I-46 | 2.965 | | |
| I-47 | 110.35 | | |
| I-48 | 2.835 | | |
| I-49 | 3.91 | | |
| I-50 | 6.625 | | |
| I-51 | 16.65 | 0.03 | |
| I-52 | 0.41 | 0.03 | |
| I-53 | 1.36 | | |
| I-54 | 0.949 | | |
| I-55 | 0.79 | 0.01 | |
| I-56 | 3 | | |
| I-57 | 17.1 | | |
| I-58 | 26.15 | | |
| I-59 | 0.709 | 0.07 | |
| I-60 | 51.4 | | |
| I-61 | 67.15 | | |
| I-62 | 130.05 | | |
| I-63 | 7.3 | | |
| I-64 | 9.87 | | |
| I-65 | 67.15 | | |
| I-66 | | | 0.036 |
| I-67 | | | 0.528 |
| I-68 | | | 0.062 |
| I-69 | | | |
| I-70 | | | |

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

All patents, patent applications and publications cited in this application are hereby incorporated by reference in their entirety for all purposes to the same extent as if each individual patent, patent application or publication were so individually denoted.

We claim:

1. A compound of formula I, II, or III 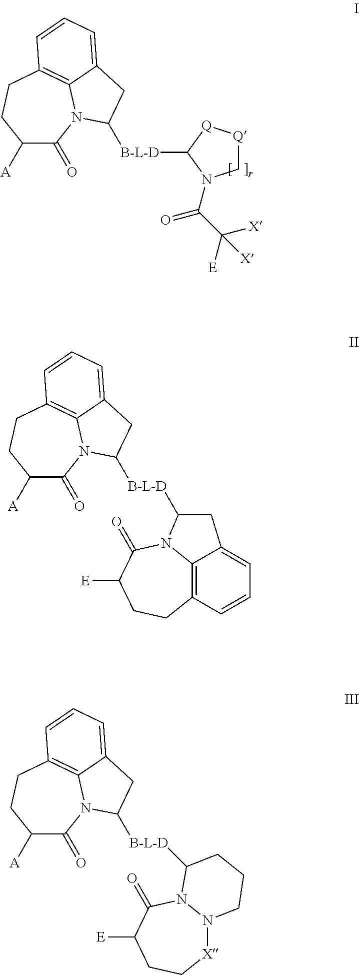
wherein:
each of A and E are independently selected from the group consisting of H, N(R)₂, NHC(═O)OR, NHCOR, and NHCONHR;
each R is independently H or lower alkyl;
B and D are independently selected from the group consisting of 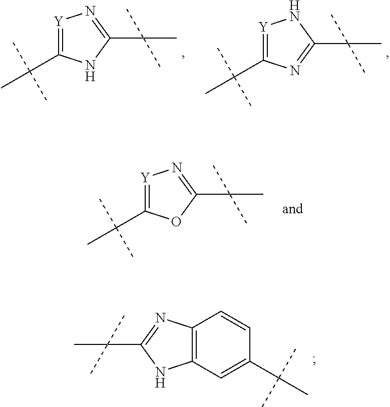
each Y is independently N or CY';
Y′ is H or halo;
L is selected from the group consisting of 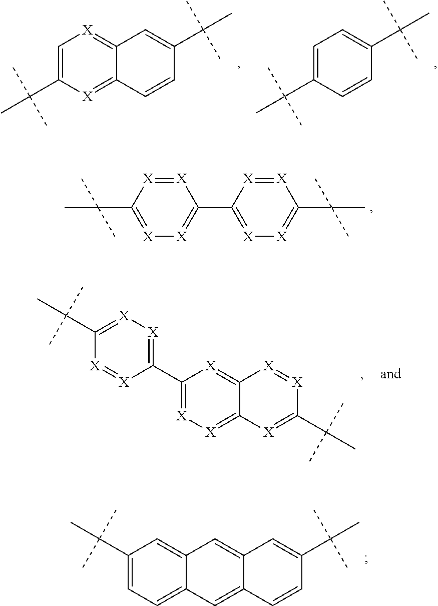
each X is independently, N, CH, C(F), C(OCF₃), or C(CN);
Q is CH₂;
Q′ is CH₂, CHOH, CHF, CH(CN), C(Q″)₂, O, or CF₂;
or Q and Q′ together form cycloalkyl;
both Q″ together form a spirocyclic heterocyclic ring;
r is 1 or 2;
each X′ is independently H, lower alkyl, lower alkoxy, hydroxy lower alkyl, heterocycloalkyl, lower alkyl sulfonyl lower alkyl, or (CH₂)ₙ-aryl;
or both X′ together form a spirocyclic heterocyclic ring;
n is 0, 1, or 2; and
X″ is CH₂ or C(═O);
or a pharmaceutically acceptable salt thereof.

2. The compound of claim 1, wherein B is 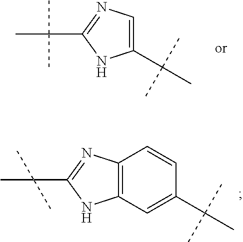
D is 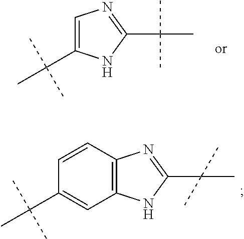
L is selected from a group consisting of

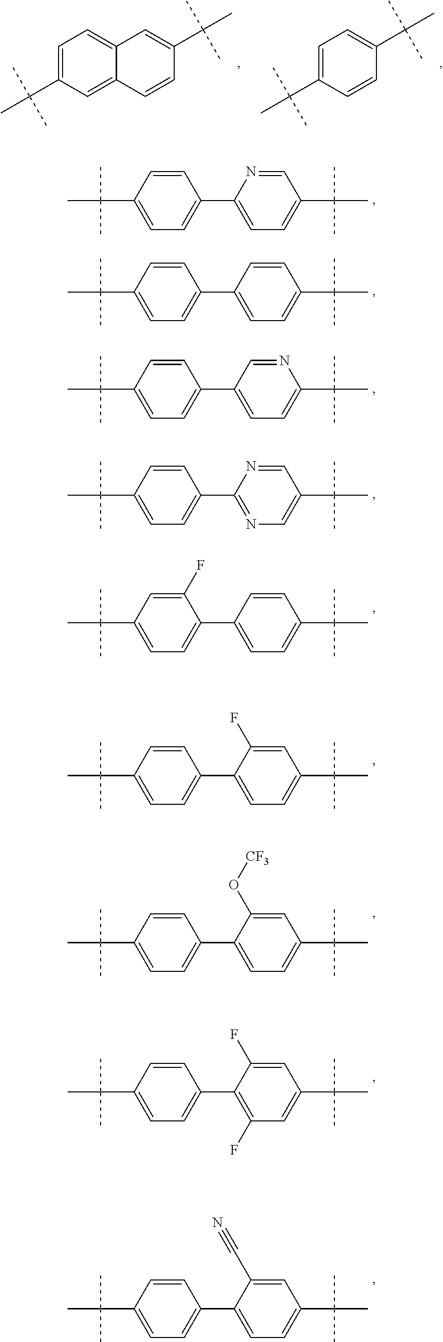

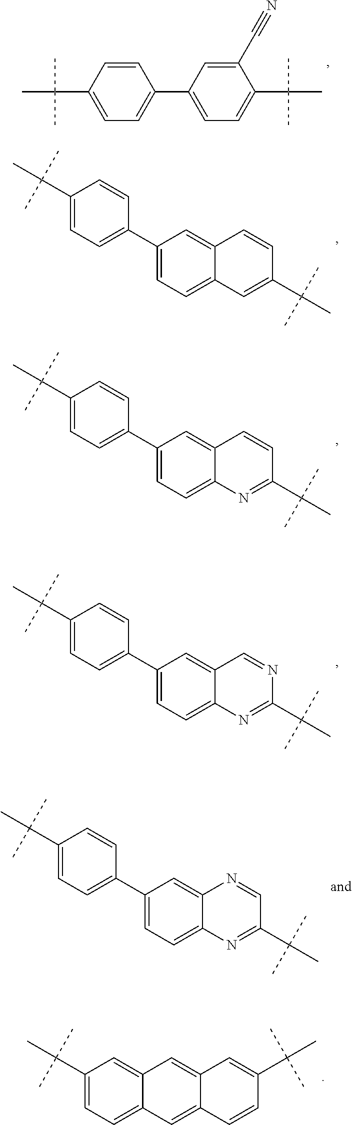

3. The compound of claim 2, wherein L is selected from a group consisting of

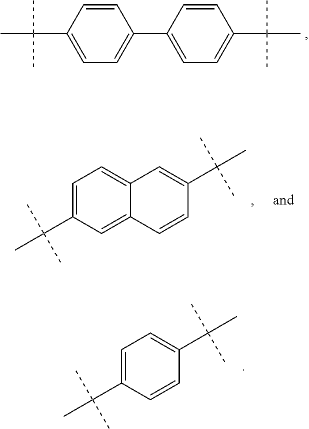

4. The compound of claim 3, wherein L is

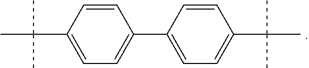

5. The compound according to claim 4, wherein B is 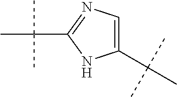
and D is

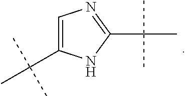

6. The compound of claim 5, wherein A is NHC(═O)OCH₃.

7. The compound of claim 6, wherein E is NHC(═O)OCH₃.

8. The compound of claim 3, wherein L is

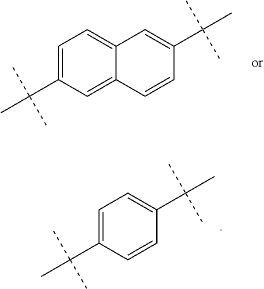

9. The compound of claim 8, wherein B is 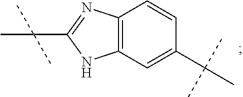
and
D is

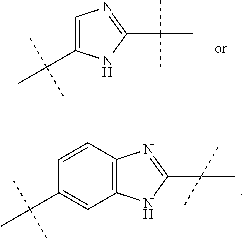

10. The compound of claim 9, wherein both A and E are NHC(═O)OCH₃.

11. The compound of claim 3, wherein B is 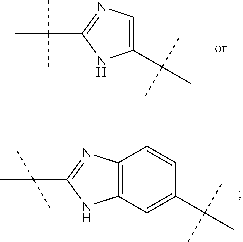
and
D is

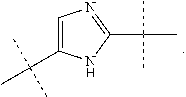

12. The compound of claim 11, wherein Q′ is CH₂, X′ is CH(CH₃)₂, and both A and E are NHC(═O)OCH₃.

13. The compound of claim 11, wherein Q′ is CF₂, X′ is CH(CH₃)₂, and both A and E are NHC(═O)OCH₃.

14. A compound selected from the group consisting of:
{(2S,5S)-2-[5-(4′-{2-[(2S,4S)-4-Fluoro-1-((S)-2-methoxycarbonylamino-3-methyl-butyryl)-pyrrolidin-2-yl]-3H-imidazol-4-yl}biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(4′-{2-[(2S,4R)-4-Fluoro-1-((S)-2-methoxycarbonylamino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(4-{2-[(S)-1-((S)-2-Methoxycarbonylamino-3-methyl-butyryl)-pyrrolidin-2-yl]-3H-benzoimidazol-5-yl}-phenyl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(4′-{2-[(S)-4,4-Difluoro-1-((S)-2-methoxycarbonylamino-3-methyl-butyryl)-pyrrolidin-2-yl]-3H-benzoimidazol-5-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[6-(4-{2-[(S)-1-((S)-2-Methoxycarbonylamino-3-methyl-butyryl)-pyrrolidin-2-yl]-3H-benzoimidazol-5-yl}-phenyl)-1H-benzoimidazol-2-yl]-4-oxo-1,2,4,5,6,7-hexahydro-azepino[3,2,1-hi]indol-5-yl}-carbamic acid methyl ester;
{(2S,5S)-2-[6-(2-{2-[(S)-1-((S)-2-Methoxycarbonylamino-3-methyl-butyryl)-pyrrolidin-2-yl]-3H-benzoimidazol-5-yl}-quinoxalin-6-yl)-1H-benzoimidazol-2-yl]-4-oxo-1,2,4,5,6,7-hexahydro-azepino[3,2,1-hi]indol-5-yl}-carbamic acid methyl ester;
((2S,5S)-2-{5-[4-(6-{2-[(S)-1-((S)-2-Methoxycarbonylamino-3-methyl-butyryl)-pyrrolidin-2-yl]-3H-benzoimidazol-5-yl}-pyridin-3-yl)-phenyl]-1H-imidazol-2-yl}-4-oxo-1,2,4,5,6,7-hexahydro-azepino[3,2,1-hi]indol-5-yl)-carbamic acid methyl ester;
{(2S,5S)-2-[5-(4′-{2-[(S)-7-((S)-2-Methoxycarbonylamino-3-methyl-butyryl)-1,4-dioxa-7-aza-spiro[4.4]non-8-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(4′-{2-[2-((S)-2-Methoxycarbonylamino-3-methyl-butyryl)-8-oxa-2-aza-spiro[4.5]dec-3-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[6-(4-{2-[(S)-1-((S)-2-Methoxycarbonylamino-3-methyl-butyryl)-pyrrolidin-2-yl]-3H-imidazol-4-yl}-phenyl)-1H-benzoimidazol-2-yl]-4-oxo-1,2,4,5,6,7-hexahydro-azepino[3,2,1-hi]indol-5-yl}-carbamic acid methyl ester;
{(2S,5S)-2-[6-(4′-{2-[(S)-1-((S)-2-Methoxycarbonylamino-3-methyl-butyryl)-pyrrolidin-2-yl]-3H-imidazol-4-yl}-biphenyl-4-yl)-1H-benzoimidazol-2-yl]-4-oxo-1,2,4,5,6,7-hexahydro-azepino[3,2,1-hi]indol-5-yl}-carbamic methyl ester;
((2S,5S)-2-{6-[6-(4-{2-[(S)-1-((S)-2-Methoxycarbonylamino-3-methyl-butyryl)-pyrrolidin-2-yl]-3H-imidazol-4-yl}-phenyl)-pyridin-3-yl]-1H-benzoimidazol-2-yl}-4-oxo-1,2,4,5,6,7-hexahydro-azepino[3,2,1-hi]indol-5-yl)-carbamic acid methyl ester;
((2S,5S)-2-{6-[6-(4-{2-[(S)-1-((S)-2-Methoxycarbonylamino-3-methyl-butyryl)-4,4-difluoro-pyrrolidin-2-yl]-3H-imidazol-4-yl}-phenyl)-pyridin-3-yl]-1H-benzoimidazol-2-yl}-4-oxo-1,2,4,5,6,7-hexahydro-azepino[3,2,1-hi]indol-5-yl)-carbamic acid methyl ester;
{(2S,5S)-2-[6-(6-{2-[(S)-1-((S)-2-Methoxycarbonylamino-3-methyl-butyryl)-pyrrolidin-2-yl]-1H-imidazol-4-yl}-quinoxalin-2-yl)-1H-benzoimidazol-2-yl]-4-oxo-1,2,4,5,6,7-hexahydro-azepino[3,2,1-hi]indol-5-yl}-carbamic acid methyl ester;
((2S,5S)-2-{5-[2-(4-{2-[(S)-1-((S)-2-Methoxycarbonylamino-3-methyl-butyryl)-pyrrolidin-2-yl]-3H-imidazol-4-yl}-phenyl)-pyrimidin-5-yl]-1H-imidazol-2-yl}-4-oxo-1,2,4,5,6,7-hexahydro-azepino[3,2,1-hi]indol-5-yl)-carbamic acid methyl ester;
((2S,5S)-2-{5-[2-(4-{2-[(S)-1-((S)-2-Methoxycarbonylamino-3-methyl-butyryl)-4,4-difluoro-pyrrolidin-2-yl]-3H-imidazol-4-yl}-phenyl)-pyrimidin-5-yl]-1H-imidazol-2-yl}-4-oxo-1,2,4,5,6,7-hexahydro-azepino[3,2,1-hi]indol-5-yl)-carbamic acid methyl ester;
((2S,5S)-2-{5-[2-(6-{2-[(S)-1-((S)-2-Methoxycarbonylamino-3-methyl-butyryl)-pyrrolidin-2-yl]-3H-imidazol-4-yl}-naphthalen-2-yl)-pyrimidin-5-yl]-1H-imidazol-2-yl}-4-oxo-1,2,4,5,6,7-hexahydro-azepino[3,2,1-hi]indol-5-yl)-carbamic acid methyl ester;
((2S,5S)-2-{5-[7-(4-{2-[(S)-1-((S)-2-Methoxycarbonylamino-3-methyl-butyryl)-pyrrolidin-2-yl]-3H-imidazol-4-yl}-phenyl)-quinolin-3-yl]-1H-imidazol-2-yl}-4-oxo-1,2,4,5,6,7-hexahydro-azepino[3,2,1-hi]indol-5-yl)-carbamic acid methyl ester;
((2S,5S)-2-{5-[7-(4-{2-[(S)-1-((S)-2-Methoxycarbonylamino-3-methyl-butyryl)-4,4-difluoro-pyrrolidin-2-yl]-3H-imidazol-4-yl}-phenyl)-quinolin-3-yl]-1H-imidazol-2-yl}-4-oxo-1,2,4,5,6,7-hexahydro-azepino[3,2,1-hi]indol-5-yl)-carbamic acid methyl ester;
((2S,5S)-2-{5-[7-(4-{5-Chloro-2-[(S)-1-((S)-2-methoxycarbonylamino-3-methyl-butyryl)-pyrrolidin-2-yl]-3H-imidazol-4-yl}-phenyl)-quinolin-3-yl]-1H-imidazol-2-yl}-4-oxo-1,2,4,5,6,7-hexahydro-azepino[3,2,1-hi]indol-5-yl)-carbamic acid methyl ester;
((2S,5S)-2-{4-[4-(6-{2-[(S)-1-((S)-2-Methoxycarbonylamino-3-methyl-butyryl)-pyrrolidin-2-yl]-1H-imidazol-4-yl}-quinolin-2-yl)-phenyl]-1H-imidazol-2-yl}-4-oxo-1,2,4,5,6,7-hexahydro-azepino[3,2,1-hi]indol-5-yl)-carbamic acid methyl ester;
((2S,5S)-2-{5-[2-(4-{2-[(S)-1-((S)-2-Methoxycarbonylamino-3-methyl-butyryl)-pyrrolidin-2-yl]-1H-imidazol-4-yl}-phenyl)-quinolin-6-yl]-1H-imidazol-2-yl}-4-oxo-1,2,4,5,6,7-hexahydro-azepino[3,2,1-hi]indol-5-yl)-carbamic acid methyl ester;
((2S,5S)-2-{4-[6-(6-{2-[(S)-1-((S)-2-Methoxycarbonylamino-3-methyl-butyryl)pyrrolidin-2-yl]-3H-imidazol-4-yl}-naphthalen-2-yl)-pyridin-3-yl]-1 Himidazol-2-yl}-4-oxo-1,2,4,5,6,7-hexahydro-azepino[3,2,1-hi]indol-5-yl)-carbamic acid methyl ester;
((2S,5S)-2-{4-[6-(5-{2-[(S)-1-((S)-2-Methoxycarbonylamino-3-methyl-butyryl)-pyrrolidin-2-yl]-3H-imidazol-4-yl}-pyridin-2-yl)-naphthalen-2-yl]-1H-imidazol-2-yl}-4-oxo-1,2,4,5,6,7-hexahydro-azepino[3,2,1-hi]indol-5-yl)-carbamic acid methyl ester;
{(2S,5S)-9-Bromo-2-[5-(4′-{2-[(R)-1-((R)-2-methoxycarbonylamino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-9-Cyano-2-[5-(4′-{2-[(R)-1-((R)-2-methoxycarbonylamino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-9-Carbamoyl-2-[5-(4′-{2-[(R)-1-((R)-2-methoxycarbonylamino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(4′-{2-[(S)-1-((S)-2-Methoxycarbonylamino-3-methyl-butyryl)-pyrrolidin-2-yl]-3H-imidazol-4-yl}-biphenyl-4-yl)-1H-imidazol-2-yl]-4,7-dioxo-1,2,4,5,6,7-hexahydro-azepino[3,2,1-hi]indol-5-yl}-carbamic acid methyl ester;
{(2S,5S)-7-Hydroxy-2-[5-(4′-{2-[(S)-1-((S)-2-methoxycarbonylamino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(4′-{5-Chloro-2-[(S)-1-((S)-2-methoxycarbonyl amino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(4-(6-{2-[(S)-1-((S)-2-Methoxycarbonylamino-3-methyl-butyryl)-pyrrolidin-2-yl]-3H-imidazol-4-yl}-naphthalen-2-yl)-phenyl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(4′-{5-Chloro-2-[(S)-4,4-difluoro-1-((S)-2-methoxycarbonyl amino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(4′-{2-[(2S,4S)-4-Hydroxy-1-((S)-2-methoxycarbonylamino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
((2S,5S)-2-{5-[4-(5-{2-[(S)-1-((S)-2-Methoxycarbonylamino-3-methyl-butyryl)-pyrrolidin-2-yl]-3H-imidazol-4-yl}-pyridin-2-yl)-phenyl]-1H-imidazol-2-yl}-4-oxo-1,2,4,5,6,7-hexahydro-azepino[3,2,1-hi]indol-5-yl)-carbamic acid methyl ester;
{(2S,5S)-2-[5-(2-Fluoro-4′-{2-[(S)-1-((S)-2-methoxycarbonylamino-3-methyl-butyryl)-pyrrolidin-2-yl]-1H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(3-Fluoro-4′-{2-[(S)-1-((S)-2-methoxycarbonylamino-3-methyl-butyryl)-pyrrolidin-2-yl]-1H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(2′-Fluoro-4′-{2-[(S)-1-((S)-2-methoxycarbonylamino-3-methyl-butyryl)-pyrrolidin-2-yl]-1H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(2′-Cyano-4′-{2-[(S)-1-((S)-2-methoxycarbonylamino-3-methyl-butyryl)-pyrrolidin-2-yl]-1H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(4′-{2-[(2S,4R)-4-Cyano-1-((S)-2-methoxycarbonylamino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(4′-{2-[(2S,4S)-4-Cyano-1-((S)-2-methoxycarbonylamino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-{5-[4-(6-{2-[(2S,4S)-4-Cyano-1-((S)-2-methoxycarbonylamino-3-methyl-butyryl)-pyrrolidin-2-yl]-3H-imidazol-4-yl}-naphthalen-2-yl)-phenyl]-1H-imidazol-2-yl}-4-oxo-1,2,4,5,6,7-hexahydro-azepino[3,2,1-hi]indol-5-yl}-carbamic acid methyl ester;
{(2S,5S)-2-{5-[4-(6-{2-[(S)-4,4-Difluoro-1-((S)-2-methoxycarbonylamino-3-methyl-butyryl)-pyrrolidin-2-yl]-3H-imidazol-4-yl}-naphthalen-2-yl)-phenyl]-1H-imidazol-2-yl}-4-oxo-1,2,4,5,6,7-hexahydro-azepino[3,2,1-hi]indol-5-yl}-carbamic acid methyl ester;
{(2S,5S)-2-[5-(4-{2-[(S)-1-((S)-3-Methoxy-2-methoxycarbonylamino-propionyl)-pyrrolidin-2-yl]-3H-imidizol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
5S)-2-[5-(4-{2-[(S)-1-((S)-2-Cyclopropyl-2-methoxycarbonylamino-acetyl)-pyrrolidin-2-yl]-3H-imidizol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(4-{2-[(S)-1-((S)-2-Methoxycarbonylamino-butyryl)-pyrrolidin-2-yl]-3H-imidizol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(4-{2-[(S)-1-((S)-4-Methoxy-2-Methoxycarbonylamino-butyryl)-pyrrolidin-2-yl]-3H-imidizol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(4-{2-[(S)-1-(3-Methoxycarbonylamino-oxetane-3-carbonyl)-pyrrolidin-2-yl]-3H-imidizol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
((2S,5S)-2-{5-[4-(2-{(S)-1-[2-Methoxycarbonylamino-2-(tetrahydro-pyran-4-yl)-pyrrolidin-2-yl]-3H-imidizol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(4-{2-[(S)-1-((S)-2-Methoxycarbonylamino-propionyl)-pyrrolidin-2-yl]-3H-imidizol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(4-{2-[(S)-1-((S)-3-Hydroxy-2-methoxycarbonylamino-butyl)-pyrrolidin-2-yl]-3H-imidizol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(4-{2-[(S)-1-((S)-4-Methanesulfonyl-2-methoxycarbonylamino-butyl)-pyrrolidin-2-yl]-3H-imidizol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
((2S,5S)-2-{5-[4-(6-{2-[(S)-1-((S)-2-Methoxycarbonylamino-3-methyl-butyryl)-pyrrolidin-2-yl]-3H-imidazol-4-yl}-quinoxalin-2-yl)-phenyl]-1H-imidazol-2-yl}-4-oxo-1,2,4,5,6,7-hexahydro-azepino[3,2,1-hi]indol-5-yl)-carbamic acid methyl ester;
((2S,5S)-2-{5-[4-(6-{2-[(S)-1-((2S,3S)-2-Methoxycarbonylamino-3-methyl-pentanoyl)-pyrrolidin-2-yl]-3H-imidazol-4-yl}-quinoxalin-2-yl)-phenyl]-1H-imidazol-2-yl}-4-oxo-1,2,4,5,6,7-hexahydroazepino[3,2,1-hi]indol-5-yl)-carbamic acid methyl ester;
((2S,5S)-2-{5-[4-(6-{2-[(S)-1-((S)-2-Methoxycarbonylamino-3,3-dimethyl-butyryl)-pyrrolidin-2-yl]-3H-imidazol-4-yl}-quinoxalin-2-yl)-phenyl]-1H-imidazol-2-yl}-4-oxo-1,2,4,5,6,7-hexahydro-azepino[3,2,1-hi]indol-5-yl)-carbamic acid methyl ester;
((2S,5S)-2-{5-[4-(6-{2-[(S)-1-((R)-2-Methoxycarbonylamino-2-phenyl-acetyl)-pyrrolidin-2-yl]-3H-imidazol-4-yl}-quinoxalin-2-yl)-phenyl]-1H-imidazol-2-yl}-4-oxo-1,2,4,5,6,7-hexahydro-azepino[3,2,1-hi]indol-5-yl)-carbamic acid methyl ester;
((2S,5S)-2-{5-[2-(4-{2-[(S)-1-((2S,3S)-2-Methoxycarbonylamino-3 methyl-pentanoyl)-pyrrolidin-2-yl]-3H-imidazol-4-yl}-phenyl) quinoxalin-6-yl]-1H-imidazol-2-yl}-4-oxo-1,2,4,5,6,7-hexahydro-azepino[3,2,1-hi]indol-5-yl)-carbamic acid methyl ester;
((2S,5S)-2-{4-[4-(5-{2-[(S)-4,4-Difluoro-1-(2-methoxycarbonylamino-3-methyl-butyryl)-pyrrolidin-2-yl]-3H-imidazol-4-yl}-pyrimidin-2-yl)-phenyl]-1H-imidazol-2-yl}-4-oxo-1,2,4,5,6,7-hexahydro-azepino[3,2,1-hi]indol-5-yl)-carbamic acid methyl ester;
((1S,2S)-1-{(S)-4,4-Difluoro-2-[5-(2-{4-[2-((2S,5S)-5-methoxycarbonylamino-4-oxo-1,2,4,5,6,7-hexahydro-azepino[3,2,1-hi]indol-2-yl)-1H-imidazol-4-yl]-phenyl}-pyrimidin-5-yl)-1H-imidazol-2-yl]-pyrrolidine-1-carbonyl}-2-methyl-butyl)-carbamic acid methyl ester;
((2S,5S)-2-{5-[2-(4-{2-[(S)-1-((S)-2-Methoxycarbonylamino-3-methyl-butyryl)-pyrrolidin-2-yl]-3H-imidazol-4-yl}-phenyl)-quinoxalin-6-yl]-1H-imidazol-2-yl}-4-oxo-1,2,4,5,6,7-hexahydro-azepino[3,2,1-hi]indol-5-yl)-carbamic acid methyl ester;
{(2S,5S)-2-[5-(4′-{5-[(S)-1-((R)-2-Methoxycarbonylamino-2-phenyl-acetyl)-pyrrolidin-2-yl]-4H-[1,2,4]triazol-3-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(4′-{5-[(S)-1-((S)-2-Methoxycarbonylamino-3-methyl-butyryl)-pyrrolidin-2-yl]-4H-[1,2,4]triazol-3-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(4′-{5-[(S)-1-((S)-2-Methoxycarbonylamino-3-methyl-butyryl)-pyrrolidin-2-yl]-1H-imidazol-2-yl}-biphenyl-4-yl)-[1,3,4]oxadiazol-2-yl]-4-oxo-1,2,4,5,6,7-hexahydro-azepino[3,2,1-hi]indol-5-yl}-carbamic acid methyl ester;
{(2S,5S)-2-[5-(4′-{2-[(R)-4-((R)-2-Methoxycarbonylamino-2-phenyl-acetyl)-morpholin-3-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(4′-{2-[(R)-4-((S)-2-Methoxycarbonylamino-3-methyl-butyryl)-morpholin-3-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(4′-{2-[(S)-3-((R)-2-Methoxycarbonylamino-2-phenyl-acetyl)-3-aza-bicyclo [3.1.0]hex-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(4′-{2-[(S)-1-((S)-2-Methoxycarbonylamino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
{(2S,5S)-2-[5-(4′-{2-[(1S,9S)-9-methoxycarbonylamino-6,10-dioxo-octahydro-pyridazino[1,2-a][1,2] diazepin-1-yl])-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester;
6-(6-{2-[(S)-1-((S)-2-Methoxycarbonylamino-3-methyl-butyryl)-pyrrolidin-2-yl]-1H-imidazol-4-yl}-naphthalen-2-yl)-2-42S,5S)-5-methoxycarbonylamino-4-oxo-1,2,4,5,6,7-hexahydro-azepino[3,2,1-hi]indol-2-yl)-benzoimidazole-1-carboxylic acid methyl ester;
N,N′-[1,4-phenylenebis[1H-benzimidazole-6,2-diyl-(2S,5S)-1,2,4,5,6,7-hexahydro-4-oxoazepino[3,2,1-hi]indole-5,2-diyl]]biscarbamic acid C,C′dimethyl ester; and
{(2S,5S)-2-[5-(4′-{2-[(S)-4,4-Difluoro-1-((S)-2-Methoxycarbonylamino-3-methyl-butyryl)-pyrrolidin-2-yl]-3H-imidazol-4-yl}-biphenyl-4-yl)-1H-imidazol-2-yl]-4-oxo-1,2,4,5,6,7-hexahydro-azepino[3,2,1-hi]indol-5-yl}-carbamic acid methyl ester.

15. A method for treating a Hepatitis C Virus (HCV) infection comprising administering to a patient in need thereof a therapeutically effective amount of a compound of claim 1.

16. The method of claim 15 further comprising administering an immune system modulator or an antiviral agent that inhibits replication of HCV, or a combination thereof.

17. The method of claim 16, wherein the immune system modulator is an interferon or chemically derivatized interferon.

18. The method of claim 16, wherein the antiviral agent is selected from the group consisting of a HCV protease inhibitor, a HCV polymerase inhibitor, a HCV helicase inhibitor, a HCV primase inhibitor, a HCV fusion inhibitor, and a combination thereof.

19. A method for inhibiting replication of HCV in a cell comprising administering a compound of claim 1.

20. A composition comprising a compound of claim 1 and a pharmaceutically acceptable excipient.
